(19) United States
(12) Patent Application Publication
(10) Pub. No.: US 2022/0384735 A1
(43) Pub. Date: Dec. 1, 2022

(54) PERI-CONDENSED HETEROCYCLIC COMPOUNDS AS MATERIALS FOR ELECTRONIC DEVICES

(71) Applicant: Merck Patent GmbH, Darmstadt (DE)

(72) Inventors: Amir Hossain PARHAM, Frankfurt am Main (DE); Christian EHRENREICH, Darmstadt (DE); Jens ENGELHART, Darmstadt (DE)

(21) Appl. No.: 17/761,313

(22) PCT Filed: Sep. 17, 2020
(86) PCT No.: PCT/EP2020/075926

§ 371 Date: Mar. 17, 2022

(30) Foreign Application Priority Data
Sep. 20, 2019 (EP) 19198695.9

(51) Int. Cl. H01L 51/00; C07D 471/06; C09K 11/06; C07D 471/16; C07D 471/22; C07D 491/16; C07D 495/16

(52) U.S. Cl. CPC H01L 51/0072; C07D 471/06; C09K 11/06; C07D 471/16; C07D 471/22; C07D 491/16; H01L 51/0067; C07D 495/16; H01L 51/0071; H01L 51/0073; H01L 51/0054; H01L 51/5076

(57) ABSTRACT

The present application relates to compounds of a formula (I), to processes for preparing the compounds, and to electronic devices comprising one or more of the compounds.

The present invention relates to materials for use in electronic devices and to electronic devices comprising these materials.

Electronic devices in the context of this application are understood to mean what are called organic electronic devices, which comprise organic semiconductor materials as functional materials. More particularly, these are understood to mean OLEDs (organic electroluminescent devices). The term OLEDs is understood to mean electronic devices which have one or more layers comprising organic compounds and emit light on application of electrical voltage. The construction and general principle of function of OLEDs are known to those skilled in the art.

Emitting materials used in OLEDs are frequently phosphorescent organometallic complexes. In general terms, there is still a need for improvement in OLEDs, especially also in OLEDs which exhibit triplet emission (phosphorescence), for example with regard to efficiency, operating voltage and lifetime. The properties of phosphorescent OLEDs are not just determined by the triplet emitters used. More particularly, the other materials used, such as matrix materials, are also of particular significance here. Improvements to these materials can thus also lead to improvements in the OLED properties. An example of a known class of materials that are used as matrix materials for triplet emitters in OLEDs is that of aromatic lactams.

It is an object of the present invention to provide compounds which are suitable for use in an OLED, especially as matrix material for phosphorescent emitters or as electron transport material, and which lead to improved properties therein.

It has been found that, surprisingly, this object is achieved by particular compounds described in detail hereinafter that are of good suitability for use in OLEDs. These OLEDs especially have a long lifetime, high efficiency and low operating voltage. The present invention therefore provides these compounds and electronic devices, especially organic electroluminescent devices, comprising these compounds.

The present application thus provides a compound of a formula (I)

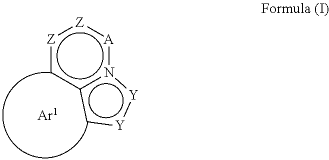

where the variables that occur are as follows:

A is C═O,C═S,C═NR⁰,P(═O)R⁰,SO or SO₂;

Y is the same or different at each instance and is selected from N and CR¹,

Ar¹ is an aromatic ring system which has 6 to 40 aromatic ring atoms and is substituted by R² radicals, and which is fused onto the rest of the formula (I) via the three carbon atoms shown in formula (I), or a heteroaromatic ring system which has 5 to 40 aromatic ring atoms and is substituted by R² radicals, and which is fused onto the rest of the formula (I) via the three carbon atoms shown in formula (I);

Z is the same or different at each instance and is selected from CR⁴ and N, or the Z—Z unit represents a unit of formula (Ar²)

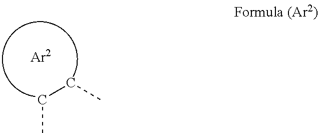

where Ar² is selected from aromatic ring systems which have 6 to 40 aromatic ring atoms and are substituted by R³ radicals, and which include the C—C unit, and heteroaromatic ring systems which have 5 to 40 aromatic ring atoms and are substituted by R³ radicals, and which include the C—C unit, and where the dotted lines are the bonds of the Z—Z unit to the rest of the formula;

R⁰ is the same or different at each instance and is selected from straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;

R¹ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —O(═O)O—, —C(═O)NR⁵—, NR⁵, P(═O)(R⁵), —O—, —S—, SO or SO₂;

R² is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more radicals selected from R², R³ and R⁴ may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;

R³ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more radicals selected from R², R³ and R⁴ may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —O(═O)O—, —C(═O)NR⁵—, NR⁵, P(═O)(R⁵), —O—, —S—, SO or SO₂;

R⁴ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R², R³ and R⁴ radicals may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;

R⁵ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁵ radicals may be joined to one another and may form a ring; where the alkyl, alkoxy, alkenyl and alkynyl groups mentioned and the aromatic ring systems and heteroaromatic ring systems mentioned are each substituted by R⁶ radicals; and where one or more CH₂ groups in the alkyl, alkoxy, alkenyl and alkynyl groups mentioned may be replaced by —R⁶C═CR⁶—, —C↓C—, Si(R⁶)₂, C═O, C═NR⁶, —O(═O)O—, —C(═O)NR⁶—, NR⁶, P(═O)(R⁶), —O—, —S—, SO or SO₂;

R⁶ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁶ radicals may be joined to one another and may form a ring; and where the alkyl, alkoxy, alkenyl and alkynyl groups, aromatic ring systems and heteroaromatic ring systems mentioned may be substituted by one or more radicals selected from F and CN;

where, when the two Y groups in formula (I) are CR¹, either
a) at least one group selected from the R¹, R², R³ and R⁴ groups is selected from aromatic ring systems which have 7 to 40 aromatic ring atoms and are each substituted by R⁵ radicals; and heteroaromatic ring systems which have 5 to 40 aromatic ring atoms and are each substituted by R⁵ radicals; or
b) at least two groups selected from the R¹, R², R³ and R⁴ groups are selected from aromatic ring systems which have 6 to 40 aromatic ring atoms and are each substituted by R⁵ radicals; and heteroaromatic ring systems which have 5 to 40 aromatic ring atoms and are each substituted by R⁵ radicals.

The C—C unit in formula (Ar²) is understood to mean two carbon atoms that are bonded directly to one another and are part of the aromatic or heteroaromatic ring.

The circles present within the rings in formula (I) and in the further generic formulae mean that the rings in question have aromaticity, and in the specific case are heteroaromatic. In a preferred embodiment, this includes existence of aromaticity only for a particular mesomeric structure, for example as shown for the embodiment of the formula (I) below in which A is C═O, Z is CR⁴, one Y is CR¹ and the other Y is N:

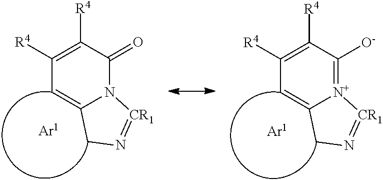

The definitions which follow are applicable to the chemical groups that are used in the present application. They are applicable unless any more specific definitions are given.

An aryl group in the context of this invention is understood to mean either a single aromatic cycle, i.e. benzene, or a fused aromatic polycycle, for example naphthalene, phenanthrene or anthracene. A fused aromatic polycycle in the context of the present application consists of two or more single aromatic cycles fused to one another. Fusion between cycles is understood here to mean that the cycles share at least one edge with one another. An aryl group in the context of this invention contains 6 to 40 aromatic ring atoms. In addition, an aryl group does not contain any heteroatom as aromatic ring atom, but only carbon atoms.

A heteroaryl group in the context of this invention is understood to mean either a single heteroaromatic cycle, for example pyridine, pyrimidine or thiophene, or a fused heteroaromatic polycycle, for example quinoline or carbazole. A fused heteroaromatic polycycle in the context of the present application consists of two or more single aromatic or heteroaromatic cycles that are fused to one another, where at least one of the aromatic and heteroaromatic cycles is a heteroaromatic cycle. Fusion between cycles is understood here to mean that the cycles share at least one edge with one another. A heteroaryl group in the context of this invention contains 5 to 40 aromatic ring atoms of which at least one is a heteroatom. The heteroatoms of the heteroaryl group are preferably selected from N, O and S.

An aryl or heteroaryl group, each of which may be substituted by the abovementioned radicals, is especially understood to mean groups derived from benzene, naphthalene, anthracene, phenanthrene, pyrene, dihydropyrene, chrysene, perylene, triphenylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, benzimidazolo[1,2-a]benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aromatic ring system in the context of this invention is a system which does not necessarily contain solely aryl groups, but which may additionally contain one or more nonaromatic rings fused to at least one aryl group. These nonaromatic rings contain exclusively carbon atoms as ring atoms. Examples of groups covered by this definition are tetrahydronaphthalene, fluorene and spirobifluorene. In addition, the term “aromatic ring system” includes systems that consist of two or more aromatic ring systems joined to one another via single bonds, for example biphenyl, terphenyl, 7-phenyl-2-fluorenyl, quaterphenyl and 3,5-diphenyl-1-phenyl. An aromatic ring system in the context of this invention contains 6 to 40 carbon atoms and no heteroatoms in the ring system. The definition of “aromatic ring system” does not include heteroaryl groups.

A heteroaromatic ring system conforms to the abovementioned definition of an aromatic ring system, except that it must contain at least one heteroatom as ring atom. As is the case for the aromatic ring system, the heteroaromatic ring system need not contain exclusively aryl groups and heteroaryl groups, but may additionally contain one or more nonaromatic rings fused to at least one aryl or heteroaryl group. The nonaromatic rings may contain exclusively carbon atoms as ring atoms, or they may additionally contain one or more heteroatoms, where the heteroatoms are preferably selected from N, O and S. One example of such a heteroaromatic ring system is benzopyranyl. In addition, the term “heteroaromatic ring system” is understood to mean systems that consist of two or more aromatic or heteroaromatic ring systems that are bonded to one another via single bonds, for example 4,6-diphenyl-2-triazinyl. A heteroaromatic ring system in the context of this invention contains 5 to 40 ring atoms selected from carbon and heteroatoms, where at least one of the ring atoms is a heteroatom. The heteroatoms of the heteroaromatic ring system are preferably selected from N, O and S.

The terms “heteroaromatic ring system” and “aromatic ring system” as defined in the present application thus differ from one another in that an aromatic ring system cannot have a heteroatom as ring atom, whereas a heteroaromatic ring system must have at least one heteroatom as ring atom. This heteroatom may be present as a ring atom of a nonaromatic heterocyclic ring or as a ring atom of an aromatic heterocyclic ring.

In accordance with the above definitions, any aryl group is covered by the term “aromatic ring system”, and any heteroaryl group is covered by the term “heteroaromatic ring system”.

An aromatic ring system having 6 to 40 aromatic ring atoms or a heteroaromatic ring system having 5 to 40 aromatic ring atoms is especially understood to mean groups derived from the groups mentioned above under aryl groups and heteroaryl groups, and from biphenyl, terphenyl, quaterphenyl, fluorene, spirobifluorene, dihydrophenanthrene, dihydropyrene, tetrahydropyrene, indenofluorene, truxene, isotruxene, spirotruxene, spiroisotruxene, indenocarbazole, or from combinations of these groups.

In the context of the present invention, a straight-chain alkyl group having 1 to 20 carbon atoms and a branched or cyclic alkyl group having 3 to 20 carbon atoms and an alkenyl or alkynyl group having 2 to 40 carbon atoms in which individual hydrogen atoms or CH₂ groups may also be substituted by the groups mentioned above in the definition of the radicals are preferably understood to mean the methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl radicals.

An alkoxy or thioalkyl group having 1 to 20 carbon atoms in which individual hydrogen atoms or CH₂ groups may also be substituted by the groups mentioned above in the definition of the radicals is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-trifluoroethoxy, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio.

The wording that two or more radicals together may form a ring, in the context of the present application, shall be understood to mean, inter alia, that the two radicals are joined to one another by a chemical bond. In addition, however, the abovementioned wording shall also be understood to mean that, if one of the two radicals is hydrogen, the second radical binds to the position to which the hydrogen atom was bonded, forming a ring.

Preferably, A is C═O or C═S, more preferably C═O.

Preferably, Ar¹ is an aromatic ring system which has 6 to 18 aromatic ring atoms and is substituted by R² radicals, and which is fused onto the rest of the formula (I) via the three carbon atoms shown in formula (I), or a heteroaromatic ring system which has 5 to 18 aromatic ring atoms and is substituted by R² radicals, and which is fused onto the rest of the formula (I) via the three carbon atoms shown in formula (I). More preferably, Ar¹ is selected from benzene, pyridine, pyrimidine, pyridazine, naphthalene, quinoline, quinazoline, phenanthrene, anthracene, triphenylene, fluorene, carbazole, dibenzofuran and dibenzothiophene, even more preferably benzene, pyridine, pyrimidine, carbazole, dibenzofuran and dibenzothiophene, most preferably benzene, in each case substituted by R² radicals and fused onto the rest of the formula (I) via the three carbon atoms shown in formula (I).

Preferably, the Z—Z unit is a unit of the formula (Ar²)

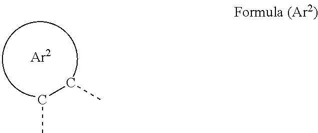

where Ar² is selected from aromatic ring systems which have 6 to 40 aromatic ring atoms and are substituted by R³ radicals, and which include the C—C unit, and heteroaromatic ring systems which have 5 to 40 aromatic ring atoms and are substituted by R³ radicals, and which include the C—C unit, and where the dotted lines are the bonds of the Z—Z unit to the rest of the formula.

Ar² is preferably selected from benzene, pyridine, pyrimidine, pyridazine, pyrazine, naphthalene, thiophene, furan, pyrrole, imidazole, thiazole, oxazole, benzothiophene, benzofuran, indole and indane, each of which is substituted by R³ radicals, and which include the C—C unit in formula (Ar²). More preferably, Ar² is selected from benzene, thiophene, furan, benzothiophene and benzofuran, even more preferably benzene, in each case substituted by R³ radicals, and including the C—C unit in formula (Ar²).

Preferably, the Y adjacent to the nitrogen atom in formula (I) is CR¹, and the other Y is N. In an alternative, likewise preferred embodiment, both Y groups are CR¹.

R¹ is preferably the same or different at each instance and is selected from H, D, F, CN, Si(R⁵)₃, N(R⁵)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁵ radicals; and where one or more CH₂ groups in the alkyl or alkoxy groups mentioned may be replaced by —C≡C—, —R⁵C═CR⁵—, Si(R⁵)₂, C═O, C═NR⁵, —NR⁵—, —O—, —S—, —C(═O)O— or —C(═O)NR⁵—. More preferably, R¹ is the same or different at each instance and is selected from H, D, F, CN, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms, where said alkyl groups, said aromatic ring systems and said heteroaromatic ring systems are each substituted by R⁵ radicals. Very preferably, R¹ is the same or different at each instance and is selected from H, N(R⁵)₂, aromatic ring systems having 6 to 40 aromatic ring atoms, and heteroaromatic ring systems having 5 to 40 aromatic ring atoms; where the aromatic ring systems mentioned and the heteroaromatic ring systems mentioned are each substituted by R⁵ radicals.

Preferred aromatic and heteroaromatic ring systems and N(R⁵)₂ radicals as R¹ groups are selected from the following groups:

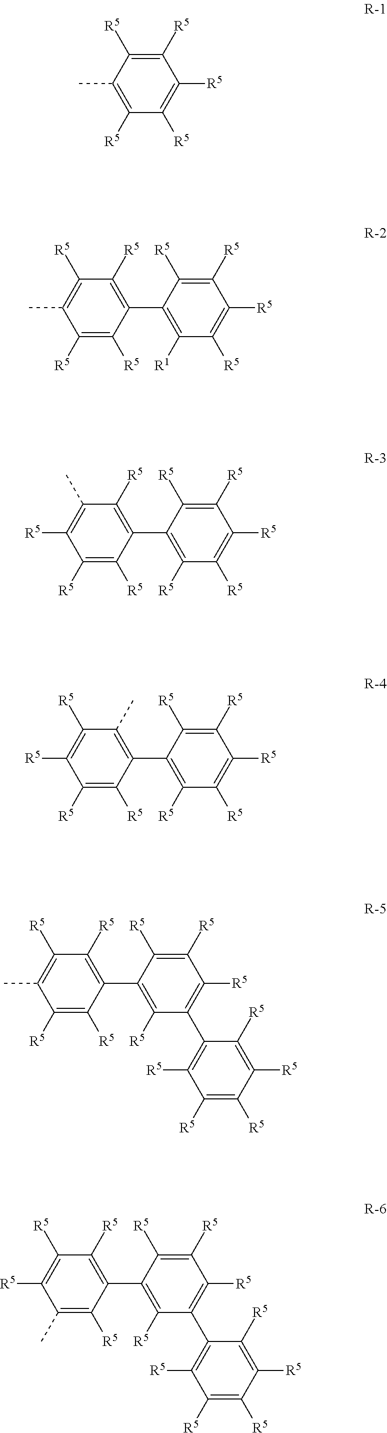

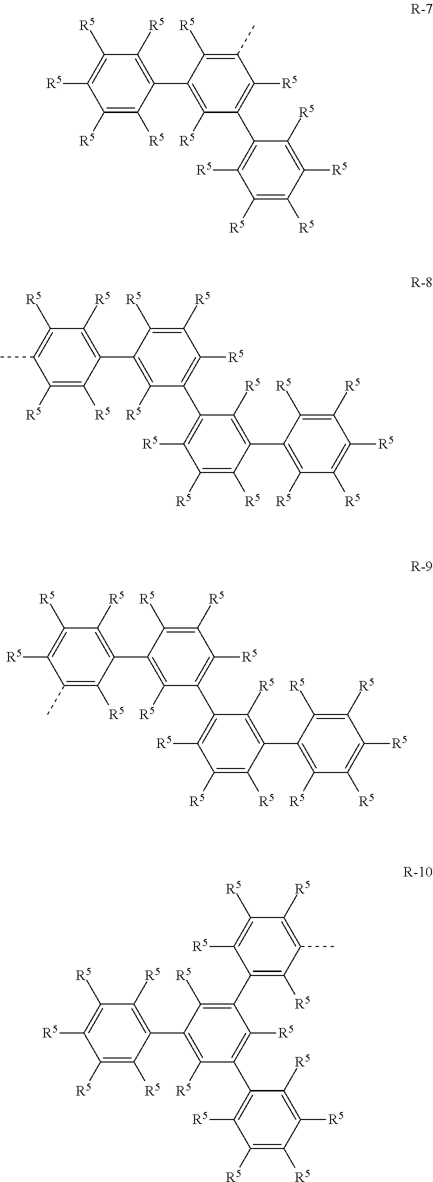

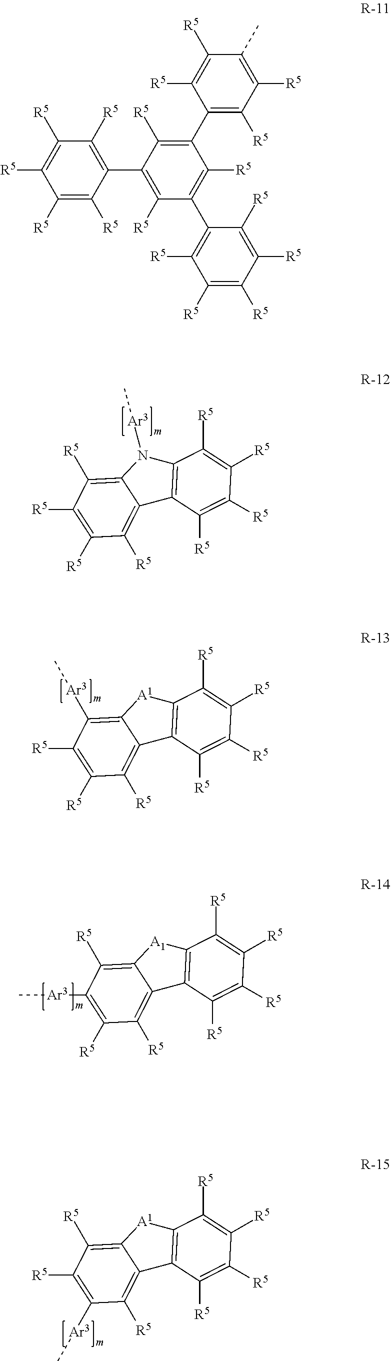

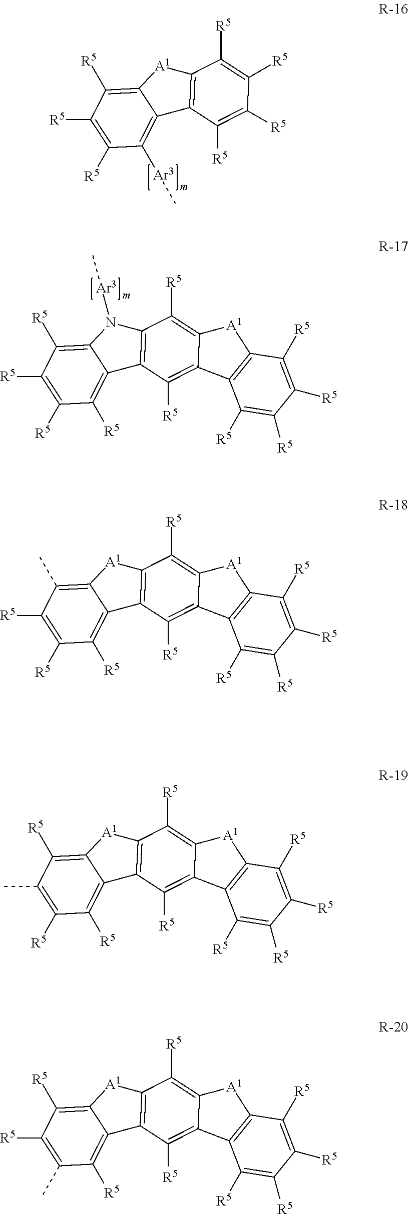

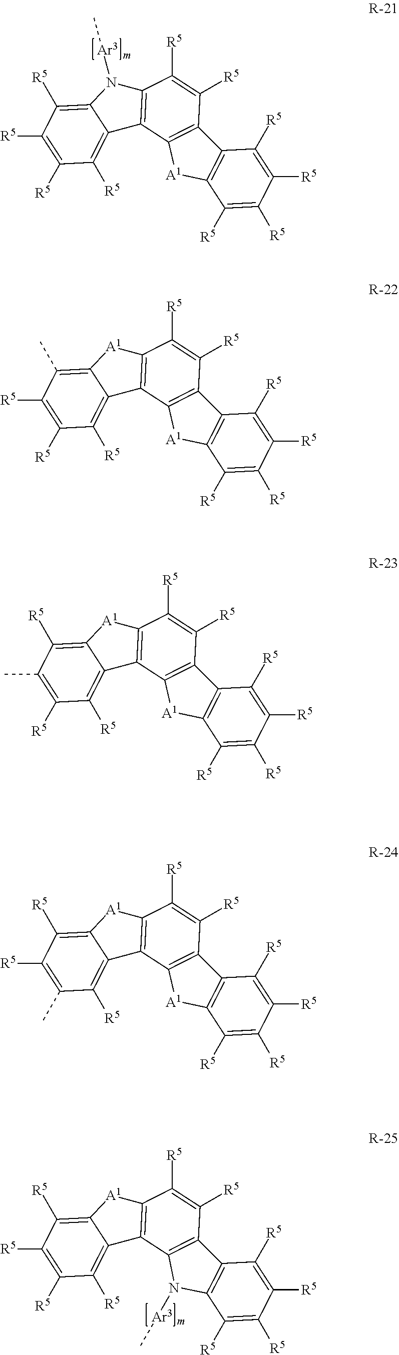

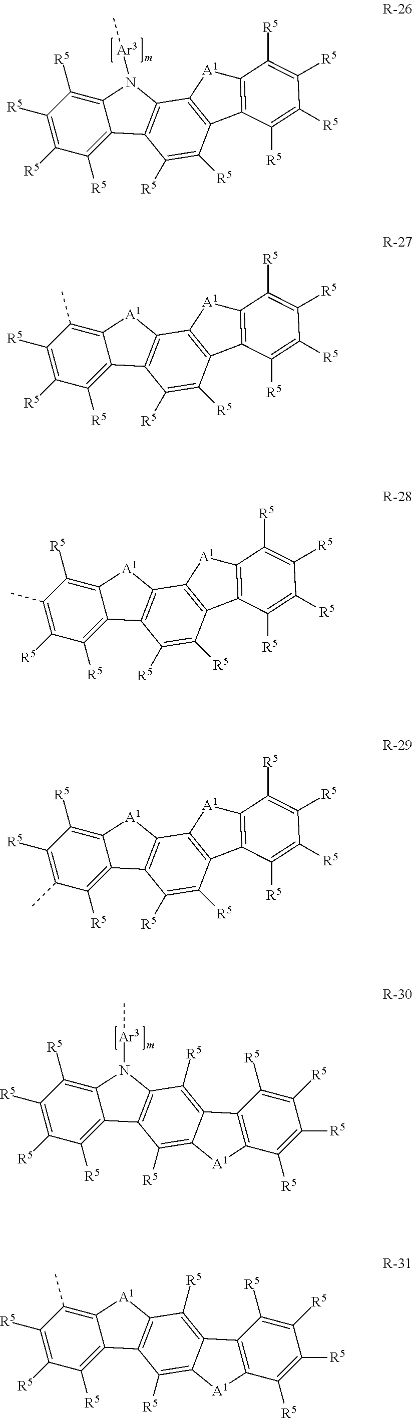

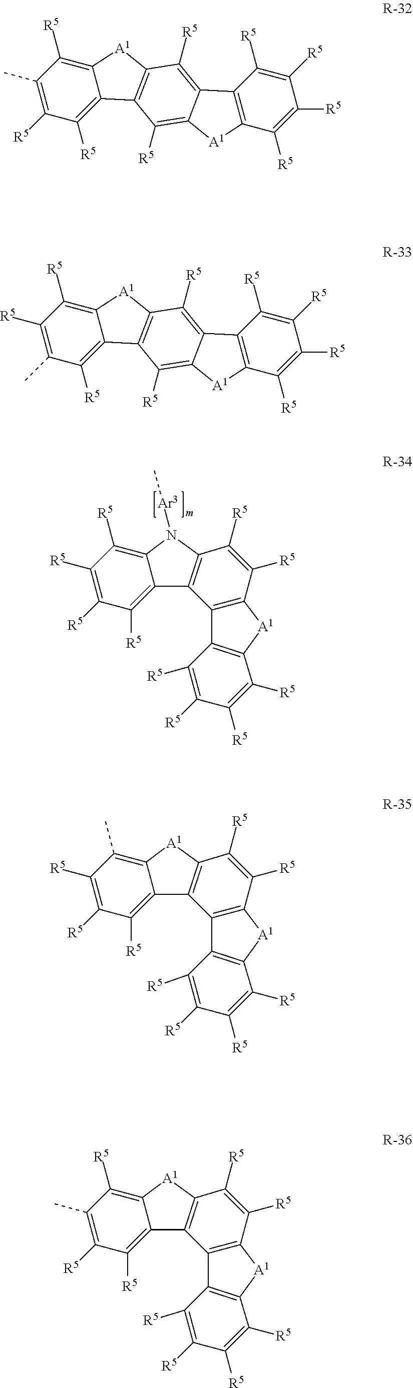

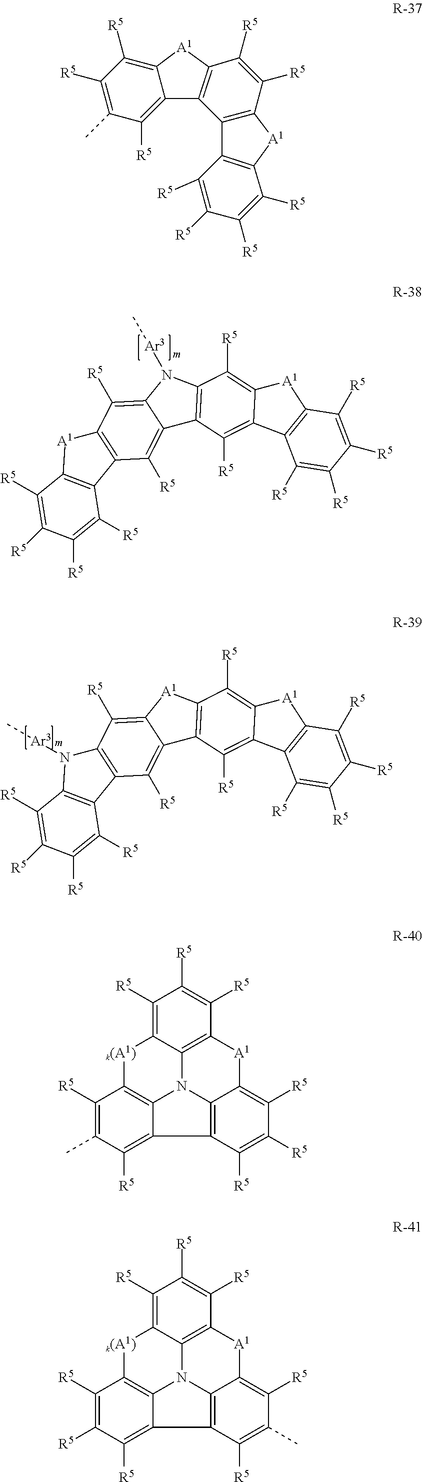

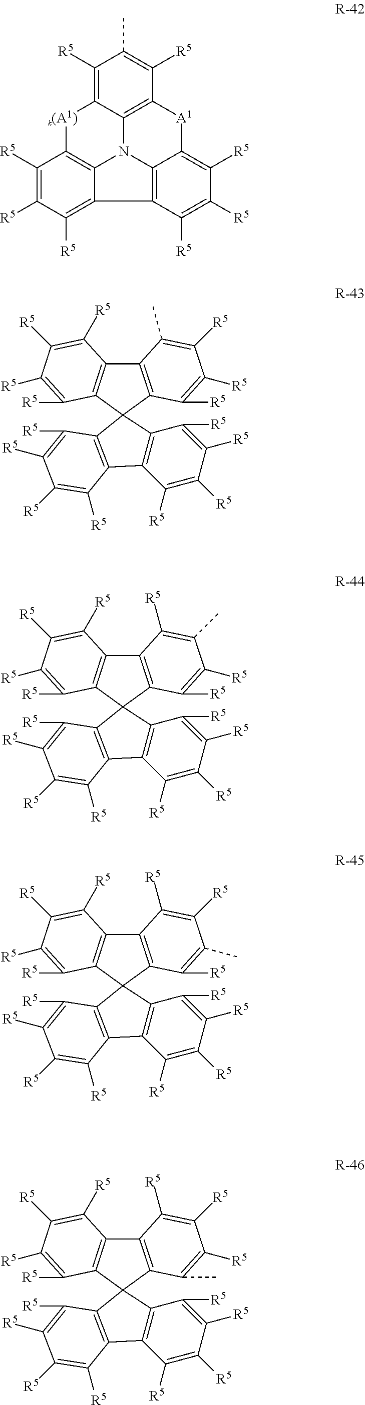

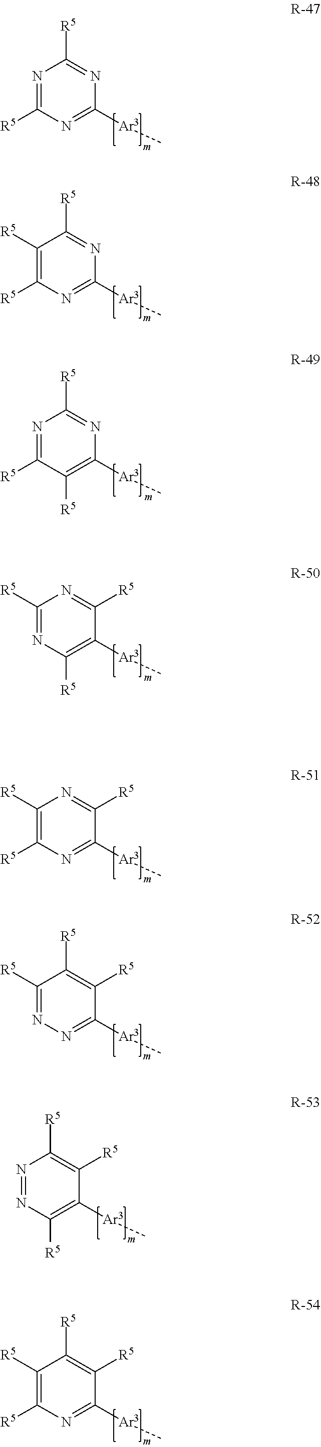

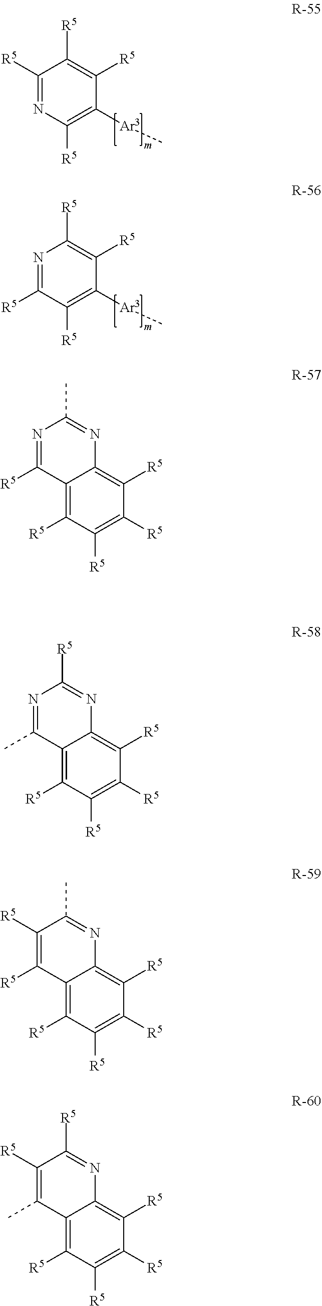

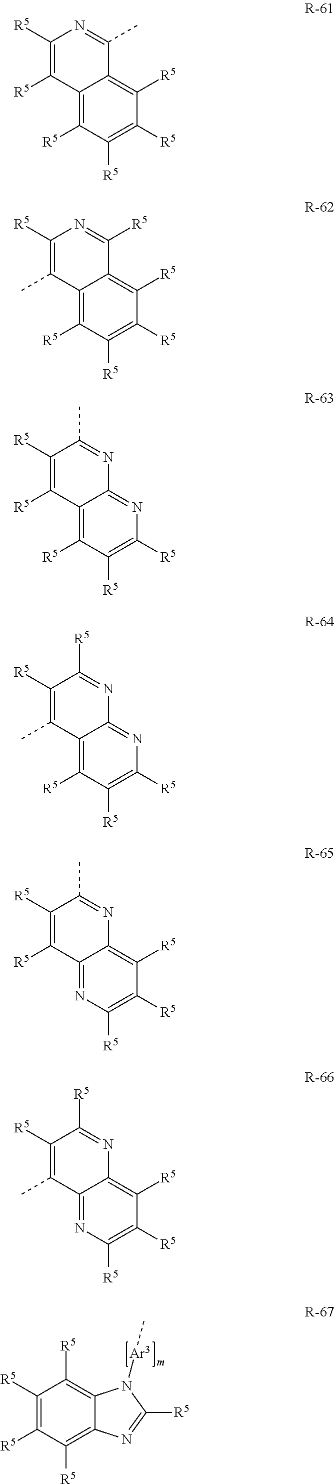

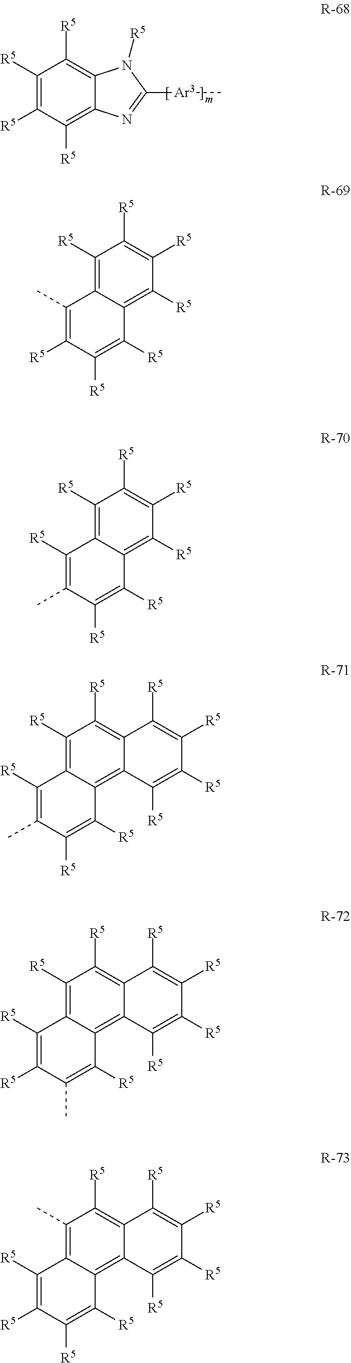

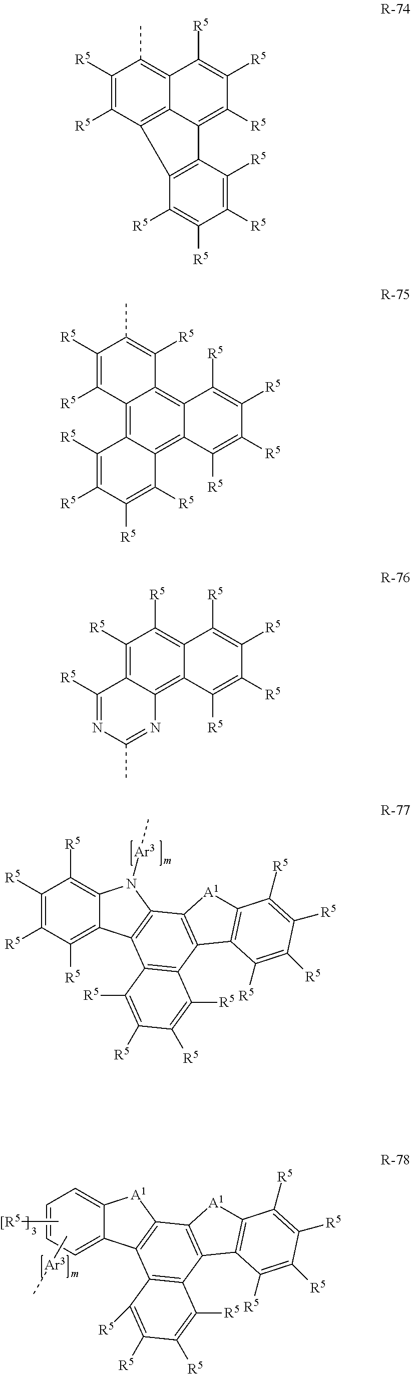

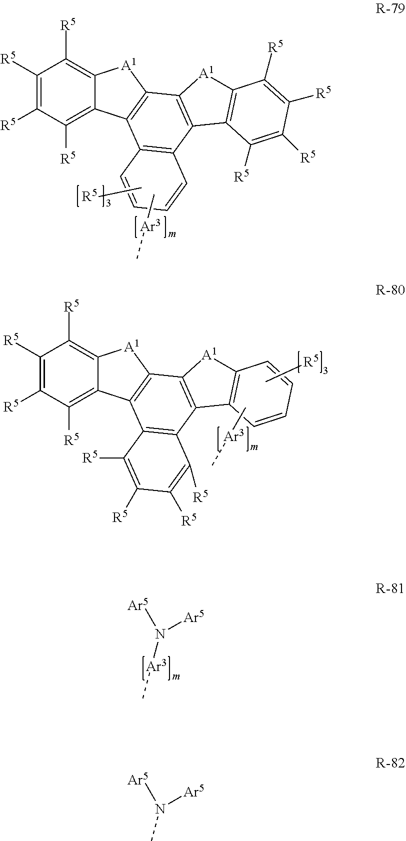

where R⁵ has the definitions given above, the dotted bond represents the bond to the base skeleton of the formula (I), and in addition:

Ar³ is the same or different at each instance and is a bivalent aromatic or heteroaromatic ring system which has 6 to 12 aromatic ring atoms and is substituted in each case by R⁵ radicals;

Ar⁵ is the same or different at each instance and is an aromatic ring system which has 6 to 40 aromatic ring atoms and is substituted by R⁶ radicals, or a heteroaromatic ring system which has 6 to 40 aromatic ring atoms and is substituted by R⁶ radicals;

A¹ is the same or different at each instance and is C(R⁵)₂, NR⁵, O or S; where, in formulae (R-78) to (R-80), preferably one A¹ in each case is NR⁵, and the other A¹ is S;

k is 0 or 1, where k=0 means that no A¹ group is bonded at this position and R⁵ radicals are bonded to the corresponding carbon atoms instead;

m is 0 or 1, where m=0 means that the Ar³ group is absent and that the corresponding aromatic or heteroaromatic group is bonded directly to the base skeleton of the formula (I).

Formula (I) preferably includes at least one R¹ group selected from N(R⁵)₂, aromatic ring systems which have 6 to 40 aromatic ring atoms and are substituted by R⁵ radicals, and heteroaromatic ring systems which have 5 to 40 aromatic ring atoms and are substituted by R⁵ radicals. More preferably, formula (I) includes at least one R¹ group selected from the abovementioned R-1 to R-82 groups.

R² is preferably the same or different at each instance and is selected from H, D, F, CN, Si(R⁵)₃, N(R⁵)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁵ radicals; and where one or more CH₂ groups in the alkyl or alkoxy groups mentioned may be replaced by —C≡C—, —R⁵C═CR⁵—, Si(R⁵)₂, C═O, C═NR⁵, —NR⁵—, —O—, —S—, —C(═O)O— or —C(═O)NR⁵—. More preferably, R² is the same or different at each instance and is selected from H, F, CN, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms, where said alkyl groups, said aromatic ring systems and said heteroaromatic ring systems are each substituted by R⁵ radicals. Very preferably, R² is the same or different at each instance and is selected from H, N(R⁵)₂, aromatic ring systems having 6 to 40 aromatic ring atoms, and heteroaromatic ring systems having 5 to 40 aromatic ring atoms; where the aromatic ring systems mentioned and the heteroaromatic ring systems mentioned are each substituted by R⁵ radicals. Preferred aromatic and heteroaromatic ring systems and N(R⁵)₂ groups as R² groups are selected from the abovementioned R-1 to R-82 groups.

Formula (I) preferably includes at least one R² group selected from N(R⁵)₂, aromatic ring systems which have 6 to 40 aromatic ring atoms and are substituted by R⁵ radicals, and heteroaromatic ring systems which have 5 to 40 aromatic ring atoms and are substituted by R⁵ radicals. More preferably, formula (I) includes at least one R² group selected from the abovementioned R-1 to R-82 groups.

R³ is preferably the same or different at each instance and is selected from H, D, F, CN, Si(R⁵)₃, N(R⁵)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁵ radicals; and where one or more CH₂ groups in the alkyl or alkoxy groups mentioned may be replaced by —C≡C—, —R⁵C═CR⁵—, Si(R⁵)₂, C═O, C═NR⁵, —NR⁵—, —O—, —S—, —C(═O)O— or —C(═O)NR⁵—. More preferably, R³ is the same or different at each instance and is selected from H, F, CN, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms, where said alkyl groups, said aromatic ring systems and said heteroaromatic ring systems are each substituted by R⁵ radicals. Very preferably, R³ is the same or different at each instance and is selected from H, N(R⁵)₂, aromatic ring systems having 6 to 40 aromatic ring atoms, and heteroaromatic ring systems having 5 to 40 aromatic ring atoms; where the aromatic ring systems mentioned and the heteroaromatic ring systems mentioned are each substituted by R⁵ radicals. Preferred aromatic and heteroaromatic ring systems and N(R⁵)₂ groups as R³ groups are selected from the abovementioned R-1 to R-82 groups.

Formula (I) preferably includes at least one R³ group selected from N(R⁵)₂, aromatic ring systems which have 6 to 40 aromatic ring atoms and are substituted by R⁵ radicals, and heteroaromatic ring systems which have 5 to 40 aromatic ring atoms and are substituted by R⁵ radicals. More preferably, formula (I) includes at least one R³ group selected from the abovementioned R-1 to R-82 groups.

R⁴ is preferably the same or different at each instance and is selected from H, D, F, CN, Si(R⁵)₃, N(R⁵)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁵ radicals; and where one or more CH₂ groups in the alkyl or alkoxy groups mentioned may be replaced by —C≡C—, —R⁵C═CR⁵—, Si(R⁵)₂, C═O, C═NR⁵, —NR⁵—, —O—, —S—, —C(═O)O— or —C(═O)NR⁵—. More preferably, R⁴ is the same or different at each instance and is selected from H, F, CN, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms, where said alkyl groups, said aromatic ring systems and said heteroaromatic ring systems are each substituted by R⁵ radicals. Very preferably, R⁴ is the same or different at each instance and is selected from H, N(R⁵)₂, aromatic ring systems having 6 to 40 aromatic ring atoms, and heteroaromatic ring systems having 5 to 40 aromatic ring atoms; where the aromatic ring systems mentioned and the heteroaromatic ring systems mentioned are each substituted by R⁵ radicals. Preferred aromatic and heteroaromatic ring systems and N(R⁵)₂ groups as R⁴ groups are selected from the abovementioned R-1 to R-82 groups.

Preferably, in formula (I), either
a) at least one group selected from the R¹, R², R³ and R⁴ groups is selected from aromatic ring systems which have 7 to 40 aromatic ring atoms and are each substituted by R⁵ radicals; and heteroaromatic ring systems which have 5 to 40 aromatic ring atoms and are each substituted by R⁵ radicals; or, in formula (I),
b) at least two groups selected from the R¹, R², R³ and R⁴ groups are selected from aromatic ring systems which have 6 to 40 aromatic ring atoms and are each substituted by R⁵ radicals; and heteroaromatic ring systems which have 5 to 40 aromatic ring atoms and are each substituted by R⁵ radicals.

Preferably, in formula (I), at least one group selected from the R¹, R², R³ and R⁴ groups is selected from aromatic ring systems which have 7 to 40 aromatic ring atoms and are each substituted by R⁵ radicals; and heteroaromatic ring systems which have 5 to 40 aromatic ring atoms and are each substituted by R⁵ radicals. More preferably, in formula (I), at least one group selected from the R¹, R², and R³ groups is selected from aromatic ring systems which have 7 to 40 aromatic ring atoms and are each substituted by R⁵ radicals; and heteroaromatic ring systems which have 5 to 40 aromatic ring atoms and are each substituted by R⁵ radicals. Even more preferably, at least one group selected from the R¹, R², and R³ groups in formula (I) is selected from the R-1 to R-81 groups as defined above. Most preferably, at least one group selected from the R¹ and R² groups in formula (I) is selected from the R-1 to R-81 groups as defined above.

Preferably, R⁵ is the same or different at each instance and is selected from H, D, F, CN, Si(R⁶)₃, N(R⁶)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁶ radicals; and where one or more CH₂ groups in the alkyl or alkoxy groups mentioned may be replaced by —C↓C—, —R⁶C═CR⁶—, Si(R⁶)₂, C═O, C═NR⁶, —NR⁶—, —O—, —S—, —C(═O)O— or —C(═O)NR⁶—.

Compounds of the Formula (I) Preferably Conform to the Formula (I-A)

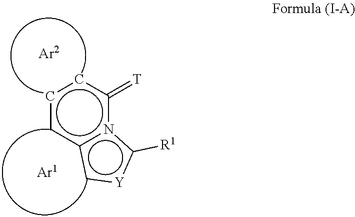

where T is O or S;

where Ar² is selected from aromatic ring systems which have 6 to 40 aromatic ring atoms and are substituted by R³ radicals, and which include the C—C unit, and heteroaromatic ring systems which have 5 to 40 aromatic ring atoms and are substituted by R³ radicals, and which include the C—C unit, and where the other variables are as defined for formula (I) above, where, when Y is CR¹, either
a) at least one group selected from the R¹, R², R³ and R⁴ groups is selected from aromatic ring systems which have 7 to 40 aromatic ring atoms and are each substituted by R⁵ radicals; and heteroaromatic ring systems which have 5 to 40 aromatic ring atoms and are each substituted by R⁵ radicals; or
b) at least two groups selected from the R¹, R², R³ and R⁴ groups are selected from aromatic ring systems which have 6 to 40 aromatic ring atoms and are each substituted by R⁵ radicals; and heteroaromatic ring systems which have 5 to 40 aromatic ring atoms and are each substituted by R⁵ radicals.

Preferably, in formula (I-A), Y is N. It is further preferable that T is O. In addition, the preferred embodiments of the variables specified above in relation to formula (I) are also applicable to formula (I-A).

Preferably, Compounds of the Formula (I-A) Conform to One of the Formulae (I-A-1) to (I-A-3)

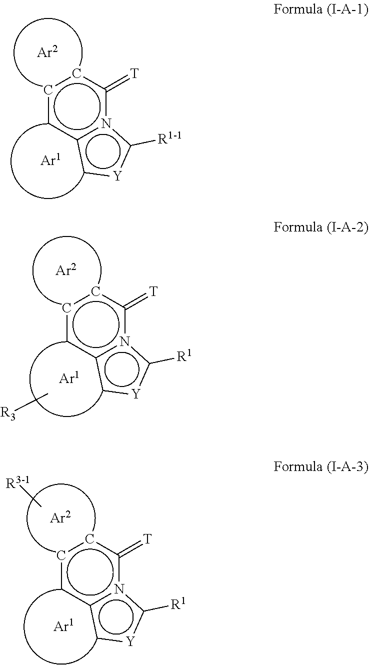

where R¹⁻¹, R²⁻¹ and R³⁻¹ are selected from aromatic ring systems which have 7 to 40 aromatic ring atoms and are each substituted by R⁵ radicals; and heteroaromatic ring systems which have 5 to 40 aromatic ring atoms and are each substituted by R⁵ radicals, and are preferably selected from one of the R-1 to R-81 groups.

Compounds of the Formula (I) Preferably Conform to One of the Formulae (I-1) to (1-8)

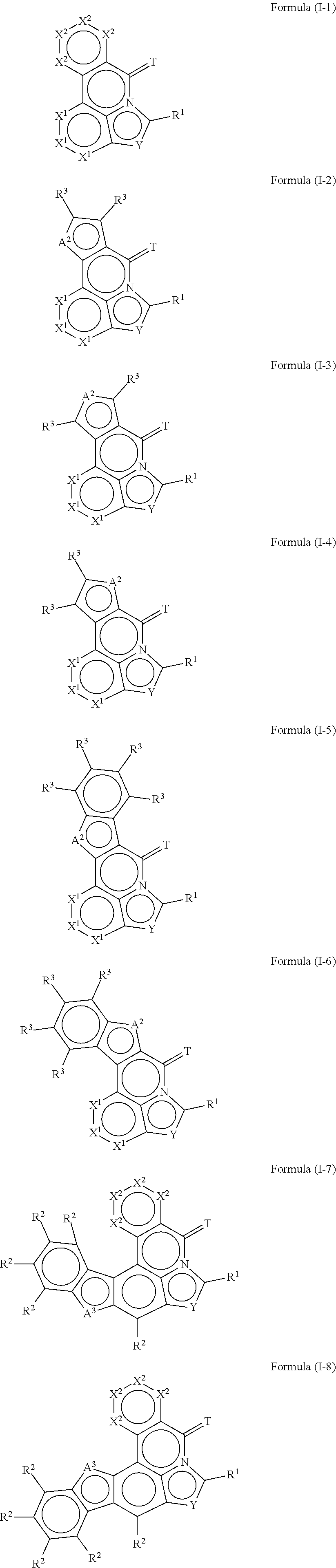

where A² is C(R³)₂, NR³, O or S; and where A³ is C(R²)₂, NR², O or S, and where X¹ is the same or different at each instance and is selected from N and CR² and is preferably CR², and where X² is the same or different at each instance and is selected from N and CR³ and is preferably CR³, and where the further variables are as defined above; and where, when Y is CR¹, either
a) at least one group selected from the R¹, R², R³ and R⁴ groups is selected from aromatic ring systems which have 7 to 40 aromatic ring atoms and are each substituted by R⁵ radicals; and heteroaromatic ring systems which have 5 to 40 aromatic ring atoms and are each substituted by R⁵ radicals; or
b) at least two groups selected from the R¹, R², R³ and R⁴ groups are selected from aromatic ring systems which have 6 to 40 aromatic ring atoms and are each substituted by R⁵ radicals; and heteroaromatic ring systems which have 5 to 40 aromatic ring atoms and are each substituted by R⁵ radicals.

For the abovementioned formulae, it is further preferable that T is O, and that Y is N.

Among the abovementioned formulae, preference is given to the formulae (I-1), (I-7) and (I-8), where it is preferable in these formulae that X¹ is CR², X² is CR³, Y is N and T is O. Most preferred is the formula (I-1), where it is preferable in this formula that X¹ is CR², X² is CR³, Y is N and T is O.

Preferably, in the formulae (I-1) to (I-8), at least one group selected from the R¹, R², and R³ groups is selected from aromatic ring systems which have 7 to 40 aromatic ring atoms and are each substituted by R⁵ radicals; and heteroaromatic ring systems which have 5 to 40 aromatic ring atoms and are each substituted by R⁵ radicals. More preferably, in the formulae (I-1) to (I-8), at least one group selected from the R¹, R², and R³ groups is selected from the R-1 to R-81 groups as defined above. Even more preferably, in the formulae (I-1) to (I-8), at least one group selected from the R¹ and R² groups is selected from the R-1 to R-81 groups as defined above.

Preferred variants of the formulae (I-1), (I-7) and (I-8) are the following formulae:

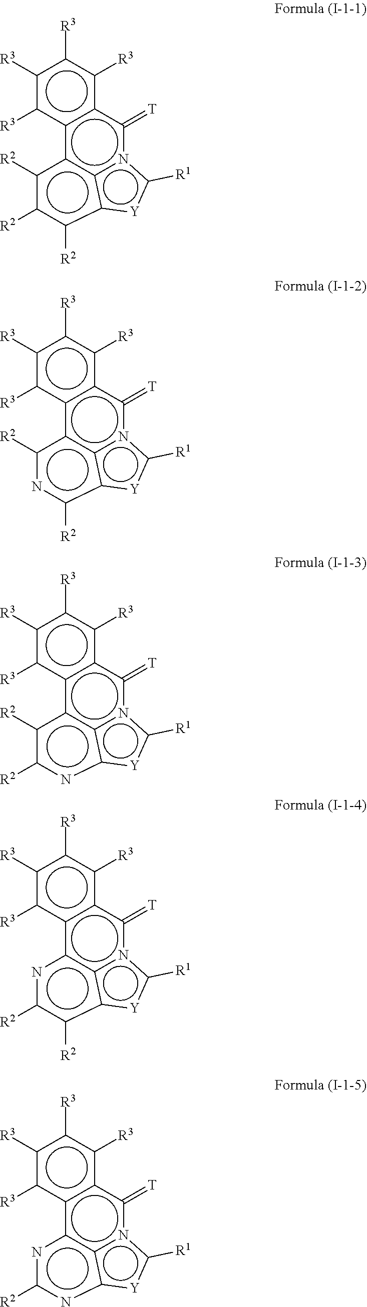

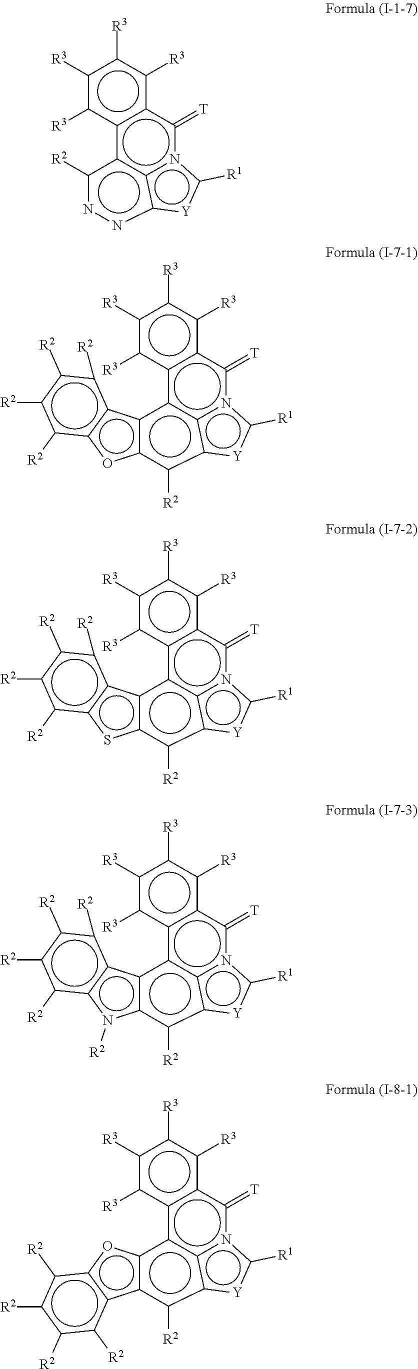

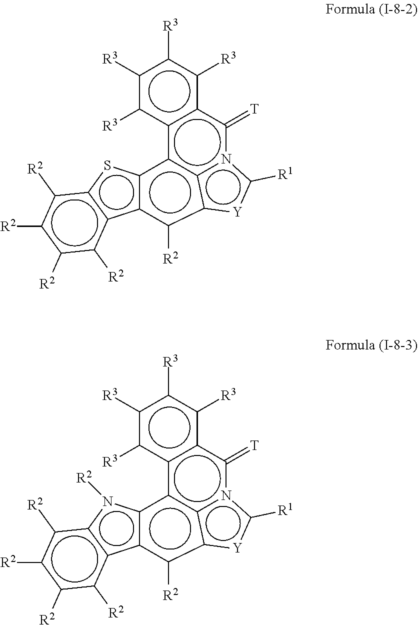

where the variables are as defined above, and where T is preferably O, and Y is preferably N, and where, when Y is CR¹, either
a) at least one group selected from the R¹, R², R³ and R⁴ groups is selected from aromatic ring systems which have 7 to 40 aromatic ring atoms and are each substituted by R⁵ radicals; and heteroaromatic ring systems which have 5 to 40 aromatic ring atoms and are each substituted by R⁵ radicals; or
b) at least two groups selected from the R¹, R², R³ and R⁴ groups are selected from aromatic ring systems which have 6 to 40 aromatic ring atoms and are each substituted by R⁵ radicals; and heteroaromatic ring systems which have 5 to 40 aromatic ring atoms and are each substituted by R⁵ radicals.

Preferably, in the abovementioned formulae, at least one group selected from the R¹, R², and R³ groups in each case is selected from aromatic ring systems which have 7 to 40 aromatic ring atoms and are each substituted by R⁵ radicals; and heteroaromatic ring systems which have 5 to 40 aromatic ring atoms and are each substituted by R⁵ radicals. More preferably, in the abovementioned formulae, at least one group selected from the R¹, R², and R³ groups is selected from the R-1 to R-81 groups as defined above. Even more preferably, in the abovementioned formulae, at least one group selected from the R¹ and R² groups is selected from the R-1 to R-81 groups as defined above.

Most preferred among the abovementioned formulae is the formula (I-1-1).

Preferred variants of the formula (I-1-1) conform to the following formulae:

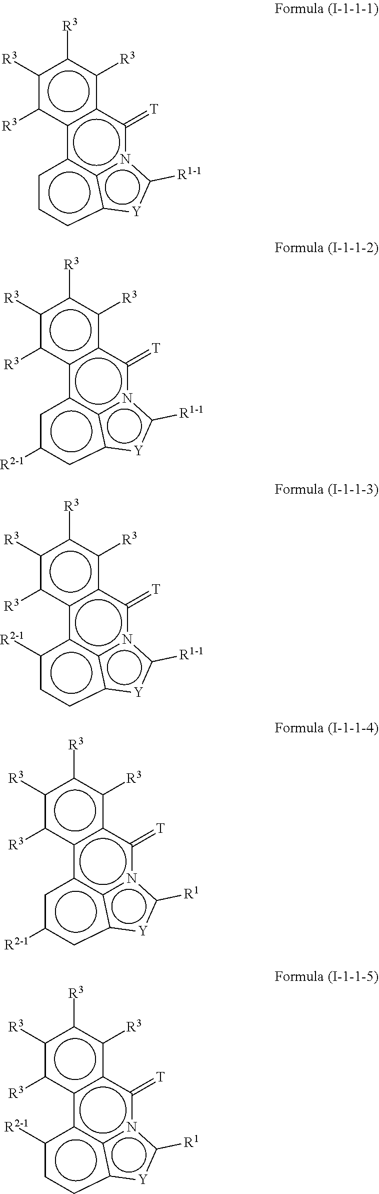

where R¹⁻¹ and R²⁻¹ are selected from aromatic ring systems which have 7 to 40 aromatic ring atoms and are each substituted by R⁵ radicals; and heteroaromatic ring systems which have 5 to 40 aromatic ring atoms and are each substituted by R⁵ radicals, and are preferably selected from one of the R-1 to R-81 groups, and where the other variables are as defined above and preferably correspond to their preferred embodiments. It is especially preferable that, in the abovementioned formulae, T is O and Y is N.

Preferred compounds of the formula (I) are shown in the following tabl:

| | |
|---|---|
| 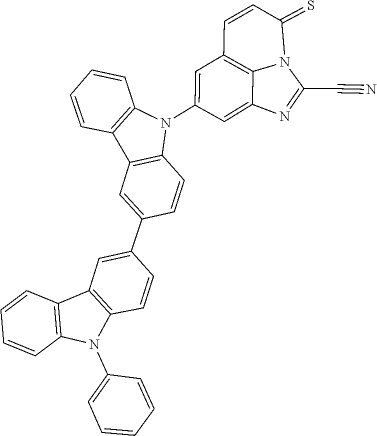 [SMILES: N#Cc1nc2cc(-n3c4ccccc4c4cc(-c5ccc6c(c5)c5ccccc5n6-c5ccccc5)ccc43)cc3ccc(=S)n1c32] | 1 |
| 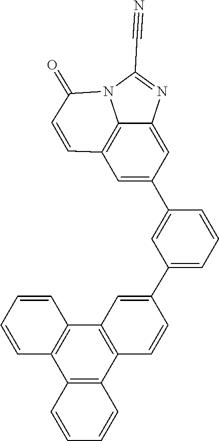 [SMILES: N#Cc1nc2cc(-c3cccc(-c4ccc5c6ccccc6c6ccccc6c5c4)c3)cc3ccc(=O)n1c32] | 2 |
| 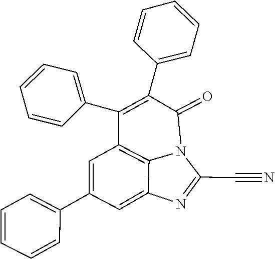 [SMILES: N#Cc1nc2cc(-c3ccccc3)cc3c(-c4ccccc4)c(-c4ccccc4)c(=O)n1c23] | 3 |
| 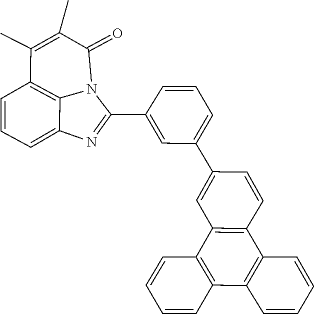 [SMILES: Cc1c(C)c2cccc3nc(-c4cccc(-c5ccc6c7ccccc7c7ccccc7c6c5)c4)n(c1=O)c32] | 4 |
| 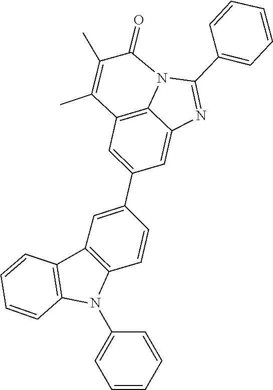 [SMILES: Cc1c(C)c2cc(-c3ccc4c(c3)c3ccccc3n4-c3ccccc3)cc3nc(-c4ccccc4)n(c1=O)c32] | 5 |
| 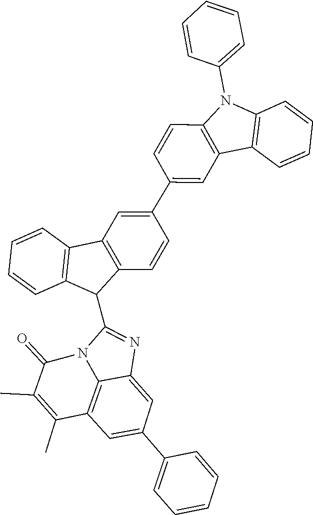 [SMILES: Cc1c(C)c2cc(-c3ccccc3)cc3nc(C4c5ccccc5-c5cc(-c6ccc7c(c6)c6ccccc6n7-c6ccccc6)ccc54)n(c1=O)c32] | 6 |
| 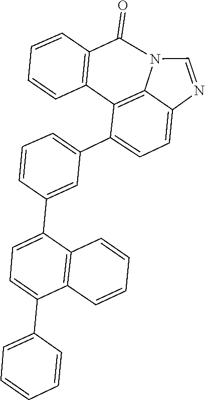 [SMILES: O=c1c2ccccc2c2c(-c3cccc(-c4ccc(-c5ccccc5)c5ccccc45)c3)ccc3ncn1c32] | 7 |
| 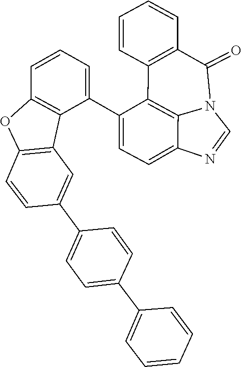 [SMILES: O=c1c2ccccc2c2c(-c3cccc4oc5ccc(-c6ccc(-c7ccccc7)cc6)cc5c34)ccc3ncn1c32] | 8 |
| 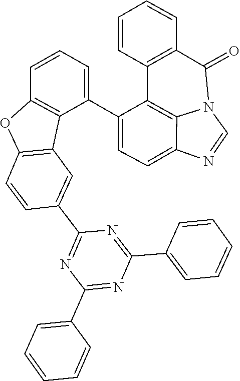 [SMILES: O=c1c2ccccc2c2c(-c3cccc4oc5ccc(-c6nc(-c7ccccc7)nc(-c7ccccc7)n6)cc5c34)ccc3ncn1c32] | 9 |
| 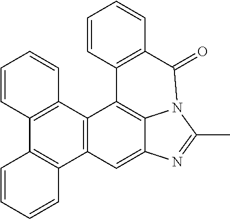 [SMILES: Cc1nc2cc3c4ccccc4c4ccccc4c3c3c4ccccc4c(=O)n1c23] | 10 |
| 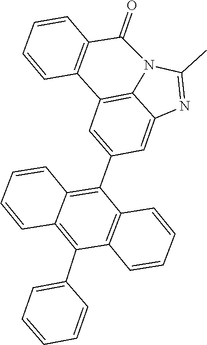 [SMILES: Cc1nc2cc(-c3c4ccccc4c(-c4ccccc4)c4ccccc34)cc3c4ccccc4c(=O)n1c23] | 11 |
| 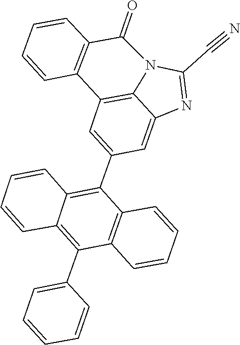 [SMILES: N#Cc1nc2cc(-c3c4ccccc4c(-c4ccccc4)c4ccccc34)cc3c4ccccc4c(=O)n1c23] | 12 |
| 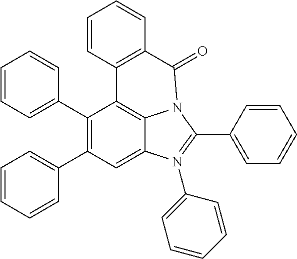 | 13 |
| 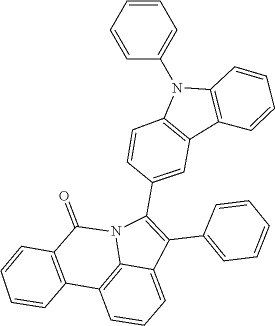 [SMILES: O=c1c2ccccc2c2cccc3c(-c4ccccc4)c(-c4ccc5c(c4)c4ccccc4n5-c4ccccc4)n1c32] | 14 |
| 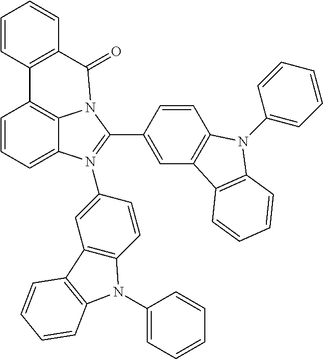 | 15 |
| 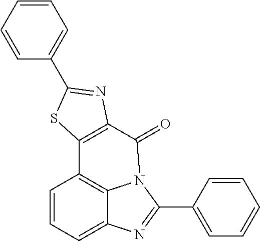 [SMILES: O=c1c2nc(-c3ccccc3)sc2c2cccc3nc(-c4ccccc4)n1c32] | 16 |
| 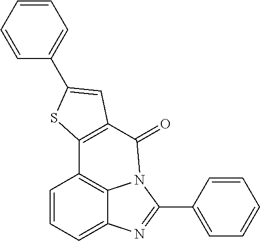 [SMILES: O=c1c2cc(-c3ccccc3)sc2c2cccc3nc(-c4ccccc4)n1c32] | 17 |
| 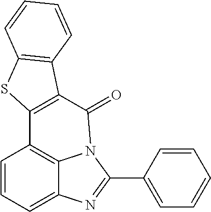 [SMILES: O=c1c2c3ccccc3sc2c2cccc3nc(-c4ccccc4)n1c32] | 18 |
| 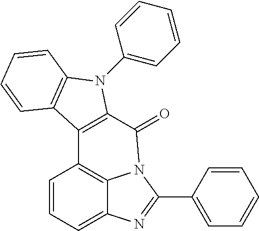 [SMILES: O=c1c2c(c3ccccc3n2-c2ccccc2)c2cccc3nc(-c4ccccc4)n1c32] | 19 |
| 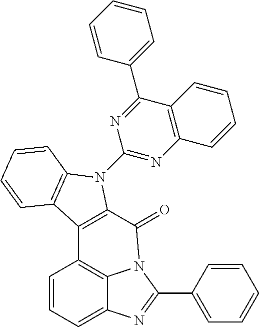 [SMILES: O=c1c2c(c3ccccc3n2-c2nc(-c3ccccc3)c3ccccc3n2)c2cccc3nc(-c4ccccc4)n1c32] | 20 |
| 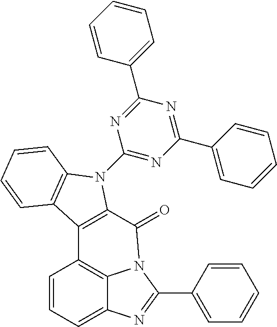 [SMILES: O=c1c2c(c3ccccc3n2-c2nc(-c3ccccc3)nc(-c3ccccc3)n2)c2cccc3nc(-c4ccccc4)n1c32] | 21 |
| 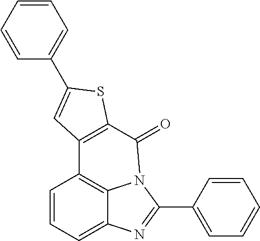 [SMILES: O=c1c2sc(-c3ccccc3)cc2c2cccc3nc(-c4ccccc4)n1c32] | 22 |
| 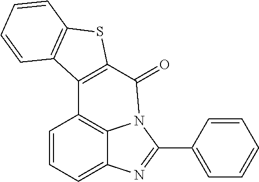 [SMILES: O=c1c2sc3ccccc3c2c2cccc3nc(-c4ccccc4)n1c32] | 23 |
| 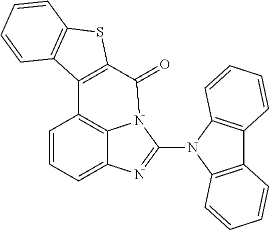 [SMILES: O=c1c2sc3ccccc3c2c2cccc3nc(-n4c5ccccc5c5ccccc54)n1c32] | 24 |
| 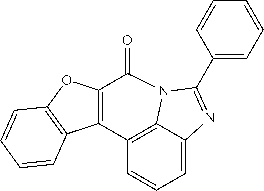 [SMILES: O=c1c2oc3ccccc3c2c2cccc3nc(-c4ccccc4)n1c32] | 25 |
| 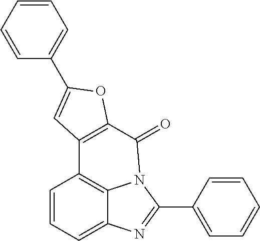 [SMILES: O=c1c2oc(-c3ccccc3)cc2c2cccc3nc(-c4ccccc4)n1c32] | 26 |
| 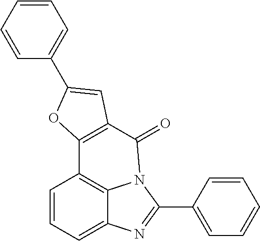 [SMILES: O=c1c2cc(-c3ccccc3)oc2c2cccc3nc(-c4ccccc4)n1c32] | 27 |
| 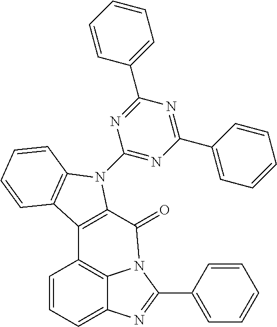 [SMILES: O=c1c2c(c3ccccc3n2-c2nc(-c3ccccc3)nc(-c3ccccc3)n2)c2cccc3nc(-c4ccccc4)n1c32] | 28 |
| 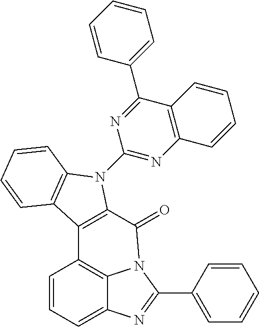 [SMILES: O=c1c2c(c3ccccc3n2-c2nc(-c3ccccc3)c3ccccc3n2)c2cccc3nc(-c4ccccc4)n1c32] | 29 |
| 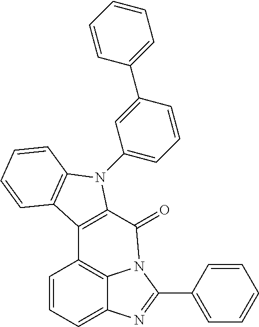 [SMILES: O=c1c2c(c3ccccc3n2-c2cccc(-c3ccccc3)c2)c2cccc3nc(-c4ccccc4)n1c32] | 30 |
| 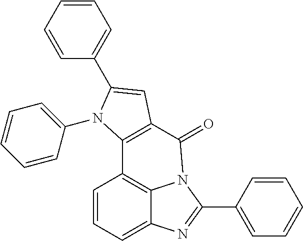 [SMILES: O=c1c2cc(-c3ccccc3)n(-c3ccccc3)c2c2cccc3nc(-c4ccccc4)n1c32] | 31 |
| 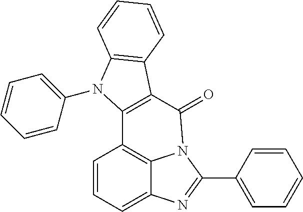 [SMILES: O=c1c2c3ccccc3n(-c3ccccc3)c2c2cccc3nc(-c4ccccc4)n1c32] | 32 |
| 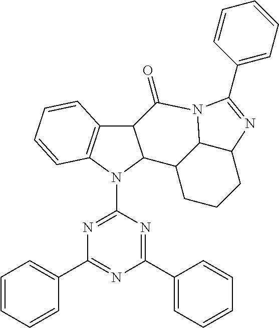 [SMILES: O=C1C2c3ccccc3N(c3nc(-c4ccccc4)nc(-c4ccccc4)n3)C2C2CCCC3N=C(c4ccccc4)N1C32] | 33 |
| 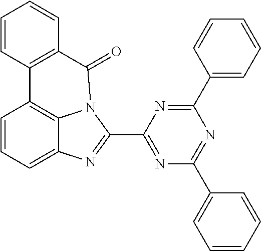 [SMILES: O=c1c2ccccc2c2cccc3nc(-c4nc(-c5ccccc5)nc(-c5ccccc5)n4)n1c32] | 34 |
| 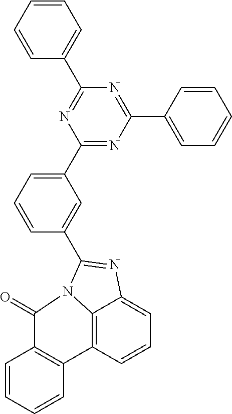 [SMILES: O=c1c2ccccc2c2cccc3nc(-c4cccc(-c5nc(-c6ccccc6)nc(-c6ccccc6)n5)c4)n1c32] | 35 |
| 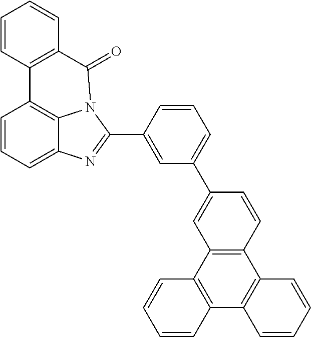 [SMILES: O=c1c2ccccc2c2cccc3nc(-c4cccc(-c5ccc6c7ccccc7c7ccccc7c6c5)c4)n1c32] | 36 |
| 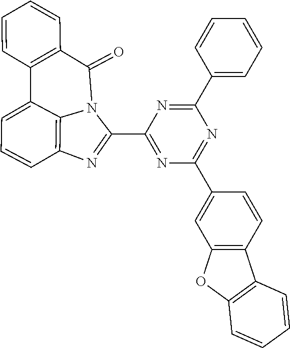 [SMILES: O=c1c2ccccc2c2cccc3nc(-c4nc(-c5ccccc5)nc(-c5ccc6c(c5)oc5ccccc56)n4)n1c32] | 37 |
| 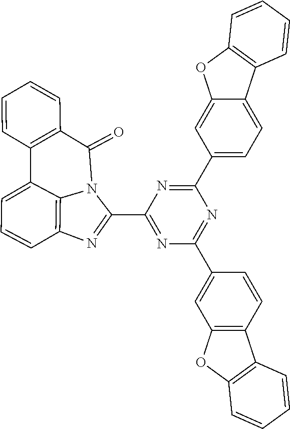 [SMILES: O=c1c2ccccc2c2cccc3nc(-c4nc(-c5ccc6c(c5)oc5ccccc56)nc(-c5ccc6c(c5)oc5ccccc56)n4)n1c32] | 38 |
| 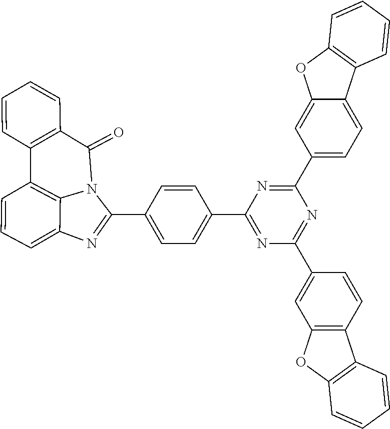 [SMILES: O=c1c2ccccc2c2cccc3nc(-c4ccc(-c5nc(-c6ccc7c(c6)oc6ccccc67)nc(-c6ccc7c(c6)oc6ccccc67)n5)cc4)n1c32] | 39 |
| 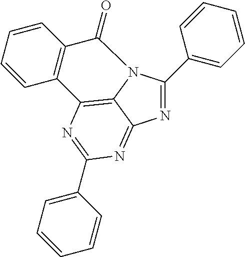 [SMILES: O=c1c2ccccc2c2nc(-c3ccccc3)nc3nc(-c4ccccc4)n1c32] | 40 |
| 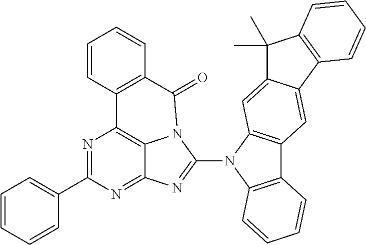 [SMILES: CC1(C)c2ccccc2-c2cc3c4ccccc4n(-c4nc5nc(-c6ccccc6)nc6c7ccccc7c(=O)n4c56)c3cc21] | 41 |
| 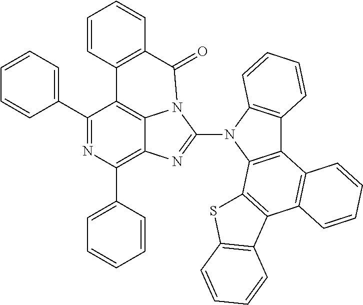 [SMILES: O=c1c2ccccc2c2c(-c3ccccc3)nc(-c3ccccc3)c3nc(-n4c5ccccc5c5c6ccccc6c6c7ccccc7sc6c54)n1c32] | 42 |
| 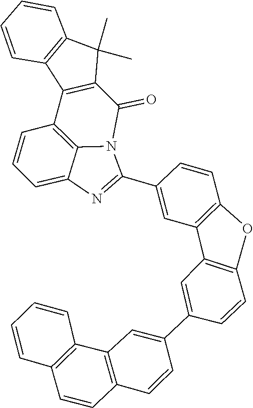 [SMILES: CC1(C)c2ccccc2-c2c1c(=O)n1c(-c3ccc4oc5ccc(-c6ccc7ccc8ccccc8c7c6)cc5c4c3)nc3cccc2c31] | 43 |
| 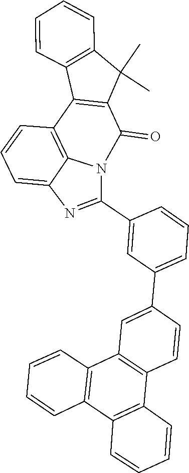 [SMILES: CC1(C)c2ccccc2-c2c1c(=O)n1c(-c3cccc(-c4ccc5c6ccccc6c6ccccc6c5c4)c3)nc3cccc2c31] | 44 |
| 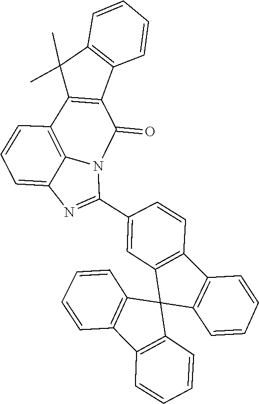 [SMILES: CC1(C)c2ccccc2-c2c1c1cccc3nc(-c4ccc5c(c4)C4(c6ccccc6-c6ccccc64)c4ccccc4-5)n(c2=O)c31] | 45 |
| 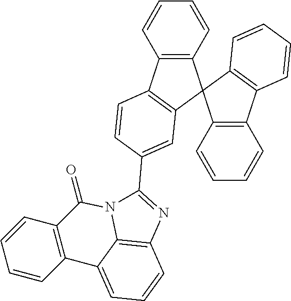 [SMILES: O=c1c2ccccc2c2cccc3nc(-c4ccc5c(c4)C4(c6ccccc6-c6ccccc64)c4ccccc4-5)n1c32] | 46 |
| 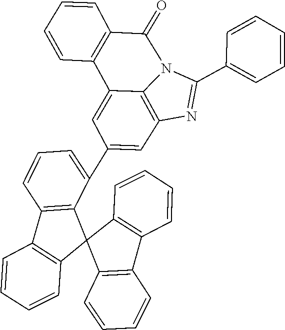 [SMILES: O=c1c2ccccc2c2cc(-c3cccc4c3C3(c5ccccc5-c5ccccc53)c3ccccc3-4)cc3nc(-c4ccccc4)n1c32] | 47 |
| 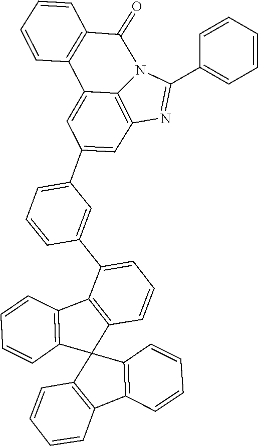 [SMILES: O=c1c2ccccc2c2cc(-c3cccc(-c4cccc5c4-c4ccccc4C54c5ccccc5-c5ccccc54)c3)cc3nc(-c4ccccc4)n1c32] | 48 |
| 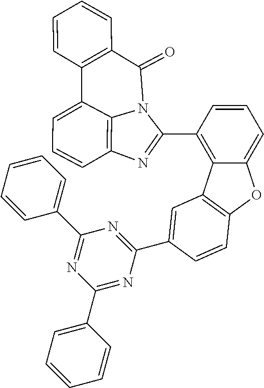 [SMILES: O=c1c2ccccc2c2cccc3nc(-c4cccc5oc6ccc(-c7nc(-c8ccccc8)nc(-c8ccccc8)n7)cc6c45)n1c32] | 49 |
| 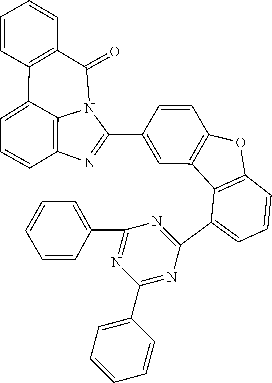 [SMILES: O=c1c2ccccc2c2cccc3nc(-c4ccc5oc6cccc(-c7nc(-c8ccccc8)nc(-c8ccccc8)n7)c6c5c4)n1c32] | 50 |
| 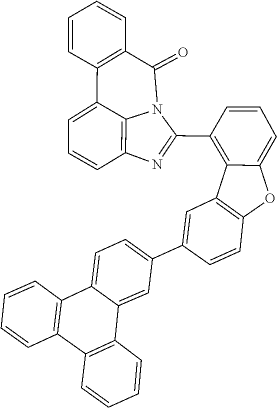 [SMILES: O=c1c2ccccc2c2cccc3nc(-c4cccc5oc6ccc(-c7ccc8c9ccccc9c9ccccc9c8c7)cc6c45)n1c32] | 51 |
| 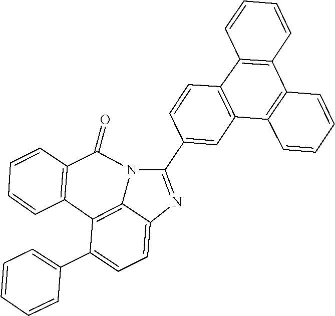 [SMILES: O=c1c2ccccc2c2c(-c3ccccc3)ccc3nc(-c4ccc5c6ccccc6c6ccccc6c5c4)n1c32] | 52 |
| 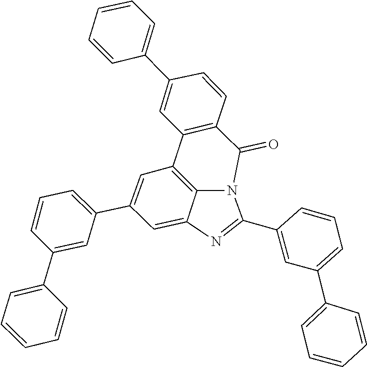 [SMILES: O=c1c2ccc(-c3ccccc3)cc2c2cc(-c3cccc(-c4ccccc4)c3)cc3nc(-c4cccc(-c5ccccc5)c4)n1c32] | 53 |
| 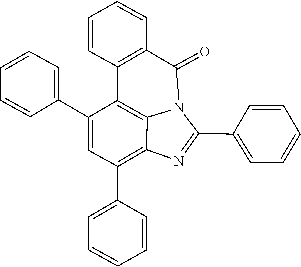 [SMILES: O=c1c2ccccc2c2c(-c3ccccc3)cc(-c3ccccc3)c3nc(-c4ccccc4)n1c32] | 54 |
| 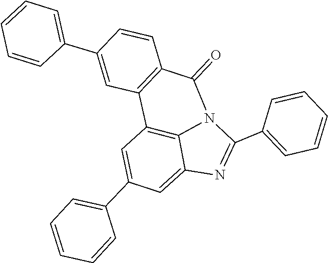 [SMILES: O=c1c2ccc(-c3ccccc3)cc2c2cc(-c3ccccc3)cc3nc(-c4ccccc4)n1c32] | 55 |
| 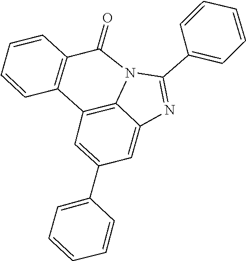 [SMILES: O=c1c2ccccc2c2cc(-c3ccccc3)cc3nc(-c4ccccc4)n1c32] | 56 |
| 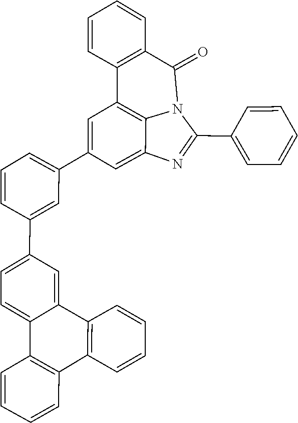 [SMILES: O=c1c2ccccc2c2cc(-c3cccc(-c4ccc5c6ccccc6c6ccccc6c5c4)c3)cc3nc(-c4ccccc4)n1c32] | 57 |
| 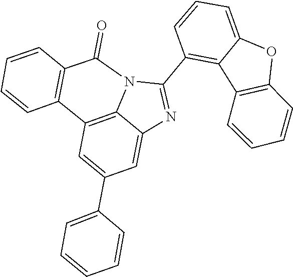 [SMILES: O=c1c2ccccc2c2cc(-c3ccccc3)cc3nc(-c4cccc5oc6ccccc6c45)n1c32] | 58 |
| 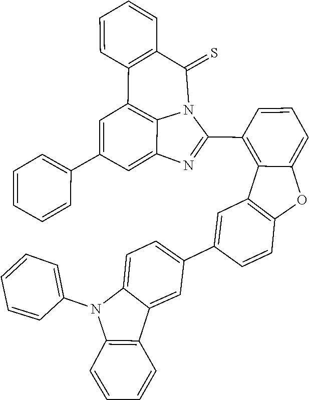 [SMILES: S=c1c2ccccc2c2cc(-c3ccccc3)cc3nc(-c4cccc5oc6ccc(-c7ccc8c(c7)c7ccccc7n8-c7ccccc7)cc6c45)n1c32] | 59 |
| 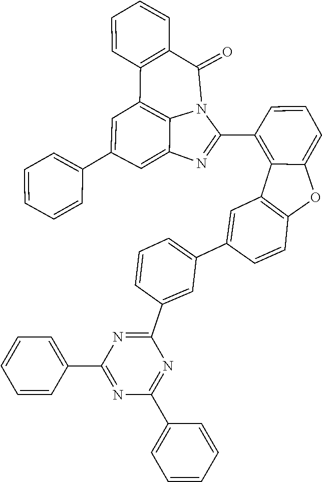 [SMILES: O=c1c2ccccc2c2cc(-c3ccccc3)cc3nc(-c4cccc5oc6ccc(-c7cccc(-c8nc(-c9ccccc9)nc(-c9ccccc9)n8)c7)cc6c45)n1c32] | 60 |
| 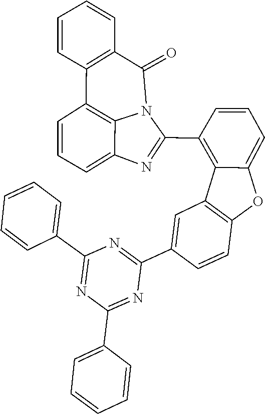 [SMILES: O=c1c2ccccc2c2cccc3nc(-c4cccc5oc6ccc(-c7nc(-c8ccccc8)nc(-c8ccccc8)n7)cc6c45)n1c32] | 61 |
| 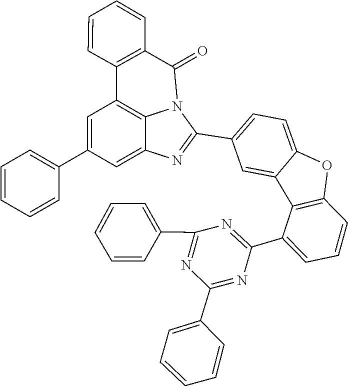 [SMILES: O=c1c2ccccc2c2cc(-c3ccccc3)cc3nc(-c4ccc5oc6cccc(-c7nc(-c8ccccc8)nc(-c8ccccc8)n7)c6c5c4)n1c32] | 62 |
| 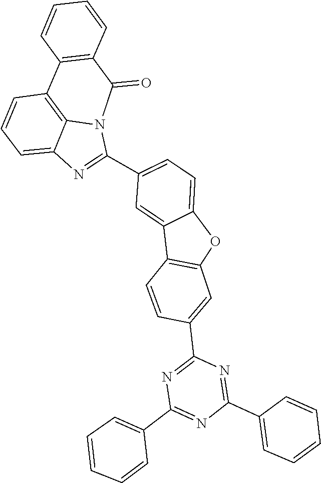 [SMILES: O=c1c2ccccc2c2cccc3nc(-c4ccc5oc6cc(-c7nc(-c8ccccc8)nc(-c8ccccc8)n7)ccc6c5c4)n1c32] | 63 |
| 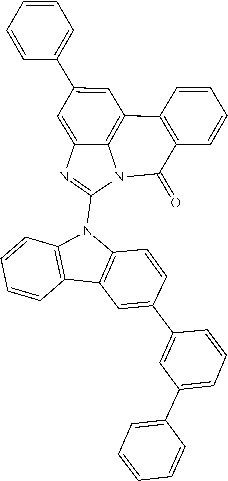 [SMILES: O=c1c2ccccc2c2cc(-c3ccccc3)cc3nc(-n4c5ccccc5c5cc(-c6cccc(-c7ccccc7)c6)ccc54)n1c32] | 64 |
| 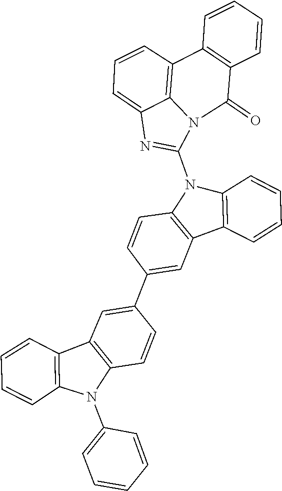 [SMILES: O=c1c2ccccc2c2cccc3nc(-n4c5ccccc5c5cc(-c6ccc7c(c6)c6ccccc6n7-c6ccccc6)ccc54)n1c32] | 65 |
| 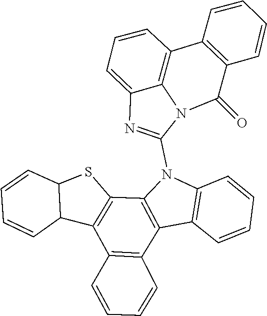 [SMILES: O=c1c2ccccc2c2cccc3nc(-n4c5ccccc5c5c6ccccc6c6c(c54)SC4C=CC=CC64)n1c32] | 66 |
| 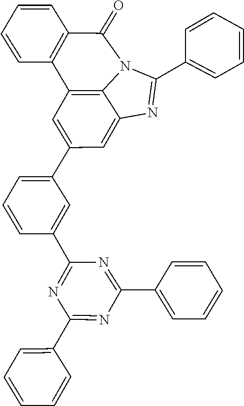 [SMILES: O=c1c2ccccc2c2cc(-c3cccc(-c4nc(-c5ccccc5)nc(-c5ccccc5)n4)c3)cc3nc(-c4ccccc4)n1c32] | 67 |
| 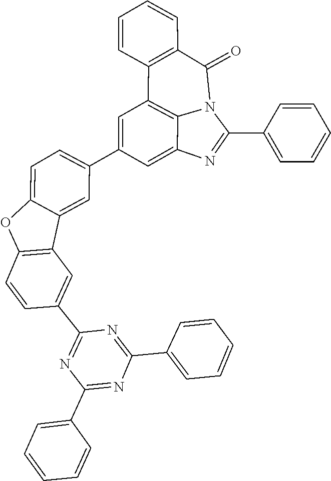 [SMILES: O=c1c2ccccc2c2cc(-c3ccc4oc5ccc(-c6nc(-c7ccccc7)nc(-c7ccccc7)n6)cc5c4c3)cc3nc(-c4ccccc4)n1c32] | 68 |
| 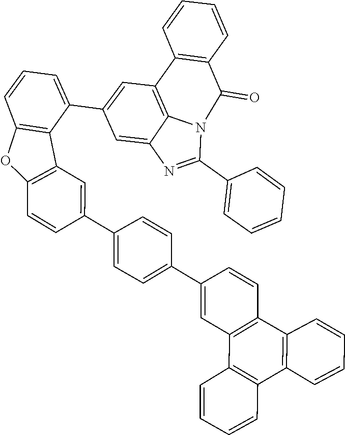 [SMILES: O=c1c2ccccc2c2cc(-c3cccc4oc5ccc(-c6ccc(-c7ccc8c9ccccc9c9ccccc9c8c7)cc6)cc5c34)cc3nc(-c4ccccc4)n1c32] | 69 |
| 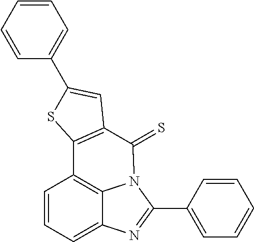 [SMILES: S=c1c2cc(-c3ccccc3)sc2c2cccc3nc(-c4ccccc4)n1c32] | 70 |
| 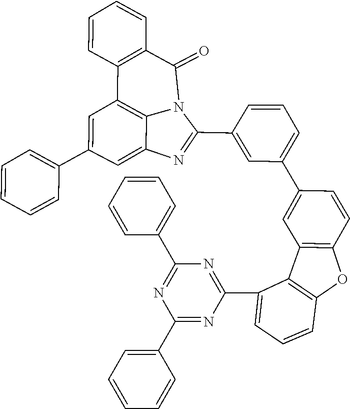 [SMILES: O=c1c2ccccc2c2cc(-c3ccccc3)cc3nc(-c4cccc(-c5ccc6oc7cccc(-c8nc(-c9ccccc9)nc(-c9ccccc9)n8)c7c6c5)c4)n1c32] | 71 |
| 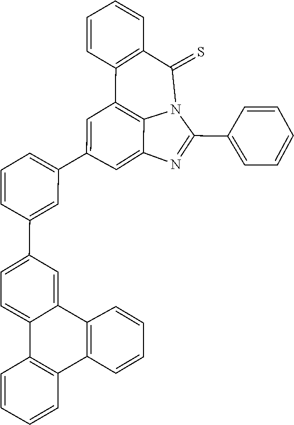 [SMILES: S=c1c2ccccc2c2cc(-c3cccc(-c4ccc5c6ccccc6c6ccccc6c5c4)c3)cc3nc(-c4ccccc4)n1c32] | 72 |
| 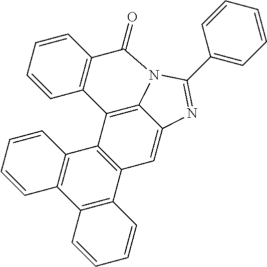 [SMILES: O=c1c2ccccc2c2c3c4ccccc4c4ccccc4c3cc3nc(-c4ccccc4)n1c32] | 73 |
| 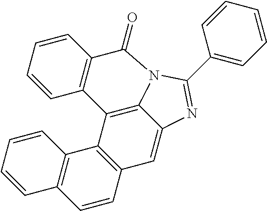 [SMILES: O=c1c2ccccc2c2c3c(ccc4ccccc43)cc3nc(-c4ccccc4)n1c32] | 74 |
| 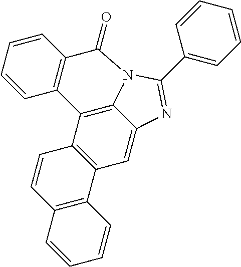 [SMILES: O=c1c2ccccc2c2c3ccc4ccccc4c3cc3nc(-c4ccccc4)n1c32] | 75 |
| 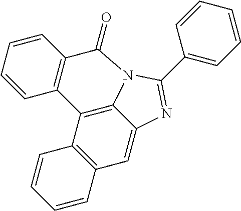 [SMILES: O=c1c2ccccc2c2c3ccccc3cc3nc(-c4ccccc4)n1c32] | 76 |
| 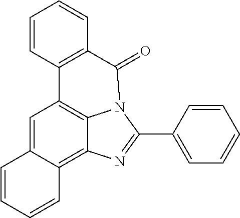 [SMILES: O=c1c2ccccc2c2cc3ccccc3c3nc(-c4ccccc4)n1c23] | 77 |
| 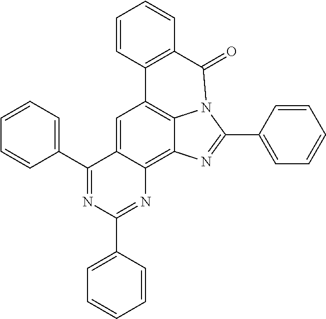 [SMILES: O=c1c2ccccc2c2cc3c(-c4ccccc4)nc(-c4ccccc4)nc3c3nc(-c4ccccc4)n1c23] | 78 |
| 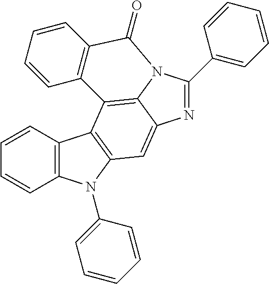 [SMILES: O=c1c2ccccc2c2c3c4ccccc4n(-c4ccccc4)c3cc3nc(-c4ccccc4)n1c32] | 79 |
| 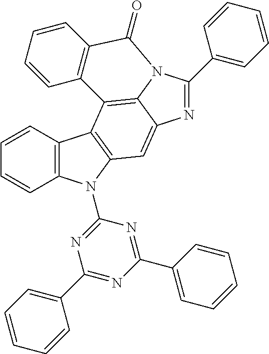 [SMILES: O=c1c2ccccc2c2c3c4ccccc4n(-c4nc(-c5ccccc5)nc(-c5ccccc5)n4)c3cc3nc(-c4ccccc4)n1c32] | 80 |
| 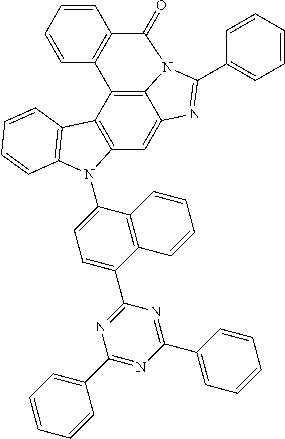 [SMILES: O=c1c2ccccc2c2c3c4ccccc4n(-c4ccc(-c5nc(-c6ccccc6)nc(-c6ccccc6)n5)c5ccccc45)c3cc3nc(-c4ccccc4)n1c32] | 81 |
| 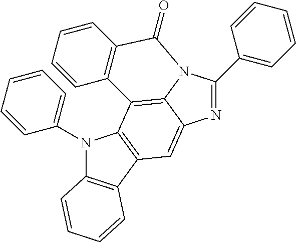 [SMILES: O=c1c2ccccc2c2c3c(cc4nc(-c5ccccc5)n1c42)c1ccccc1n3-c1ccccc1] | 82 |
| 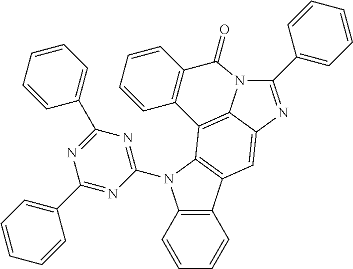 [SMILES: O=c1c2ccccc2c2c3c(cc4nc(-c5ccccc5)n1c42)c1ccccc1n3-c1nc(-c2ccccc2)nc(-c2ccccc2)n1] | 83 |
| 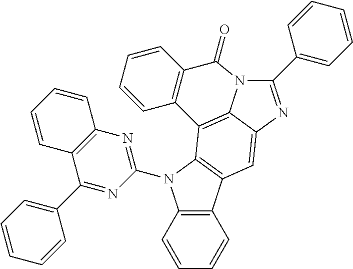 [SMILES: O=c1c2ccccc2c2c3c(cc4nc(-c5ccccc5)n1c42)c1ccccc1n3-c1nc(-c2ccccc2)c2ccccc2n1] | 84 |
| 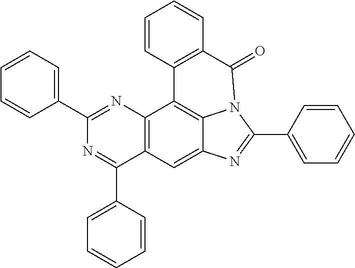 [SMILES: O=c1c2ccccc2c2c3nc(-c4ccccc4)nc(-c4ccccc4)c3cc3nc(-c4ccccc4)n1c32] | 85 |
| 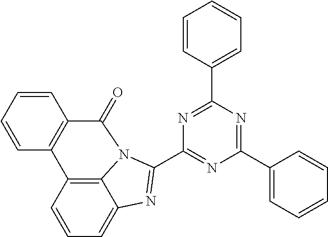 [SMILES: O=c1c2ccccc2c2cccc3nc(-c4nc(-c5ccccc5)nc(-c5ccccc5)n4)n1c32] | 86 |
| 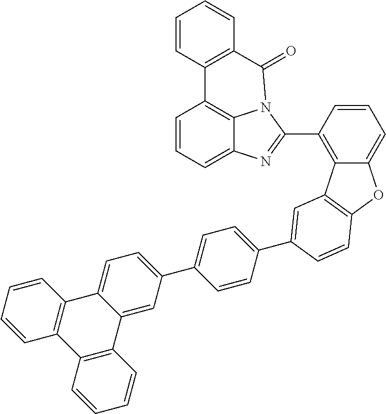 [SMILES: O=c1c2ccccc2c2cccc3nc(-c4cccc5oc6ccc(-c7ccc(-c8ccc9c%10ccccc%10c%10ccccc%10c9c8)cc7)cc6c45)n1c32] | 87 |
| 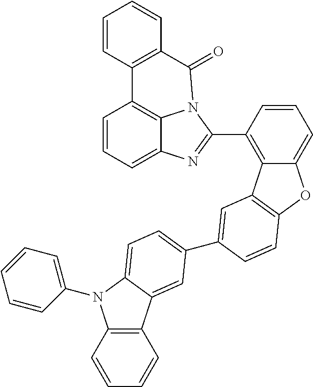 [SMILES: O=c1c2ccccc2c2cccc3nc(-c4cccc5oc6ccc(-c7ccc8c(c7)c7ccccc7n8-c7ccccc7)cc6c45)n1c32] | 88 |
| 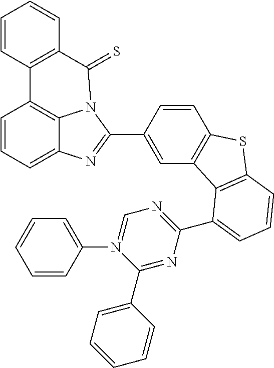 | 89 |
| 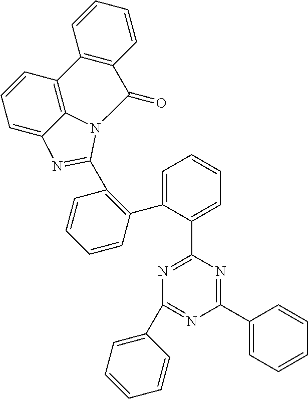 [SMILES: O=c1c2ccccc2c2cccc3nc(-c4ccccc4-c4ccccc4-c4nc(-c5ccccc5)nc(-c5ccccc5)n4)n1c32] | 90 |
| 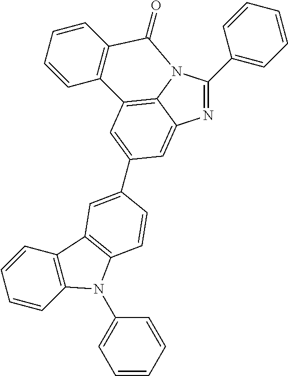 [SMILES: O=c1c2ccccc2c2cc(-c3ccc4c(c3)c3ccccc3n4-c3ccccc3)cc3nc(-c4ccccc4)n1c32] | 91 |
| 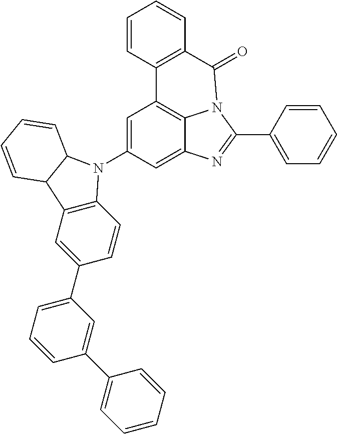 [SMILES: O=c1c2ccccc2c2cc(N3c4ccc(-c5cccc(-c6ccccc6)c5)cc4C4C=CC=CC43)cc3nc(-c4ccccc4)n1c32] | 92 |
| 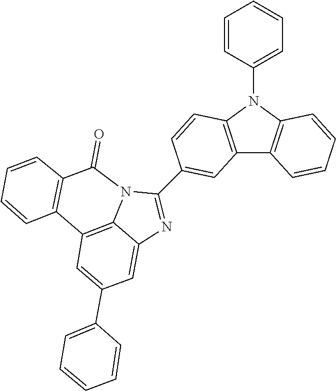 [SMILES: O=c1c2ccccc2c2cc(-c3ccccc3)cc3nc(-c4ccc5c(c4)c4ccccc4n5-c4ccccc4)n1c32] | 93 |
| 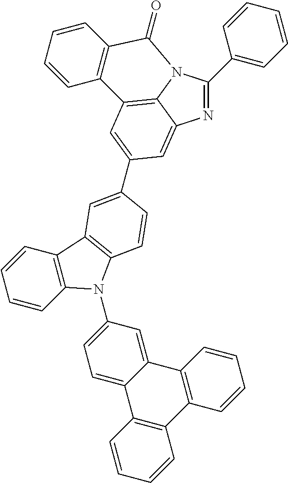 [SMILES: O=c1c2ccccc2c2cc(-c3ccc4c(c3)c3ccccc3n4-c3ccc4c5ccccc5c5ccccc5c4c3)cc3nc(-c4ccccc4)n1c32] | 94 |
| 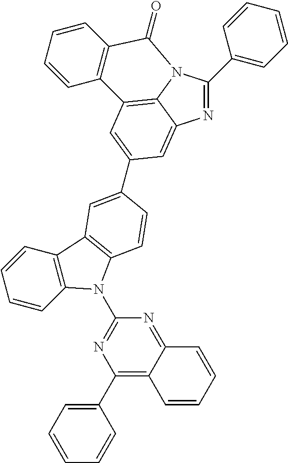 [SMILES: O=c1c2ccccc2c2cc(-c3ccc4c(c3)c3ccccc3n4-c3nc(-c4ccccc4)c4ccccc4n3)cc3nc(-c4ccccc4)n1c32] | 95 |
| 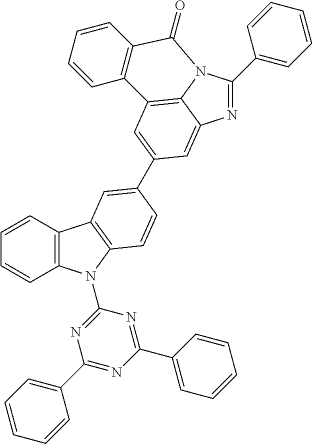 [SMILES: O=c1c2ccccc2c2cc(-c3ccc4c(c3)c3ccccc3n4-c3nc(-c4ccccc4)nc(-c4ccccc4)n3)cc3nc(-c4ccccc4)n1c32] | 96 |
| 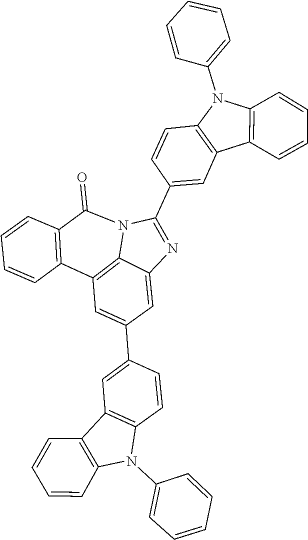 [SMILES: O=c1c2ccccc2c2cc(-c3ccc4c(c3)c3ccccc3n4-c3ccccc3)cc3nc(-c4ccc5c(c4)c4ccccc4n5-c4ccccc4)n1c32] | 97 |
| 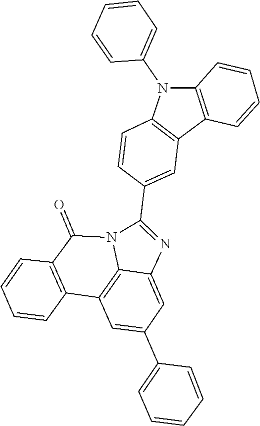 [SMILES: O=c1c2ccccc2c2cc(-c3ccccc3)cc3nc(-c4ccc5c(c4)c4ccccc4n5-c4ccccc4)n1c32] | 98 |
| 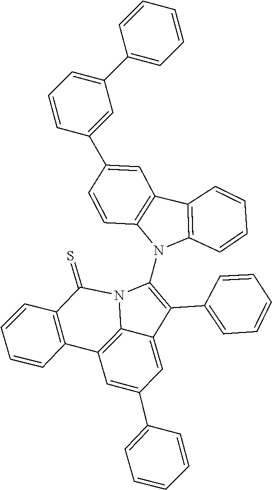 [SMILES: S=c1c2ccccc2c2cc(-c3ccccc3)cc3c(-c4ccccc4)c(-n4c5ccccc5c5cc(-c6cccc(-c7ccccc7)c6)ccc54)n1c32] | 99 |
| 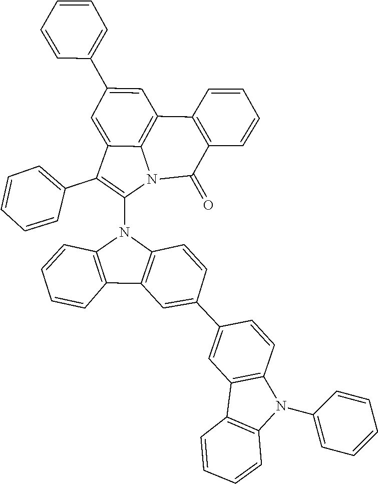 [SMILES: O=c1c2ccccc2c2cc(-c3ccccc3)cc3c(-c4ccccc4)c(-n4c5ccccc5c5cc(-c6ccc7c(c6)c6ccccc6n7-c6ccccc6)ccc54)n1c32] | 100 |
| 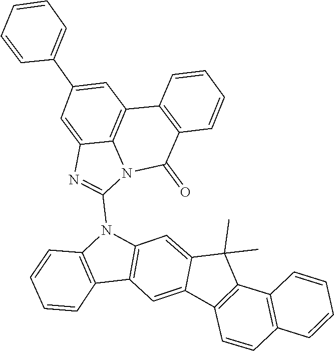 [SMILES: CC1(C)c2cc3c(cc2-c2ccc4ccccc4c21)c1ccccc1n3-c1nc2cc(-c3ccccc3)cc3c4ccccc4c(=O)n1c23] | 101 |
| 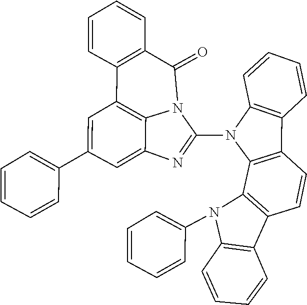 [SMILES: O=c1c2ccccc2c2cc(-c3ccccc3)cc3nc(-n4c5ccccc5c5ccc6c7ccccc7n(-c7ccccc7)c6c54)n1c32] | 102 |
| 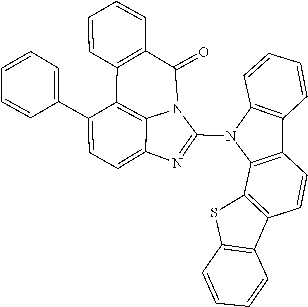 [SMILES: O=c1c2ccccc2c2c(-c3ccccc3)ccc3nc(-n4c5ccccc5c5ccc6c7ccccc7sc6c54)n1c32] | 103 |
| 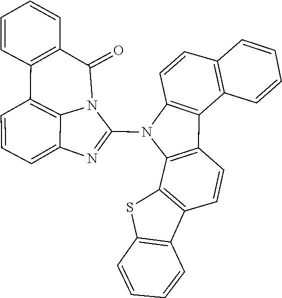 [SMILES: O=c1c2ccccc2c2cccc3nc(-n4c5ccc6ccccc6c5c5ccc6c7ccccc7sc6c54)n1c32] | 104 |
| 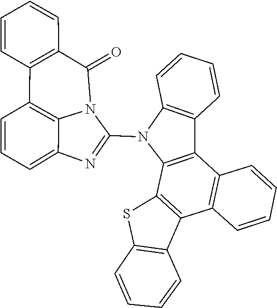 [SMILES: O=c1c2ccccc2c2cccc3nc(-n4c5ccccc5c5c6ccccc6c6c7ccccc7sc6c54)n1c32] | 105 |
| 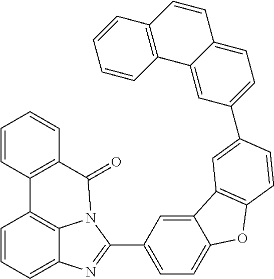 [SMILES: O=c1c2ccccc2c2cccc3nc(-c4ccc5oc6ccc(-c7ccc8ccc9ccccc9c8c7)cc6c5c4)n1c32] | 106 |
| 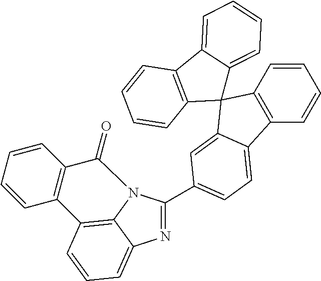 [SMILES: O=c1c2ccccc2c2cccc3nc(-c4ccc5c(c4)C4(c6ccccc6-c6ccccc64)c4ccccc4-5)n1c32] | 107 |
| 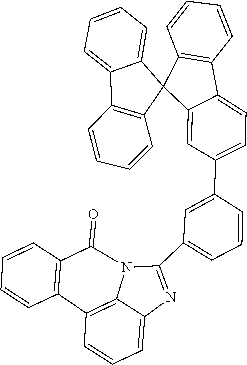 [SMILES: O=c1c2ccccc2c2cccc3nc(-c4cccc(-c5ccc6c(c5)C5(c7ccccc7-c7ccccc75)c5ccccc5-6)c4)n1c32] | 108 |
| 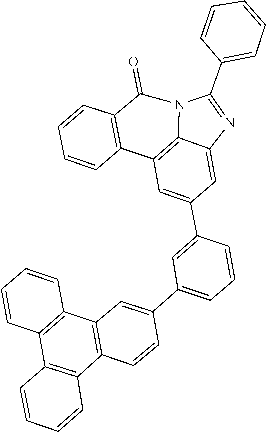 [SMILES: O=c1c2ccccc2c2cc(-c3cccc(-c4ccc5c6ccccc6c6ccccc6c5c4)c3)cc3nc(-c4ccccc4)n1c32] | 109 |
| 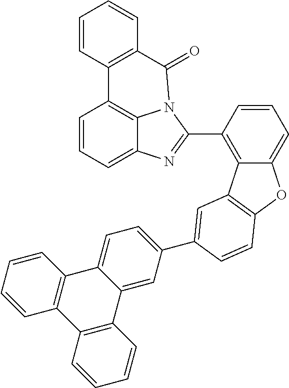 [SMILES: O=c1c2ccccc2c2cccc3nc(-c4cccc5oc6ccc(-c7ccc8c9ccccc9c9ccccc9c8c7)cc6c45)n1c32] | 110 |
| 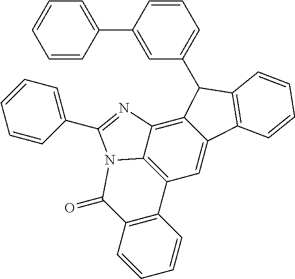 [SMILES: O=c1c2ccccc2c2cc3c(c4nc(-c5ccccc5)n1c42)C(c1cccc(-c2ccccc2)c1)c1ccccc1-3] | 111 |
| 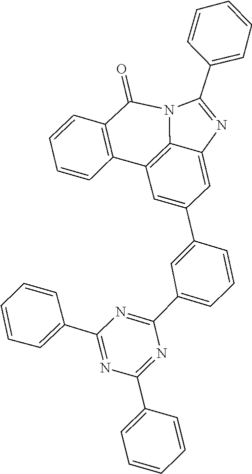 [SMILES: O=c1c2ccccc2c2cc(-c3cccc(-c4nc(-c5ccccc5)nc(-c5ccccc5)n4)c3)cc3nc(-c4ccccc4)n1c32] | 112 |
| 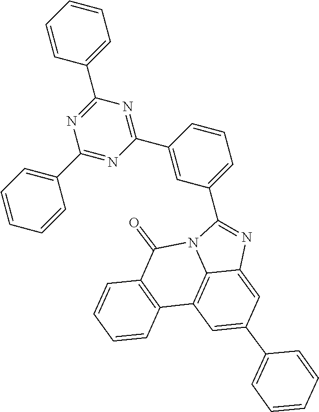 [SMILES: O=c1c2ccccc2c2cc(-c3ccccc3)cc3nc(-c4cccc(-c5nc(-c6ccccc6)nc(-c6ccccc6)n5)c4)n1c32] | 113 |
| 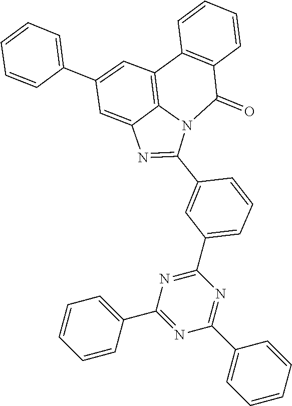 [SMILES: O=c1c2ccccc2c2cc(-c3ccccc3)cc3nc(-c4cccc(-c5nc(-c6ccccc6)nc(-c6ccccc6)n5)c4)n1c32] | 114 |
| 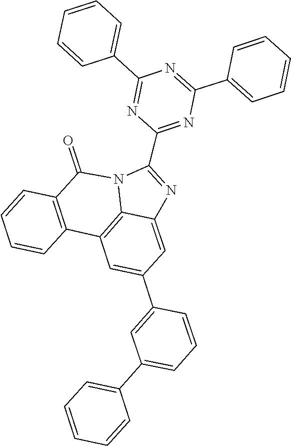 [SMILES: O=c1c2ccccc2c2cc(-c3cccc(-c4ccccc4)c3)cc3nc(-c4nc(-c5ccccc5)nc(-c5ccccc5)n4)n1c32] | 115 |
| 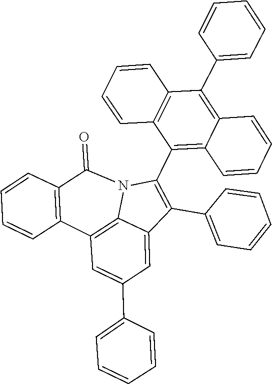 [SMILES: O=c1c2ccccc2c2cc(-c3ccccc3)cc3c(-c4ccccc4)c(-c4c5ccccc5c(-c5ccccc5)c5ccccc45)n1c32] | 116 |
| 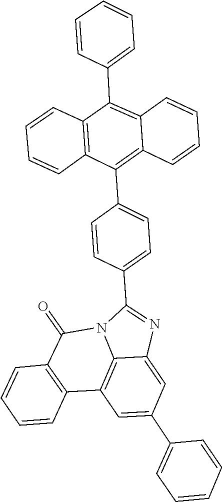 [SMILES: O=c1c2ccccc2c2cc(-c3ccccc3)cc3nc(-c4ccc(-c5c6ccccc6c(-c6ccccc6)c6ccccc56)cc4)n1c32] | 117 |
| 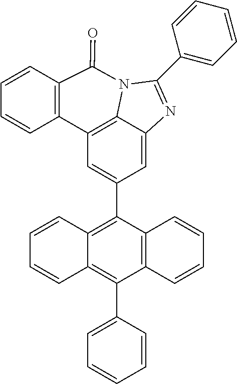 [SMILES: O=c1c2ccccc2c2cc(-c3c4ccccc4c(-c4ccccc4)c4ccccc34)cc3nc(-c4ccccc4)n1c32] | 118 |
| 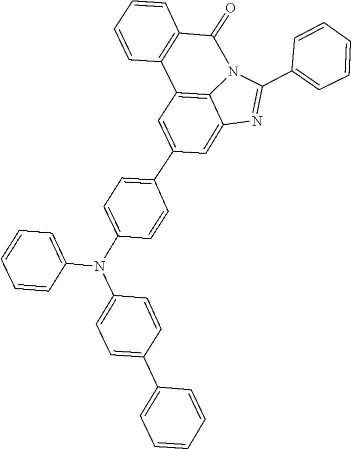 [SMILES: O=c1c2ccccc2c2cc(-c3ccc(N(c4ccccc4)c4ccc(-c5ccccc5)cc4)cc3)cc3nc(-c4ccccc4)n1c32] | 119 |
| 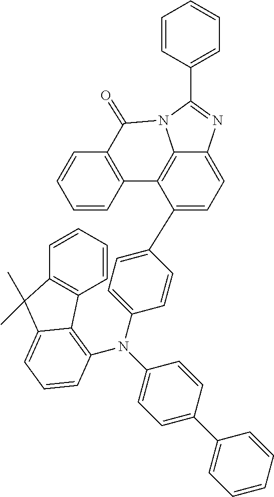 [SMILES: CC1(C)c2ccccc2-c2c(N(c3ccc(-c4ccccc4)cc3)c3ccc(-c4ccc5nc(-c6ccccc6)n6c(=O)c7ccccc7c4c56)cc3)cccc21] | 120 |
| 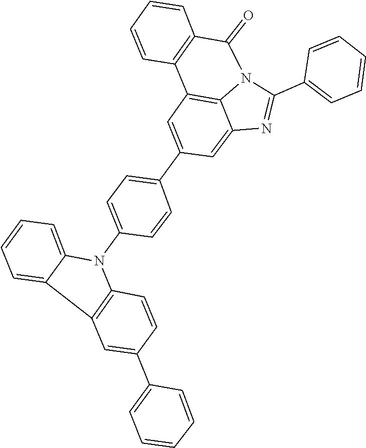 [SMILES: O=c1c2ccccc2c2cc(-c3ccc(-n4c5ccccc5c5cc(-c6ccccc6)ccc54)cc3)cc3nc(-c4ccccc4)n1c32] | 121 |
| 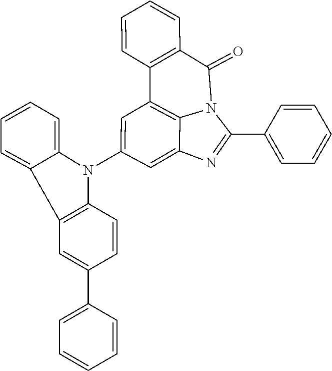 [SMILES: O=c1c2ccccc2c2cc(-n3c4ccccc4c4cc(-c5ccccc5)ccc43)cc3nc(-c4ccccc4)n1c32] | 122 |
| 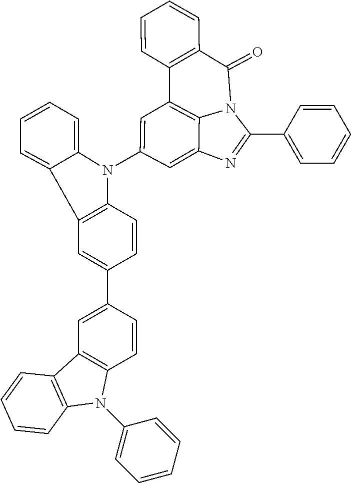 [SMILES: O=c1c2ccccc2c2cc(-n3c4ccccc4c4cc(-c5ccc6c(c5)c5ccccc5n6-c5ccccc5)ccc43)cc3nc(-c4ccccc4)n1c32] | 123 |
| 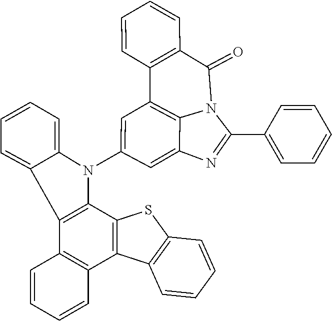 [SMILES: O=c1c2ccccc2c2cc(-n3c4ccccc4c4c5ccccc5c5c6ccccc6sc5c43)cc3nc(-c4ccccc4)n1c32] | 124 |
| 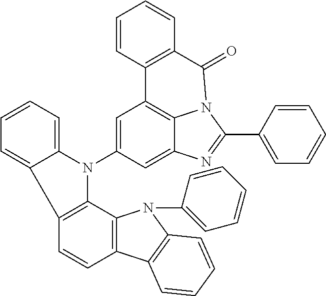 [SMILES: O=c1c2ccccc2c2cc(-n3c4ccccc4c4ccc5c6ccccc6n(-c6ccccc6)c5c43)cc3nc(-c4ccccc4)n1c32] | 125 |
| 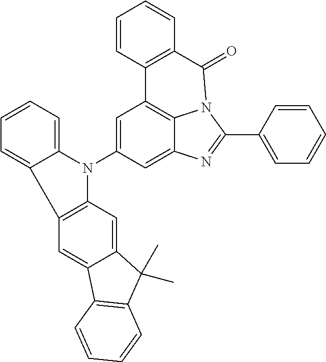 [SMILES: CC1(C)c2ccccc2-c2cc3c4ccccc4n(-c4cc5nc(-c6ccccc6)n6c(=O)c7ccccc7c(c4)c56)c3cc21] | 126 |
| 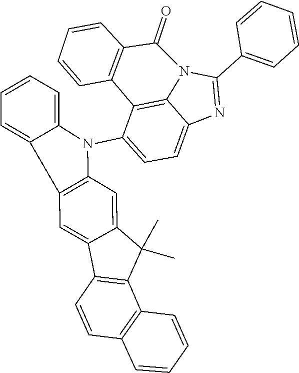 [SMILES: CC1(C)c2cc3c(cc2-c2ccc4ccccc4c21)c1ccccc1n3-c1ccc2nc(-c3ccccc3)n3c(=O)c4ccccc4c1c23] | 127 |
| 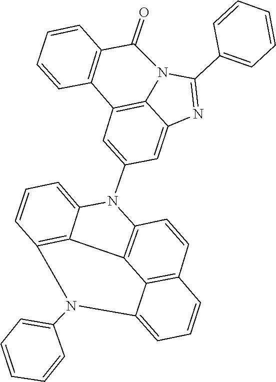 [SMILES: O=c1c2ccccc2c2cc(-n3c4cccc5c4c4c6c(cccc6ccc43)N5c3ccccc3)cc3nc(-c4ccccc4)n1c32] | 128 |
| 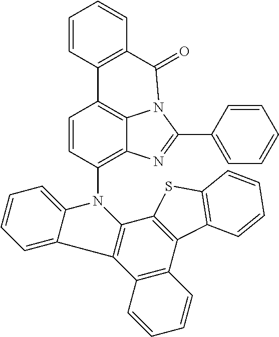 [SMILES: O=c1c2ccccc2c2ccc(-n3c4ccccc4c4c5ccccc5c5c6ccccc6sc5c43)c3nc(-c4ccccc4)n1c32] | 129 |
| 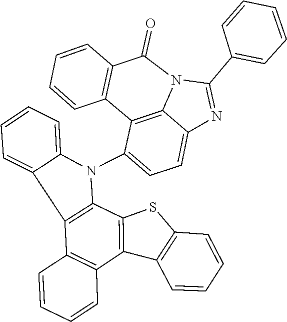 [SMILES: O=c1c2ccccc2c2c(-n3c4ccccc4c4c5ccccc5c5c6ccccc6sc5c43)ccc3nc(-c4ccccc4)n1c32] | 130 |
| 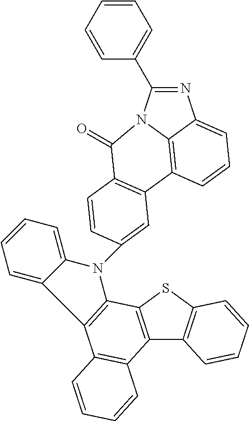 [SMILES: O=c1c2ccc(-n3c4ccccc4c4c5ccccc5c5c6ccccc6sc5c43)cc2c2cccc3nc(-c4ccccc4)n1c32] | 131 |
| 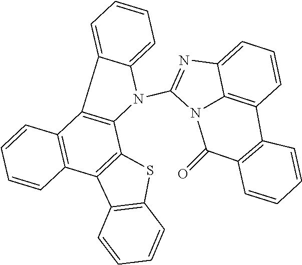 [SMILES: O=c1c2ccccc2c2cccc3nc(-n4c5ccccc5c5c6ccccc6c6c7ccccc7sc6c54)n1c32] | 132 |
| 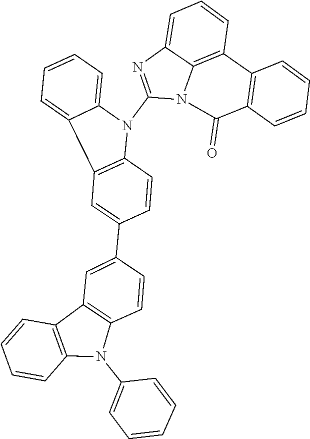 [SMILES: O=c1c2ccccc2c2cccc3nc(-n4c5ccccc5c5cc(-c6ccc7c(c6)c6ccccc6n7-c6ccccc6)ccc54)n1c32] | 133 |
| 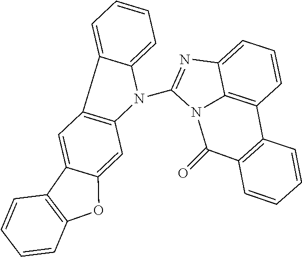 [SMILES: O=c1c2ccccc2c2cccc3nc(-n4c5ccccc5c5cc6c(cc54)oc4ccccc46)n1c32] | 134 |
| 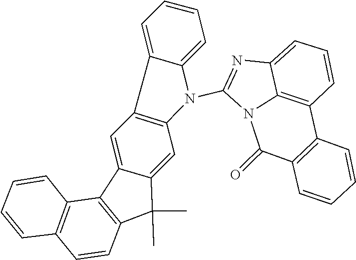 [SMILES: CC1(C)c2cc3c(cc2-c2c1ccc1ccccc21)c1ccccc1n3-c1nc2cccc3c4ccccc4c(=O)n1c23] | 135 |
| 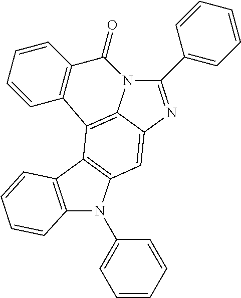 [SMILES: O=c1c2ccccc2c2c3c4ccccc4n(-c4ccccc4)c3cc3nc(-c4ccccc4)n1c32] | 136 |
| 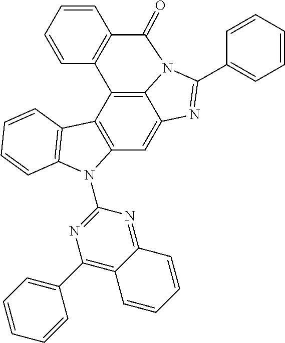 [SMILES: O=c1c2ccccc2c2c3c4ccccc4n(-c4nc(-c5ccccc5)c5ccccc5n4)c3cc3nc(-c4ccccc4)n1c32] | 137 |
| 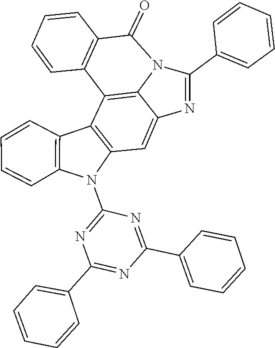 [SMILES: O=c1c2ccccc2c2c3c4ccccc4n(-c4nc(-c5ccccc5)nc(-c5ccccc5)n4)c3cc3nc(-c4ccccc4)n1c32] | 138 |
| 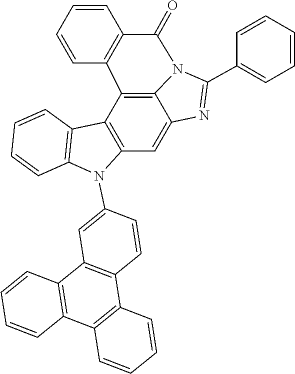 [SMILES: O=c1c2ccccc2c2c3c4ccccc4n(-c4ccc5c6ccccc6c6ccccc6c5c4)c3cc3nc(-c4ccccc4)n1c32] | 139 |
| 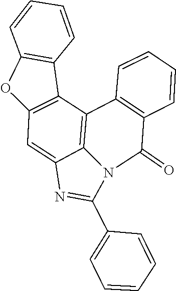 [SMILES: O=c1c2ccccc2c2c3c(cc4nc(-c5ccccc5)n1c42)oc1ccccc13] | 140 |
| 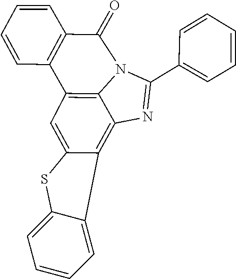 [SMILES: O=c1c2ccccc2c2cc3sc4ccccc4c3c3nc(-c4ccccc4)n1c23] | 141 |
| 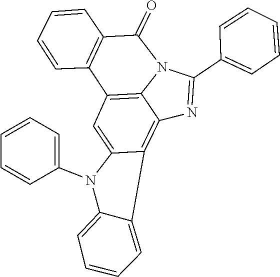 [SMILES: O=c1c2ccccc2c2cc3c(c4ccccc4n3-c3ccccc3)c3nc(-c4ccccc4)n1c23] | 142 |
| 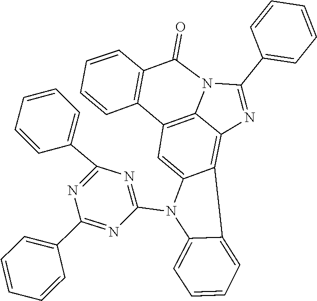 [SMILES: O=c1c2ccccc2c2cc3c(c4ccccc4n3-c3nc(-c4ccccc4)nc(-c4ccccc4)n3)c3nc(-c4ccccc4)n1c23] | 143 |
| 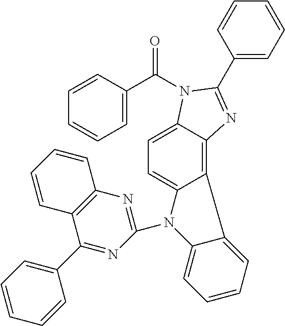 [SMILES: O=C(c1ccccc1)n1c(-c2ccccc2)nc2c3c4ccccc4n(-c4nc(-c5ccccc5)c5ccccc5n4)c3ccc21] | 144 |
| 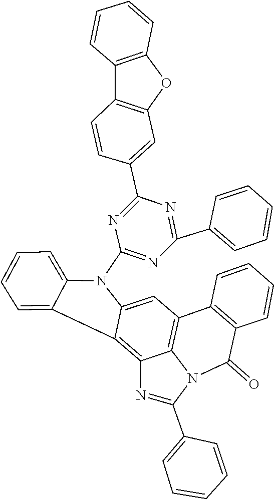 [SMILES: O=c1c2ccccc2c2cc3c(c4ccccc4n3-c3nc(-c4ccccc4)nc(-c4ccc5c(c4)oc4ccccc45)n3)c3nc(-c4ccccc4)n1c23] | 145 |
| 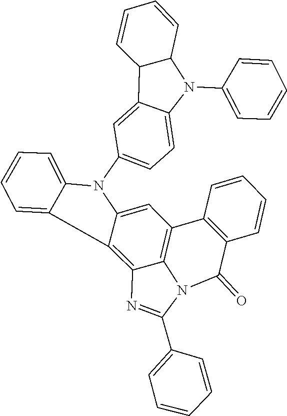 [SMILES: O=c1c2ccccc2c2cc3c(c4ccccc4n3-c3ccc4c(c3)C3C=CC=CC3N4c3ccccc3)c3nc(-c4ccccc4)n1c23] | 146 |
| 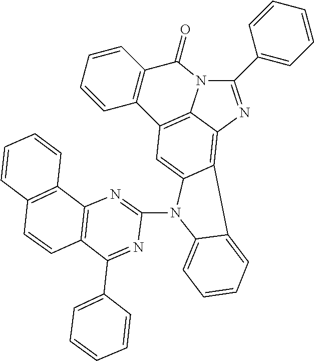 [SMILES: O=c1c2ccccc2c2cc3c(c4ccccc4n3-c3nc(-c4ccccc4)c4ccc5ccccc5c4n3)c3nc(-c4ccccc4)n1c23] | 147 |
| 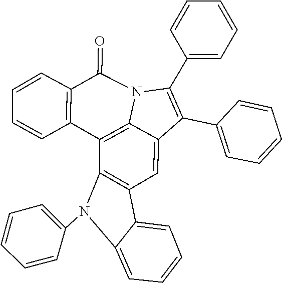 [SMILES: O=c1c2ccccc2c2c3c(cc4c(-c5ccccc5)c(-c5ccccc5)n1c42)c1ccccc1n3-c1ccccc1] | 148 |
| 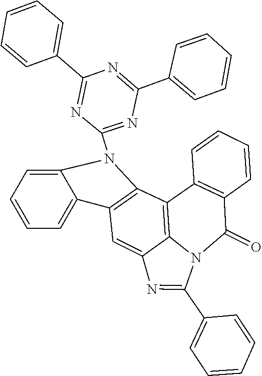 [SMILES: O=c1c2ccccc2c2c3c(cc4nc(-c5ccccc5)n1c42)c1ccccc1n3-c1nc(-c2ccccc2)nc(-c2ccccc2)n1] | 149 |
| 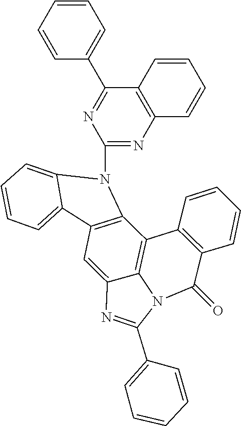 [SMILES: O=c1c2ccccc2c2c3c(cc4nc(-c5ccccc5)n1c42)c1ccccc1n3-c1nc(-c2ccccc2)c2ccccc2n1] | 150 |
| 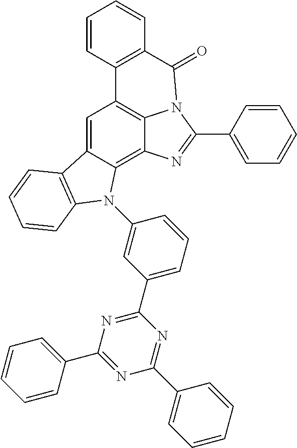 [SMILES: O=c1c2ccccc2c2cc3c4ccccc4n(-c4cccc(-c5nc(-c6ccccc6)nc(-c6ccccc6)n5)c4)c3c3nc(-c4ccccc4)n1c23] | 151 |
| 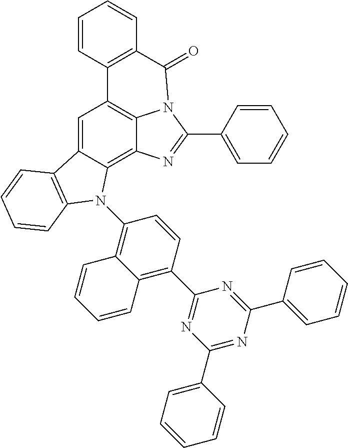 [SMILES: O=c1c2ccccc2c2cc3c4ccccc4n(-c4ccc(-c5nc(-c6ccccc6)nc(-c6ccccc6)n5)c5ccccc45)c3c3nc(-c4ccccc4)n1c23] | 152 |
| 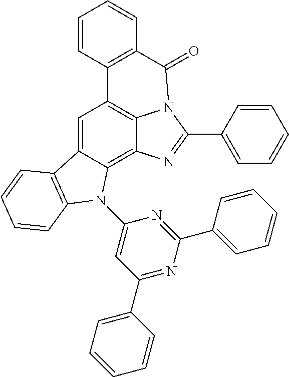 [SMILES: O=c1c2ccccc2c2cc3c4ccccc4n(-c4cc(-c5ccccc5)nc(-c5ccccc5)n4)c3c3nc(-c4ccccc4)n1c23] | 153 |
| 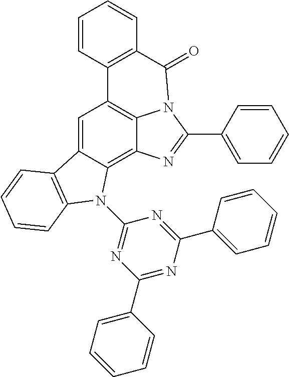 [SMILES: O=c1c2ccccc2c2cc3c4ccccc4n(-c4nc(-c5ccccc5)nc(-c5ccccc5)n4)c3c3nc(-c4ccccc4)n1c23] | 154 |
| 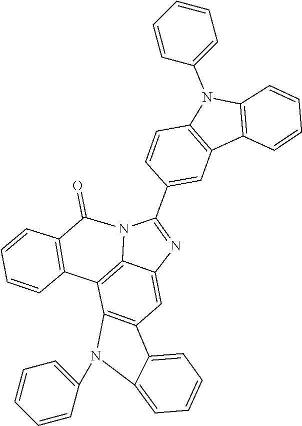 [SMILES: O=c1c2ccccc2c2c3c(cc4nc(-c5ccc6c(c5)c5ccccc5n6-c5ccccc5)n1c42)c1ccccc1n3-c1ccccc1] | 155 |
| 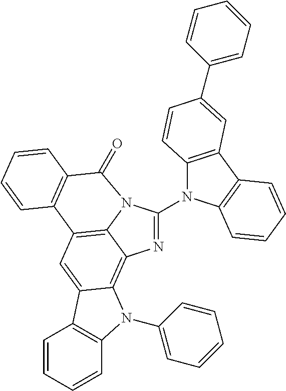 [SMILES: O=c1c2ccccc2c2cc3c4ccccc4n(-c4ccccc4)c3c3nc(-n4c5ccccc5c5cc(-c6ccccc6)ccc54)n1c23] | 156 |
| 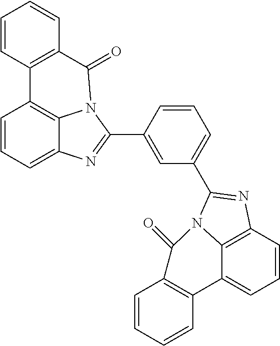 [SMILES: O=c1c2ccccc2c2cccc3nc(-c4cccc(-c5nc6cccc7c8ccccc8c(=O)n5c67)c4)n1c32] | 157 |
| 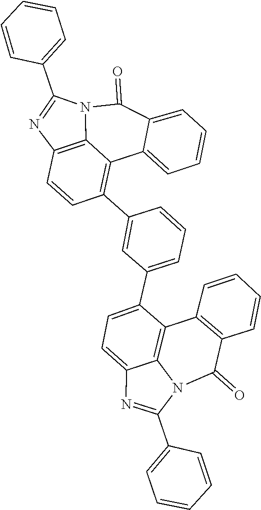 [SMILES: O=c1c2ccccc2c2c(-c3cccc(-c4ccc5nc(-c6ccccc6)n6c(=O)c7ccccc7c4c56)c3)ccc3nc(-c4ccccc4)n1c32] | 158 |
| 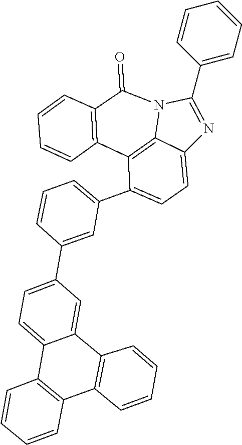 [SMILES: O=c1c2ccccc2c2c(-c3cccc(-c4ccc5c6ccccc6c6ccccc6c5c4)c3)ccc3nc(-c4ccccc4)n1c32] | 159 |
| 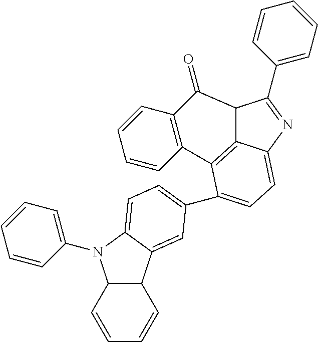 [SMILES: O=C1c2ccccc2-c2c(-c3ccc4c(c3)C3C=CC=CC3N4c3ccccc3)ccc3c2C1C(c1ccccc1)=N3] | 160 |
| 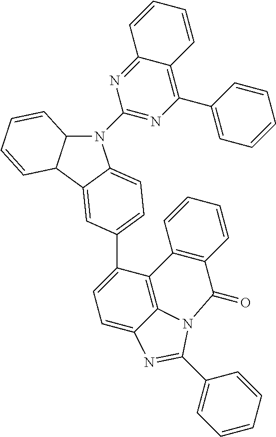 [SMILES: O=c1c2ccccc2c2c(-c3ccc4c(c3)C3C=CC=CC3N4c3nc(-c4ccccc4)c4ccccc4n3)ccc3nc(-c4ccccc4)n1c32] | 161 |
| 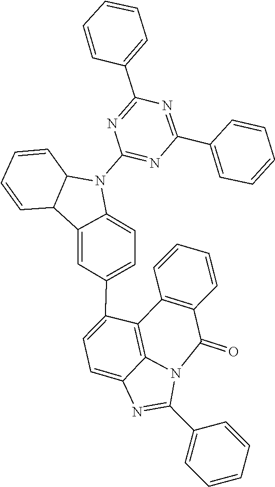 [SMILES: O=c1c2ccccc2c2c(-c3ccc4c(c3)C3C=CC=CC3N4c3nc(-c4ccccc4)nc(-c4ccccc4)n3)ccc3nc(-c4ccccc4)n1c32] | 162 |
| 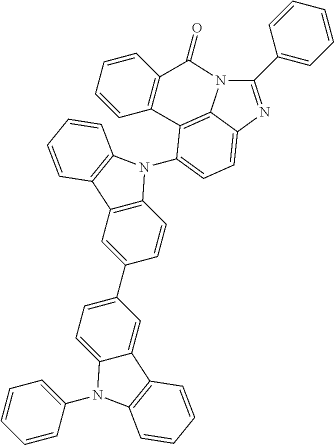 [SMILES: O=c1c2ccccc2c2c(-n3c4ccccc4c4cc(-c5ccc6c(c5)c5ccccc5n6-c5ccccc5)ccc43)ccc3nc(-c4ccccc4)n1c32] | 163 |
| 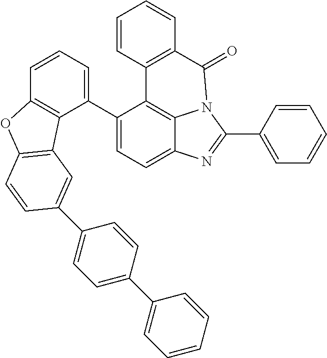 [SMILES: O=c1c2ccccc2c2c(-c3cccc4oc5ccc(-c6ccc(-c7ccccc7)cc6)cc5c34)ccc3nc(-c4ccccc4)n1c32] | 164 |
| 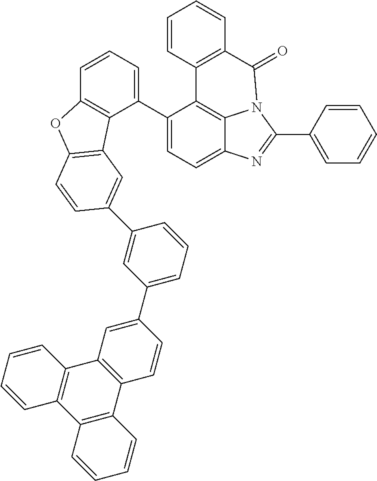 [SMILES: O=c1c2ccccc2c2c(-c3cccc4oc5ccc(-c6cccc(-c7ccc8c9ccccc9c9ccccc9c8c7)c6)cc5c34)ccc3nc(-c4ccccc4)n1c32] | 165 |
| 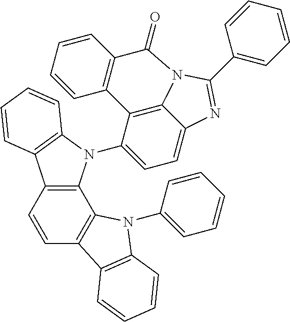 [SMILES: O=c1c2ccccc2c2c(-n3c4ccccc4c4ccc5c6ccccc6n(-c6ccccc6)c5c43)ccc3nc(-c4ccccc4)n1c32] | 166 |
| 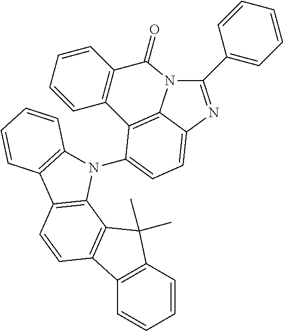 [SMILES: CC1(C)c2ccccc2-c2ccc3c4ccccc4n(-c4ccc5nc(-c6ccccc6)n6c(=O)c7ccccc7c4c56)c3c21] | 167 |
| 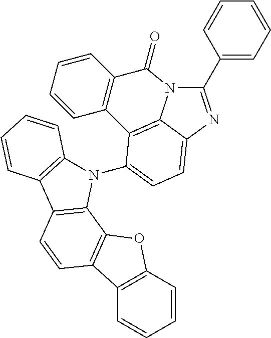 [SMILES: O=c1c2ccccc2c2c(-n3c4ccccc4c4ccc5c6ccccc6oc5c43)ccc3nc(-c4ccccc4)n1c32] | 168 |
| 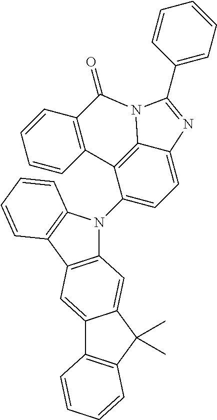 [SMILES: CC1(C)c2ccccc2-c2cc3c4ccccc4n(-c4ccc5nc(-c6ccccc6)n6c(=O)c7ccccc7c4c56)c3cc21] | 169 |
| 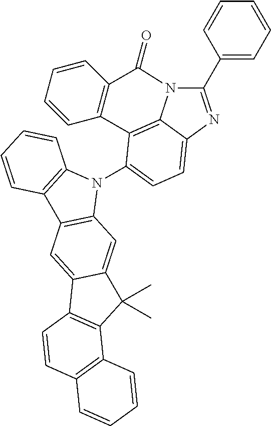 [SMILES: CC1(C)c2cc3c(cc2-c2ccc4ccccc4c21)c1ccccc1n3-c1ccc2nc(-c3ccccc3)n3c(=O)c4ccccc4c1c23] | 170 |
| 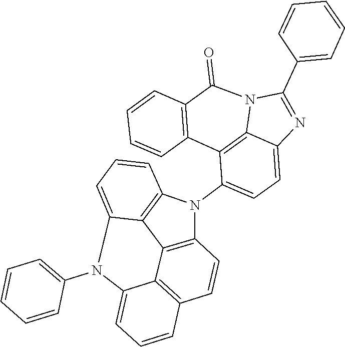 [SMILES: O=c1c2ccccc2c2c(-n3c4cccc5c4c4c6c(cccc6ccc43)N5c3ccccc3)ccc3nc(-c4ccccc4)n1c32] | 171 |
| 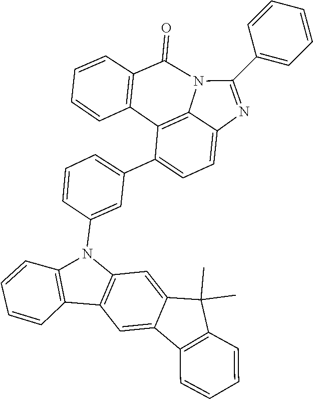 [SMILES: CC1(C)c2ccccc2-c2cc3c4ccccc4n(-c4cccc(-c5ccc6nc(-c7ccccc7)n7c(=O)c8ccccc8c5c67)c4)c3cc21] | 172 |
| 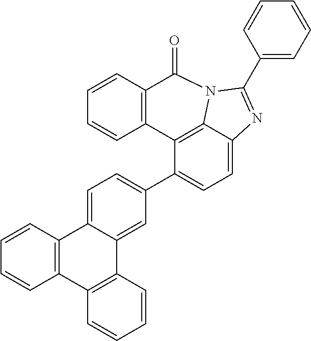 [SMILES: O=c1c2ccccc2c2c(-c3ccc4c5ccccc5c5ccccc5c4c3)ccc3nc(-c4ccccc4)n1c32] | 173 |
| 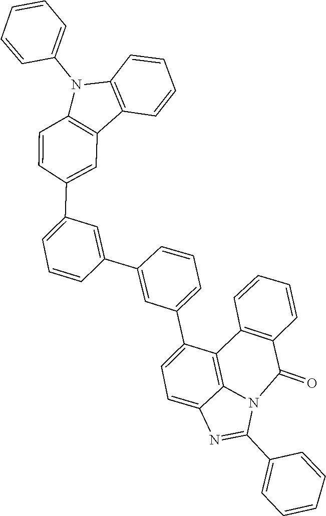 [SMILES: O=c1c2ccccc2c2c(-c3cccc(-c4cccc(-c5ccc6c(c5)c5ccccc5n6-c5ccccc5)c4)c3)ccc3nc(-c4ccccc4)n1c32] | 174 |
| 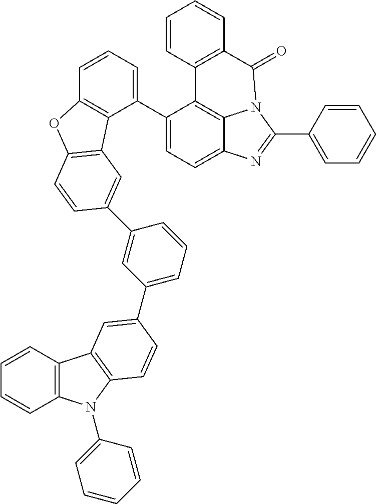 [SMILES: O=c1c2ccccc2c2c(-c3cccc4oc5ccc(-c6cccc(-c7ccc8c(c7)c7ccccc7n8-c7ccccc7)c6)cc5c34)ccc3nc(-c4ccccc4)n1c32] | 175 |
| 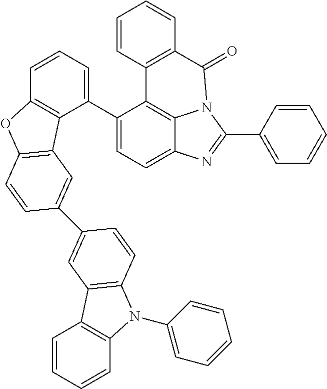 [SMILES: O=c1c2ccccc2c2c(-c3cccc4oc5ccc(-c6ccc7c(c6)c6ccccc6n7-c6ccccc6)cc5c34)ccc3nc(-c4ccccc4)n1c32] | 176 |
| 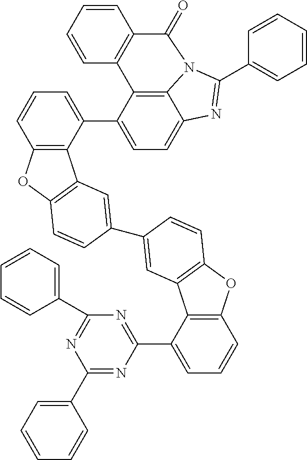 [SMILES: O=c1c2ccccc2c2c(-c3cccc4oc5ccc(-c6ccc7oc8cccc(-c9nc(-c%10ccccc%10)nc(-c%10ccccc%10)n9)c8c7c6)cc5c34)ccc3nc(-c4ccccc4)n1c32] | 177 |
| 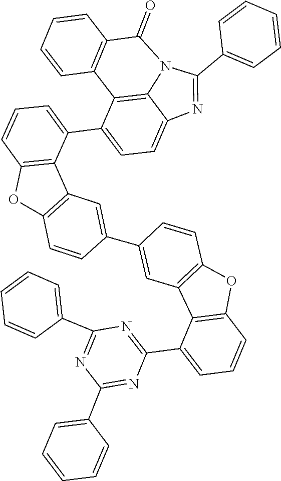 [SMILES: O=c1c2ccccc2c2c(-c3cccc4oc5ccc(-c6ccc7oc8cccc(-c9nc(-c%10ccccc%10)nc(-c%10ccccc%10)n9)c8c7c6)cc5c34)ccc3nc(-c4ccccc4)n1c32] | 178 |
| 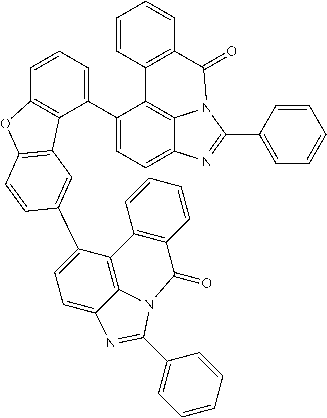 [SMILES: O=c1c2ccccc2c2c(-c3ccc4oc5cccc(-c6ccc7nc(-c8ccccc8)n8c(=O)c9ccccc9c6c78)c5c4c3)ccc3nc(-c4ccccc4)n1c32] | 179 |
| 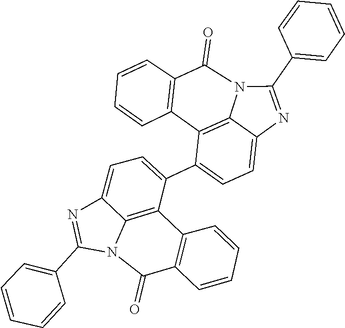 [SMILES: O=c1c2ccccc2c2c(-c3ccc4nc(-c5ccccc5)n5c(=O)c6ccccc6c3c45)ccc3nc(-c4ccccc4)n1c32] | 180 |
| 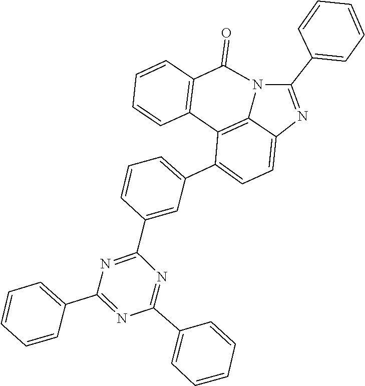 [SMILES: O=c1c2ccccc2c2c(-c3cccc(-c4nc(-c5ccccc5)nc(-c5ccccc5)n4)c3)ccc3nc(-c4ccccc4)n1c32] | 181 |
| 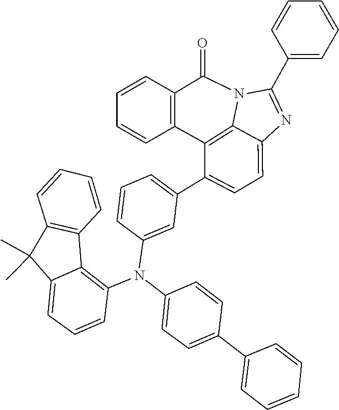 [SMILES: CC1(C)c2ccccc2-c2c(N(c3ccc(-c4ccccc4)cc3)c3cccc(-c4ccc5nc(-c6ccccc6)n6c(=O)c7ccccc7c4c56)c3)cccc21] | 182 |
| 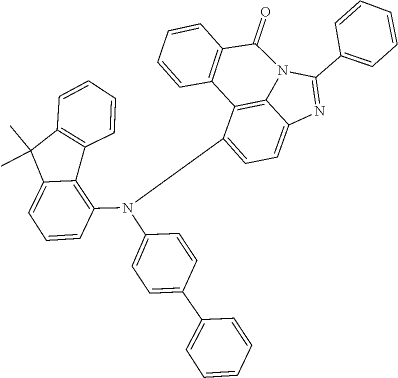 [SMILES: CC1(C)c2ccccc2-c2c(N(c3ccc(-c4ccccc4)cc3)c3ccc4nc(-c5ccccc5)n5c(=O)c6ccccc6c3c45)cccc21] | 183 |
| 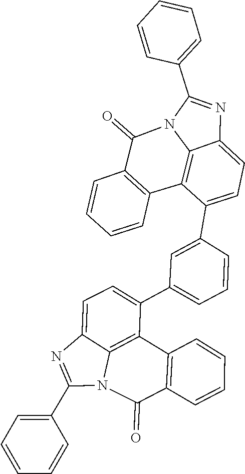 [SMILES: O=c1c2ccccc2c2c(-c3cccc(-c4ccc5nc(-c6ccccc6)n6c(=O)c7ccccc7c4c56)c3)ccc3nc(-c4ccccc4)n1c32] | 184 |
| 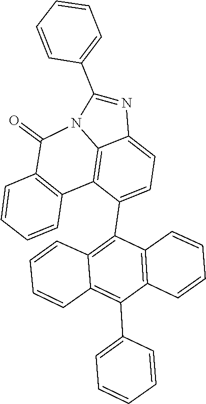 [SMILES: O=c1c2ccccc2c2c(-c3c4ccccc4c(-c4ccccc4)c4ccccc34)ccc3nc(-c4ccccc4)n1c32] | 185 |
| 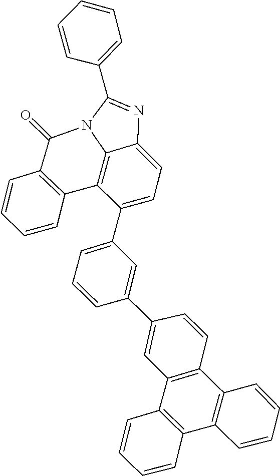 [SMILES: O=c1c2ccccc2c2c(-c3cccc(-c4ccc5c6ccccc6c6ccccc6c5c4)c3)ccc3nc(-c4ccccc4)n1c32] | 186 |
| 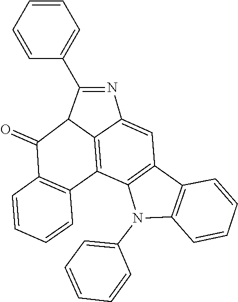 [SMILES: O=C1c2ccccc2-c2c3c(cc4c5ccccc5n(-c5ccccc5)c24)N=C(c2ccccc2)C13] | 187 |
| 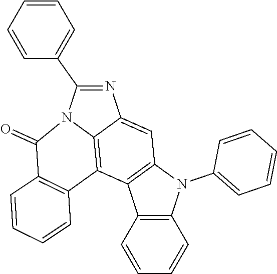 [SMILES: O=c1c2ccccc2c2c3c4ccccc4n(-c4ccccc4)c3cc3nc(-c4ccccc4)n1c32] | 188 |
| 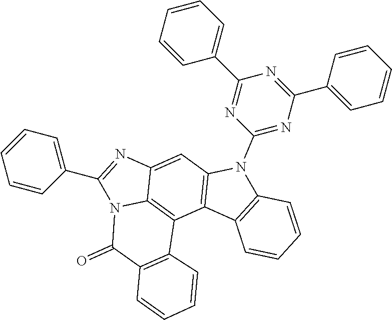 [SMILES: O=c1c2ccccc2c2c3c4ccccc4n(-c4nc(-c5ccccc5)nc(-c5ccccc5)n4)c3cc3nc(-c4ccccc4)n1c32] | 189 |
| 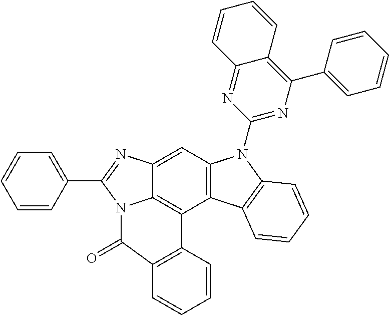 [SMILES: O=c1c2ccccc2c2c3c4ccccc4n(-c4nc(-c5ccccc5)c5ccccc5n4)c3cc3nc(-c4ccccc4)n1c32] | 190 |
| 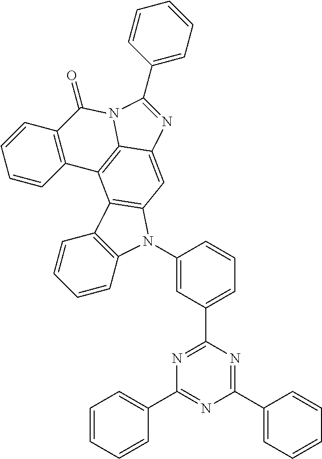 [SMILES: O=c1c2ccccc2c2c3c4ccccc4n(-c4cccc(-c5nc(-c6ccccc6)nc(-c6ccccc6)n5)c4)c3cc3nc(-c4ccccc4)n1c32] | 191 |
| 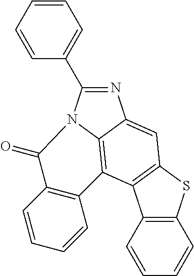 [SMILES: O=c1c2ccccc2c2c3c(cc4nc(-c5ccccc5)n1c42)sc1ccccc13] | 192 |
| 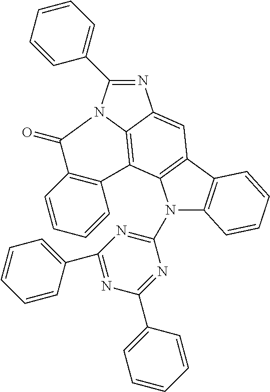 [SMILES: O=c1c2ccccc2c2c3c(cc4nc(-c5ccccc5)n1c42)c1ccccc1n3-c1nc(-c2ccccc2)nc(-c2ccccc2)n1] | 193 |
| 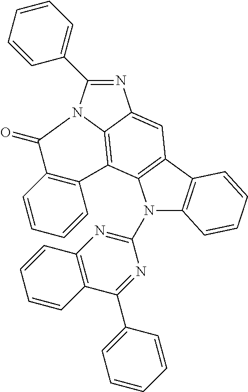 [SMILES: O=c1c2ccccc2c2c3c(cc4nc(-c5ccccc5)n1c42)c1ccccc1n3-c1nc(-c2ccccc2)c2ccccc2n1] | 194 |
| 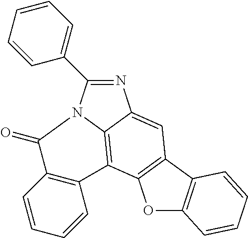 [SMILES: O=c1c2ccccc2c2c3oc4ccccc4c3cc3nc(-c4ccccc4)n1c32] | 195 |
| 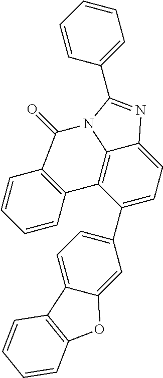 [SMILES: O=c1c2ccccc2c2c(-c3ccc4c(c3)oc3ccccc34)ccc3nc(-c4ccccc4)n1c32] | 196 |
| 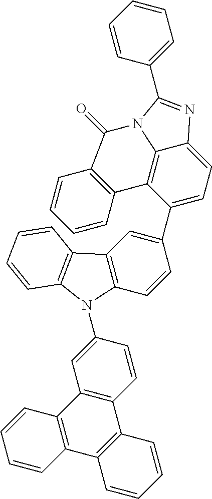 [SMILES: O=c1c2ccccc2c2c(-c3ccc4c(c3)c3ccccc3n4-c3ccc4c5ccccc5c5ccccc5c4c3)ccc3nc(-c4ccccc4)n1c32] | 197 |
| 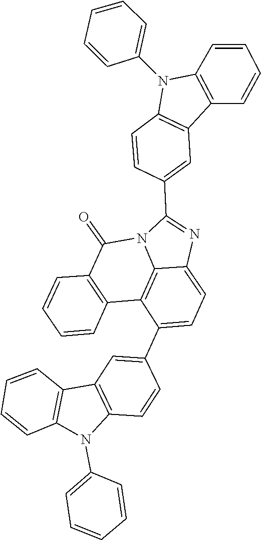 [SMILES: O=c1c2ccccc2c2c(-c3ccc4c(c3)c3ccccc3n4-c3ccccc3)ccc3nc(-c4ccc5c(c4)c4ccccc4n5-c4ccccc4)n1c32] | 198 |
| 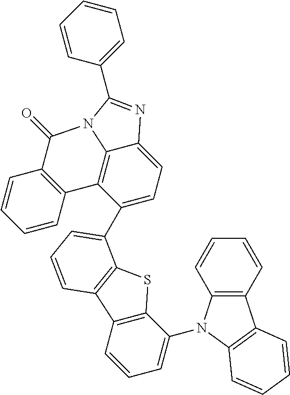 [SMILES: O=c1c2ccccc2c2c(-c3cccc4c3sc3c(-n5c6ccccc6c6ccccc65)cccc34)ccc3nc(-c4ccccc4)n1c32] | 199 |

The compounds of the formula (I) can be prepared by means of known synthesis steps from organic chemistry, for example bromination, Suzuki coupling and Hartwig-Buchwald coupling. Some preferred synthesis methods are shown below by way of example. These can be modified by the person skilled in the art within the scope of their common knowledge and should not be interpreted in a limiting manner.

As shown in scheme 1, compounds of the formula (E-1) can be reacted with an aryl radical in a Suzuki coupling. This step is optional. In a subsequent step, the NH group in the heteroaromatic ring of the compound is reacted with an aromatic having a halogen atom in a benzyl position. Subsequently, a ring closure reaction is conducted with Pd catalysis, and then the methylene group is oxidized with an oxidizing agent to a carbonyl group. Subsequently, it is optionally possible to conduct a halogenation reaction, preferably a bromination, and thereafter a coupling reaction, preferably a Suzuki or Hartwig-Buchwald coupling.

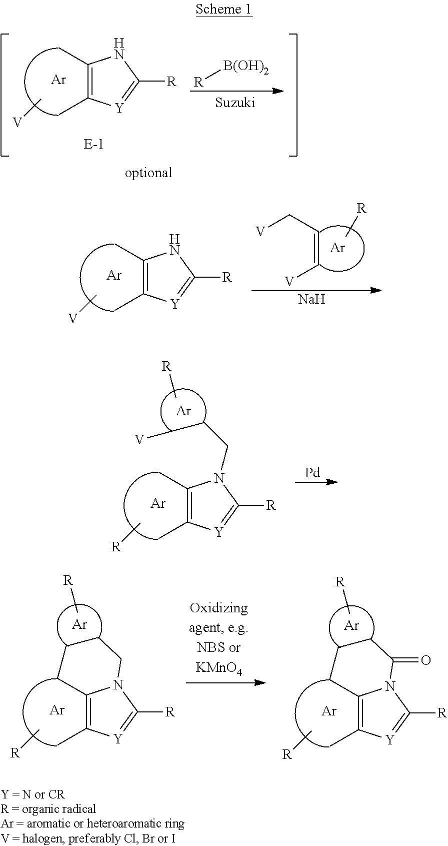

As shown in scheme 2, compounds of the formula (E-1) can be reacted with an aryl radical in a Suzuki coupling. This step is optional. In a subsequent step, the NH group in the heteroaromatic ring of the compound is reacted with an aryl or heteroaryl-substituted acid halide. Subsequently, a ring closure reaction is conducted, preferably with tributyltin hydride (Bu₃SnH) or Pd(PPh₃)₄ plus base, e.g. potassium acetate. This may optionally be followed by performance of a halogenation reaction, preferably a bromination, and thereafter a coupling reaction, preferably a Suzuki or Hartwig-Buchwald coupling.

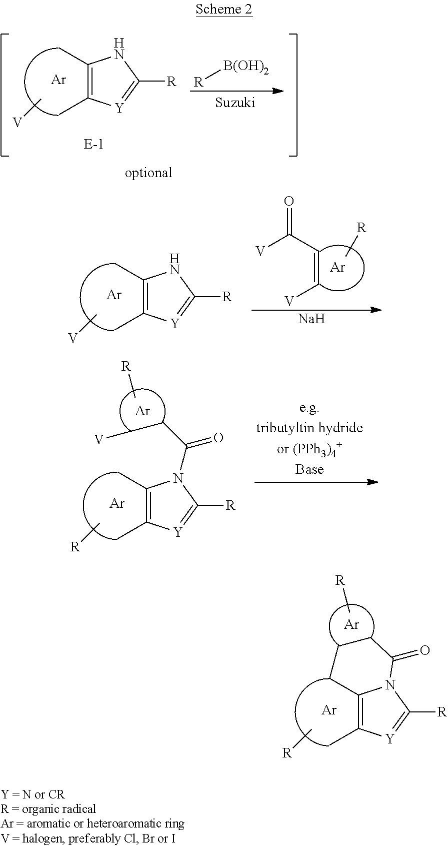

Compounds of the formula (E-3), some of which are commercially available, can be reacted directly in a Suzuki coupling with a boronic acid to give a compound of the formula (I) in which both Z groups are CR⁴ (Scheme 3).

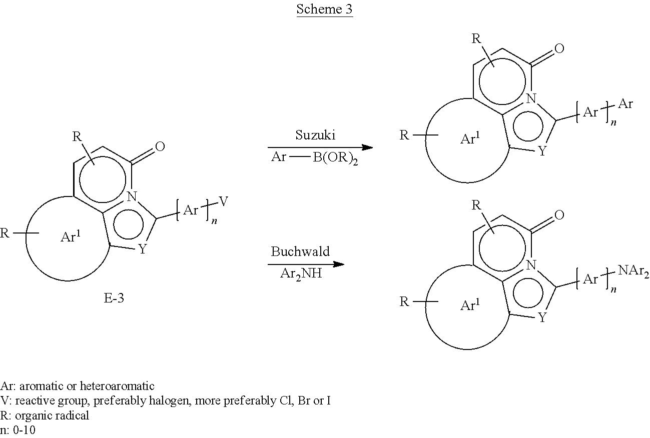

The present application thus provides a process for preparing a compound of the formula (I), characterized in that i) an imidazole or benzimidazole derivative is reacted with an aryl or heteroaryl compound having a halogen, preferably CI, Br or I, in a benzyl position, and ii) a ring closure reaction is conducted under Pd catalysis, and iii) a methylene group in the ring formed is oxidized to a carbonyl group.

Preferably, the aryl or heteroaryl compound, in the halogen in the benzyl position, has a further halogen substituent bonded directly to the aromatic or heteroaromatic ring, preferably in ortho position to the group bonded to the halogen atom in the benzyl position.

Preferably, steps i) to iii) are conducted in the sequence specified and in direct succession.

The present application further provides an alternative process for preparing a compound of the formula (I), characterized in that iv) an imidazole or benzimidazole derivative is reacted with an aryl or heteroaryl compound having a carbonyl halide group, preferably a carbonyl chloride group, carbonyl bromide group or carbonyl iodide group, and v) a ring closure reaction is conducted, preferably with a tin organyl or with Pd⁰.

Preferably, the aryl or heteroaryl compound, in the carbonyl halide group, has a halogen substituent bonded directly to the aromatic or heteroaromatic ring, preferably in ortho position to the carbonyl halide group.

Preferably, steps iv) and v) are conducted in the sequence specified and in direct succession.

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (-)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, 2-methylbiphenyl, 3-methylbiphenyl, 1-methylnaphthalene, 1-ethylnaphthalene, ethyl octanoate, diethyl sebacate, octyl octanoate, heptylbenzene, menthyl isovalerate, cyclohexyl hexanoate or mixtures of these solvents.

The present invention therefore further provides a formulation comprising a compound of the invention and at least one further compound. The further compound may, for example, be a solvent, especially one of the abovementioned solvents or a mixture of these solvents. The further compound may alternatively be at least one further organic or inorganic compound which is likewise used in the electronic device, for example an emitting compound and/or a further matrix material. Suitable emitting compounds and further matrix materials are listed at the back in connection with the organic electroluminescent device. This further compound may also be polymeric.

The compounds of the invention are suitable for use in an electronic device, especially in an organic electroluminescent device.

The present invention therefore further provides for the use of a compound of the invention in an electronic device, especially in an organic electroluminescent device. The compound of the invention is defined as follows: Compound of a formula (I)

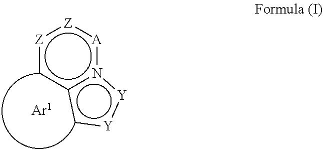

where the variables that occur are as follows:

A is C═O,C═S,C═NR⁰,P(═O)R⁰,SO or SO₂;

Y is the same or different at each instance and is selected from N and CR¹;

Ar¹ is an aromatic ring system which has 6 to 40 aromatic ring atoms and is substituted by R² radicals, and which is fused onto the rest of the formula (I) via the three carbon atoms shown in formula (I), or a heteroaromatic ring system which has 5 to 40 aromatic ring atoms and is substituted by R² radicals, and which is fused onto the rest of the formula (I) via the three carbon atoms shown in formula (I);

Z is the same or different at each instance and is selected from CR⁴ and N, or the Z—Z unit represents a unit of formula (Ar²)

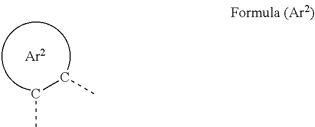

where Ar² is selected from aromatic ring systems which have 6 to 40 aromatic ring atoms and are substituted by R³ radicals, and which include the C—C unit, and heteroaromatic ring systems which have 5 to 40 aromatic ring atoms and are substituted by R³ radicals, and which include the C—C unit, and where the dotted lines are the bonds of the Z—Z unit to the rest of the formula;

R⁰ is the same or different at each instance and is selected from straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C↓C—, Si(R⁵)₂, C═O, C═NR⁵, —C(═O)O—, —C(═O)NR⁵—, NR⁵, P(═O)(R⁵), —O—, —S—, SO or SO₂;

R¹ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C↓C—, Si(R⁵)₂, C═O, C═NR⁵, —C(═O)O—, —C(═O)NR⁵—, NR⁵, P(═O)(R⁵), —O—, —S—, SO or SO₂;

R² is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more radicals selected from R², R³ and R⁴ may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;

R³ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more radicals selected from R², R³ and R⁴ may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;

R⁴ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R², R³ and R⁴ radicals may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;

R⁵ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁵ radicals may be joined to one another and may form a ring; where the alkyl, alkoxy, alkenyl and alkynyl groups mentioned and the aromatic ring systems and heteroaromatic ring systems mentioned are each substituted by R⁶ radicals; and where one or more CH₂ groups in the alkyl, alkoxy, alkenyl and alkynyl groups mentioned may be replaced by —R⁶C═CR⁶—, —C↓C—, Si(R⁶)₂, C═O, C═NR⁶, —C(═O)O—, —C(═O)NR⁶—, NR⁶, P(═O)(R⁶), —O—, —S—, SO or SO₂;

R⁶ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁶ radicals may be joined to one another and may form a ring; and where the alkyl, alkoxy, alkenyl and alkynyl groups, aromatic ring systems and heteroaromatic ring systems mentioned may be substituted by one or more radicals selected from F and CN.

The present invention still further provides an electronic device comprising at least one compound of the formula (I), as defined above.

An electronic device in the context of the present invention is a device comprising at least one layer comprising at least one organic compound.

This component may also comprise inorganic materials or else layers formed entirely from inorganic materials.

The electronic device is preferably selected from the group consisting of organic electroluminescent devices (OLEDs), organic integrated circuits (O—ICs), organic field-effect transistors (O-FETs), organic thin-film transistors (O-TFTs), organic light-emitting transistors (O-LETs), organic solar cells (O—SCs), dye-sensitized organic solar cells (DSSCs), organic optical detectors, organic photoreceptors, organic field-quench devices (O-FQDs), light-emitting electrochemical cells (LECs), organic laser diodes (O-lasers) and organic plasmon emitting devices, but preferably organic electroluminescent devices (OLEDs), more preferably phosphorescent OLEDs.

The organic electroluminescent device comprises cathode, anode and at least one emitting layer. Apart from these layers, it may also comprise further layers, for example in each case one or more hole injection layers, hole transport layers, hole blocker layers, electron transport layers, electron injection layers, exciton blocker layers, electron blocker layers and/or charge generation layers. It is likewise possible for interlayers having an exciton-blocking function, for example, to be introduced between two emitting layers. However, it should be pointed out that not necessarily every one of these layers need be present. In this case, it is possible for the organic electroluminescent device to contain an emitting layer, or for it to contain a plurality of emitting layers. If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are systems having three emitting layers, where the three layers show blue, green and orange or red emission. The organic electroluminescent device of the invention may also be a tandem OLED, especially for white-emitting OLEDs.

The compound of the invention according to the above-detailed embodiments may be used in different layers, according to the exact structure. Preference is given to an organic electroluminescent device comprising a compound of formula (I) or the above-recited preferred embodiments in an emitting layer as matrix material for phosphorescent emitters or for emitters that exhibit TADF (thermally activated delayed fluorescence), especially for phosphorescent emitters. In this case, the organic electroluminescent device may contain an emitting layer, or it may contain a plurality of emitting layers, where at least one emitting layer contains at least one compound of the invention as matrix material. In addition, the compound of the invention can also be used in an electron transport layer and/or in a hole blocker layer and/or in a hole transport layer and/or in an exciton blocker layer.

When the compound of the invention is used as matrix material for a phosphorescent compound in an emitting layer, it is preferably used in combination with one or more phosphorescent materials (triplet emitters). Phosphorescence in the context of this invention is understood to mean luminescence from an excited state having higher spin multiplicity, i.e. a spin state >1, especially from an excited triplet state. In the context of this application, all luminescent complexes with transition metals or lanthanides, especially all iridium, platinum and copper complexes, shall be regarded as phosphorescent compounds.

The mixture of the compound of the invention and the emitting compound contains between 99% and 1% by volume, preferably between 98% and 10% by volume, more preferably between 97% and 60% by volume and especially between 95% and 80% by volume of the compound of the invention, based on the overall mixture of emitter and matrix material. Correspondingly, the mixture contains between 1% and 99% by volume, preferably between 2% and 90% by volume, more preferably between 3% and 40% by volume and especially between 5% and 20% by volume of the emitter, based on the overall mixture of emitter and matrix material.

A further preferred embodiment of the present invention is the use of the compound of the invention as matrix material for a phosphorescent emitter in combination with a further matrix material. Suitable matrix materials which can be used in combination with the inventive compounds are aromatic ketones, aromatic phosphine oxides or aromatic sulfoxides or sulfones, for example according to WO 2004/013080, WO 2004/093207, WO 2006/005627 or WO 2010/006680, triarylamines, carbazole derivatives, e.g. CBP (N,N-biscarbazolylbiphenyl) or the carbazole derivatives disclosed in WO 2005/039246, US 2005/0069729, JP 2004/288381, EP 1205527, WO 2008/086851 or WO 2013/041176, indolocarbazole derivatives, for example according to WO 2007/063754 or WO 2008/056746, indenocarbazole derivatives, for example according to WO 2010/136109, WO 2011/000455, WO 2013/041176 or WO 2013/056776, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2007/063754, WO 2008/056746, WO 2010/015306, WO 2011/057706, WO 2011/060859 or WO 2011/060877, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to WO 2011/042107, WO 2011/060867, WO 2011/088877 and WO 2012/143080, triphenylene derivatives, for example according to WO 2012/048781, or dibenzofuran derivatives, for example according to WO 2015/169412, WO 2016/015810, WO 2016/023608, WO 2017/148564 or WO 2017/148565. It is likewise possible for a further phosphorescent emitter having shorter-wavelength emission than the actual emitter to be present as co-host in the mixture, or a compound not involved in charge transport to a significant extent, if at all, as described, for example, in WO 2010/108579.

In a preferred embodiment of the invention, the materials are used in combination with a further matrix material. Preferred co-matrix materials, especially when the compound of the invention is substituted by an electron-deficient heteroaromatic ring system, are selected from the group of the biscarbazoles, the bridged carbazoles, the triarylamines, the dibenzofuranyl-carbazole derivatives or dibenzofuranyl-amine derivatives and the carbazoleamines.

Preferred biscarbazoles are the structures of the following formulae (21) and (22):

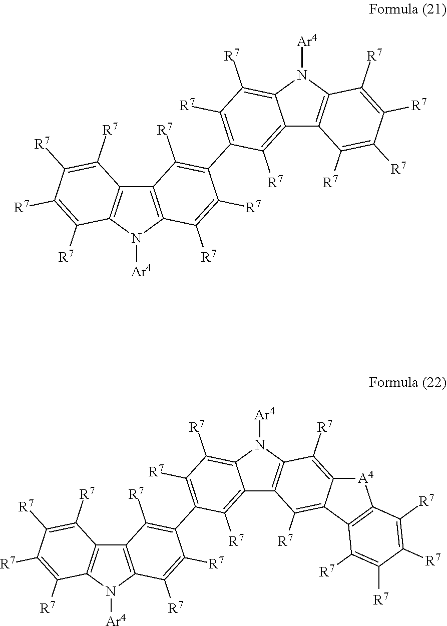

where Ar⁴ is the same or different at each instance and is selected from aromatic ring systems which have 6 to 40 aromatic ring atoms and are substituted by R⁵ radicals, and heteroaromatic ring systems which have 5 to 40 aromatic ring atoms and are substituted by R⁵ radicals; and

A⁴ is selected from C(R⁵)₂, NR⁵, O or S, and is preferably C(R⁵)₂; and

R⁷ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁷ radicals may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C↓C—, Si(R⁵)₂, C═O, C═NR⁵, —C(═O)O—, —C(═O)NR⁵—, NR⁵, P(═O)(R⁵), —O—, —S—, SO or SO₂; and

where the other groups are as defined above.

Preferred embodiments of the compounds of the formulae (21) and (22) are the compounds of the following formulae (21a) and (22a):

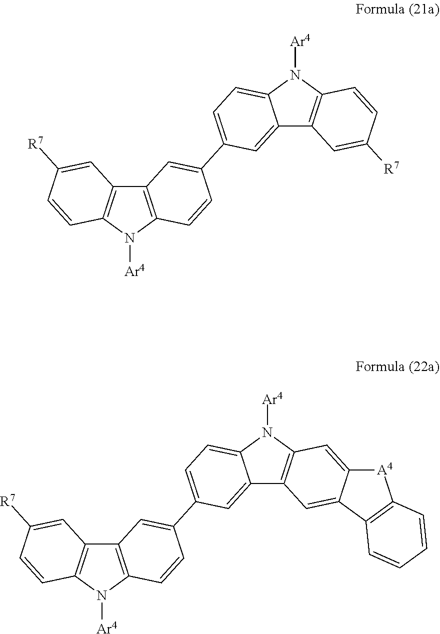

where the symbols used have the definitions given above.

Examples of suitable compounds of formulae (21) and (22) are the compounds depicted below:

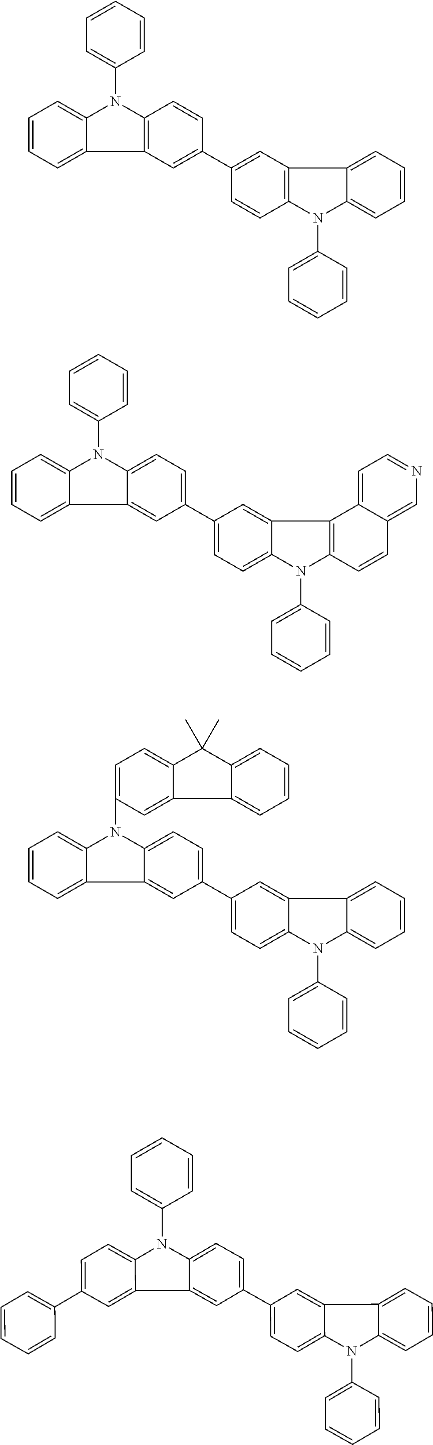 [SMILES: c1ccc(-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4c6ccncc6ccc4n5-c4ccccc4)ccc32)cc1 ; CC1(C)c2ccccc2-c2cc(-n3c4ccccc4c4cc(-c5ccc6c(c5)c5ccccc5n6-c5ccccc5)ccc43)ccc21 ; c1ccc(-c2ccc3c(c2)c2cc(-c4ccc5c(c4)c4ccccc4n5-c4ccccc4)ccc2n3-c2ccccc2)cc1]

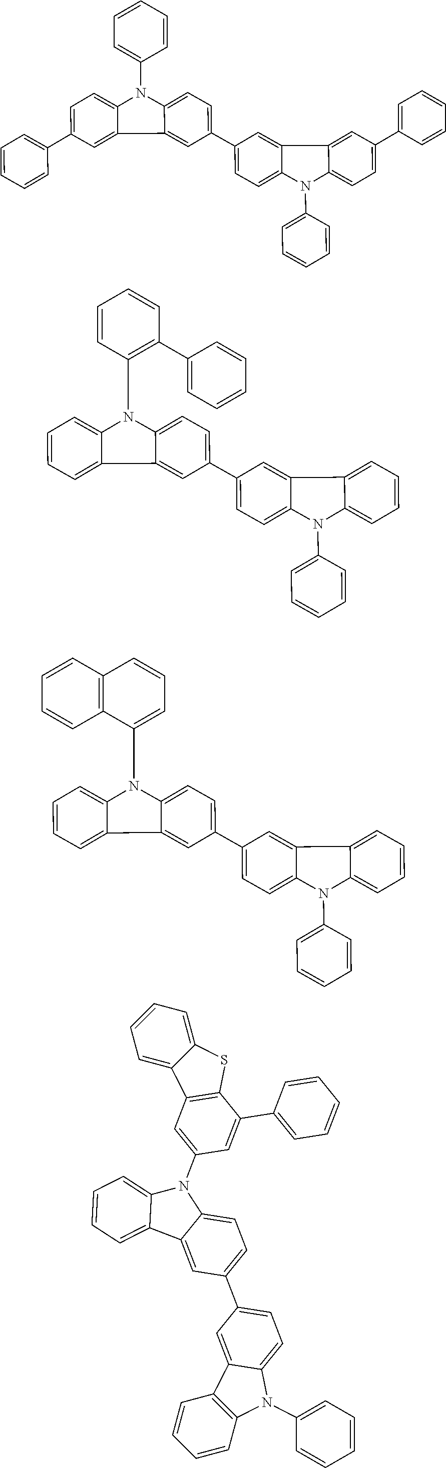 [SMILES: c1ccc(-c2ccc3c(c2)c2cc(-c4ccc5c(c4)c4cc(-c6ccccc6)ccc4n5-c4ccccc4)ccc2n3-c2ccccc2)cc1 ; c1ccc(-c2ccccc2-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5ccccc45)ccc32)cc1 ; c1ccc(-c2cc(-n3c4ccccc4c4cc(-c5ccc6c(c5)c5ccccc5n6-c5ccccc5)ccc43)cc3c2sc2ccccc23)cc1]

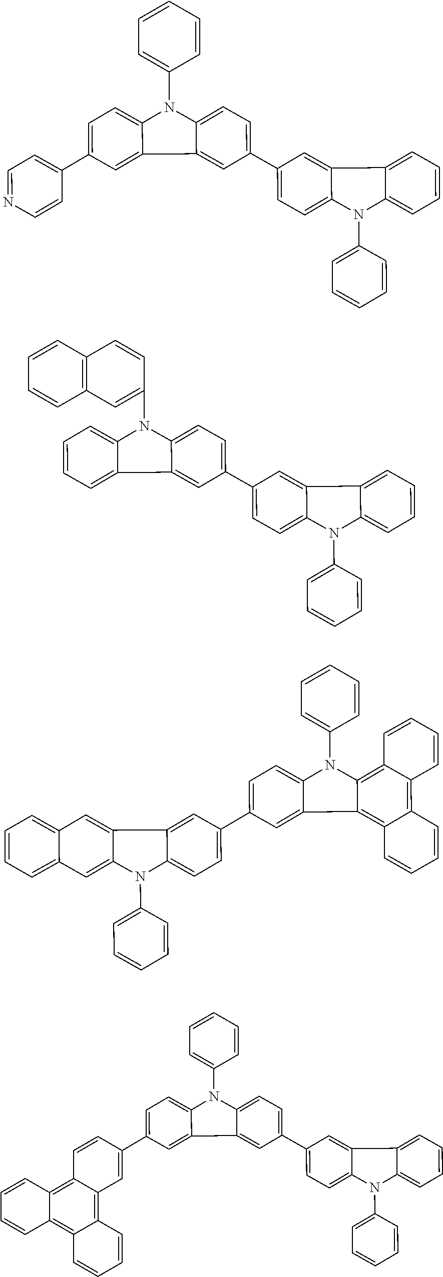

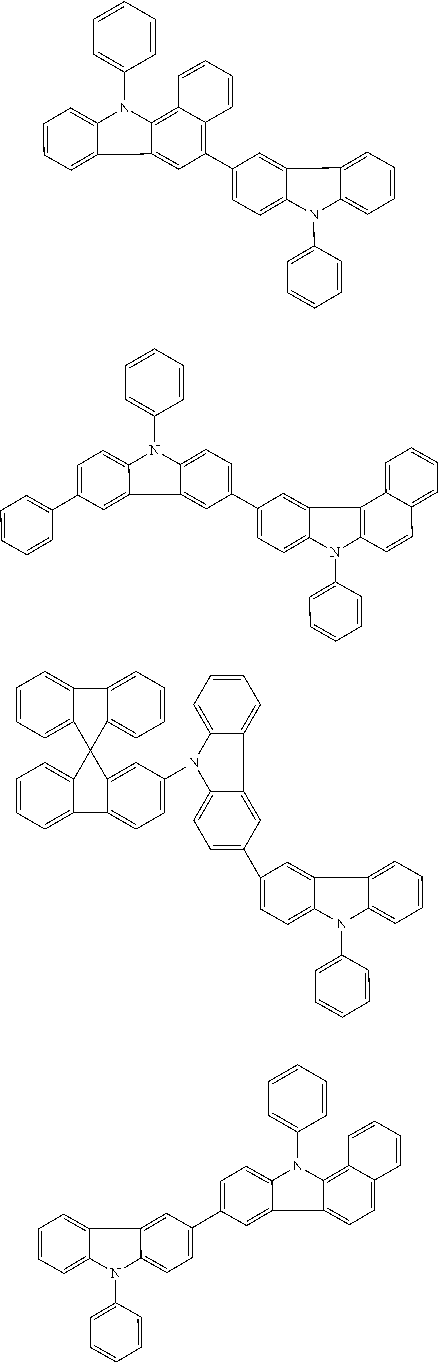 [SMILES: c1ccc(-n2c3ccccc3c3cc(-c4cc5c6ccccc6n(-c6ccccc6)c5c5ccccc45)ccc32)cc1 ; c1ccc(-c2ccc3c(c2)c2cc(-c4ccc5c(c4)c4c6ccccc6ccc4n5-c4ccccc4)ccc2n3-c2ccccc2)cc1 ; c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1 ; c1ccc(-n2c3ccccc3c3cc(-c4ccc5c(c4)c4ccc6ccccc6c4n5-c4ccccc4)ccc32)cc1]

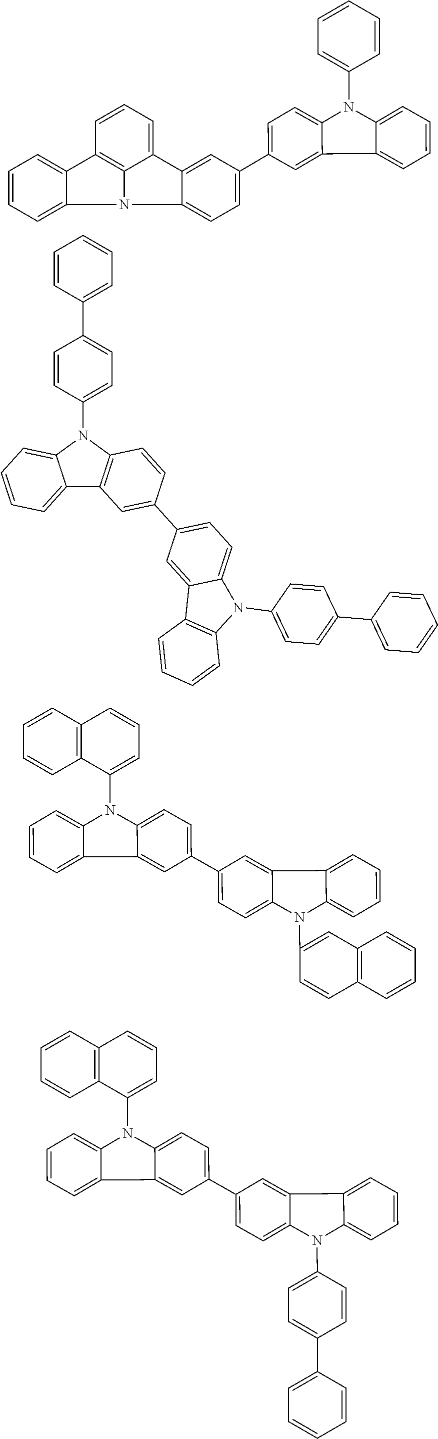

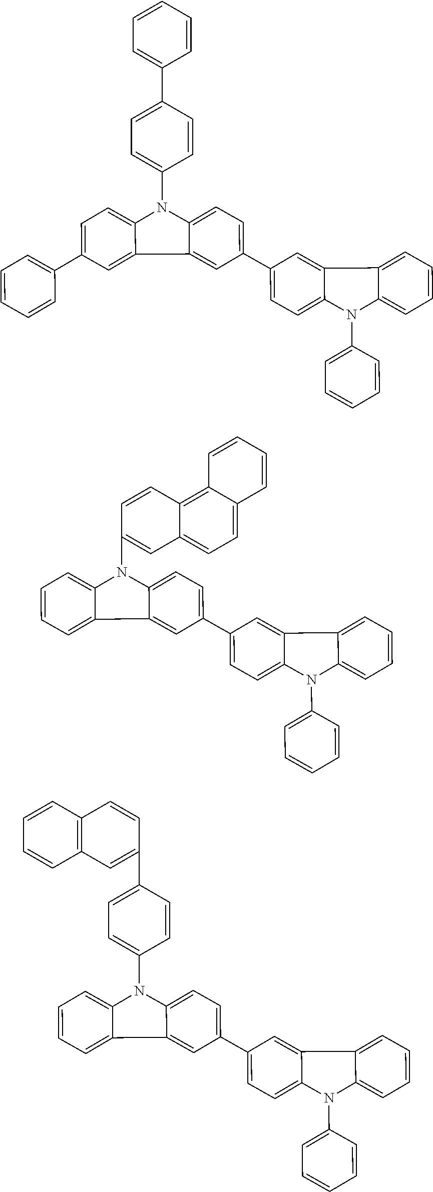 [SMILES: c1ccc(-c2ccc(-n3c4ccc(-c5ccccc5)cc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc5c(ccc6ccccc65)c4)ccc32)cc1 ; c1ccc(-n2c3ccccc3c3cc(-c4ccc5c(c4)c4ccccc4n5-c4ccc(-c5ccc6ccccc6c5)cc4)ccc32)cc1]

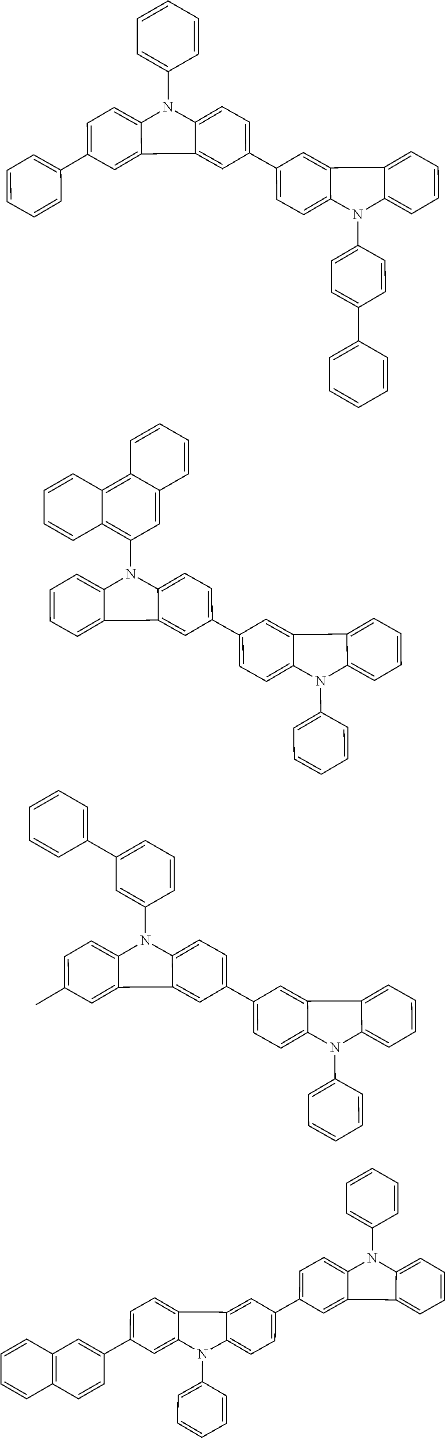 [SMILES: c1ccc(-c2ccc(-n3c4ccccc4c4cc(-c5ccc6c(c5)c5cc(-c7ccccc7)ccc5n6-c5ccccc5)ccc43)cc2)cc1 ; c1ccc(-n2c3ccccc3c3cc(-c4ccc5c(c4)c4ccccc4n5-c4cc5ccccc5c5ccccc45)ccc32)cc1 ; Cc1ccc2c(c1)c1cc(-c3ccc4c(c3)c3ccccc3n4-c3ccccc3)ccc1n2-c1cccc(-c2ccccc2)c1 ; c1ccc(-n2c3ccccc3c3cc(-c4ccc5c(c4)c4ccc(-c6ccc7ccccc7c6)cc4n5-c4ccccc4)ccc32)cc1]

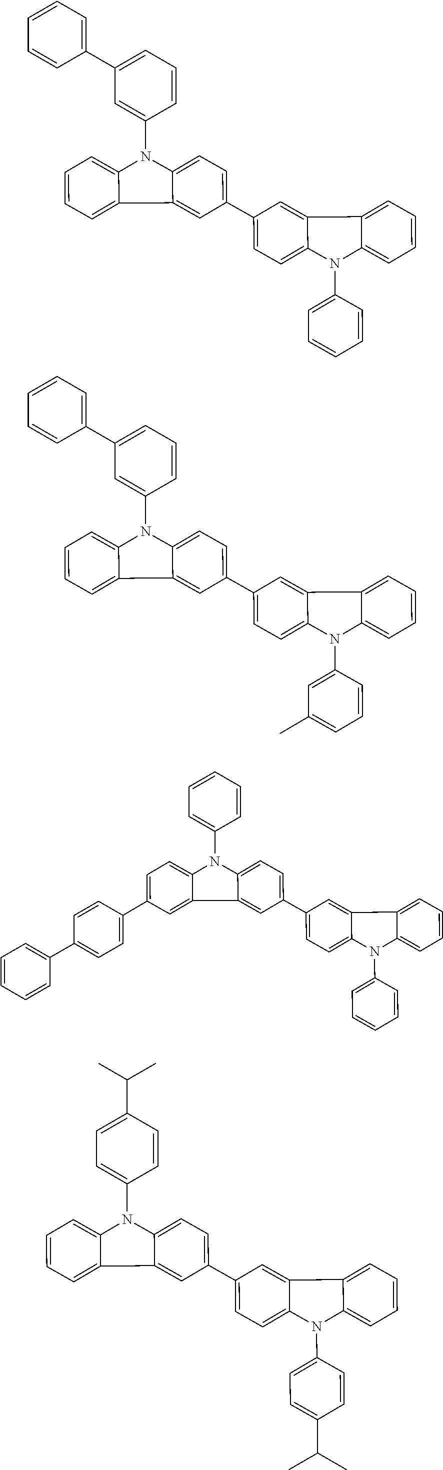 [SMILES: c1ccc(-c2cccc(-n3c4ccccc4c4cc(-c5ccc6c(c5)c5ccccc5n6-c5ccccc5)ccc43)c2)cc1 ; Cc1cccc(-n2c3ccccc3c3cc(-c4ccc5c(c4)c4ccccc4n5-c4cccc(-c5ccccc5)c4)ccc32)c1 ; c1ccc(-c2ccc(-c3ccc4c(c3)c3cc(-c5ccc6c(c5)c5ccccc5n6-c5ccccc5)ccc3n4-c3ccccc3)cc2)cc1 ; CC(C)c1ccc(-n2c3ccccc3c3cc(-c4ccc5c(c4)c4ccccc4n5-c4ccc(C(C)C)cc4)ccc32)cc1]

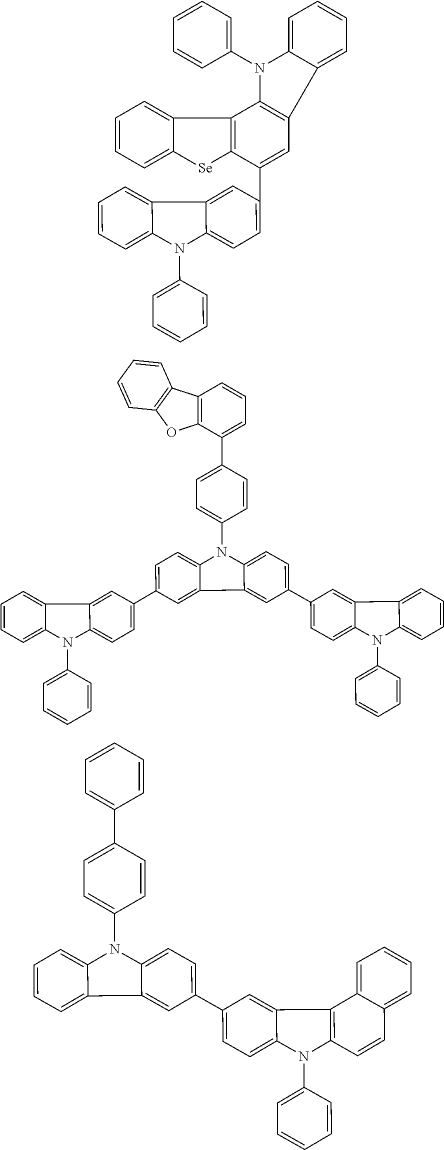

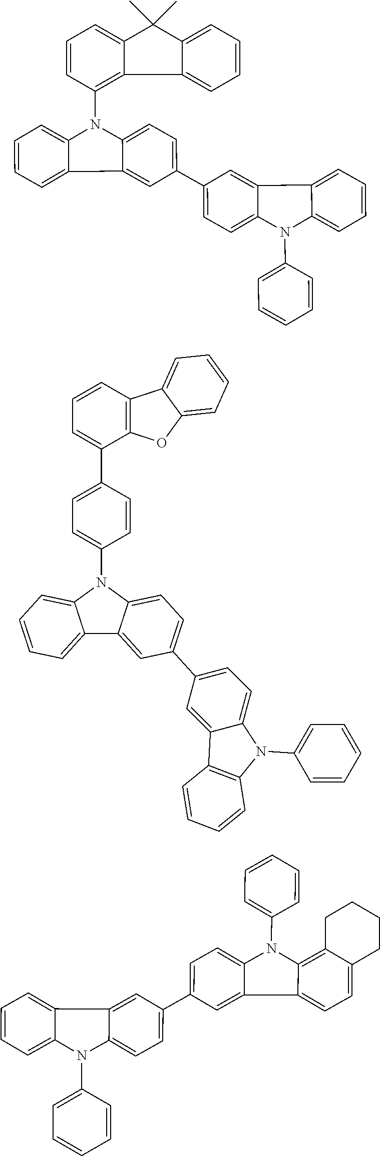 [SMILES: CC1(C)c2ccccc2-c2c(-n3c4ccccc4c4cc(-c5ccc6c(c5)c5ccccc5n6-c5ccccc5)ccc43)cccc21 ; c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccc6c(c4n5-c4ccccc4)CCCC6)ccc32)cc1]

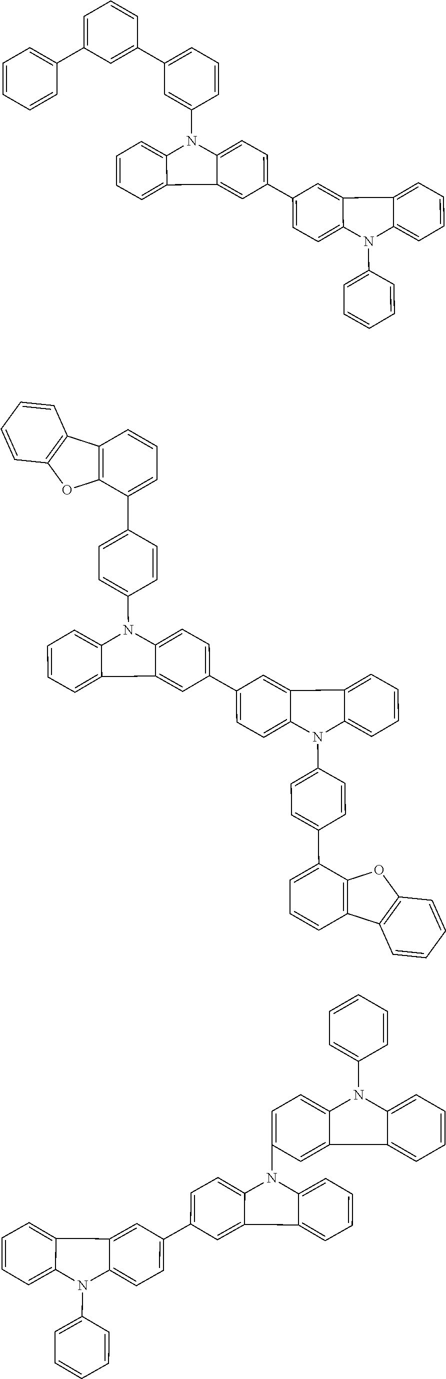 [SMILES: c1ccc(-c2cccc(-c3cccc(-n4c5ccccc5c5cc(-c6ccc7c(c6)c6ccccc6n7-c6ccccc6)ccc54)c3)c2)cc1 ; c1ccc2c(c1)oc1c(-c3ccc(-n4c5ccccc5c5cc(-c6ccc7c(c6)c6ccccc6n7-c6ccc(-c7cccc8c7oc7ccccc78)cc6)ccc54)cc3)cccc12 ; c1ccc(-n2c3ccccc3c3cc(-c4ccc5c(c4)c4ccccc4n5-c4ccc5c(c4)c4ccccc4n5-c4ccccc4)ccc32)cc1]

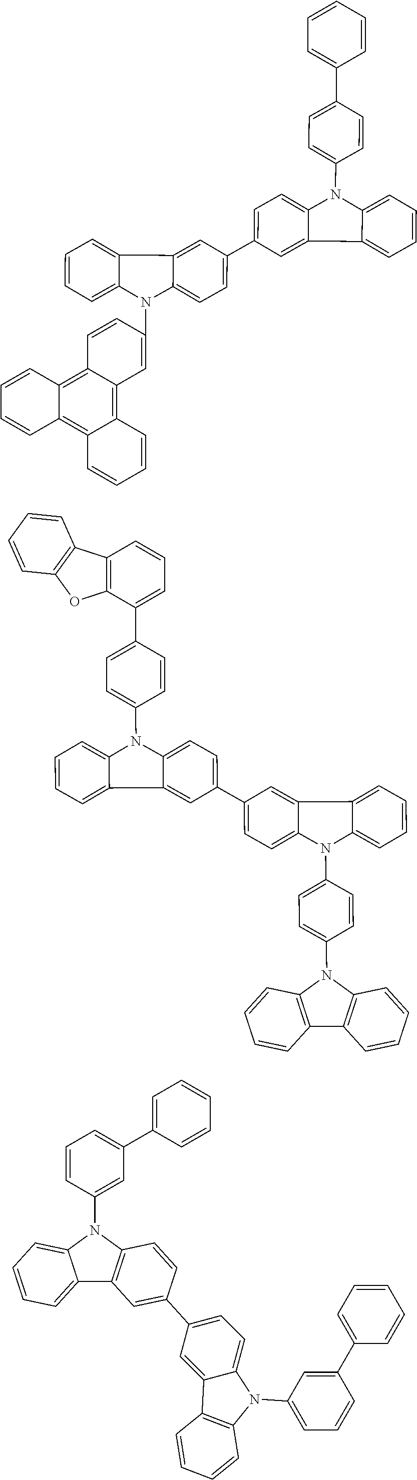 [SMILES: c1ccc(-c2ccc(-n3c4ccccc4c4cc(-c5ccc6c(c5)c5ccccc5n6-c5ccc6c7ccccc7c7ccccc7c6c5)ccc43)cc2)cc1 ; c1ccc2c(c1)oc1c(-c3ccc(-n4c5ccccc5c5cc(-c6ccc7c(c6)c6ccccc6n7-c6ccc(-n7c8ccccc8c8ccccc87)cc6)ccc54)cc3)cccc12 ; c1ccc(-c2cccc(-n3c4ccccc4c4cc(-c5ccc6c(c5)c5ccccc5n6-c5cccc(-c6ccccc6)c5)ccc43)c2)cc1]

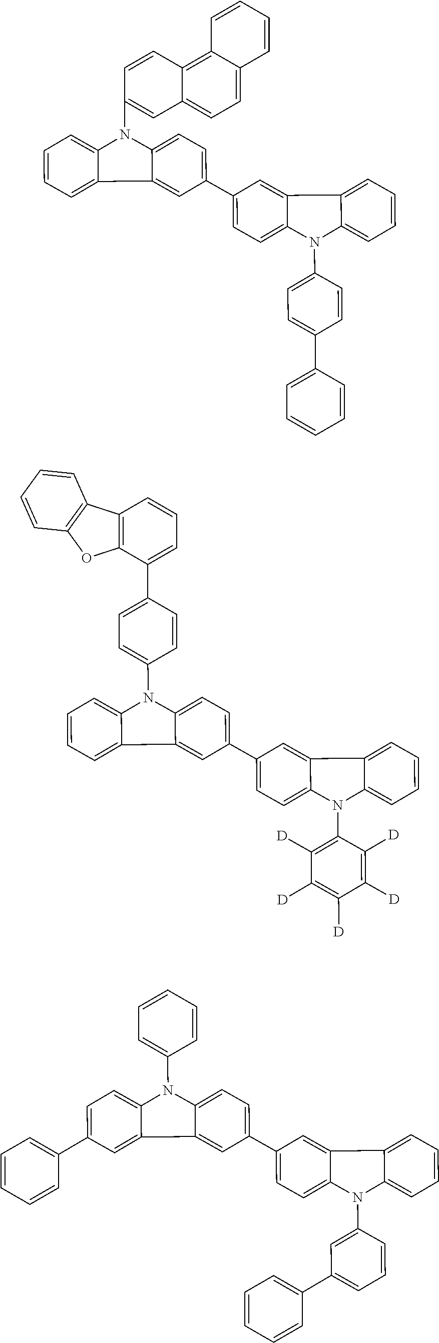 [SMILES: c1ccc(-c2ccc(-n3c4ccccc4c4cc(-c5ccc6c(c5)c5ccccc5n6-c5ccc6c(ccc7ccccc76)c5)ccc43)cc2)cc1 ; [2H]c1c([2H])c([2H])c(-n2c3ccccc3c3cc(-c4ccc5c(c4)c4ccccc4n5-c4ccc(-c5cccc6c5oc5ccccc56)cc4)ccc32)c([2H])c1[2H] ; c1ccc(-c2cccc(-n3c4ccccc4c4cc(-c5ccc6c(c5)c5cc(-c7ccccc7)ccc5n6-c5ccccc5)ccc43)c2)cc1]

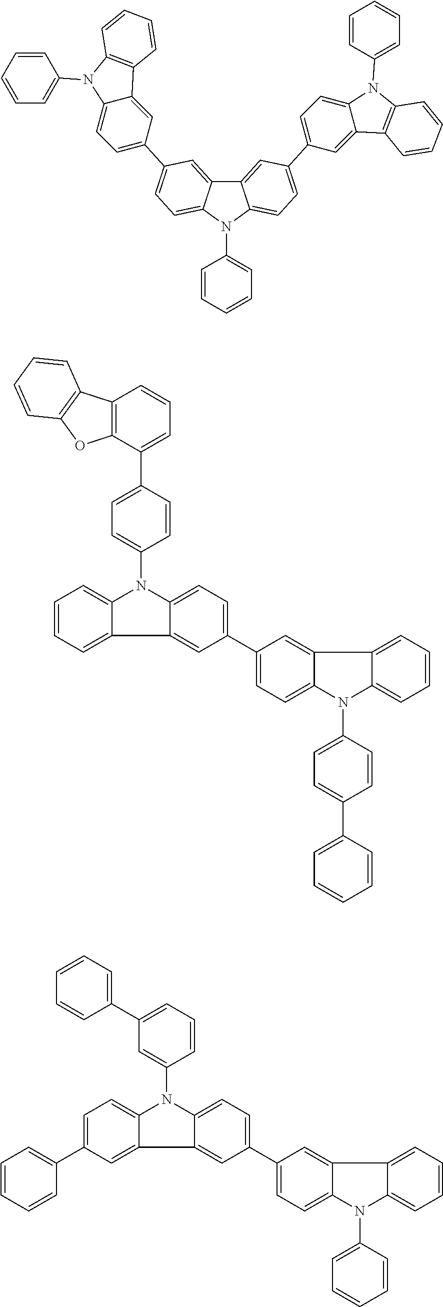 [SMILES: c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; c1ccc(-c2ccc(-n3c4ccccc4c4cc(-c5ccc6c(c5)c5ccccc5n6-c5ccc(-c6cccc7c6oc6ccccc67)cc5)ccc43)cc2)cc1 ; c1ccc(-c2cccc(-n3c4ccc(-c5ccccc5)cc4c4cc(-c5ccc6c(c5)c5ccccc5n6-c5ccccc5)ccc43)c2)cc1]

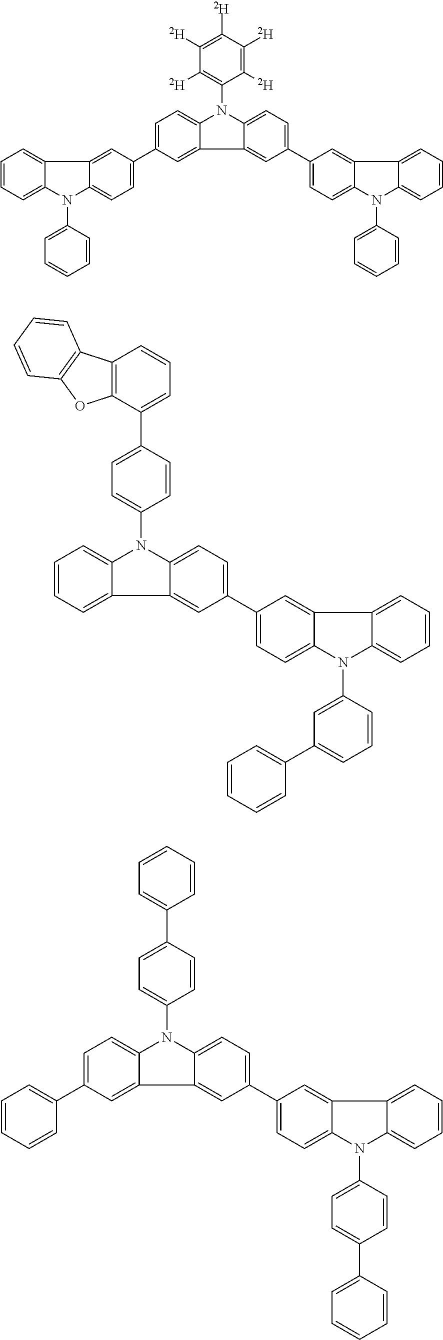 [SMILES: [2H]c1c([2H])c([2H])c(-n2c3ccc(-c4ccc5c(c4)c4ccccc4n5-c4ccccc4)cc3c3cc(-c4ccc5c(c4)c4ccccc4n5-c4ccccc4)ccc32)c([2H])c1[2H] ; c1ccc(-c2cccc(-n3c4ccccc4c4cc(-c5ccc6c(c5)c5ccccc5n6-c5ccc(-c6cccc7c6oc6ccccc67)cc5)ccc43)c2)cc1 ; c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1]

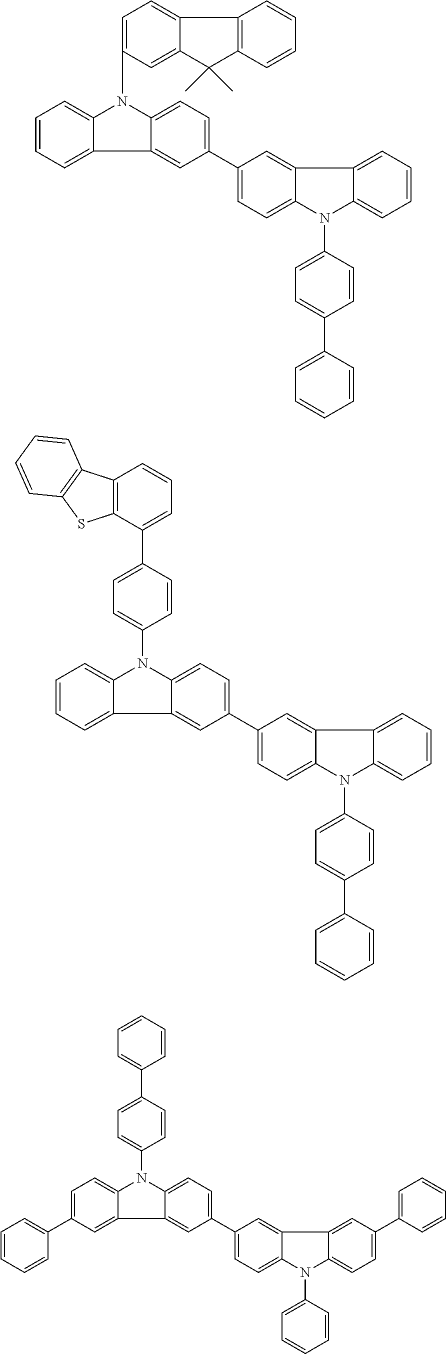 [SMILES: CC1(C)c2ccccc2-c2ccc(-n3c4ccccc4c4cc(-c5ccc6c(c5)c5ccccc5n6-c5ccc(-c6ccccc6)cc5)ccc43)cc21 ; c1ccc(-c2ccc(-n3c4ccccc4c4cc(-c5ccc6c(c5)c5ccccc5n6-c5ccc(-c6cccc7c6sc6ccccc67)cc5)ccc43)cc2)cc1 ; c1ccc(-c2ccc(-n3c4ccc(-c5ccccc5)cc4c4cc(-c5ccc6c(c5)c5cc(-c7ccccc7)ccc5n6-c5ccccc5)ccc43)cc2)cc1]

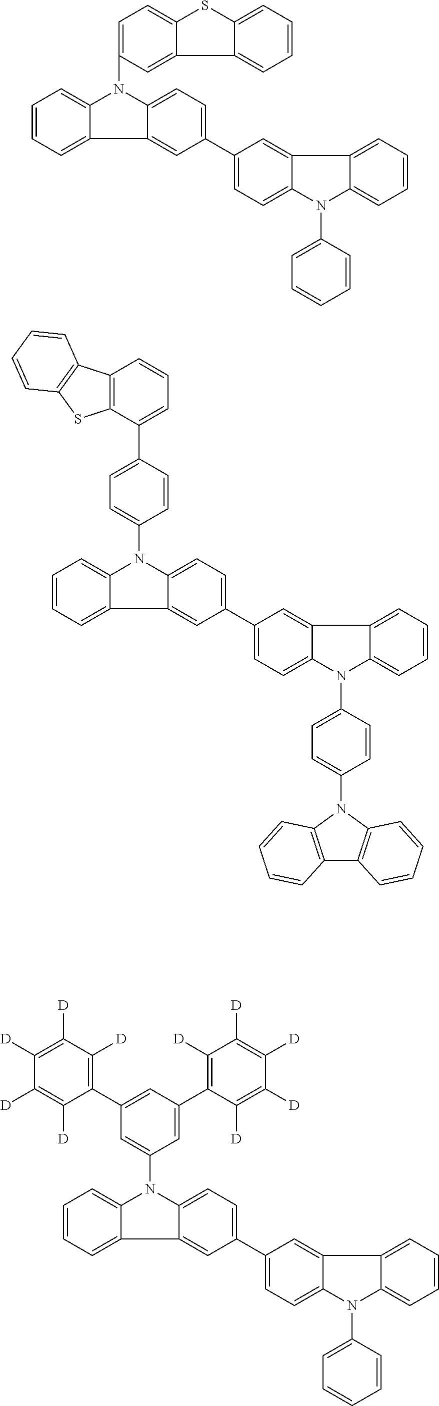 [SMILES: c1ccc(-n2c3ccccc3c3cc(-c4ccc5c(c4)c4ccccc4n5-c4ccc5sc6ccccc6c5c4)ccc32)cc1 ; c1ccc2c(c1)sc1c(-c3ccc(-n4c5ccccc5c5cc(-c6ccc7c(c6)c6ccccc6n7-c6ccc(-n7c8ccccc8c8ccccc87)cc6)ccc54)cc3)cccc12 ; [2H]c1c([2H])c([2H])c(-c2cc(-c3c([2H])c([2H])c([2H])c([2H])c3[2H])cc(-n3c4ccccc4c4cc(-c5ccc6c(c5)c5ccccc5n6-c5ccccc5)ccc43)c2)c([2H])c1[2H]]

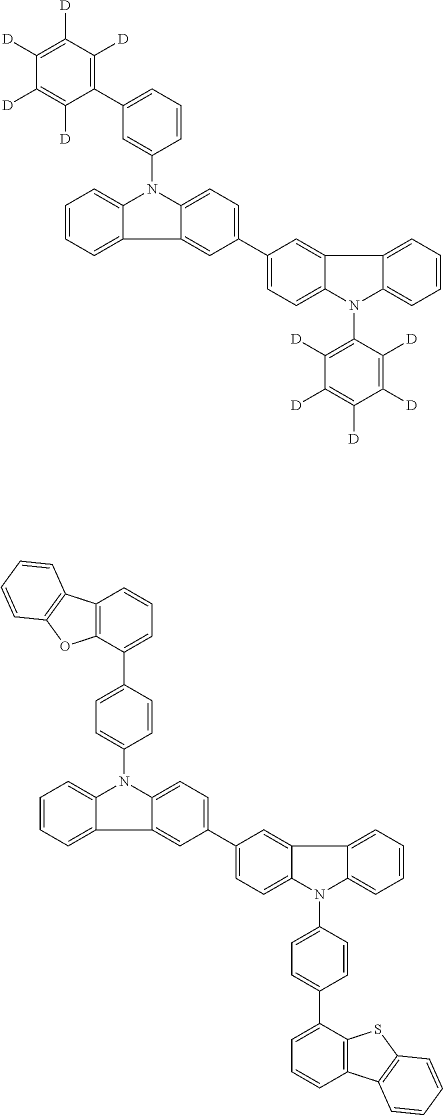 [SMILES: [2H]c1c([2H])c([2H])c(-c2cccc(-n3c4ccccc4c4cc(-c5ccc6c(c5)c5ccccc5n6-c5c([2H])c([2H])c([2H])c([2H])c5[2H])ccc43)c2)c([2H])c1[2H] ; c1ccc2c(c1)oc1c(-c3ccc(-n4c5ccccc5c5cc(-c6ccc7c(c6)c6ccccc6n7-c6ccc(-c7cccc8c7sc7ccccc78)cc6)ccc54)cc3)cccc12]

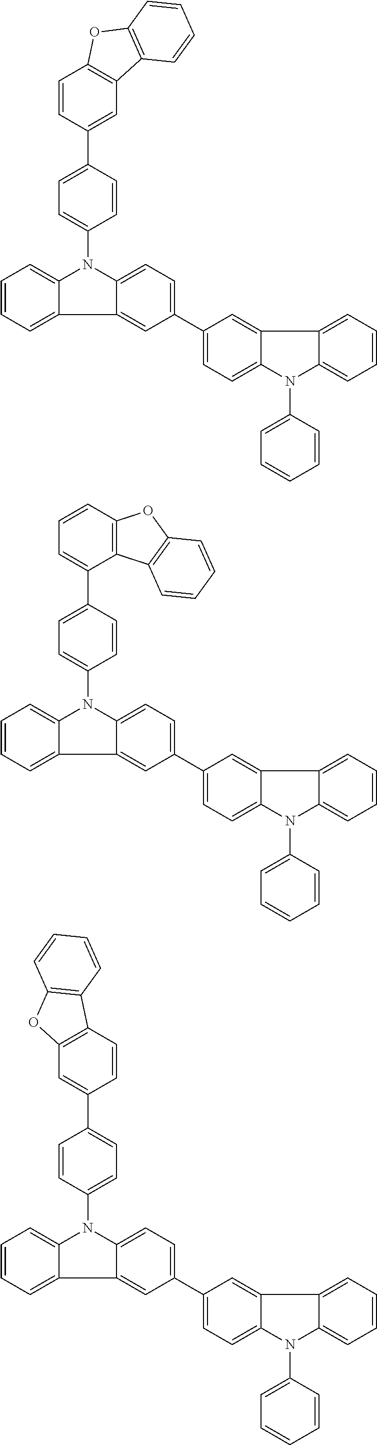 [SMILES: c1ccc(-n2c3ccccc3c3cc(-c4ccc5c(c4)c4ccccc4n5-c4ccc(-c5ccc6oc7ccccc7c6c5)cc4)ccc32)cc1 ; c1ccc(-n2c3ccccc3c3cc(-c4ccc5c(c4)c4ccccc4n5-c4ccc(-c5cccc6oc7ccccc7c56)cc4)ccc32)cc1 ; c1ccc(-n2c3ccccc3c3cc(-c4ccc5c(c4)c4ccccc4n5-c4ccc(-c5ccc6c(c5)oc5ccccc56)cc4)ccc32)cc1]

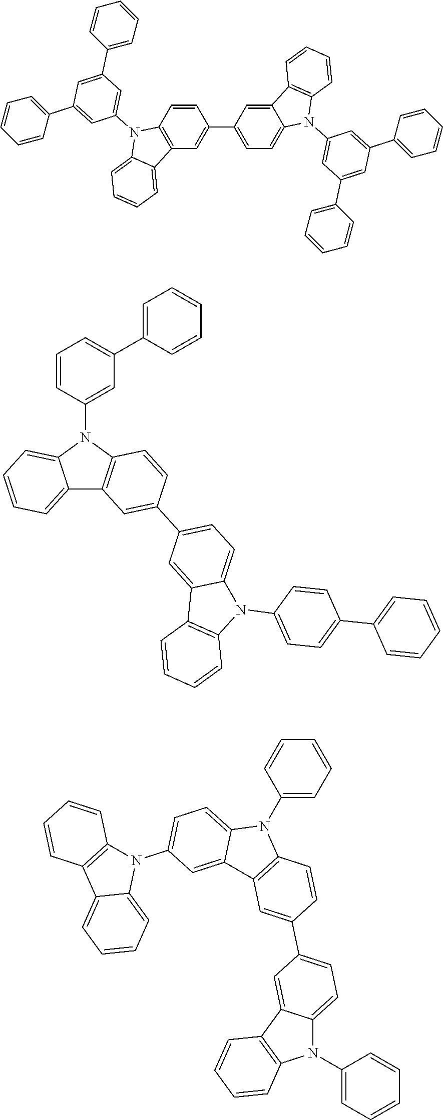 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-n2c3ccccc3c3cc(-c4ccc5c(c4)c4cc(-n6c7ccccc7c7ccccc76)ccc4n5-c4ccccc4)ccc32)cc1]

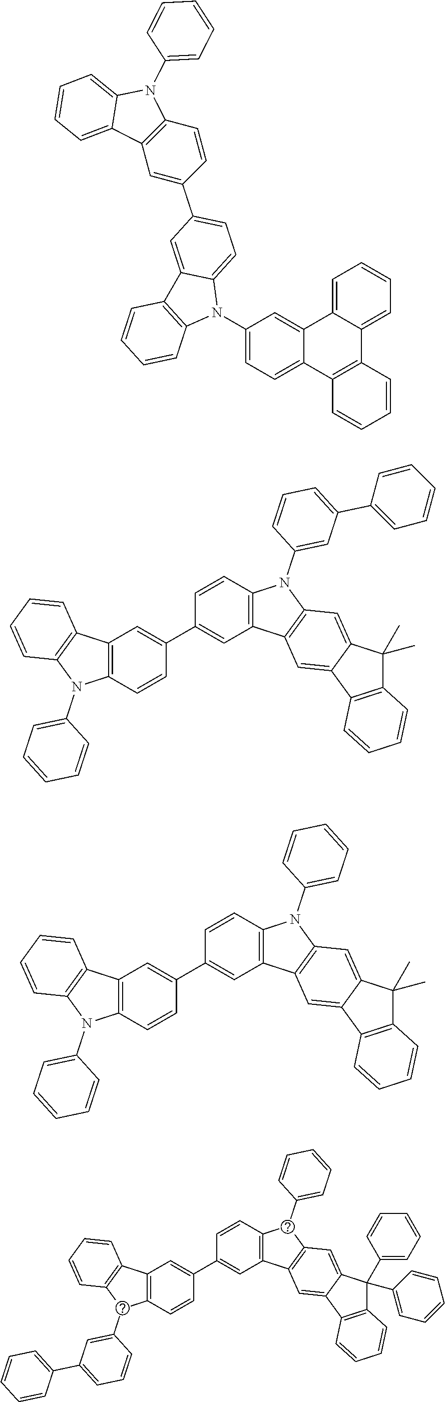

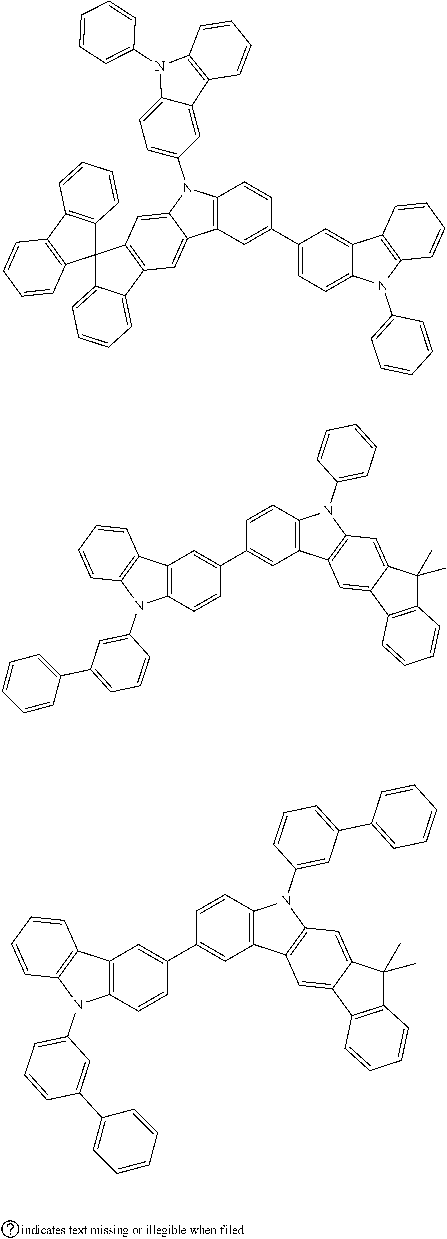 [SMILES: c1ccc(-n2c3ccccc3c3cc(-c4ccc5c(c4)c4cc6c(cc4n5-c4ccc5c(c4)c4ccccc4n5-c4ccccc4)C4(c5ccccc5-c5ccccc54)c4ccccc4-6)ccc32)cc1 ; CC1(C)c2ccccc2-c2cc3c4cc(-c5ccc6c(c5)c5ccccc5n6-c5cccc(-c6ccccc6)c5)ccc4n(-c4ccccc4)c3cc21 ; CC1(C)c2ccccc2-c2cc3c4cc(-c5ccc6c(c5)c5ccccc5n6-c5cccc(-c6ccccc6)c5)ccc4n(-c4cccc(-c5ccccc5)c4)c3cc21]

Preferred bridged carbazoles are the structures of the following formula (23):

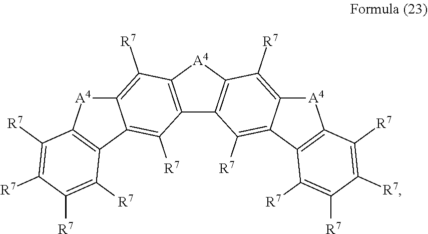

where A⁴ and R⁷ have the definitions given above and A⁴ is preferably the same or different at each instance and is selected from the group consisting of NR⁵ and C(R⁵)₂.

Preferred dibenzofuran derivatives are the compounds of the following formula (24):

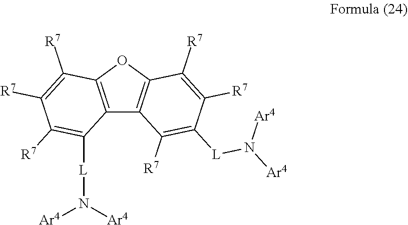

where the oxygen may also be replaced by sulfur so as to form a dibenzothiophene, L is a single bond or an aromatic or heteroaromatic ring system which has 5 to 30 aromatic ring atoms and is substituted by R⁵ radicals, and R⁷ and Ar⁴ have the definitions given above. It is also possible here for the two Ar⁴ groups that bind to the same nitrogen atom, or for one Ar⁴ group and one L group that bind to the same nitrogen atom, to be bonded to one another, for example to give a carbazole.

Examples of suitable dibenzofuran derivatives are the compounds depicted below.

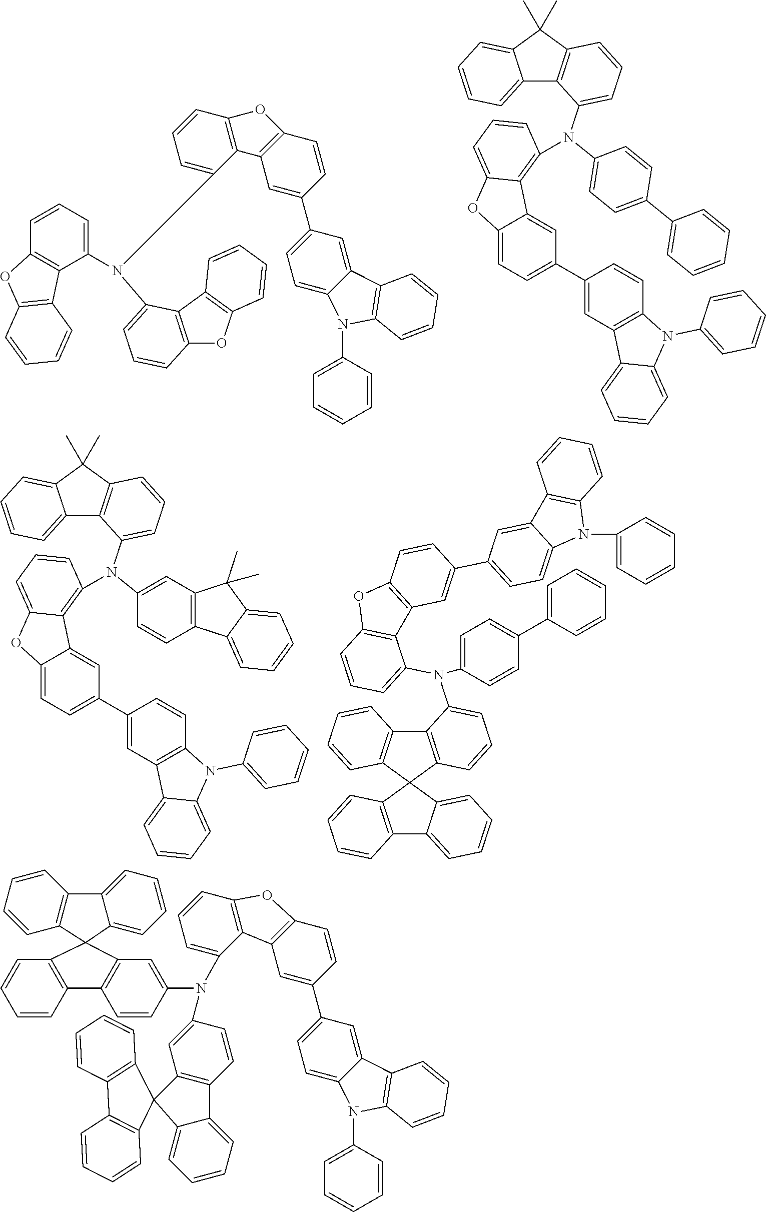

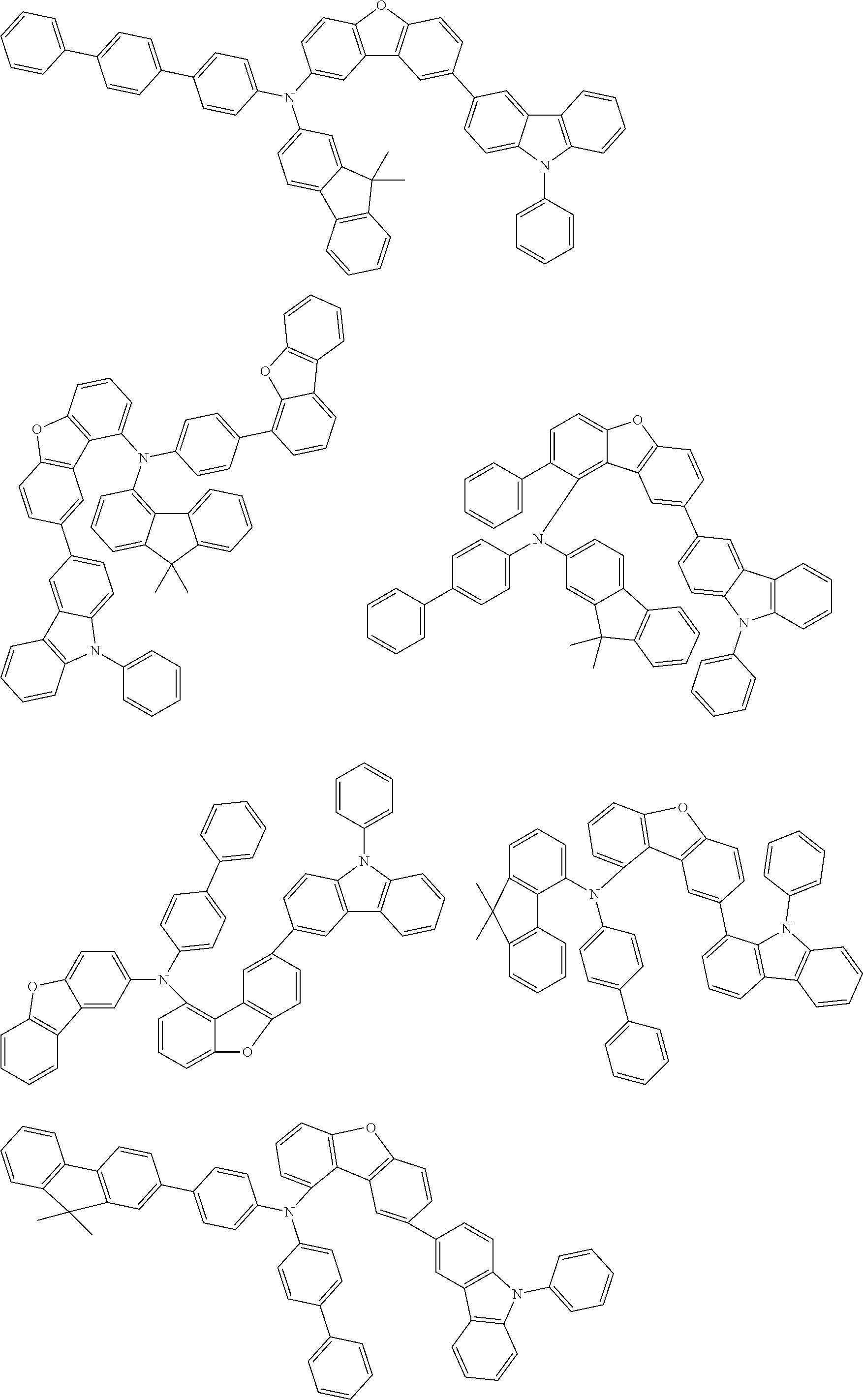

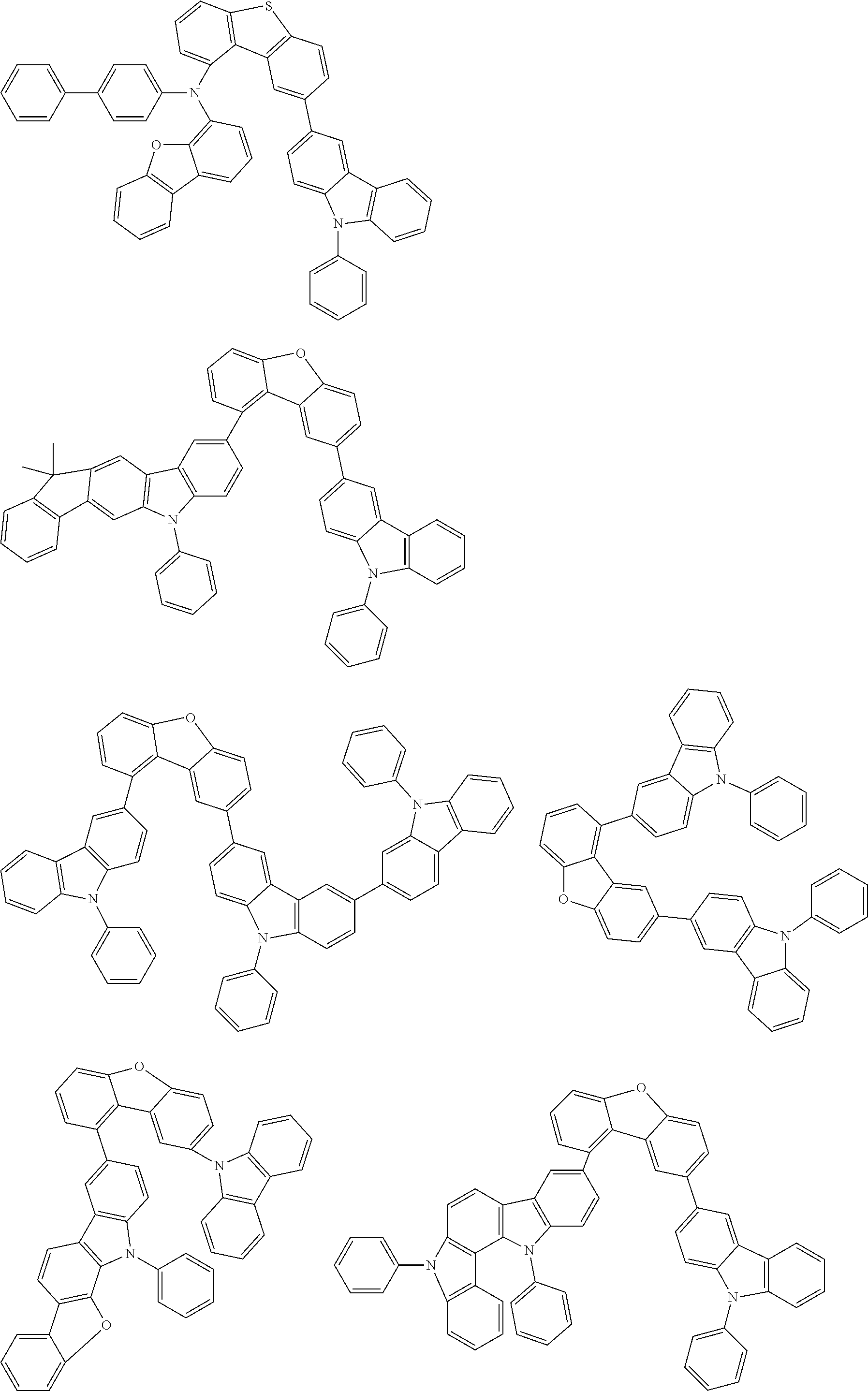

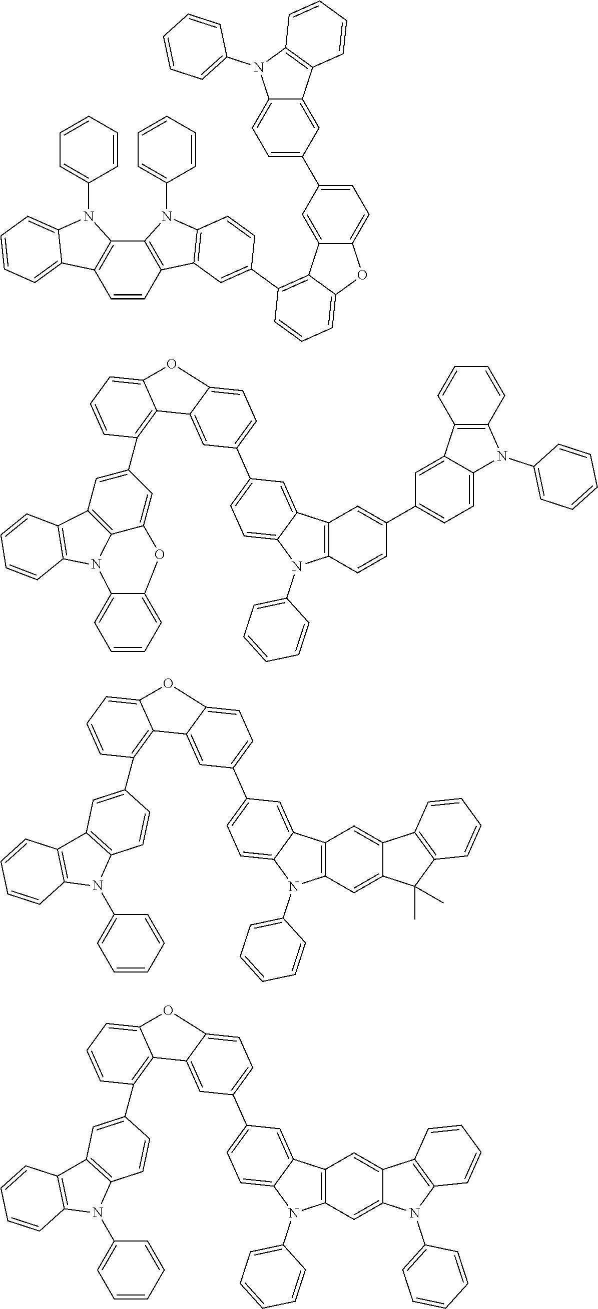

Preferred carbazoleamines are the structures of the following formulae (25), (26) and (27):

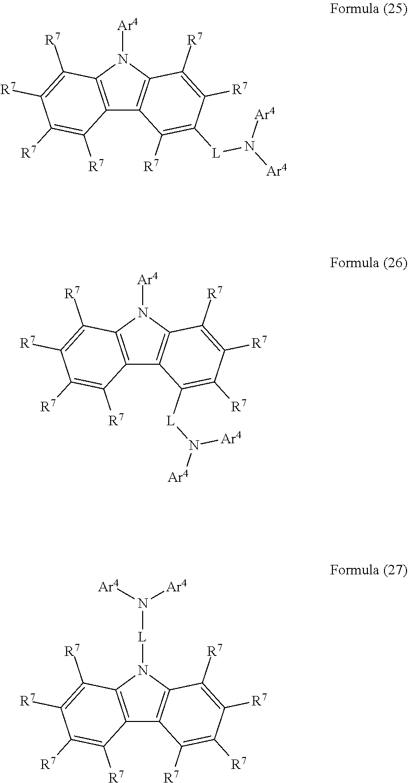

where L is an aromatic or heteroaromatic ring system which has 5 to 30 aromatic ring atoms and is substituted by R⁵ radicals, and R⁵, R⁷ and Ar⁴ have the definitions given above.

Examples of suitable carbazoleamine derivatives are the compounds depicted below.

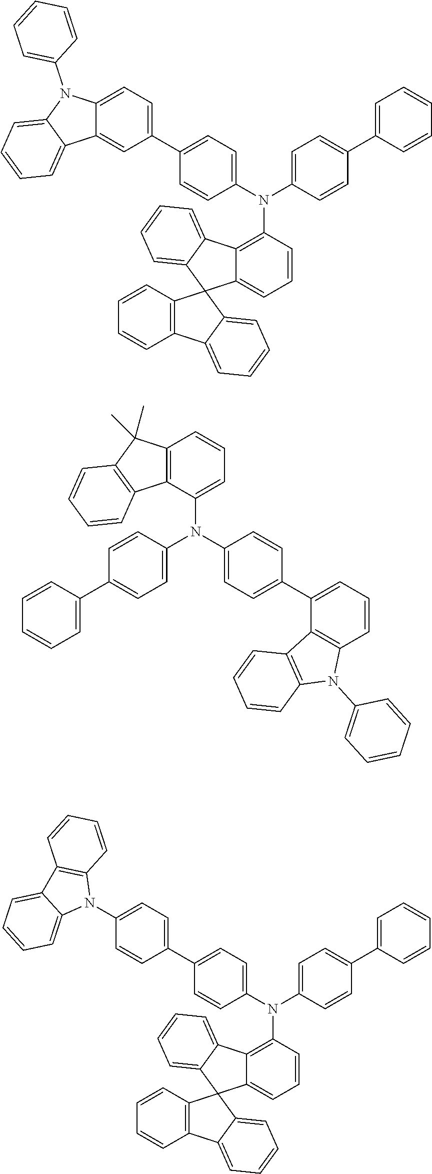 [SMILES: c1ccc(-c2ccc(N(c3ccc(-c4ccc5c(c4)c4ccccc4n5-c4ccccc4)cc3)c3cccc4c3-c3ccccc3C43c4ccccc4-c4ccccc43)cc2)cc1 ; CC1(C)c2ccccc2-c2c(N(c3ccc(-c4ccccc4)cc3)c3ccc(-c4cccc5c4c4ccccc4n5-c4ccccc4)cc3)cccc21 ; c1ccc(-c2ccc(N(c3ccc(-c4ccc(-n5c6ccccc6c6ccccc65)cc4)cc3)c3cccc4c3-c3ccccc3C43c4ccccc4-c4ccccc43)cc2)cc1]

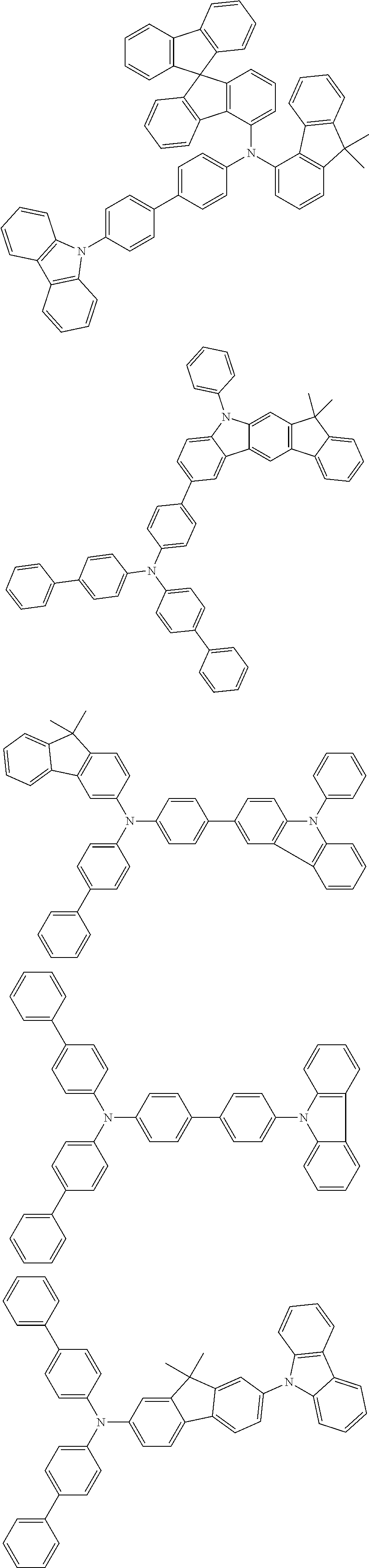

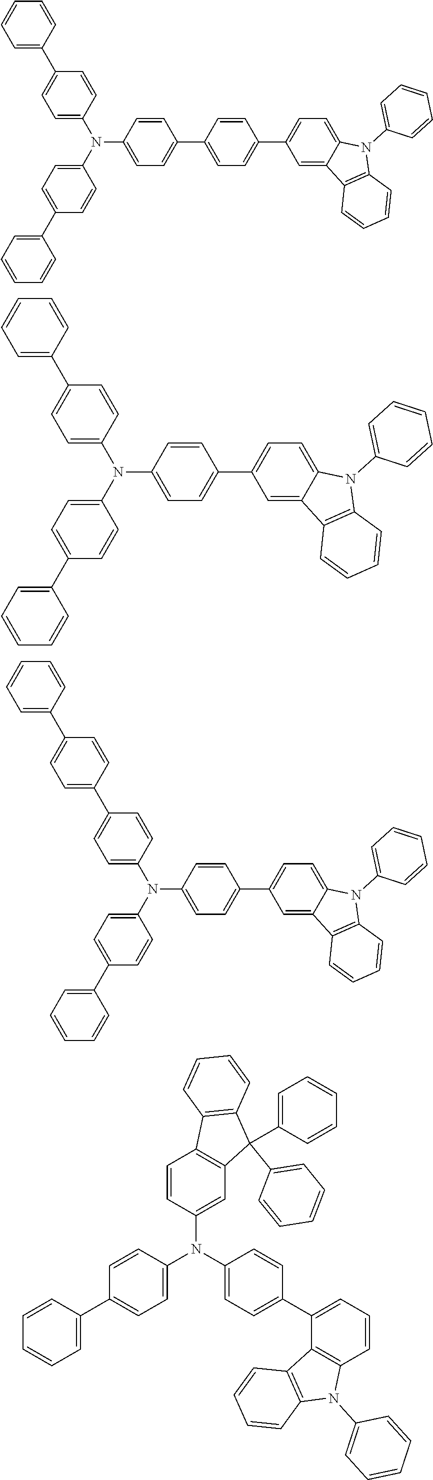

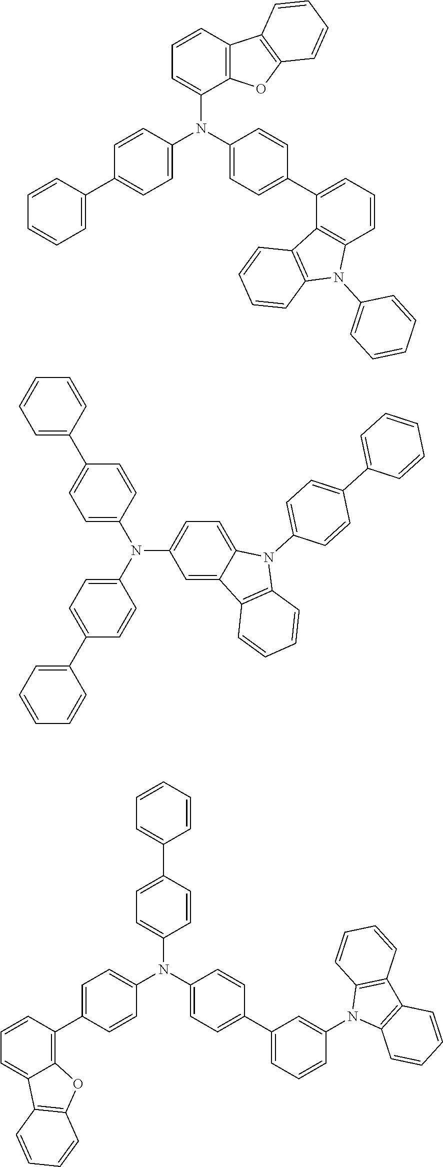 [SMILES: c1ccc(-c2ccc(N(c3ccc(-c4cccc5c4c4ccccc4n5-c4ccccc4)cc3)c3cccc4c3oc3ccccc34)cc2)cc1 ; c1ccc(-c2ccc(N(c3ccc(-c4ccccc4)cc3)c3ccc4c(c3)c3ccccc3n4-c3ccc(-c4ccccc4)cc3)cc2)cc1 ; c1ccc(-c2ccc(N(c3ccc(-c4cccc(-n5c6ccccc6c6ccccc65)c4)cc3)c3ccc(-c4cccc5c4oc4ccccc45)cc3)cc2)cc1]

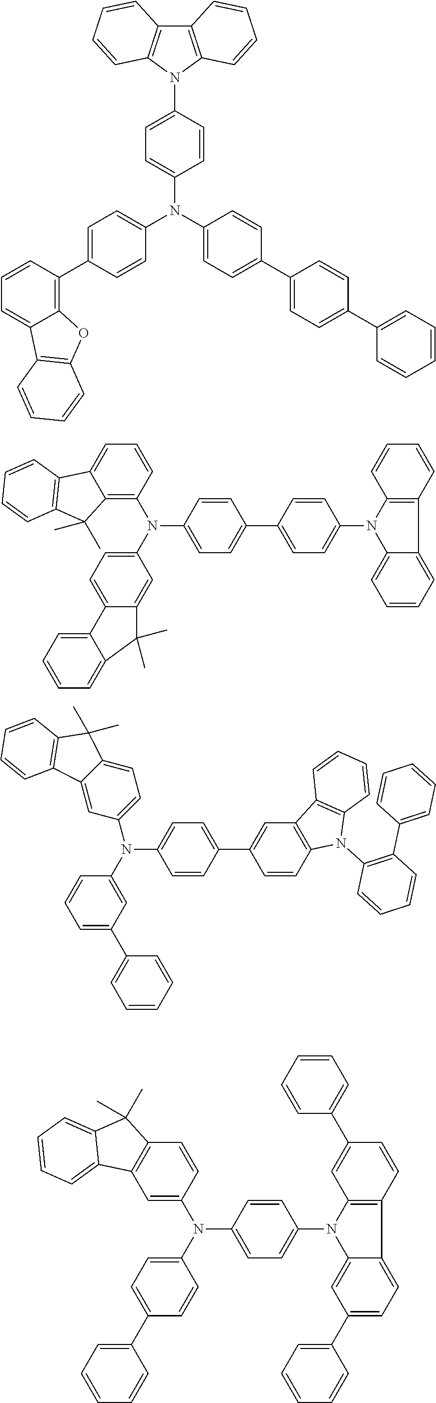

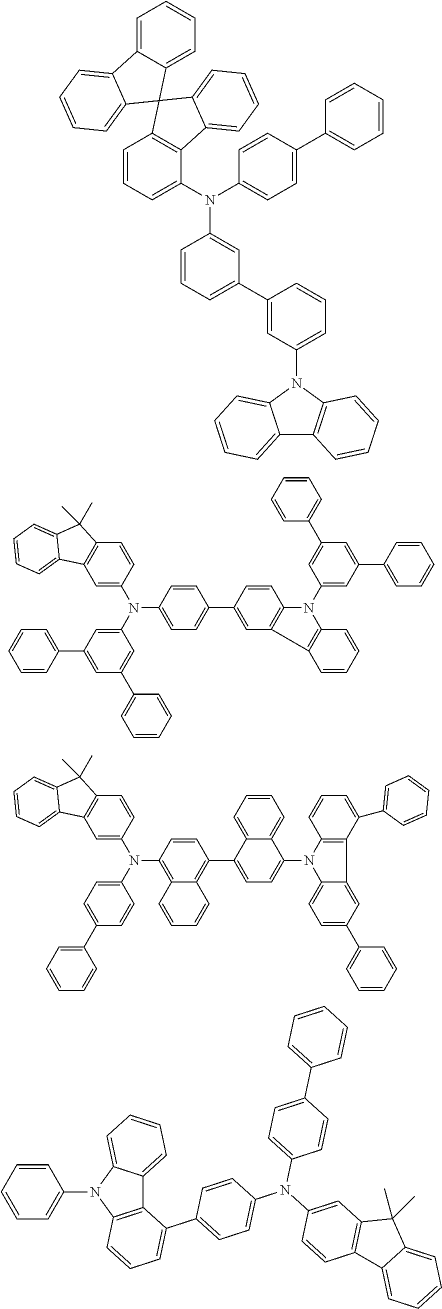

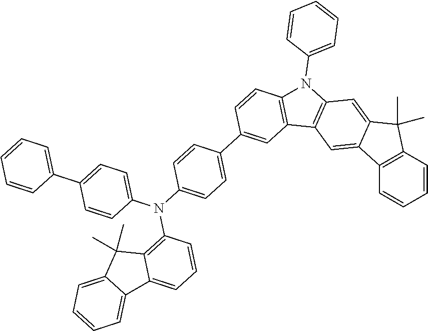 [SMILES: CC1(C)c2ccccc2-c2cc3c4cc(-c5ccc(N(c6ccc(-c7ccccc7)cc6)c6cccc7c6C(C)(C)c6ccccc6-7)cc5)ccc4n(-c4ccccc4)c3cc21]

Preferred co-matrix materials, especially when the compound of the invention is substituted by an electron-rich heteroaromatic ring system, for example a carbazole group, are also selected from the group consisting of triazine derivatives, pyrimidine derivatives and quinazoline derivatives. Preferred triazine, quinazoline or pyrimidine derivatives that can be used as a mixture together with the compounds of the invention are the compounds of the following formulae (28), (29) and (30):

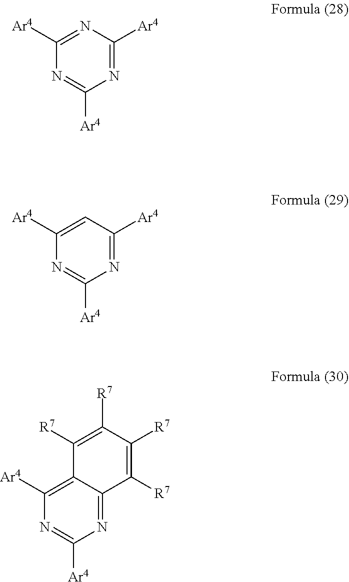

where Ar⁴ and R⁷ have the definitions given above.

Particular preference is given to the triazine derivatives of the formula (28) and the quinazoline derivatives of the formula (30), especially the triazine derivatives of the formula (28).

In a preferred embodiment of the invention, Ar⁴ in the formulae (28), (29) and (30) is the same or different at each instance and is an aromatic or heteroaromatic ring system which has 6 to 30 aromatic ring atoms, especially 6 to 24 aromatic ring atoms, and is substituted by R⁵ radicals. Suitable aromatic or heteroaromatic ring systems Ar⁴ here are the same as listed above as structures R-1 to R-81.

Examples of suitable triazine compounds that may be used as matrix materials together with the compounds of the invention are the compounds depicted in the following table:

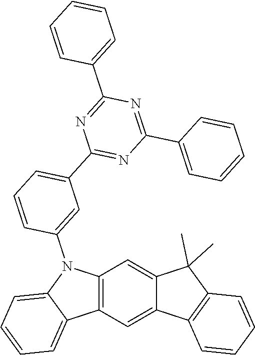 [SMILES: CC1(C)c2ccccc2-c2cc3c4ccccc4n(-c4cccc(-c5nc(-c6ccccc6)nc(-c6ccccc6)n5)c4)c3cc21]

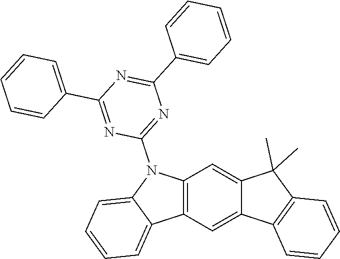 [SMILES: CC1(C)c2ccccc2-c2cc3c4ccccc4n(-c4nc(-c5ccccc5)nc(-c5ccccc5)n4)c3cc21]

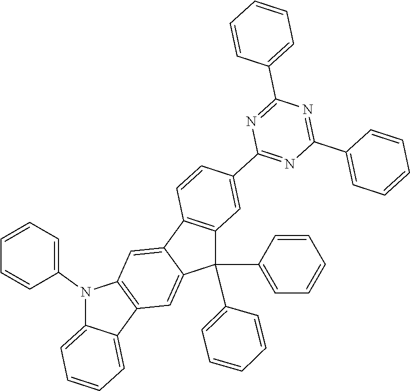 [SMILES: c1ccc(-c2nc(-c3ccccc3)nc(-c3ccc4c(c3)C(c3ccccc3)(c3ccccc3)c3cc5c6ccccc6n(-c6ccccc6)c5cc3-4)n2)cc1]

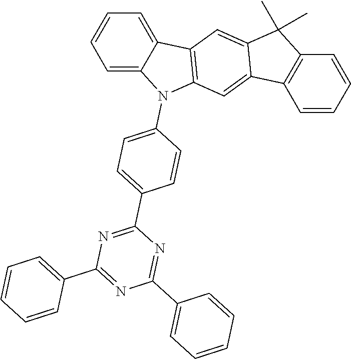 [SMILES: CC1(C)c2ccccc2-c2cc3c(cc21)c1ccccc1n3-c1ccc(-c2nc(-c3ccccc3)nc(-c3ccccc3)n2)cc1]

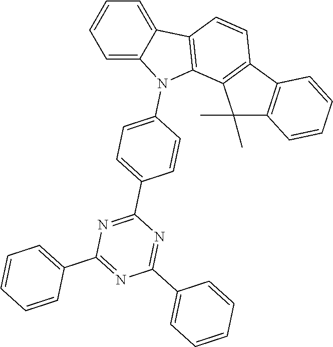 [SMILES: CC1(C)c2ccccc2-c2ccc3c4ccccc4n(-c4ccc(-c5nc(-c6ccccc6)nc(-c6ccccc6)n5)cc4)c3c21]

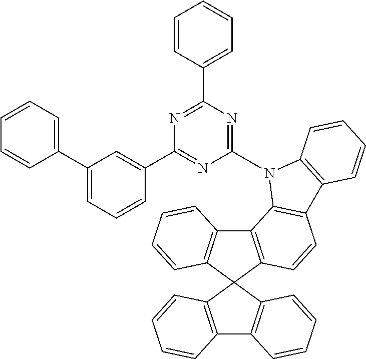 [SMILES: c1ccc(-c2cccc(-c3nc(-c4ccccc4)nc(-n4c5ccccc5c5ccc6c(c54)-c4ccccc4C64c5ccccc5-c5ccccc54)n3)c2)cc1]

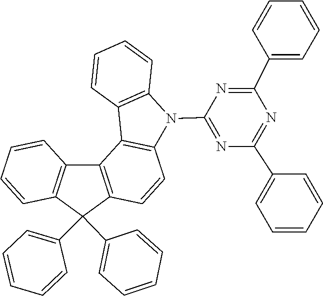 [SMILES: c1ccc(-c2nc(-c3ccccc3)nc(-n3c4ccccc4c4c5c(ccc43)C(c3ccccc3)(c3ccccc3)c3ccccc3-5)n2)cc1]

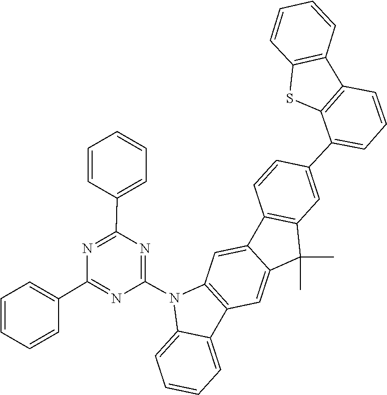 [SMILES: CC1(C)c2cc(-c3cccc4c3sc3ccccc34)ccc2-c2cc3c(cc21)c1ccccc1n3-c1nc(-c2ccccc2)nc(-c2ccccc2)n1]

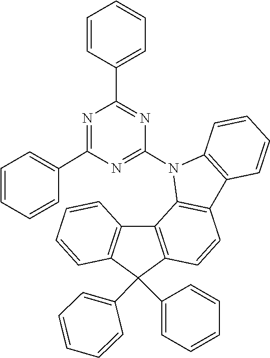 [SMILES: c1ccc(-c2nc(-c3ccccc3)nc(-n3c4ccccc4c4ccc5c(c43)-c3ccccc3C5(c3ccccc3)c3ccccc3)n2)cc1]

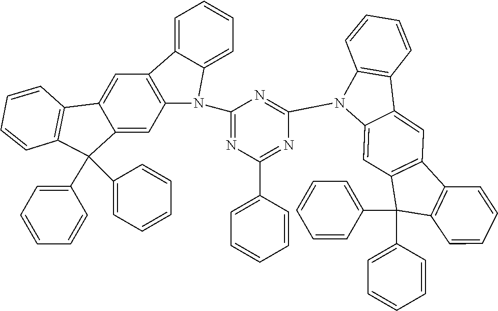 [SMILES: c1ccc(-c2nc(-n3c4ccccc4c4cc5c(cc43)C(c3ccccc3)(c3ccccc3)c3ccccc3-5)nc(-n3c4ccccc4c4cc5c(cc43)C(c3ccccc3)(c3ccccc3)c3ccccc3-5)n2)cc1]

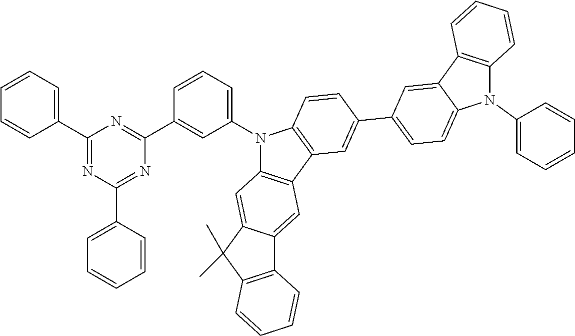 [SMILES: CC1(C)c2ccccc2-c2cc3c4cc(-c5ccc6c(c5)c5ccccc5n6-c5ccccc5)ccc4n(-c4cccc(-c5nc(-c6ccccc6)nc(-c6ccccc6)n5)c4)c3cc21]

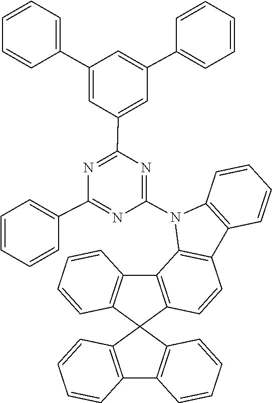 [SMILES: c1ccc(-c2cc(-c3ccccc3)cc(-c3nc(-c4ccccc4)nc(-n4c5ccccc5c5ccc6c(c54)-c4ccccc4C64c5ccccc5-c5ccccc54)n3)c2)cc1]

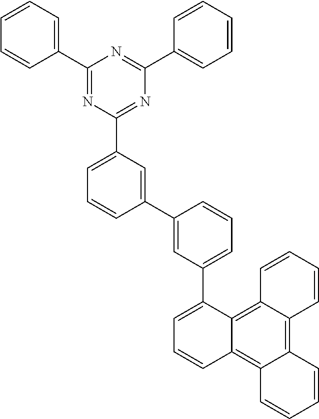 [SMILES: c1ccc(-c2nc(-c3ccccc3)nc(-c3cccc(-c4cccc(-c5cccc6c7ccccc7c7ccccc7c56)c4)c3)n2)cc1]

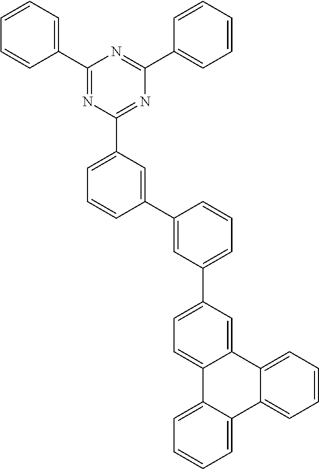 [SMILES: c1ccc(-c2nc(-c3ccccc3)nc(-c3cccc(-c4cccc(-c5ccc6c7ccccc7c7ccccc7c6c5)c4)c3)n2)cc1]

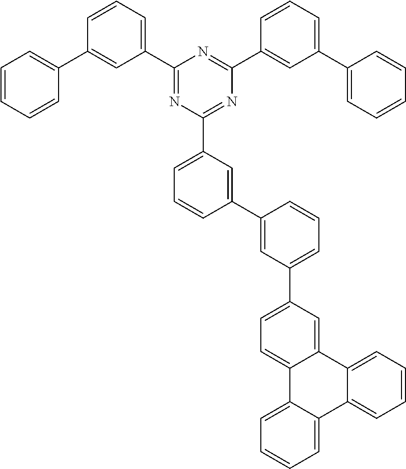 [SMILES: c1ccc(-c2cccc(-c3nc(-c4cccc(-c5ccccc5)c4)nc(-c4cccc(-c5cccc(-c6ccc7c8ccccc8c8ccccc8c7c6)c5)c4)n3)c2)cc1]

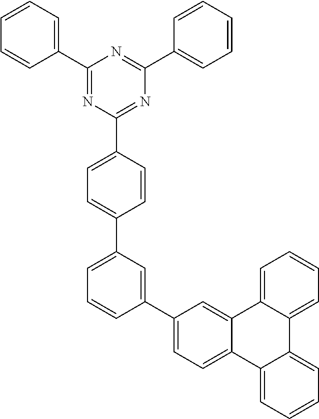 [SMILES: c1ccc(-c2nc(-c3ccccc3)nc(-c3ccc(-c4cccc(-c5ccc6c7ccccc7c7ccccc7c6c5)c4)cc3)n2)cc1]

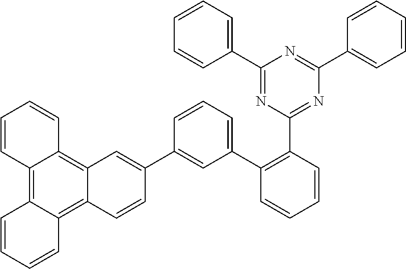 [SMILES: c1ccc(-c2nc(-c3ccccc3)nc(-c3ccccc3-c3cccc(-c4ccc5c6ccccc6c6ccccc6c5c4)c3)n2)cc1]

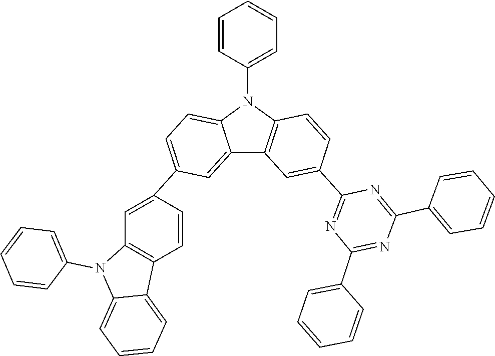 [SMILES: c1ccc(-c2nc(-c3ccccc3)nc(-c3ccc4c(c3)c3cc(-c5ccc6c7ccccc7n(-c7ccccc7)c6c5)ccc3n4-c3ccccc3)n2)cc1]

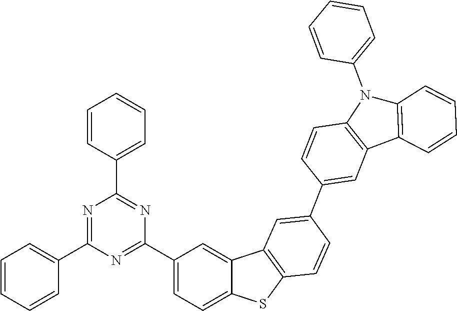 [SMILES: c1ccc(-c2nc(-c3ccccc3)nc(-c3ccc4sc5ccc(-c6ccc7c(c6)c6ccccc6n7-c6ccccc6)cc5c4c3)n2)cc1]

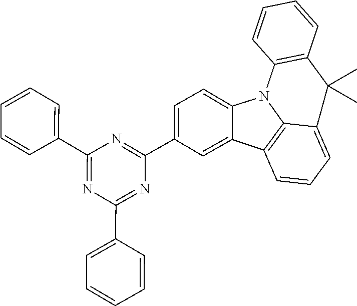 [SMILES: CC1(C)c2ccccc2-n2c3ccc(-c4nc(-c5ccccc5)nc(-c5ccccc5)n4)cc3c3cccc1c32]

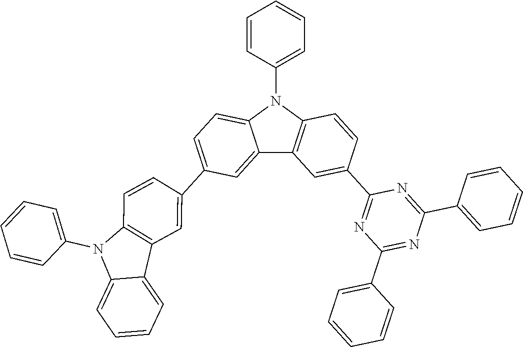 [SMILES: c1ccc(-c2nc(-c3ccccc3)nc(-c3ccc4c(c3)c3cc(-c5ccc6c(c5)c5ccccc5n6-c5ccccc5)ccc3n4-c3ccccc3)n2)cc1]

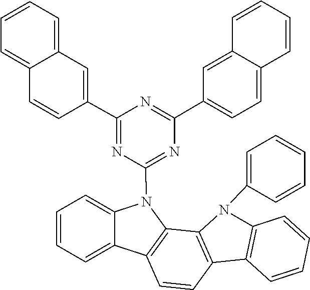 [SMILES: c1ccc(-n2c3ccccc3c3ccc4c5ccccc5n(-c5nc(-c6ccc7ccccc7c6)nc(-c6ccc7ccccc7c6)n5)c4c32)cc1]

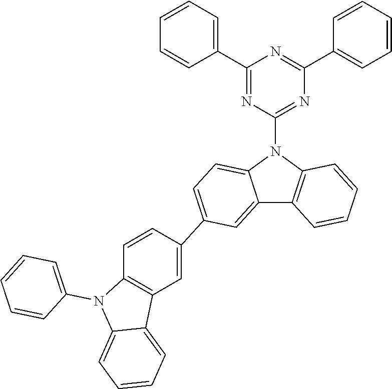 [SMILES: c1ccc(-c2nc(-c3ccccc3)nc(-n3c4ccccc4c4cc(-c5ccc6c(c5)c5ccccc5n6-c5ccccc5)ccc43)n2)cc1]

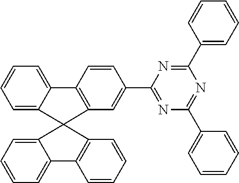 [SMILES: c1ccc(-c2nc(-c3ccccc3)nc(-c3ccc4c(c3)C3(c5ccccc5-c5ccccc53)c3ccccc3-4)n2)cc1]

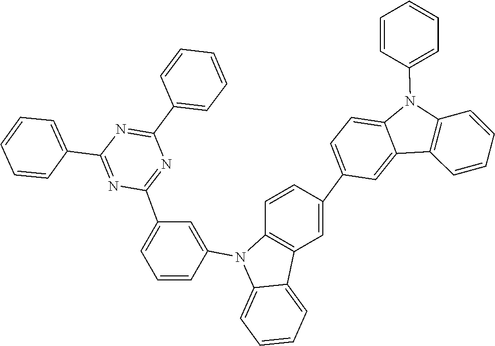 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1]

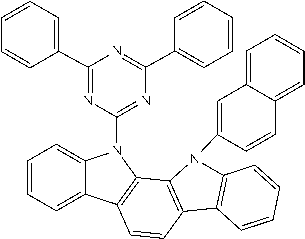 [SMILES: c1ccc(-c2nc(-c3ccccc3)nc(-n3c4ccccc4c4ccc5c6ccccc6n(-c6ccc7ccccc7c6)c5c43)n2)cc1]

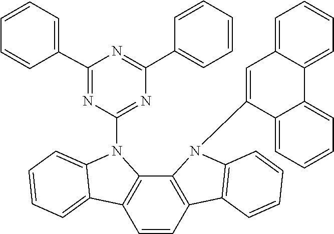 [SMILES: c1ccc(-c2nc(-c3ccccc3)nc(-n3c4ccccc4c4ccc5c6ccccc6n(-c6cc7ccccc7c7ccccc67)c5c43)n2)cc1]

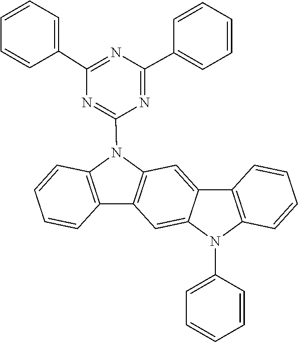 [SMILES: c1ccc(-c2nc(-c3ccccc3)nc(-n3c4ccccc4c4cc5c(cc43)c3ccccc3n5-c3ccccc3)n2)cc1]

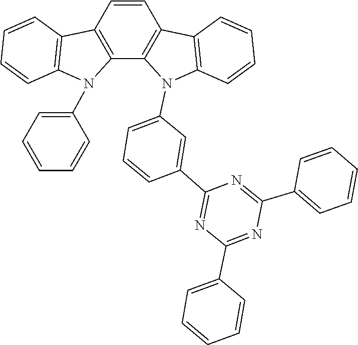 [SMILES: c1ccc(-c2nc(-c3ccccc3)nc(-c3cccc(-n4c5ccccc5c5ccc6c7ccccc7n(-c7ccccc7)c6c54)c3)n2)cc1]

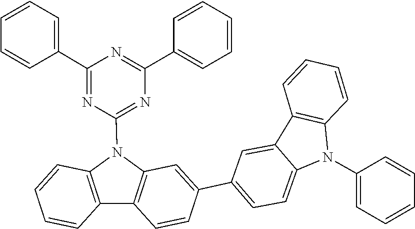 [SMILES: c1ccc(-c2nc(-c3ccccc3)nc(-n3c4ccccc4c4ccc(-c5ccc6c(c5)c5ccccc5n6-c5ccccc5)cc43)n2)cc1]

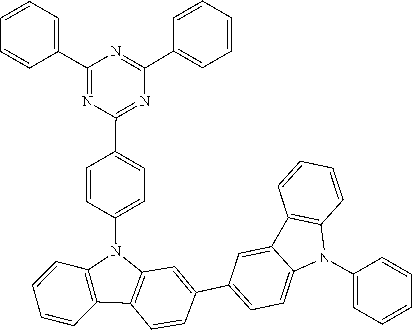 [SMILES: c1ccc(-c2nc(-c3ccccc3)nc(-c3ccc(-n4c5ccccc5c5ccc(-c6ccc7c(c6)c6ccccc6n7-c6ccccc6)cc54)cc3)n2)cc1]

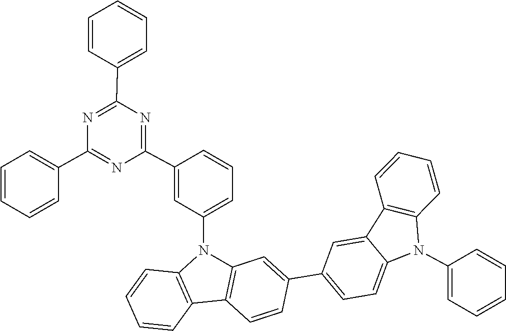 [SMILES: c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1]

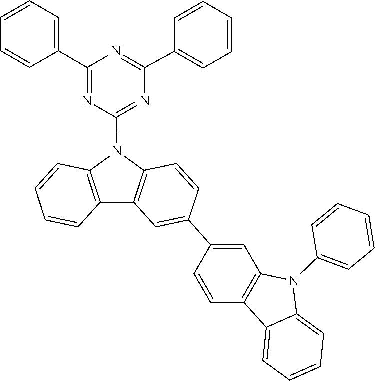 [SMILES: c1ccc(-c2nc(-c3ccccc3)nc(-n3c4ccccc4c4cc(-c5ccc6c7ccccc7n(-c7ccccc7)c6c5)ccc43)n2)cc1]

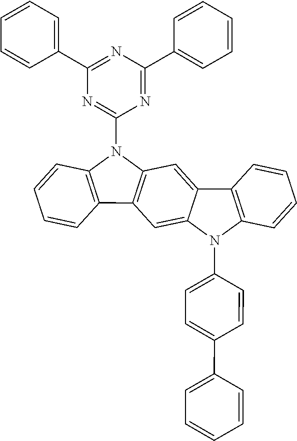 [SMILES: c1ccc(-c2ccc(-n3c4ccccc4c4cc5c(cc43)c3ccccc3n5-c3nc(-c4ccccc4)nc(-c4ccccc4)n3)cc2)cc1]

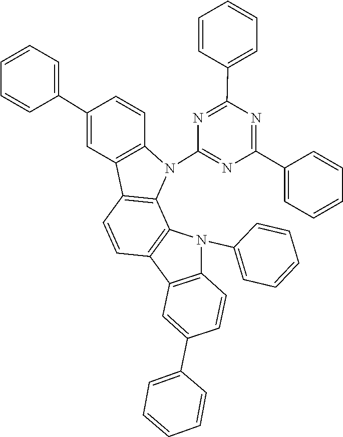 [SMILES: c1ccc(-c2ccc3c(c2)c2ccc4c5cc(-c6ccccc6)ccc5n(-c5nc(-c6ccccc6)nc(-c6ccccc6)n5)c4c2n3-c2ccccc2)cc1]

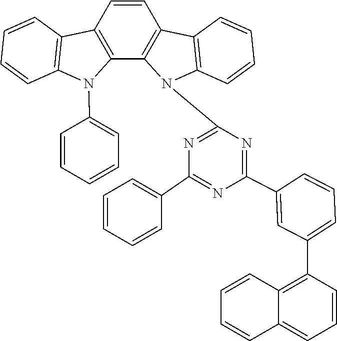 [SMILES: c1ccc(-c2nc(-c3cccc(-c4cccc5ccccc45)c3)nc(-n3c4ccccc4c4ccc5c6ccccc6n(-c6ccccc6)c5c43)n2)cc1]

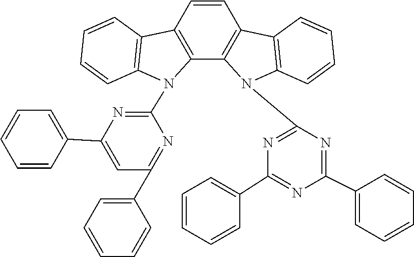 [SMILES: c1ccc(-c2cc(-c3ccccc3)nc(-n3c4ccccc4c4ccc5c6ccccc6n(-c6nc(-c7ccccc7)nc(-c7ccccc7)n6)c5c43)n2)cc1]

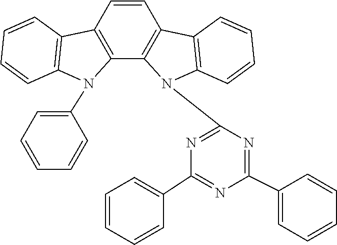 [SMILES: c1ccc(-c2nc(-c3ccccc3)nc(-n3c4ccccc4c4ccc5c6ccccc6n(-c6ccccc6)c5c43)n2)cc1]

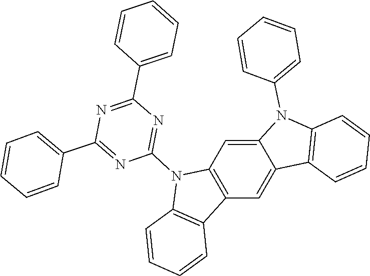 [SMILES: c1ccc(-c2nc(-c3ccccc3)nc(-n3c4ccccc4c4cc5c6ccccc6n(-c6ccccc6)c5cc43)n2)cc1]

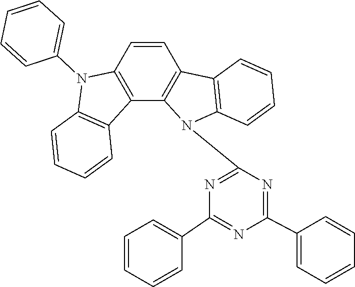 [SMILES: c1ccc(-c2nc(-c3ccccc3)nc(-n3c4ccccc4c4ccc5c(c6ccccc6n5-c5ccccc5)c43)n2)cc1]

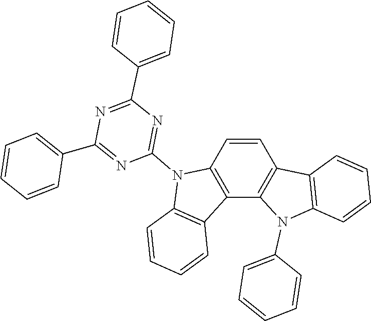 [SMILES: c1ccc(-c2nc(-c3ccccc3)nc(-n3c4ccccc4c4c3ccc3c5ccccc5n(-c5ccccc5)c34)n2)cc1]

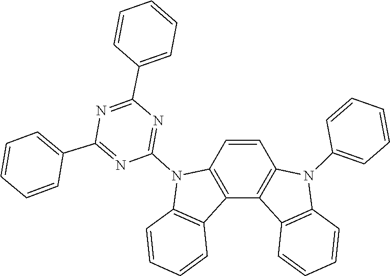 [SMILES: c1ccc(-c2nc(-c3ccccc3)nc(-n3c4ccccc4c4c5c6ccccc6n(-c6ccccc6)c5ccc43)n2)cc1]

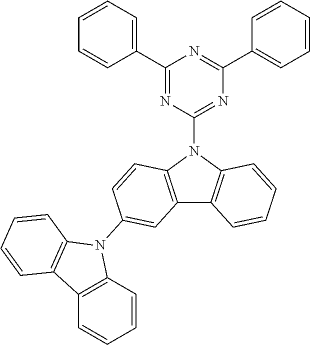 [SMILES: c1ccc(-c2nc(-c3ccccc3)nc(-n3c4ccccc4c4cc(-n5c6ccccc6c6ccccc65)ccc43)n2)cc1]

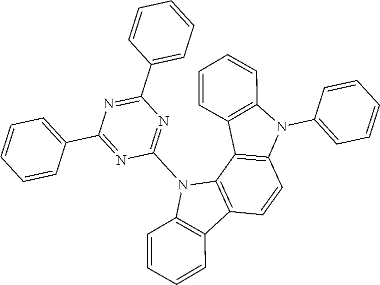 [SMILES: c1ccc(-c2nc(-c3ccccc3)nc(-n3c4ccccc4c4ccc5c(c6ccccc6n5-c5ccccc5)c43)n2)cc1]

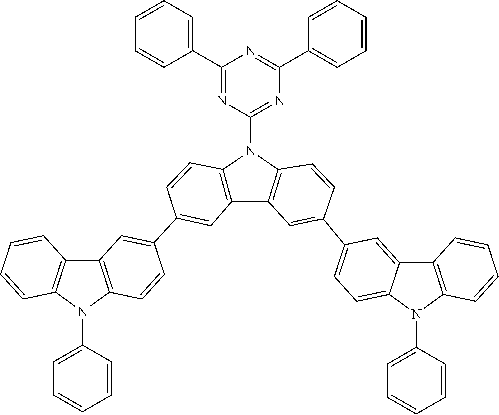 [SMILES: C1=CC(c2ccc3c(c2)c2ccccc2n3-c2ccccc2)C=Cc2c1c1cc(-c3ccc4c(c3)c3ccccc3n4-c3ccccc3)ccc1n2-c1nc(-c2ccccc2)nc(-c2ccccc2)n1]

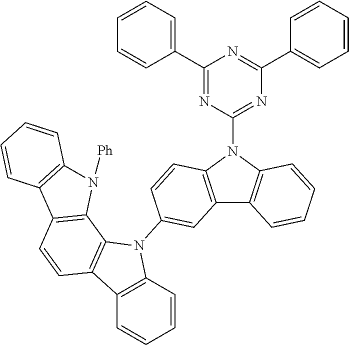 [SMILES: c1ccc(-c2nc(-c3ccccc3)nc(-n3c4ccccc4c4cc(-n5c6ccccc6c6ccc7c8ccccc8n(-c8ccccc8)c7c65)ccc43)n2)cc1]

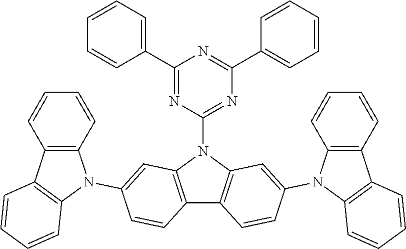 [SMILES: c1ccc(-c2nc(-c3ccccc3)nc(-n3c4cc(-n5c6ccccc6c6ccccc65)ccc4c4ccc(-n5c6ccccc6c6ccccc65)cc43)n2)cc1]

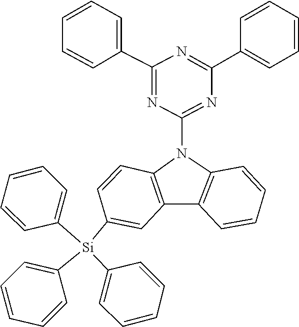 [SMILES: c1ccc(-c2nc(-c3ccccc3)nc(-n3c4ccccc4c4cc([Si](c5ccccc5)(c5ccccc5)c5ccccc5)ccc43)n2)cc1]

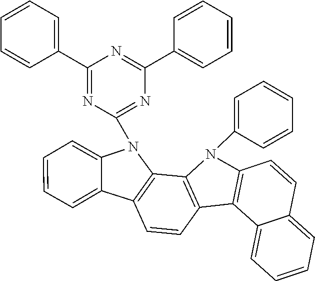 [SMILES: c1ccc(-c2nc(-c3ccccc3)nc(-n3c4ccccc4c4ccc5c6c7ccccc7ccc6n(-c6ccccc6)c5c43)n2)cc1]

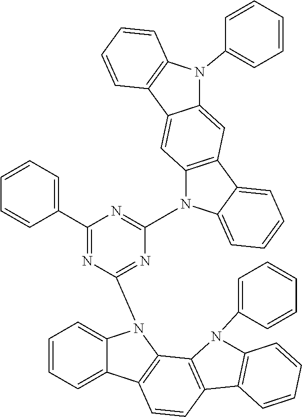 [SMILES: c1ccc(-c2nc(-n3c4ccccc4c4cc5c(cc43)c3ccccc3n5-c3ccccc3)nc(-n3c4ccccc4c4ccc5c6ccccc6n(-c6ccccc6)c5c43)n2)cc1]

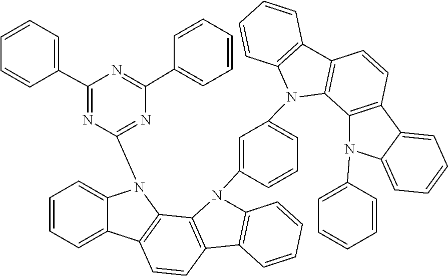 [SMILES: c1ccc(-c2nc(-c3ccccc3)nc(-n3c4ccccc4c4ccc5c6ccccc6n(-c6cccc(-n7c8ccccc8c8ccc9c%10ccccc%10n(-c%10ccccc%10)c9c87)c6)c5c43)n2)cc1]

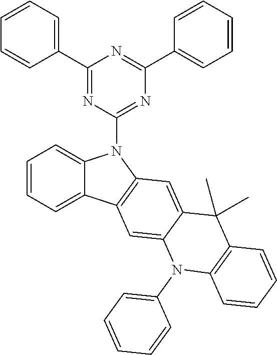 [SMILES: CC1(C)c2ccccc2N(c2ccccc2)c2cc3c4ccccc4n(-c4nc(-c5ccccc5)nc(-c5ccccc5)n4)c3cc21]

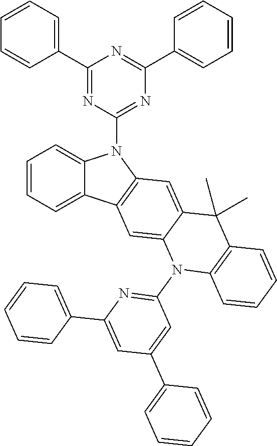 [SMILES: CC1(C)c2ccccc2N(c2cc(-c3ccccc3)cc(-c3ccccc3)n2)c2cc3c4ccccc4n(-c4nc(-c5ccccc5)nc(-c5ccccc5)n4)c3cc21]

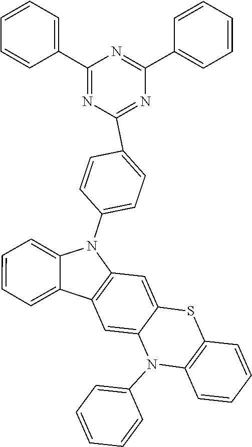 [SMILES: c1ccc(-c2nc(-c3ccccc3)nc(-c3ccc(-n4c5ccccc5c5cc6c(cc54)Sc4ccccc4N6c4ccccc4)cc3)n2)cc1]

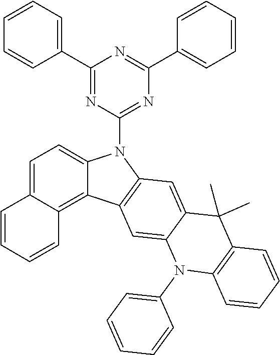 [SMILES: CC1(C)c2ccccc2N(c2ccccc2)c2cc3c4c5ccccc5ccc4n(-c4nc(-c5ccccc5)nc(-c5ccccc5)n4)c3cc21]

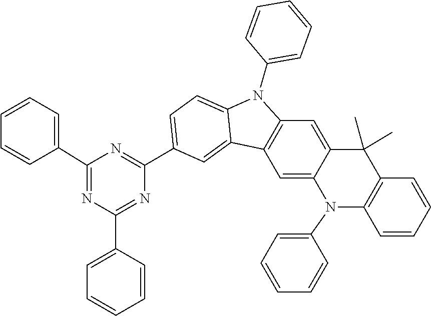 [SMILES: CC1(C)c2ccccc2N(c2ccccc2)c2cc3c4cc(-c5nc(-c6ccccc6)nc(-c6ccccc6)n5)ccc4n(-c4ccccc4)c3cc21]

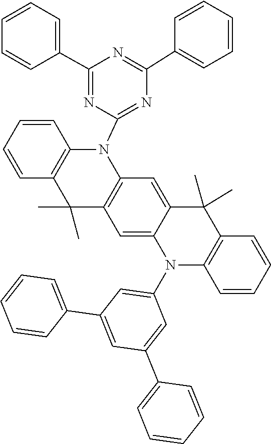 [SMILES: CC1(C)c2ccccc2N(c2cc(-c3ccccc3)cc(-c3ccccc3)c2)c2cc3c(cc21)N(c1nc(-c2ccccc2)nc(-c2ccccc2)n1)c1ccccc1C3(C)C]

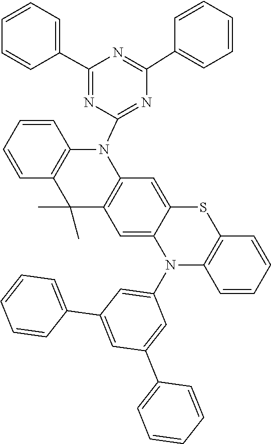 [SMILES: CC1(C)c2ccccc2N(c2nc(-c3ccccc3)nc(-c3ccccc3)n2)c2cc3c(cc21)N(c1cc(-c2ccccc2)cc(-c2ccccc2)c1)c1ccccc1S3]

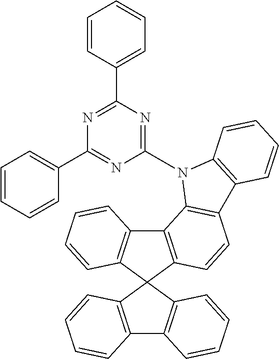 [SMILES: c1ccc(-c2nc(-c3ccccc3)nc(-n3c4ccccc4c4ccc5c(c43)-c3ccccc3C53c4ccccc4-c4ccccc43)n2)cc1]

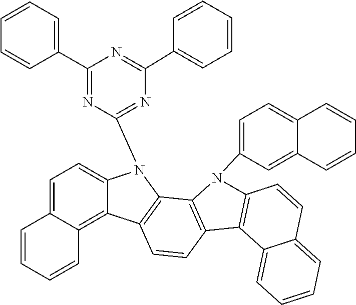 [SMILES: c1ccc(-c2nc(-c3ccccc3)nc(-n3c4ccc5ccccc5c4c4ccc5c6c7ccccc7ccc6n(-c6ccc7ccccc7c6)c5c43)n2)cc1]

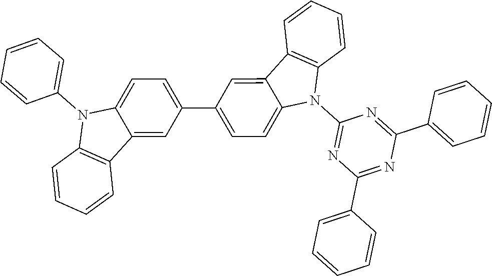 [SMILES: c1ccc(-c2nc(-c3ccccc3)nc(-n3c4ccccc4c4cc(-c5ccc6c(c5)c5ccccc5n6-c5ccccc5)ccc43)n2)cc1]

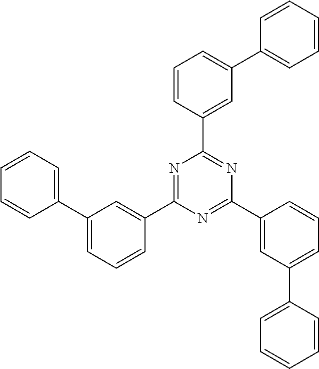 [SMILES: c1ccc(-c2cccc(-c3nc(-c4cccc(-c5ccccc5)c4)nc(-c4cccc(-c5ccccc5)c4)n3)c2)cc1]

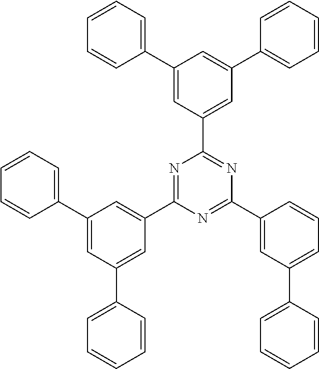 [SMILES: c1ccc(-c2cccc(-c3nc(-c4cc(-c5ccccc5)cc(-c5ccccc5)c4)nc(-c4cc(-c5ccccc5)cc(-c5ccccc5)c4)n3)c2)cc1]

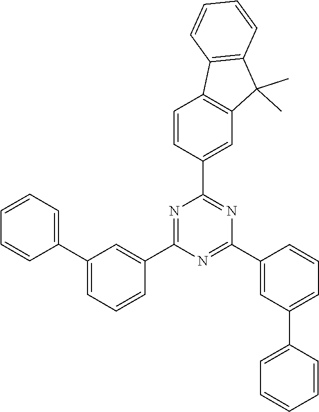 [SMILES: CC1(C)c2ccccc2-c2ccc(-c3nc(-c4cccc(-c5ccccc5)c4)nc(-c4cccc(-c5ccccc5)c4)n3)cc21]

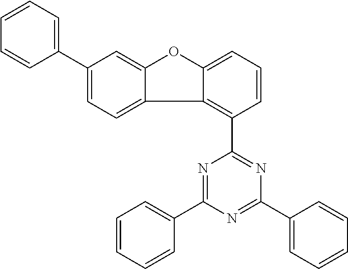 [SMILES: c1ccc(-c2ccc3c(c2)oc2cccc(-c4nc(-c5ccccc5)nc(-c5ccccc5)n4)c23)cc1]

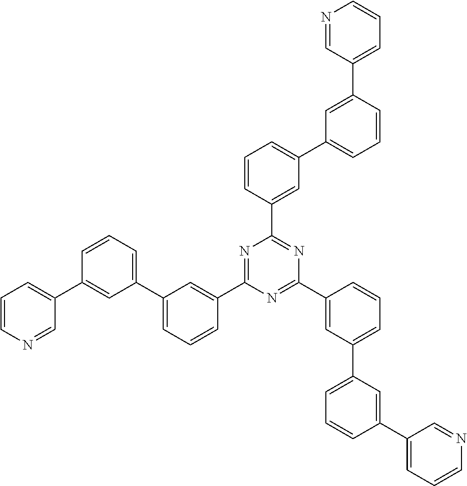 [SMILES: c1cncc(-c2cccc(-c3cccc(-c4nc(-c5cccc(-c6cccc(-c7cccnc7)c6)c5)nc(-c5cccc(-c6cccc(-c7cccnc7)c6)c5)n4)c3)c2)c1]

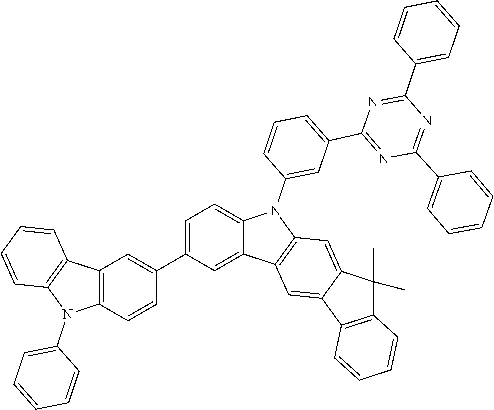 [SMILES: CC1(C)c2ccccc2-c2cc3c4cc(-c5ccc6c(c5)c5ccccc5n6-c5ccccc5)ccc4n(-c4cccc(-c5nc(-c6ccccc6)nc(-c6ccccc6)n5)c4)c3cc21]

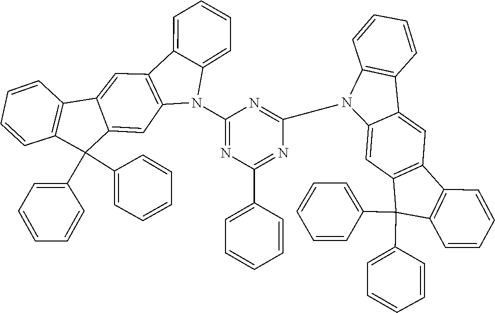 [SMILES: c1ccc(-c2nc(-n3c4ccccc4c4cc5c(cc43)C(c3ccccc3)(c3ccccc3)c3ccccc3-5)nc(-n3c4ccccc4c4cc5c(cc43)C(c3ccccc3)(c3ccccc3)c3ccccc3-5)n2)cc1]

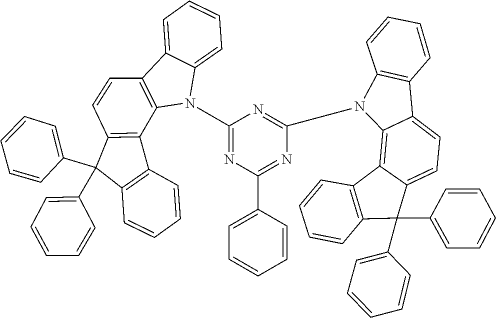 [SMILES: c1ccc(-c2nc(-n3c4ccccc4c4ccc5c(c43)-c3ccccc3C5(c3ccccc3)c3ccccc3)nc(-n3c4ccccc4c4ccc5c(c43)-c3ccccc3C5(c3ccccc3)c3ccccc3)n2)cc1]

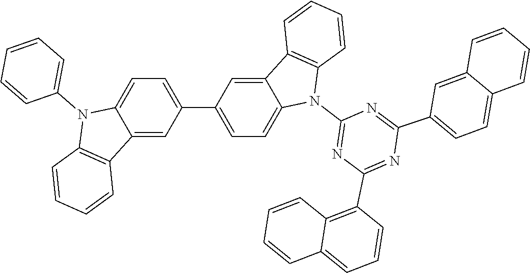 [SMILES: c1ccc(-n2c3ccccc3c3cc(-c4ccc5c(c4)c4ccccc4n5-c4nc(-c5ccc6ccccc6c5)nc(-c5cccc6ccccc56)n4)ccc32)cc1]

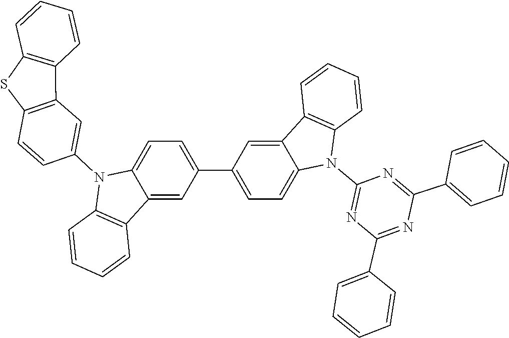 [SMILES: c1ccc(-c2nc(-c3ccccc3)nc(-n3c4ccccc4c4cc(-c5ccc6c(c5)c5ccccc5n6-c5ccc6sc7ccccc7c6c5)ccc43)n2)cc1]

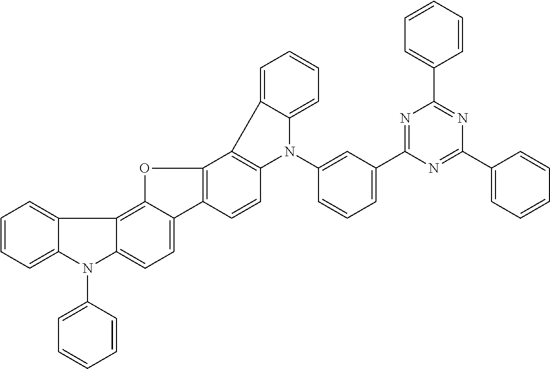 [SMILES: c1ccc(-c2nc(-c3ccccc3)nc(-c3cccc(-n4c5ccccc5c5c6oc7c(ccc8c7c7ccccc7n8-c7ccccc7)c6ccc54)c3)n2)cc1]

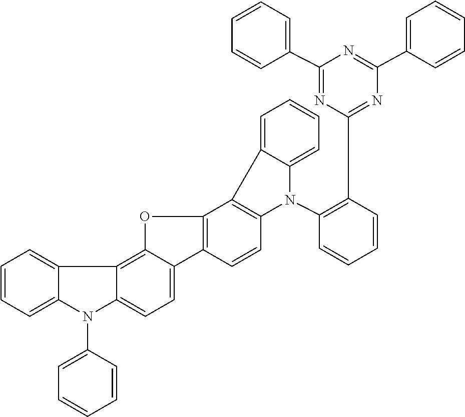 [SMILES: c1ccc(-c2nc(-c3ccccc3)nc(-c3ccccc3-n3c4ccccc4c4c5oc6c(ccc7c6c6ccccc6n7-c6ccccc6)c5ccc43)n2)cc1]

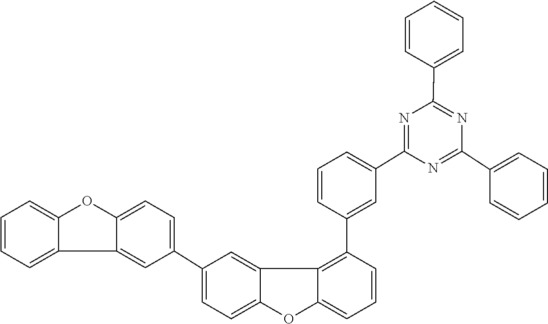 [SMILES: c1ccc(-c2nc(-c3ccccc3)nc(-c3cccc(-c4cccc5oc6ccc(-c7ccc8oc9ccccc9c8c7)cc6c45)c3)n2)cc1]

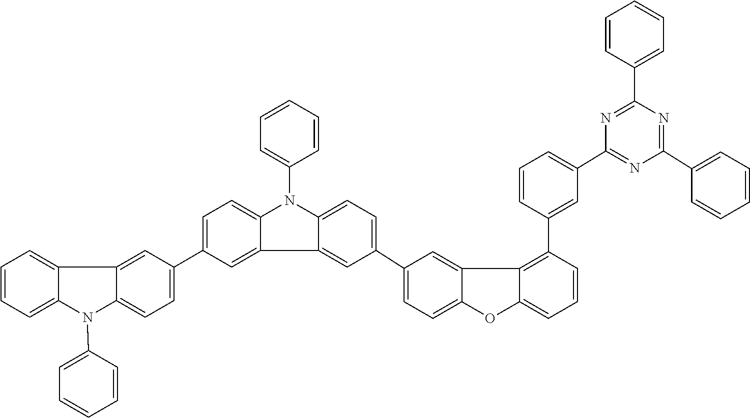 [SMILES: c1ccc(-c2nc(-c3ccccc3)nc(-c3cccc(-c4cccc5oc6ccc(-c7ccc8c(c7)c7cc(-c9ccc%10c(c9)c9ccccc9n%10-c9ccccc9)ccc7n8-c7ccccc7)cc6c45)c3)n2)cc1]

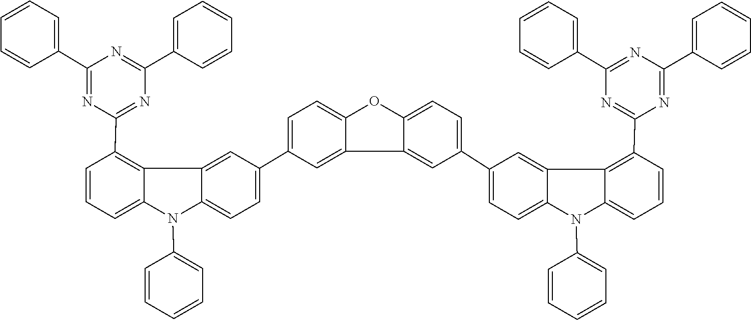 [SMILES: c1ccc(-c2nc(-c3ccccc3)nc(-c3cccc4c3c3cc(-c5ccc6oc7ccc(-c8ccc9c(c8)c8c(-c%10nc(-c%11ccccc%11)nc(-c%11ccccc%11)n%10)cccc8n9-c8ccccc8)cc7c6c5)ccc3n4-c3ccccc3)n2)cc1]

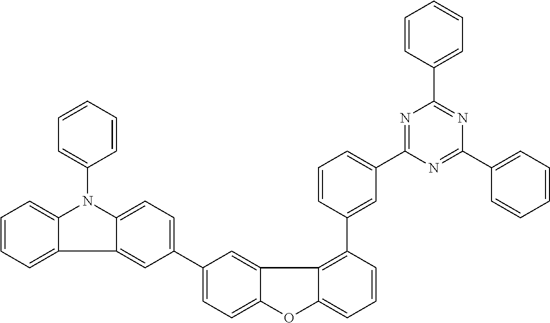 [SMILES: c1ccc(-c2nc(-c3ccccc3)nc(-c3cccc(-c4cccc5oc6ccc(-c7ccc8c(c7)c7ccccc7n8-c7ccccc7)cc6c45)c3)n2)cc1]

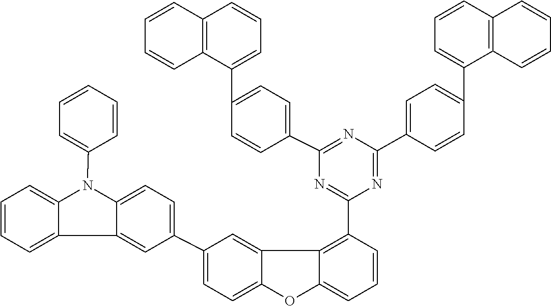 [SMILES: c1ccc(-n2c3ccccc3c3cc(-c4ccc5oc6cccc(-c7nc(-c8ccc(-c9cccc%10ccccc9%10)cc8)nc(-c8ccc(-c9cccc%10ccccc9%10)cc8)n7)c6c5c4)ccc32)cc1]

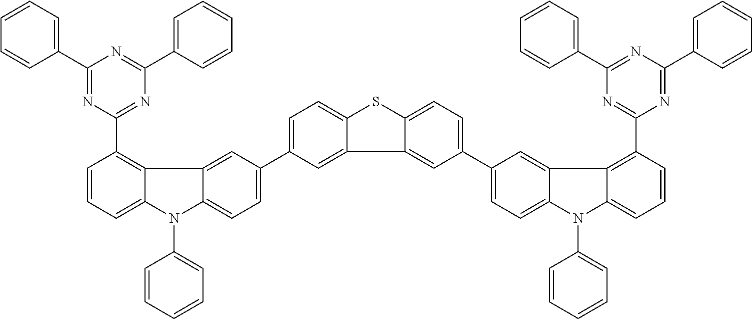 [SMILES: c1ccc(-c2nc(-c3ccccc3)nc(-c3cccc4c3c3cc(-c5ccc6sc7ccc(-c8ccc9c(c8)c8c(-c%10nc(-c%11ccccc%11)nc(-c%11ccccc%11)n%10)cccc8n9-c8ccccc8)cc7c6c5)ccc3n4-c3ccccc3)n2)cc1]

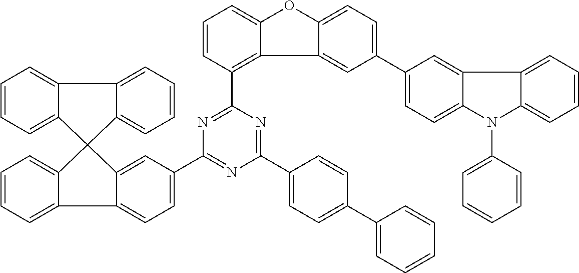 [SMILES: c1ccc(-c2ccc(-c3nc(-c4ccc5c(c4)C4(c6ccccc6-c6ccccc64)c4ccccc4-5)nc(-c4cccc5oc6ccc(-c7ccc8c(c7)c7ccccc7n8-c7ccccc7)cc6c45)n3)cc2)cc1]

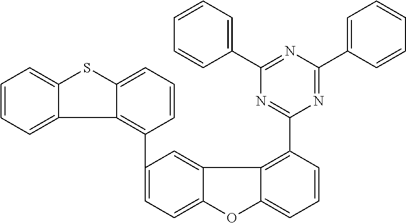 [SMILES: c1ccc(-c2nc(-c3ccccc3)nc(-c3cccc4oc5ccc(-c6cccc7sc8ccccc8c67)cc5c34)n2)cc1]

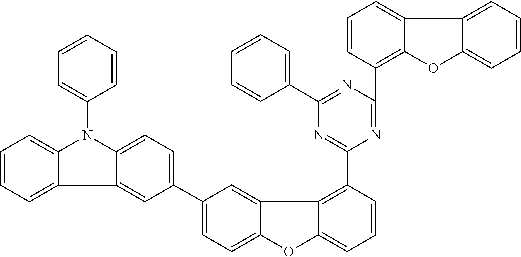 [SMILES: c1ccc(-c2nc(-c3cccc4c3oc3ccccc34)nc(-c3cccc4oc5ccc(-c6ccc7c(c6)c6ccccc6n7-c6ccccc6)cc5c34)n2)cc1]

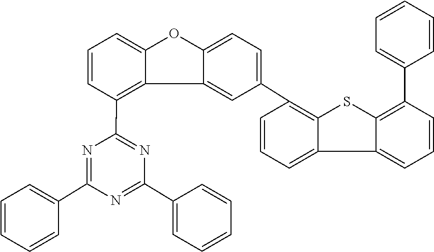 [SMILES: c1ccc(-c2nc(-c3ccccc3)nc(-c3cccc4oc5ccc(-c6cccc7c6sc6c(-c8ccccc8)cccc67)cc5c34)n2)cc1]

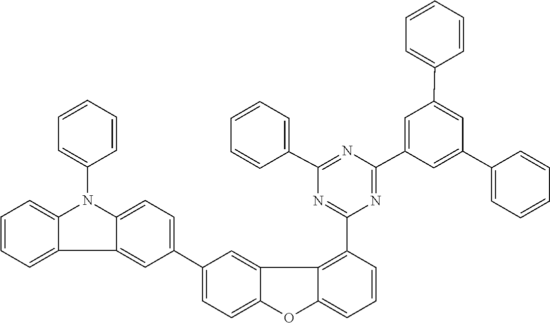 [SMILES: c1ccc(-c2cc(-c3ccccc3)cc(-c3nc(-c4ccccc4)nc(-c4cccc5oc6ccc(-c7ccc8c(c7)c7ccccc7n8-c7ccccc7)cc6c45)n3)c2)cc1]

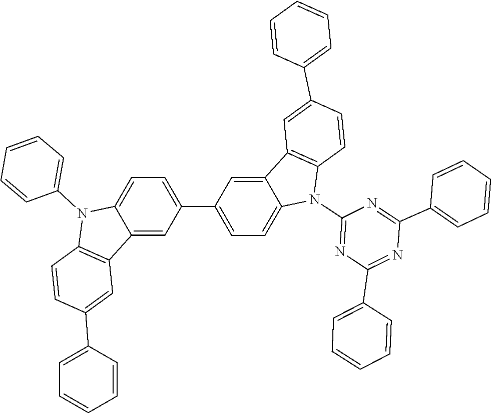 [SMILES: c1ccc(-c2ccc3c(c2)c2cc(-c4ccc5c(c4)c4cc(-c6ccccc6)ccc4n5-c4nc(-c5ccccc5)nc(-c5ccccc5)n4)ccc2n3-c2ccccc2)cc1]

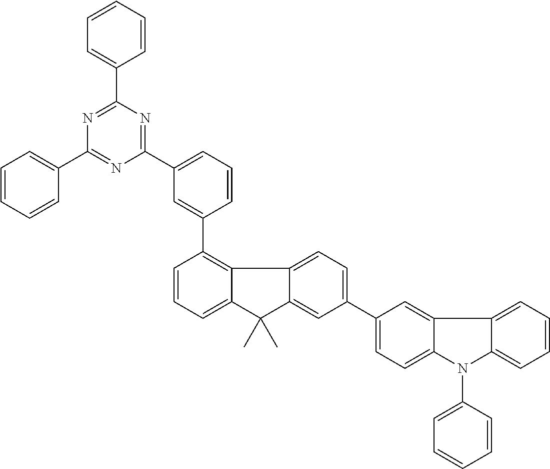 [SMILES: CC1(C)c2cc(-c3ccc4c(c3)c3ccccc3n4-c3ccccc3)ccc2-c2c(-c3cccc(-c4nc(-c5ccccc5)nc(-c5ccccc5)n4)c3)cccc21]

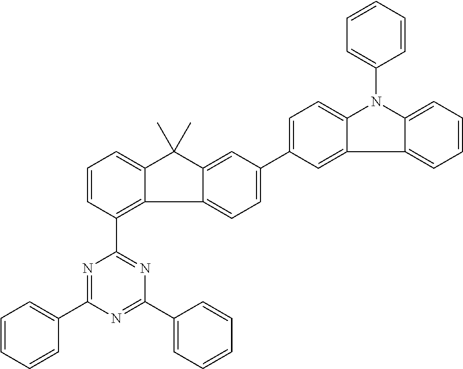 [SMILES: CC1(C)c2cc(-c3ccc4c(c3)c3ccccc3n4-c3ccccc3)ccc2-c2c(-c3nc(-c4ccccc4)nc(-c4ccccc4)n3)cccc21]

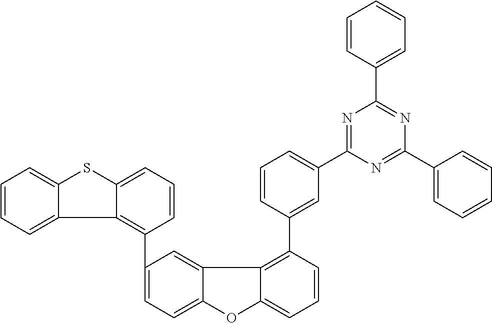 [SMILES: c1ccc(-c2nc(-c3ccccc3)nc(-c3cccc(-c4cccc5oc6ccc(-c7cccc8sc9ccccc9c78)cc6c45)c3)n2)cc1]

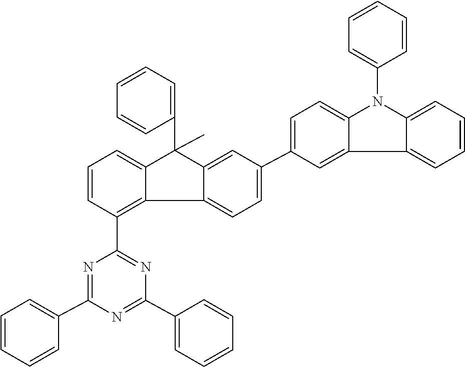 [SMILES: CC1(c2ccccc2)c2cc(-c3ccc4c(c3)c3ccccc3n4-c3ccccc3)ccc2-c2c(-c3nc(-c4ccccc4)nc(-c4ccccc4)n3)cccc21]

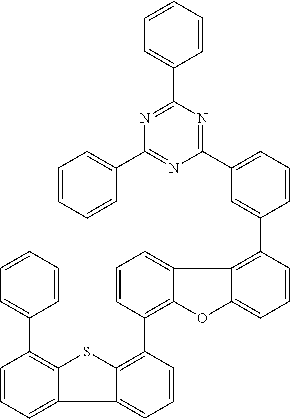 [SMILES: c1ccc(-c2nc(-c3ccccc3)nc(-c3cccc(-c4cccc5oc6c(-c7cccc8c7sc7c(-c9ccccc9)cccc78)cccc6c45)c3)n2)cc1]

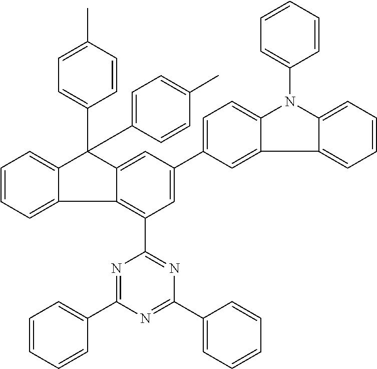 [SMILES: Cc1ccc(C2(c3ccc(C)cc3)c3ccccc3-c3c(-c4nc(-c5ccccc5)nc(-c5ccccc5)n4)cc(-c4ccc5c(c4)c4ccccc4n5-c4ccccc4)cc32)cc1]

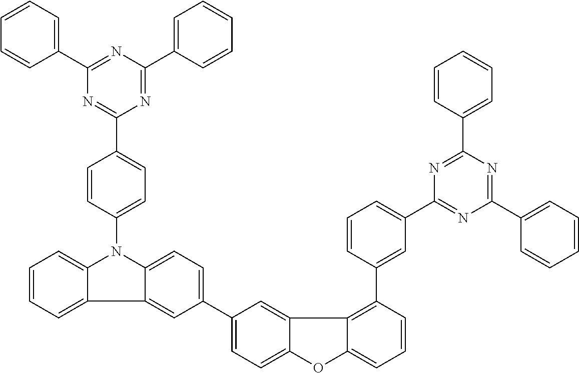 [SMILES: c1ccc(-c2nc(-c3ccccc3)nc(-c3ccc(-n4c5ccccc5c5cc(-c6ccc7oc8cccc(-c9cccc(-c%10nc(-c%11ccccc%11)nc(-c%11ccccc%11)n%10)c9)c8c7c6)ccc54)cc3)n2)cc1]

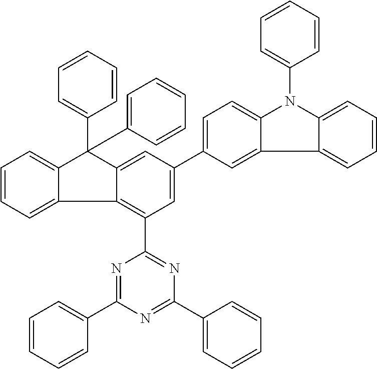 [SMILES: c1ccc(-c2nc(-c3ccccc3)nc(-c3cc(-c4ccc5c(c4)c4ccccc4n5-c4ccccc4)cc4c3-c3ccccc3C4(c3ccccc3)c3ccccc3)n2)cc1]

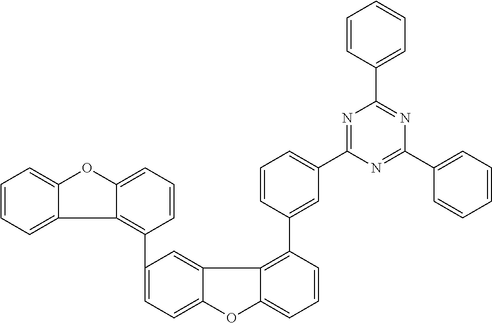 [SMILES: c1ccc(-c2nc(-c3ccccc3)nc(-c3cccc(-c4cccc5oc6ccc(-c7cccc8oc9ccccc9c78)cc6c45)c3)n2)cc1]

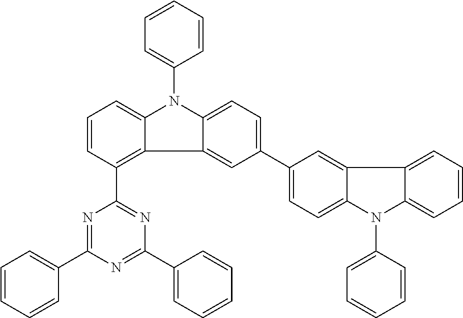 [SMILES: c1ccc(-c2nc(-c3ccccc3)nc(-c3cccc4c3c3cc(-c5ccc6c(c5)c5ccccc5n6-c5ccccc5)ccc3n4-c3ccccc3)n2)cc1]

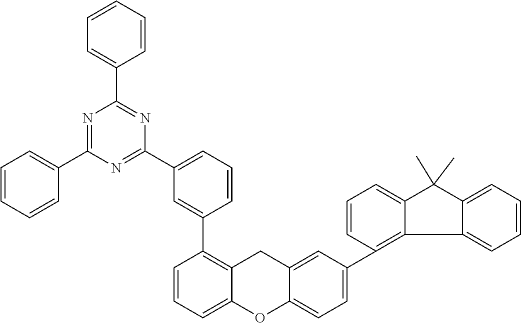 [SMILES: CC1(C)c2ccccc2-c2c(-c3ccc4c(c3)Cc3c(cccc3-c3cccc(-c5nc(-c6ccccc6)nc(-c6ccccc6)n5)c3)O4)cccc21]

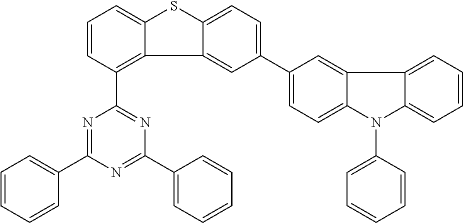 [SMILES: c1ccc(-c2nc(-c3ccccc3)nc(-c3cccc4sc5ccc(-c6ccc7c(c6)c6ccccc6n7-c6ccccc6)cc5c34)n2)cc1]

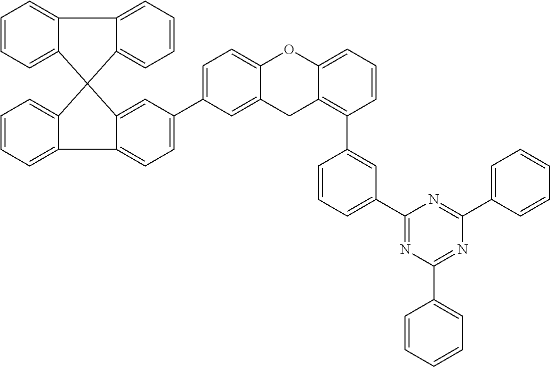 [SMILES: c1ccc(-c2nc(-c3ccccc3)nc(-c3cccc(-c4cccc5c4Cc4cc(-c6ccc7c(c6)C6(c8ccccc8-c8ccccc86)c6ccccc6-7)ccc4O5)c3)n2)cc1]

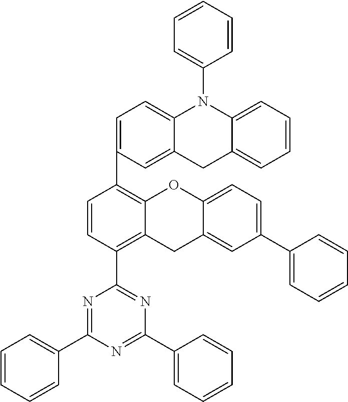 [SMILES: c1ccc(-c2ccc3c(c2)Cc2c(-c4nc(-c5ccccc5)nc(-c5ccccc5)n4)ccc(-c4ccc5c(c4)Cc4ccccc4N5c4ccccc4)c2O3)cc1]

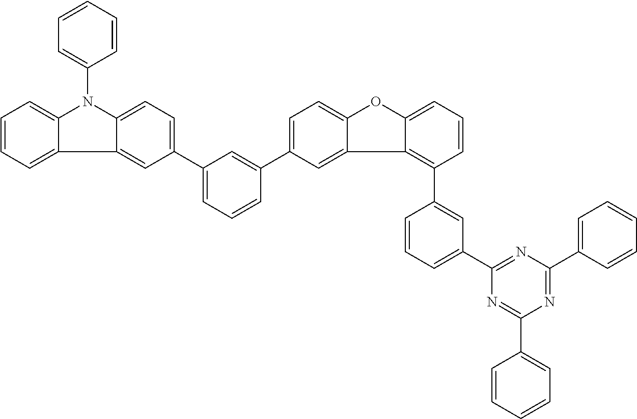 [SMILES: c1ccc(-c2nc(-c3ccccc3)nc(-c3cccc(-c4cccc5oc6ccc(-c7cccc(-c8ccc9c(c8)c8ccccc8n9-c8ccccc8)c7)cc6c45)c3)n2)cc1]

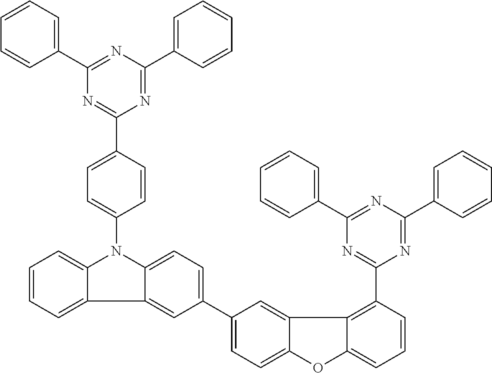 [SMILES: c1ccc(-c2nc(-c3ccccc3)nc(-c3ccc(-n4c5ccccc5c5cc(-c6ccc7oc8cccc(-c9nc(-c%10ccccc%10)nc(-c%10ccccc%10)n9)c8c7c6)ccc54)cc3)n2)cc1]

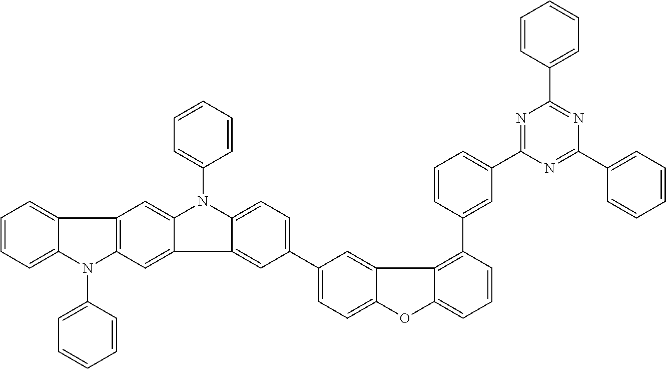 [SMILES: c1ccc(-c2nc(-c3ccccc3)nc(-c3cccc(-c4cccc5oc6ccc(-c7ccc8c(c7)c7cc9c(cc7n8-c7ccccc7)c7ccccc7n9-c7ccccc7)cc6c45)c3)n2)cc1]

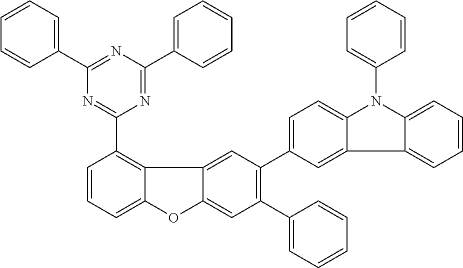 [SMILES: c1ccc(-c2nc(-c3ccccc3)nc(-c3cccc4oc5cc(-c6ccccc6)c(-c6ccc7c(c6)c6ccccc6n7-c6ccccc6)cc5c34)n2)cc1]

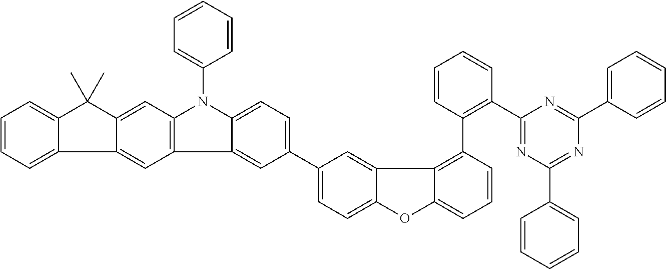 [SMILES: CC1(C)c2ccccc2-c2cc3c4cc(-c5ccc6oc7cccc(-c8ccccc8-c8nc(-c9ccccc9)nc(-c9ccccc9)n8)c7c6c5)ccc4n(-c4ccccc4)c3cc21]

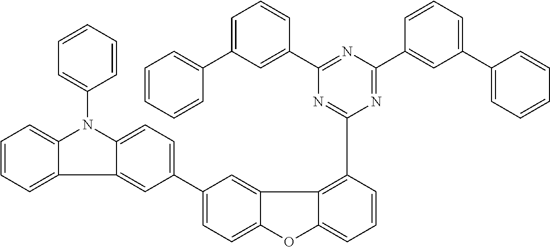 [SMILES: c1ccc(-c2cccc(-c3nc(-c4cccc(-c5ccccc5)c4)nc(-c4cccc5oc6ccc(-c7ccc8c(c7)c7ccccc7n8-c7ccccc7)cc6c45)n3)c2)cc1]

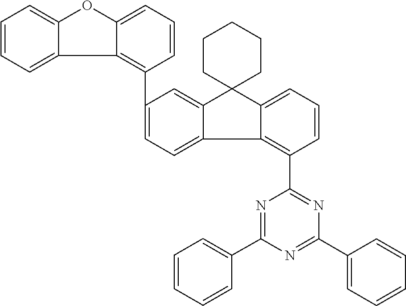 [SMILES: c1ccc(-c2nc(-c3ccccc3)nc(-c3cccc4c3-c3ccc(-c5cccc6oc7ccccc7c56)cc3C43CCCCC3)n2)cc1]

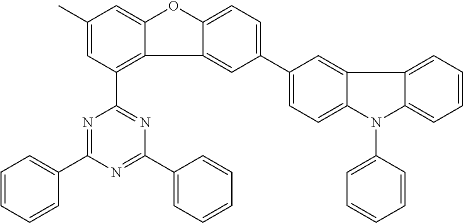 [SMILES: Cc1cc(-c2nc(-c3ccccc3)nc(-c3ccccc3)n2)c2c(c1)oc1ccc(-c3ccc4c(c3)c3ccccc3n4-c3ccccc3)cc12]

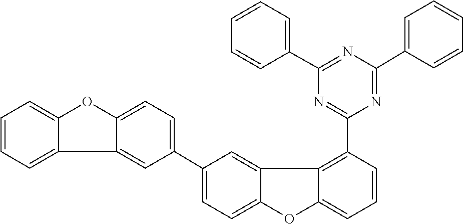 [SMILES: c1ccc(-c2nc(-c3ccccc3)nc(-c3cccc4oc5ccc(-c6ccc7oc8ccccc8c7c6)cc5c34)n2)cc1]

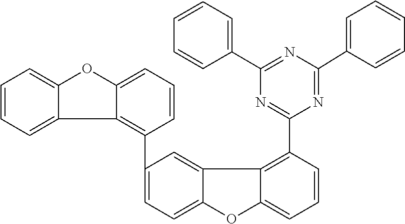 [SMILES: c1ccc(-c2nc(-c3ccccc3)nc(-c3cccc4oc5ccc(-c6cccc7oc8ccccc8c67)cc5c34)n2)cc1]

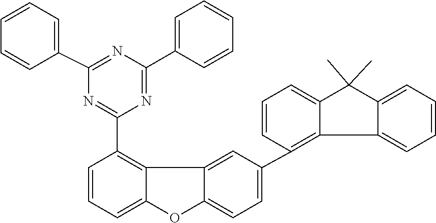 [SMILES: CC1(C)c2ccccc2-c2c(-c3ccc4oc5cccc(-c6nc(-c7ccccc7)nc(-c7ccccc7)n6)c5c4c3)cccc21]

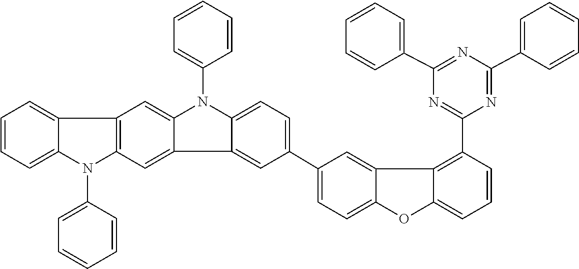 [SMILES: c1ccc(-c2nc(-c3ccccc3)nc(-c3cccc4oc5ccc(-c6ccc7c(c6)c6cc8c(cc6n7-c6ccccc6)c6ccccc6n8-c6ccccc6)cc5c34)n2)cc1]

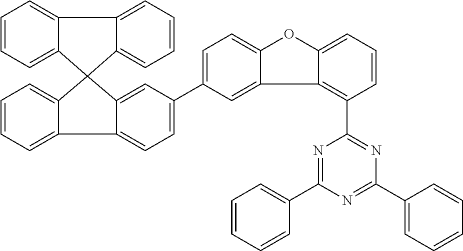 [SMILES: c1ccc(-c2nc(-c3ccccc3)nc(-c3cccc4oc5ccc(-c6ccc7c(c6)C6(c8ccccc8-c8ccccc86)c6ccccc6-7)cc5c34)n2)cc1]

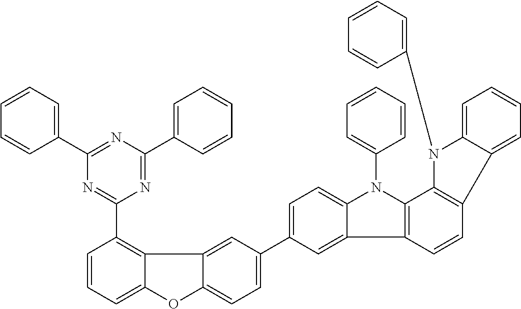 [SMILES: c1ccc(-c2nc(-c3ccccc3)nc(-c3cccc4oc5ccc(-c6ccc7c(c6)c6ccc8c9ccccc9n(-c9ccccc9)c8c6n7-c6ccccc6)cc5c34)n2)cc1]

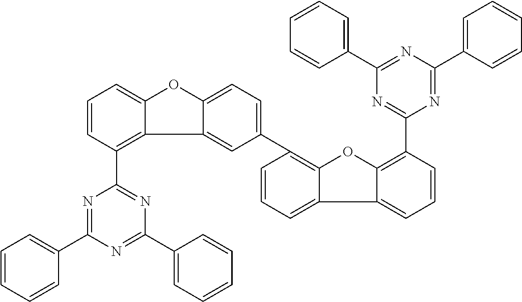 [SMILES: c1ccc(-c2nc(-c3ccccc3)nc(-c3cccc4c3oc3c(-c5ccc6oc7cccc(-c8nc(-c9ccccc9)nc(-c9ccccc9)n8)c7c6c5)cccc34)n2)cc1]

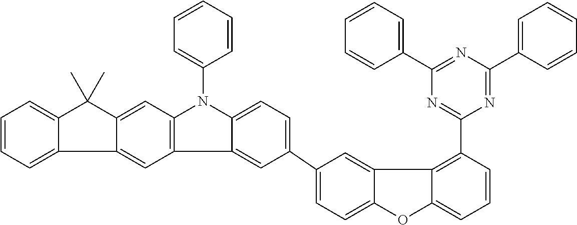 [SMILES: CC1(C)c2ccccc2-c2cc3c4cc(-c5ccc6oc7cccc(-c8nc(-c9ccccc9)nc(-c9ccccc9)n8)c7c6c5)ccc4n(-c4ccccc4)c3cc21]

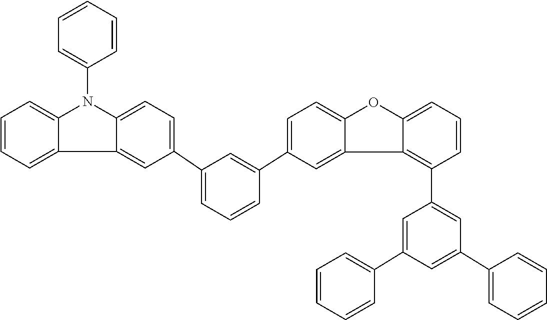 [SMILES: c1ccc(-c2cc(-c3ccccc3)cc(-c3cccc4oc5ccc(-c6cccc(-c7ccc8c(c7)c7ccccc7n8-c7ccccc7)c6)cc5c34)c2)cc1]

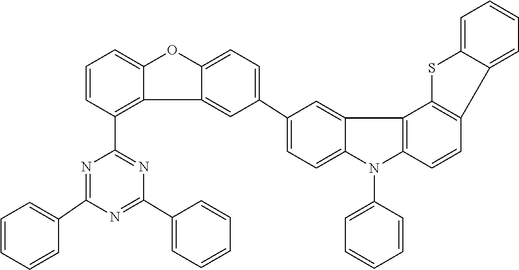 [SMILES: c1ccc(-c2nc(-c3ccccc3)nc(-c3cccc4oc5ccc(-c6ccc7c(c6)c6c8sc9ccccc9c8ccc6n7-c6ccccc6)cc5c34)n2)cc1]

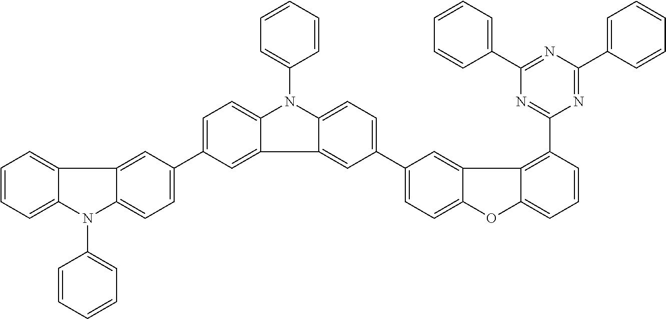 [SMILES: c1ccc(-c2nc(-c3ccccc3)nc(-c3cccc4oc5ccc(-c6ccc7c(c6)c6cc(-c8ccc9c(c8)c8ccccc8n9-c8ccccc8)ccc6n7-c6ccccc6)cc5c34)n2)cc1]

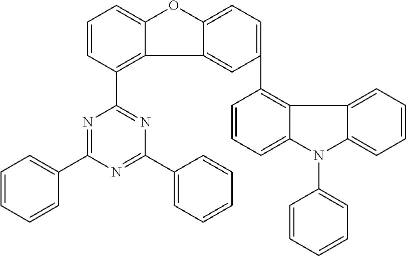 [SMILES: c1ccc(-c2nc(-c3ccccc3)nc(-c3cccc4oc5ccc(-c6cccc7c6c6ccccc6n7-c6ccccc6)cc5c34)n2)cc1]

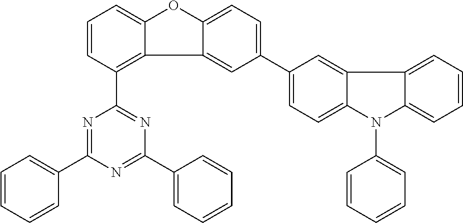 [SMILES: c1ccc(-c2nc(-c3ccccc3)nc(-c3cccc4oc5ccc(-c6ccc7c(c6)c6ccccc6n7-c6ccccc6)cc5c34)n2)cc1]

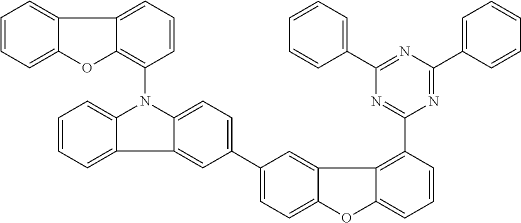 [SMILES: c1ccc(-c2nc(-c3ccccc3)nc(-c3cccc4oc5ccc(-c6ccc7c(c6)c6ccccc6n7-c6cccc7c6oc6ccccc67)cc5c34)n2)cc1]

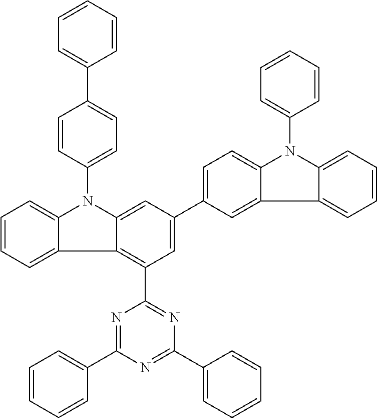 [SMILES: c1ccc(-c2ccc(-n3c4ccccc4c4c(-c5nc(-c6ccccc6)nc(-c6ccccc6)n5)cc(-c5ccc6c(c5)c5ccccc5n6-c5ccccc5)cc43)cc2)cc1]

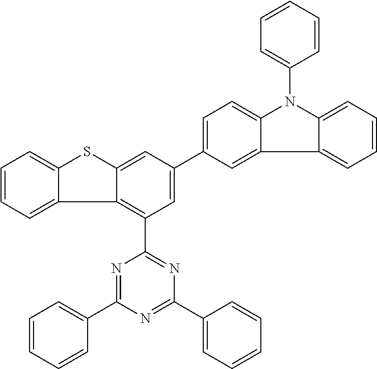 [SMILES: c1ccc(-c2nc(-c3ccccc3)nc(-c3cc(-c4ccc5c(c4)c4ccccc4n5-c4ccccc4)cc4sc5ccccc5c34)n2)cc1]

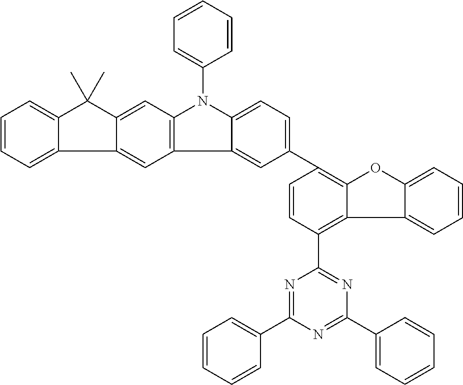 [SMILES: CC1(C)c2ccccc2-c2cc3c4cc(-c5ccc(-c6nc(-c7ccccc7)nc(-c7ccccc7)n6)c6c5oc5ccccc56)ccc4n(-c4ccccc4)c3cc21]

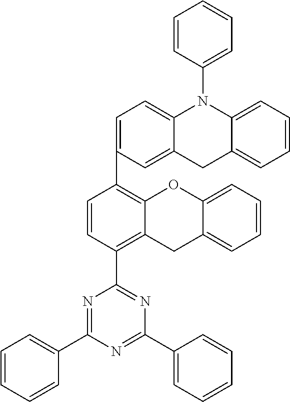 [SMILES: c1ccc(-c2nc(-c3ccccc3)nc(-c3ccc(-c4ccc5c(c4)Cc4ccccc4N5c4ccccc4)c4c3Cc3ccccc3O4)n2)cc1]

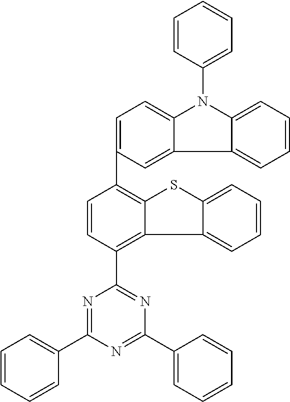 [SMILES: c1ccc(-c2nc(-c3ccccc3)nc(-c3ccc(-c4ccc5c(c4)c4ccccc4n5-c4ccccc4)c4sc5ccccc5c34)n2)cc1]

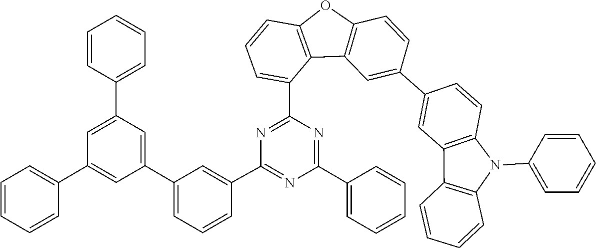 [SMILES: c1ccc(-c2cc(-c3ccccc3)cc(-c3cccc(-c4nc(-c5ccccc5)nc(-c5cccc6oc7ccc(-c8ccc9c(c8)c8ccccc8n9-c8ccccc8)cc7c56)n4)c3)c2)cc1]

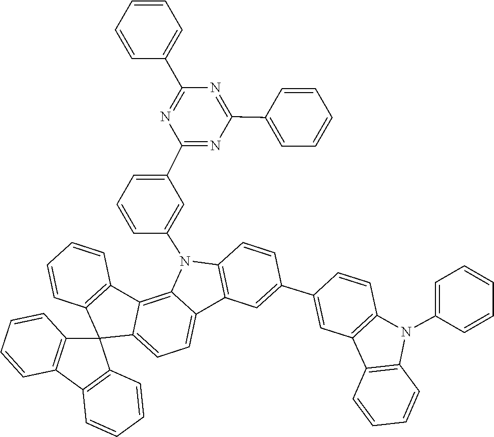 [SMILES: c1ccc(-c2nc(-c3ccccc3)nc(-c3cccc(-n4c5ccc(-c6ccc7c(c6)c6ccccc6n7-c6ccccc6)cc5c5ccc6c(c54)-c4ccccc4C64c5ccccc5-c5ccccc54)c3)n2)cc1]

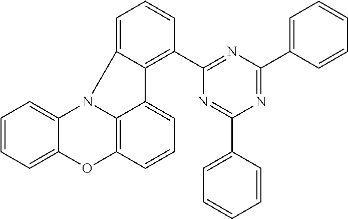 [SMILES: c1ccc(-c2nc(-c3ccccc3)nc(-c3cccc4c3c3cccc5c3n4-c3ccccc3O5)n2)cc1]

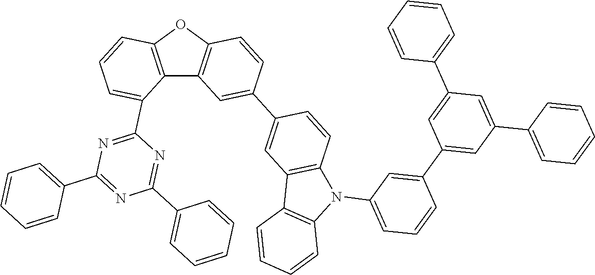 [SMILES: c1ccc(-c2cc(-c3ccccc3)cc(-c3cccc(-n4c5ccccc5c5cc(-c6ccc7oc8cccc(-c9nc(-c%10ccccc%10)nc(-c%10ccccc%10)n9)c8c7c6)ccc54)c3)c2)cc1]

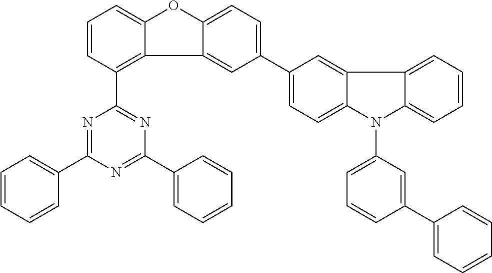 [SMILES: c1ccc(-c2cccc(-n3c4ccccc4c4cc(-c5ccc6oc7cccc(-c8nc(-c9ccccc9)nc(-c9ccccc9)n8)c7c6c5)ccc43)c2)cc1]

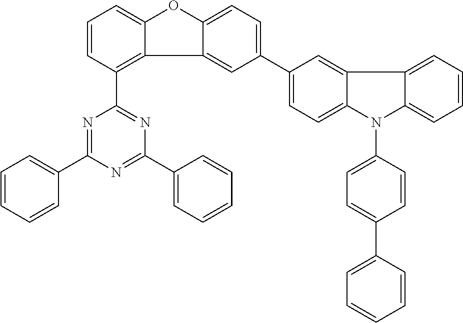 [SMILES: c1ccc(-c2ccc(-n3c4ccccc4c4cc(-c5ccc6oc7cccc(-c8nc(-c9ccccc9)nc(-c9ccccc9)n8)c7c6c5)ccc43)cc2)cc1]

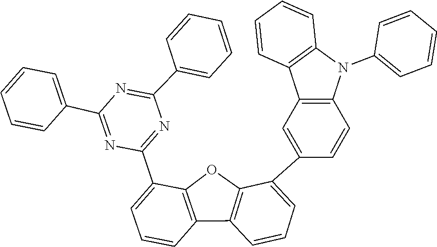 [SMILES: c1ccc(-c2nc(-c3ccccc3)nc(-c3cccc4c3oc3c(-c5ccc6c(c5)c5ccccc5n6-c5ccccc5)cccc34)n2)cc1]

Examples of suitable quinazoline compounds are the compounds depicted in the following table:

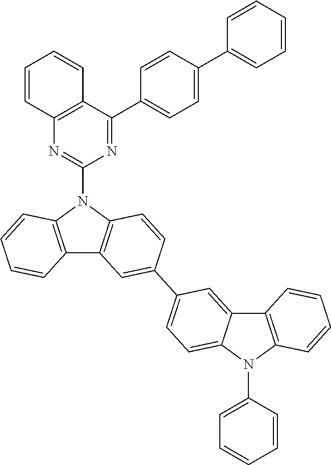 [SMILES: c1ccc(-c2ccc(-c3nc(-n4c5ccccc5c5cc(-c6ccc7c(c6)c6ccccc6n7-c6ccccc6)ccc54)nc4ccccc34)cc2)cc1]

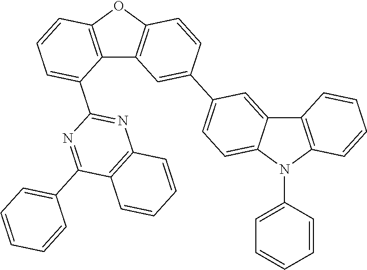 [SMILES: c1ccc(-c2nc(-c3cccc4oc5ccc(-c6ccc7c(c6)c6ccccc6n7-c6ccccc6)cc5c34)nc3ccccc23)cc1]

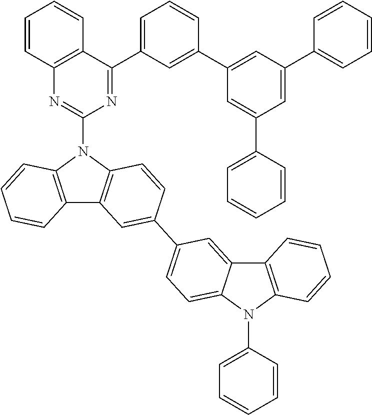 [SMILES: c1ccc(-c2cc(-c3ccccc3)cc(-c3cccc(-c4nc(-n5c6ccccc6c6cc(-c7ccc8c(c7)c7ccccc7n8-c7ccccc7)ccc65)nc5ccccc45)c3)c2)cc1]

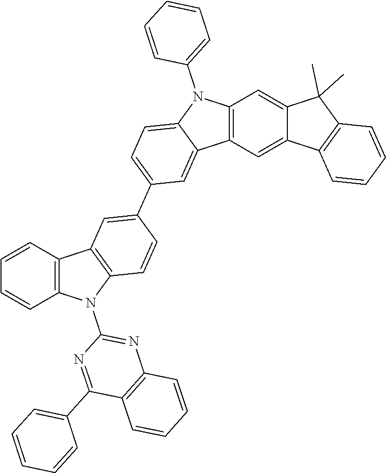 [SMILES: CC1(C)c2ccccc2-c2cc3c4cc(-c5ccc6c(c5)c5ccccc5n6-c5nc(-c6ccccc6)c6ccccc6n5)ccc4n(-c4ccccc4)c3cc21]

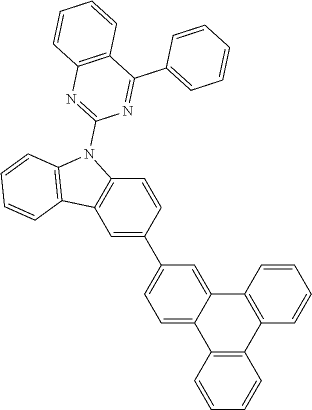 [SMILES: c1ccc(-c2nc(-n3c4ccccc4c4cc(-c5ccc6c7ccccc7c7ccccc7c6c5)ccc43)nc3ccccc23)cc1]

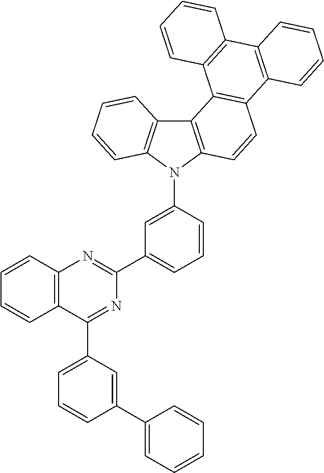 [SMILES: c1ccc(-c2cccc(-c3nc(-c4cccc(-n5c6ccccc6c6c7c8ccccc8c8ccccc8c7ccc65)c4)nc4ccccc34)c2)cc1]

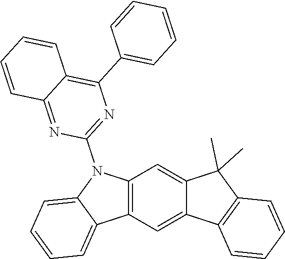 [SMILES: CC1(C)c2ccccc2-c2cc3c4ccccc4n(-c4nc(-c5ccccc5)c5ccccc5n4)c3cc21]

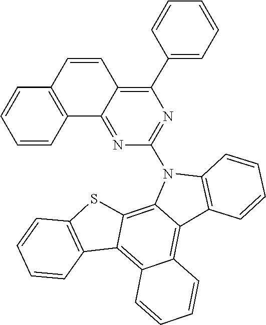 [SMILES: c1ccc(-c2nc(-n3c4ccccc4c4c5ccccc5c5c6ccccc6sc5c43)nc3c2ccc2ccccc23)cc1]

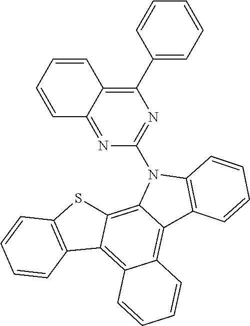 [SMILES: c1ccc(-c2nc(-n3c4ccccc4c4c5ccccc5c5c6ccccc6sc5c43)nc3ccccc23)cc1]

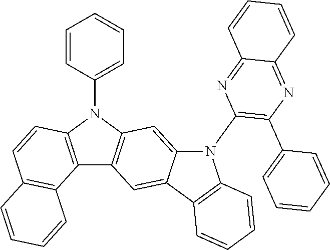 [SMILES: c1ccc(-c2nc3ccccc3nc2-n2c3ccccc3c3cc4c5c6ccccc6ccc5n(-c5ccccc5)c4cc32)cc1]

Suitable phosphorescent compounds (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum.

Examples of the emitters described above can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2017/032439, WO 2018/011186 and WO 2018/041769, WO 2019/020538, WO 2018/178001 and as yet unpublished patent applications EP 17206950.2 and EP 18156388.3. In general, all phosphorescent complexes as used for phosphorescent OLEDs according to the prior art and as known to those skilled in the art in the field of organic electroluminescence are suitable, and the person skilled in the art will be able to use further phosphorescent complexes without exercising inventive skill.

Examples of phosphorescent dopants are adduced below.

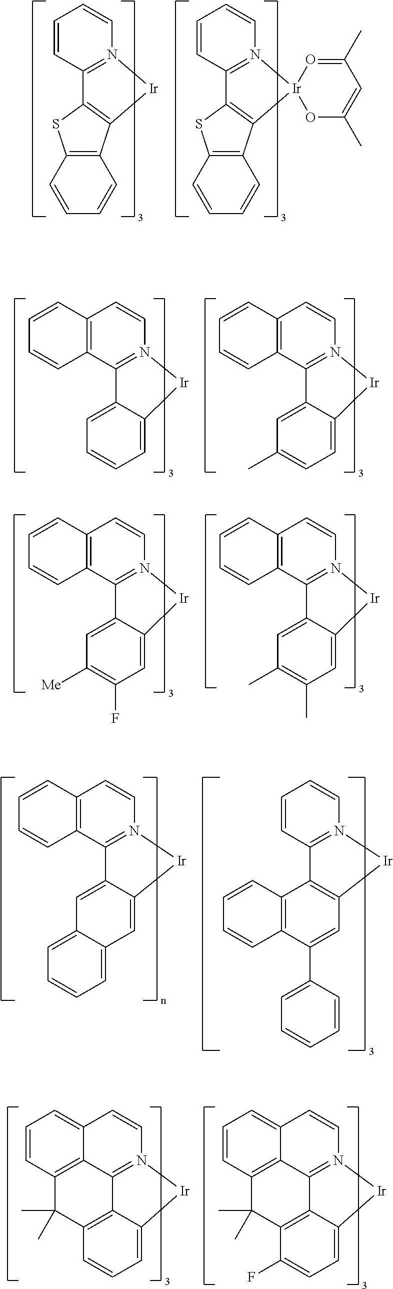

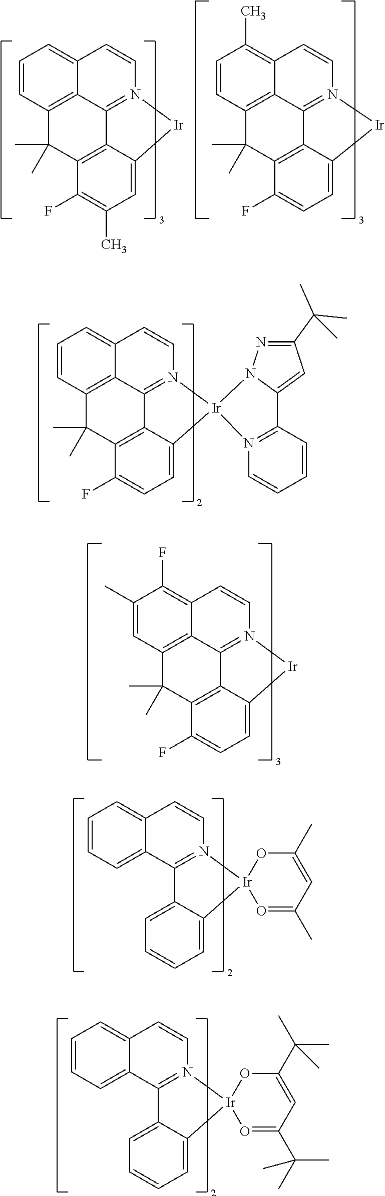

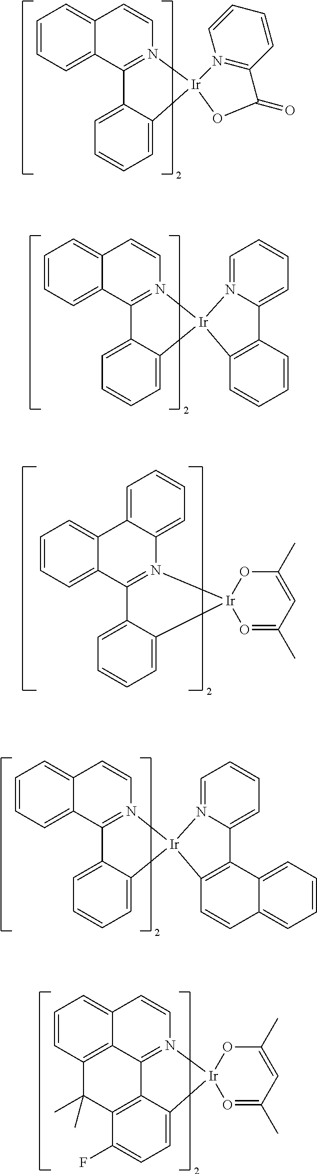

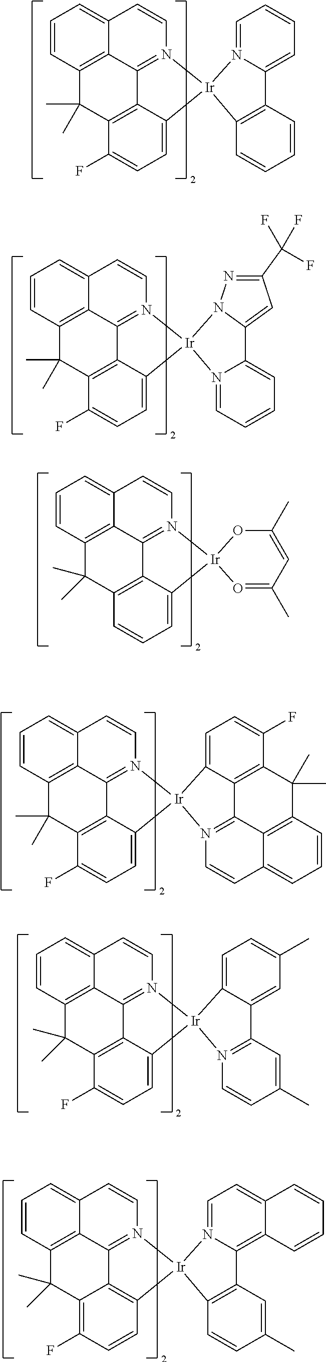

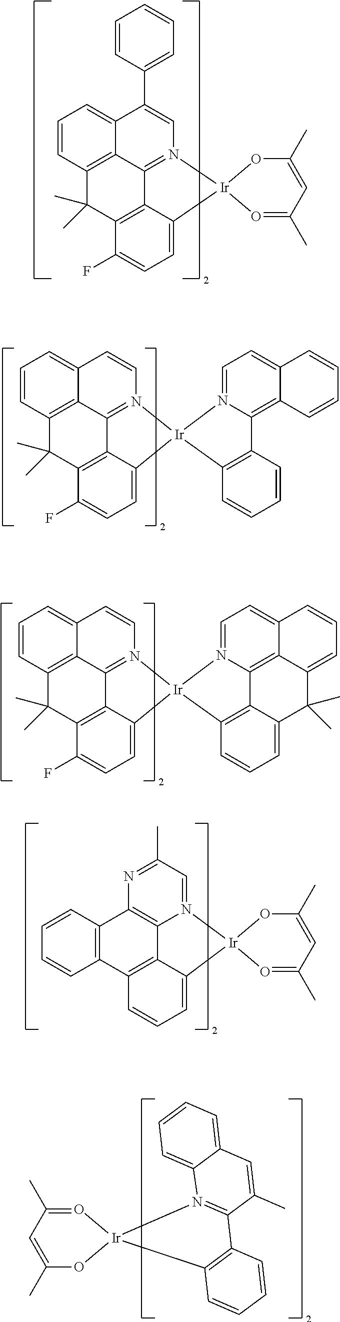

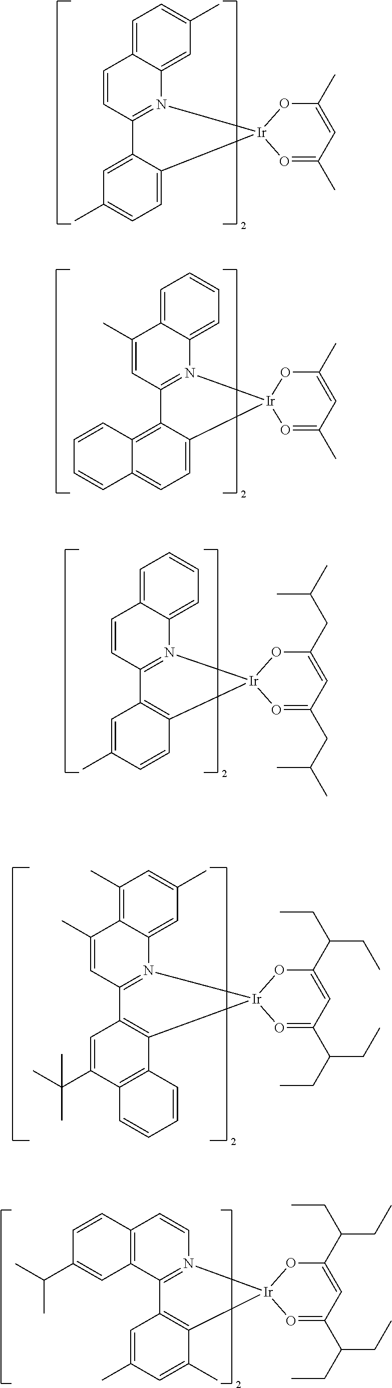

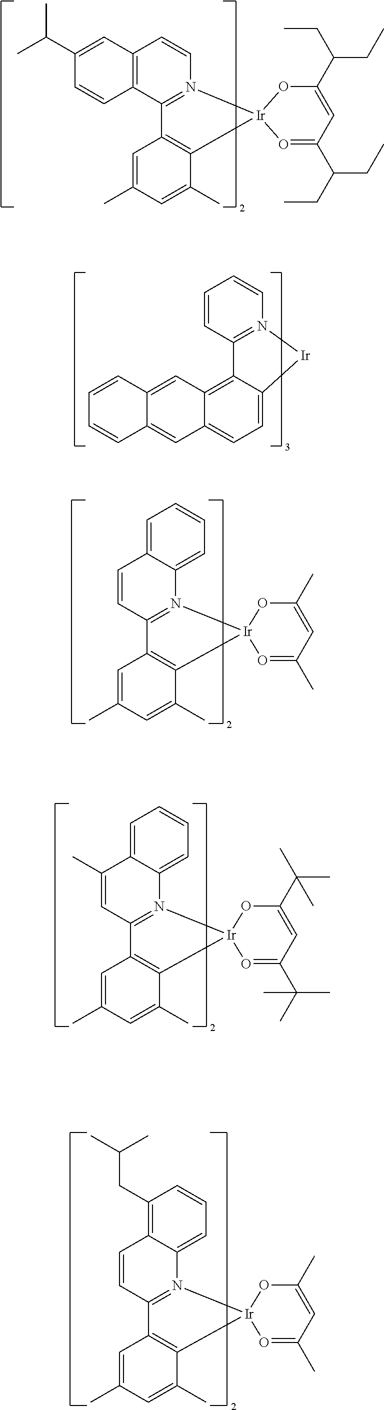

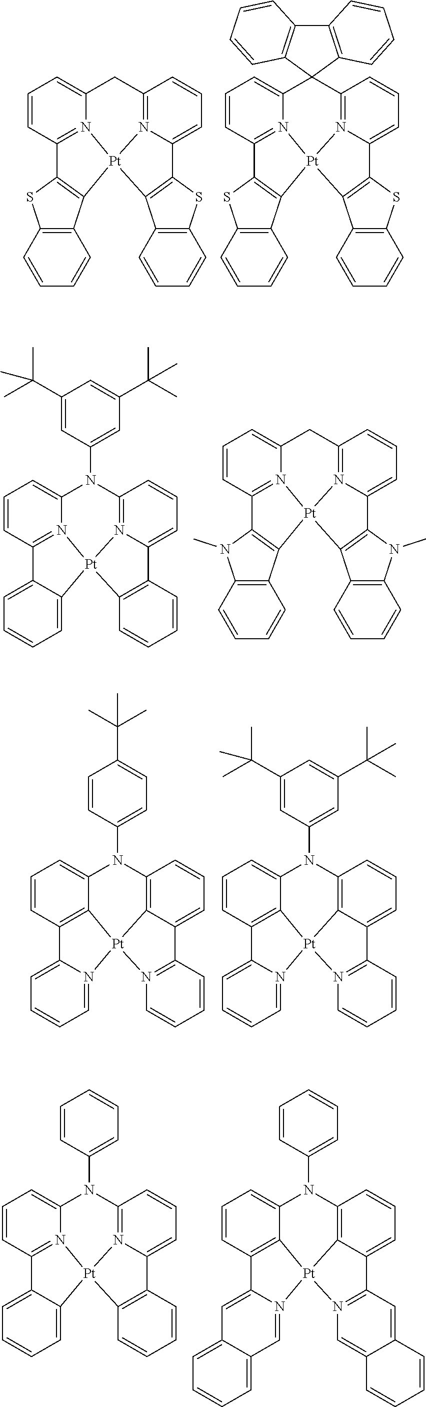

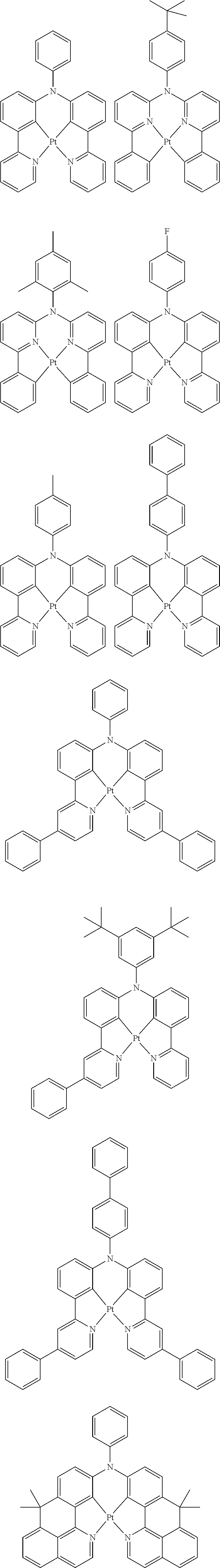

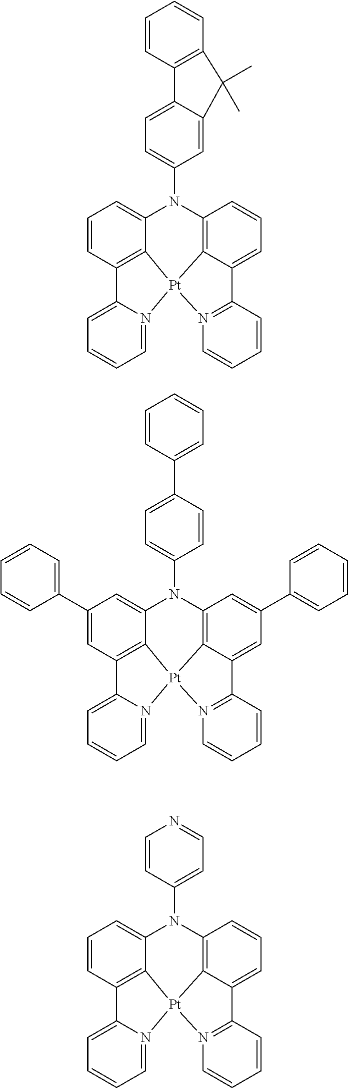

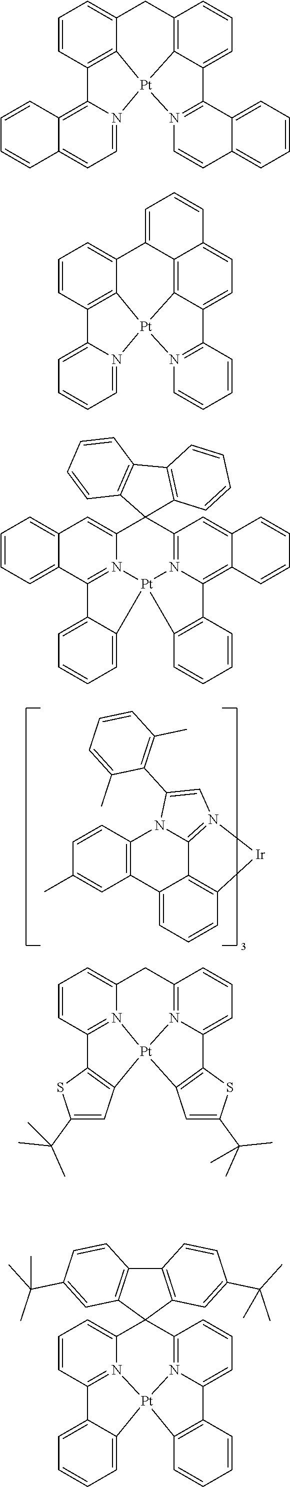

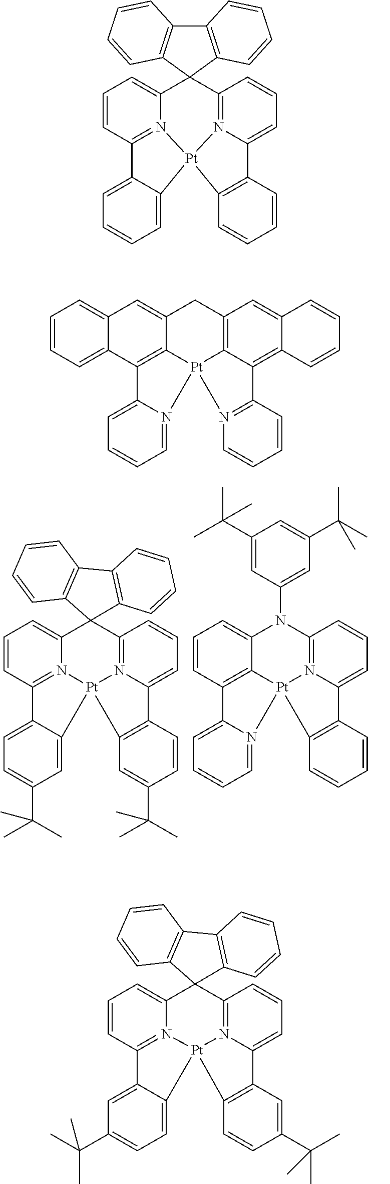

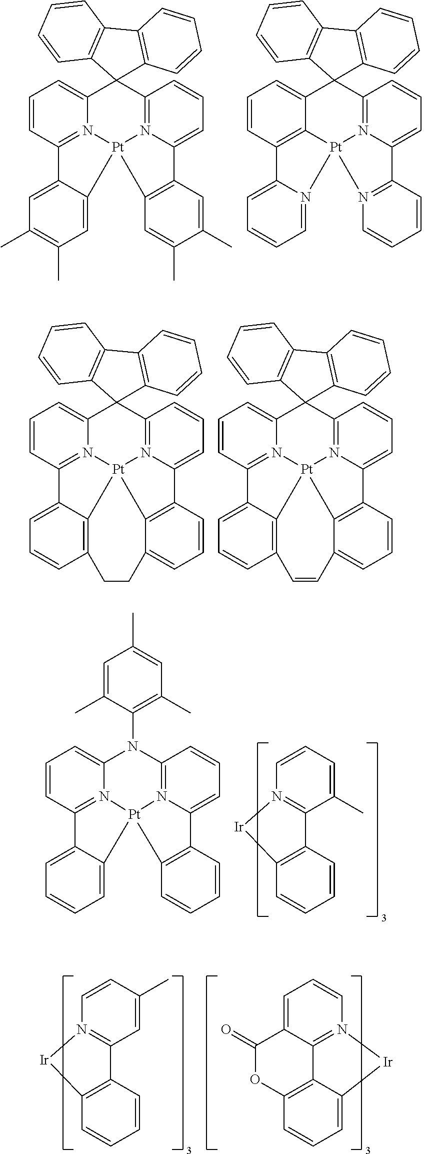

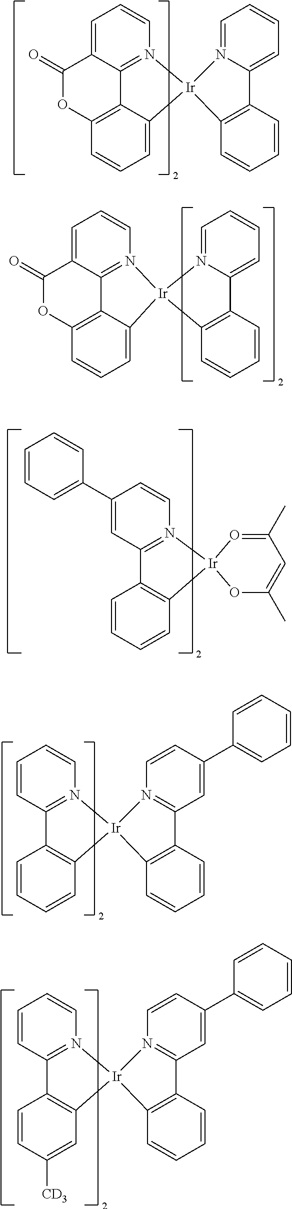

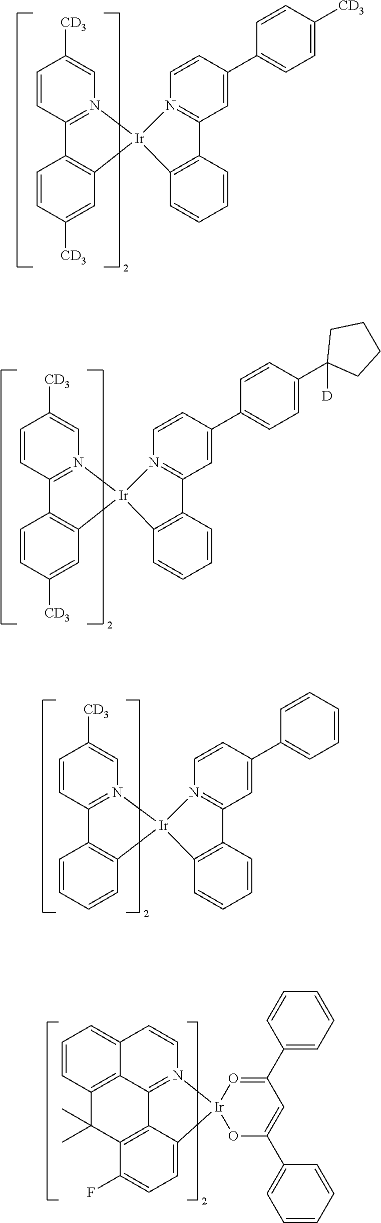

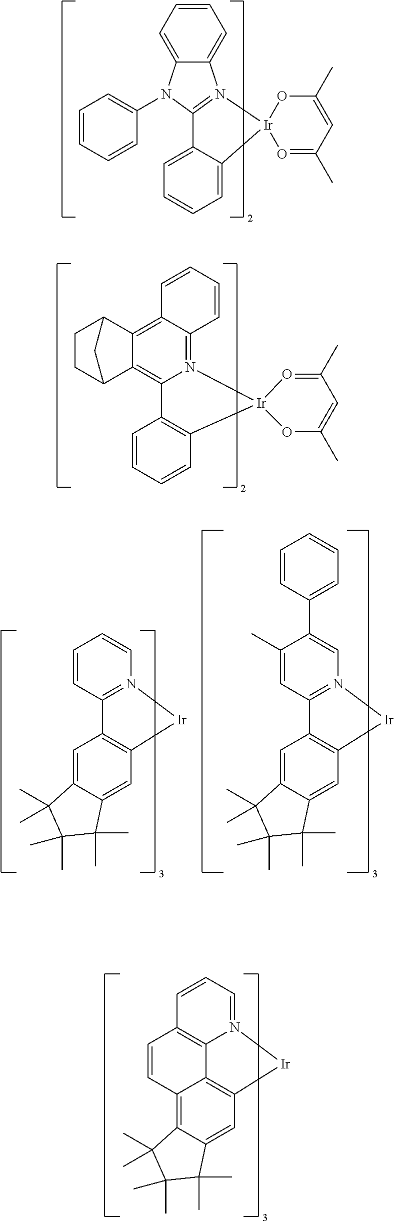

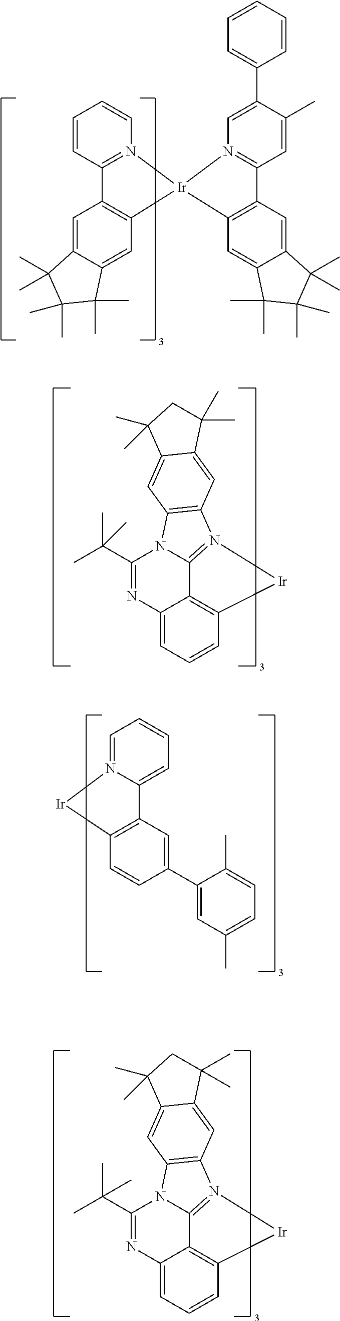

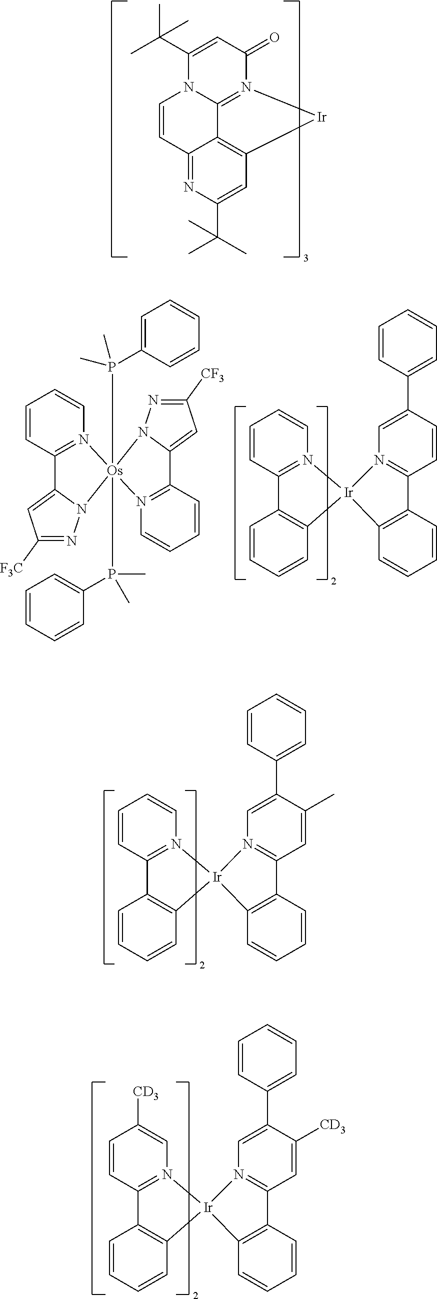

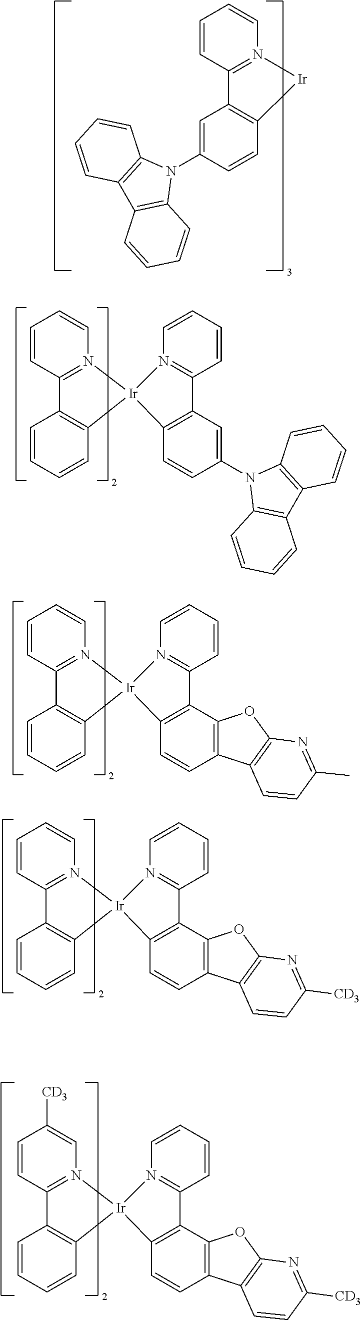

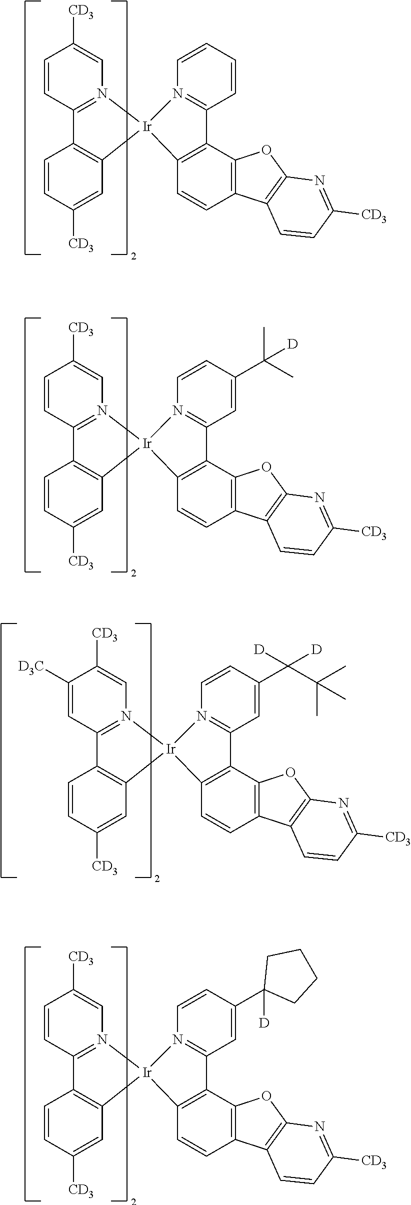

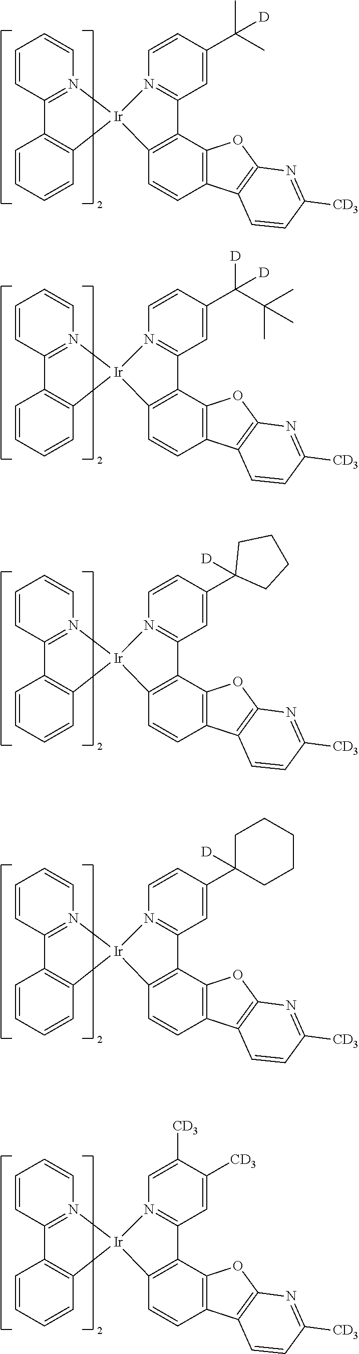

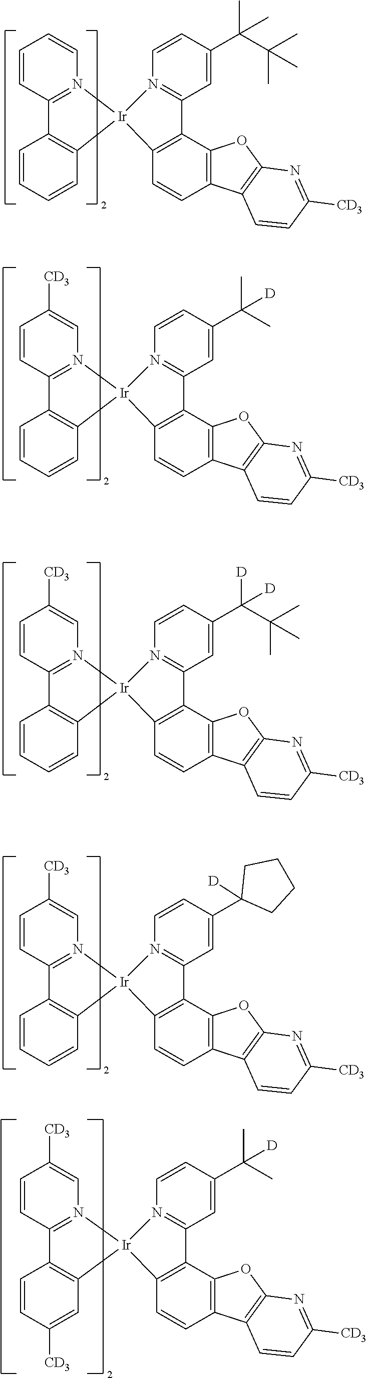

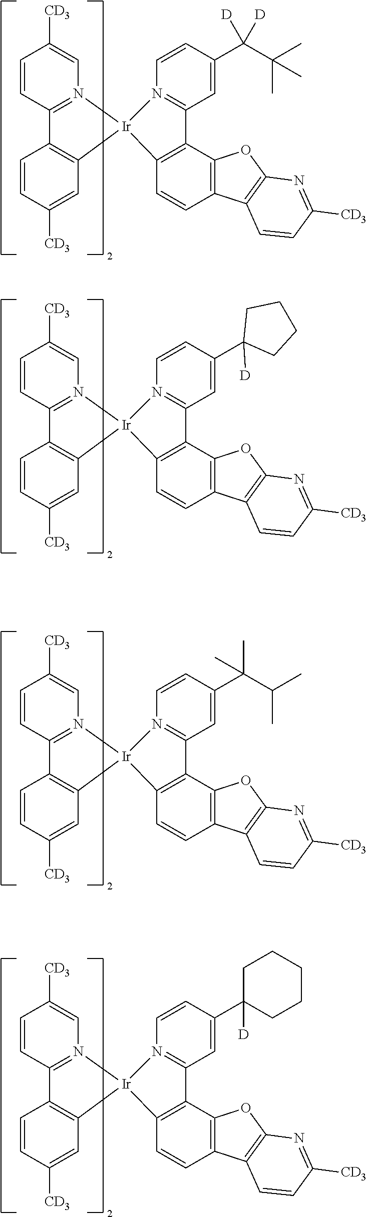

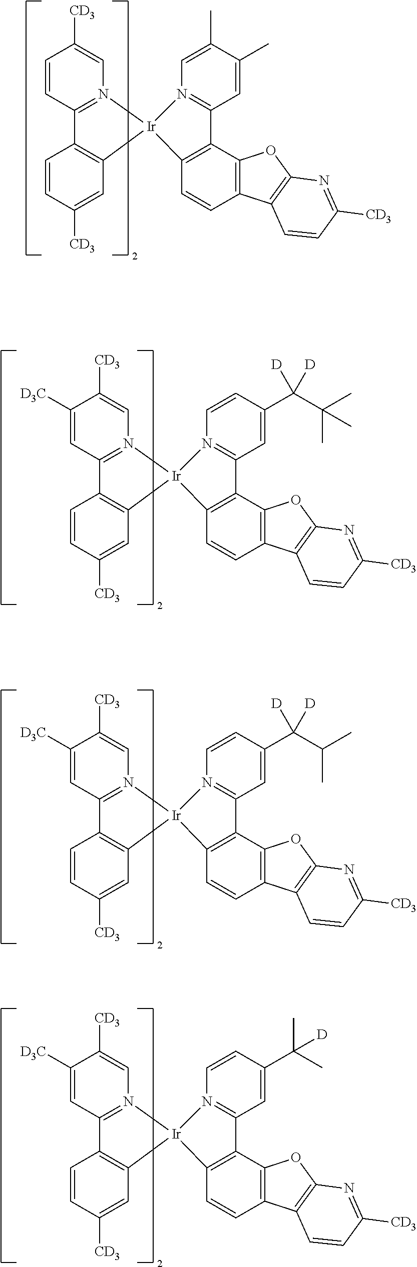

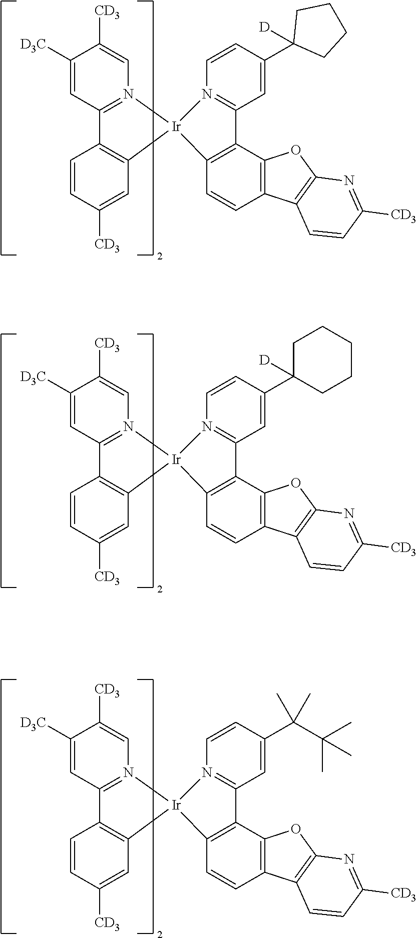

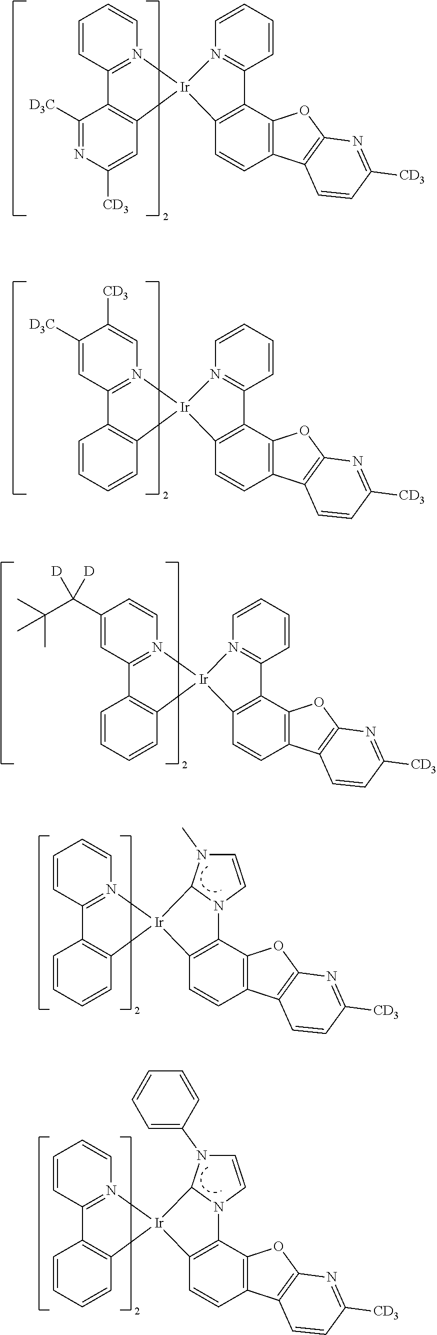

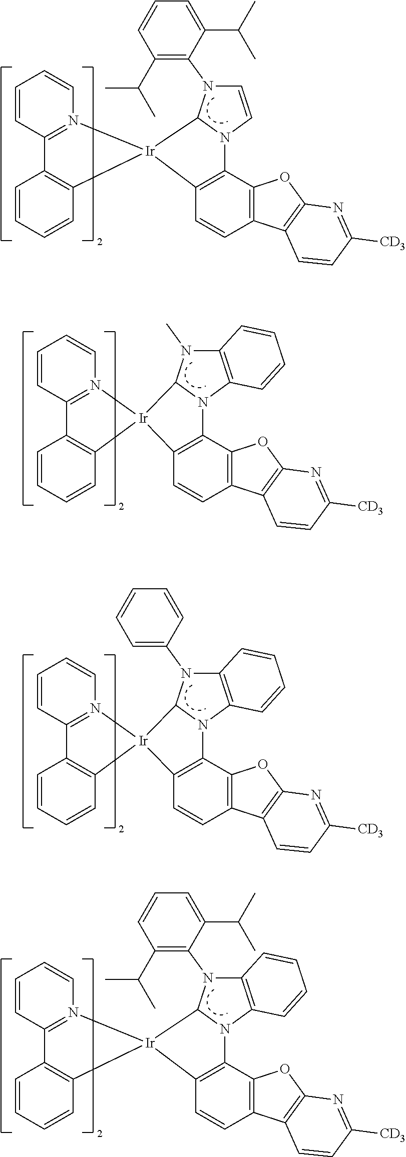

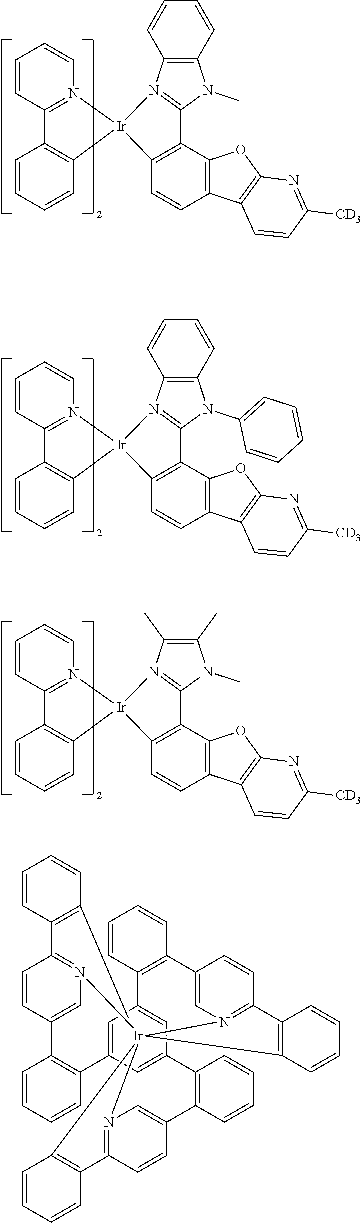

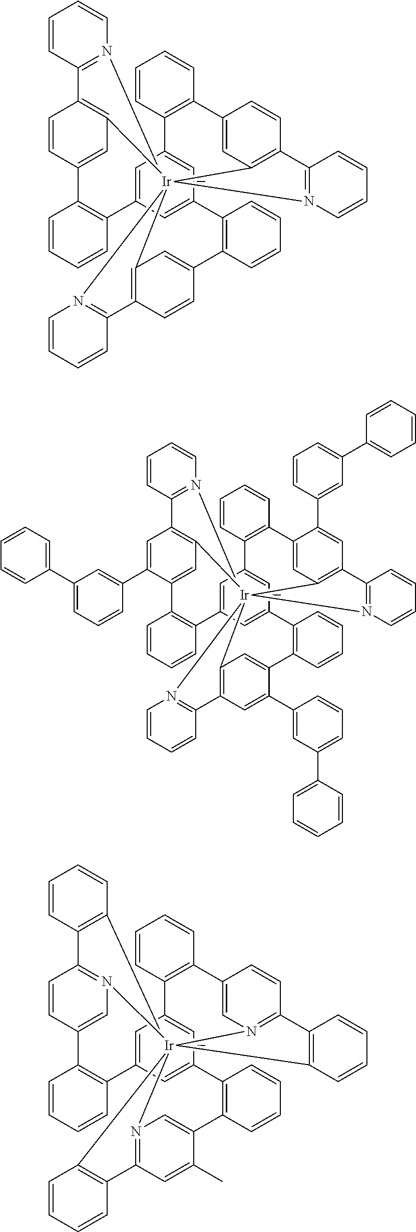

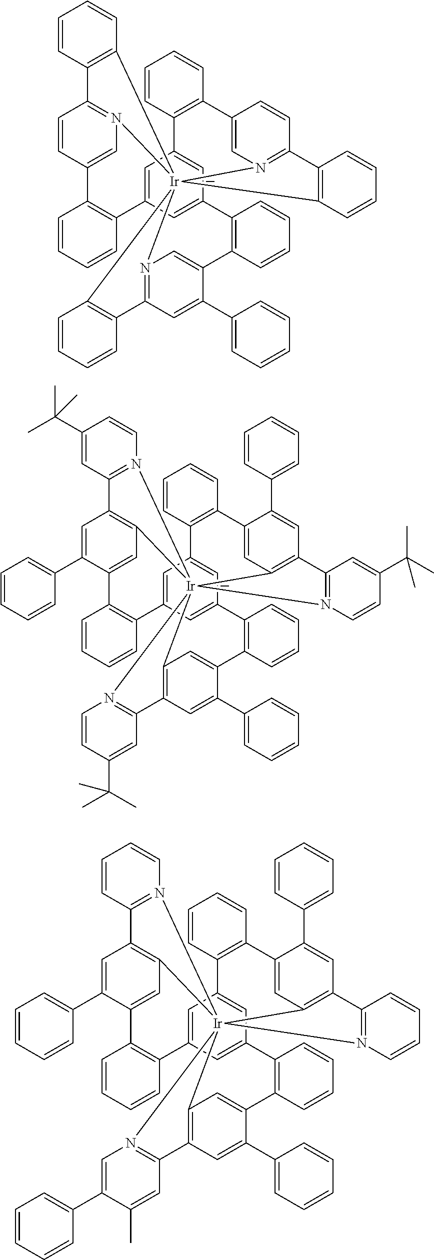

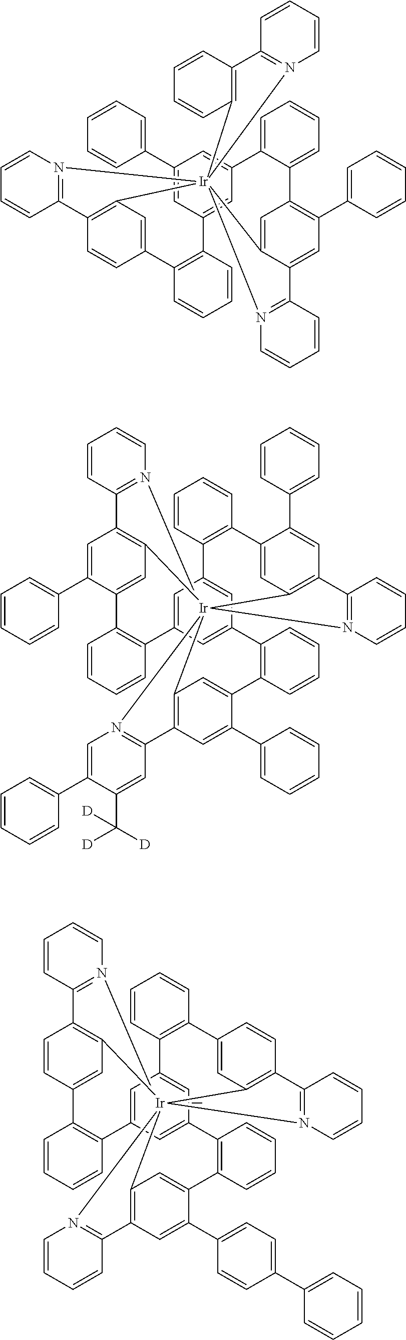

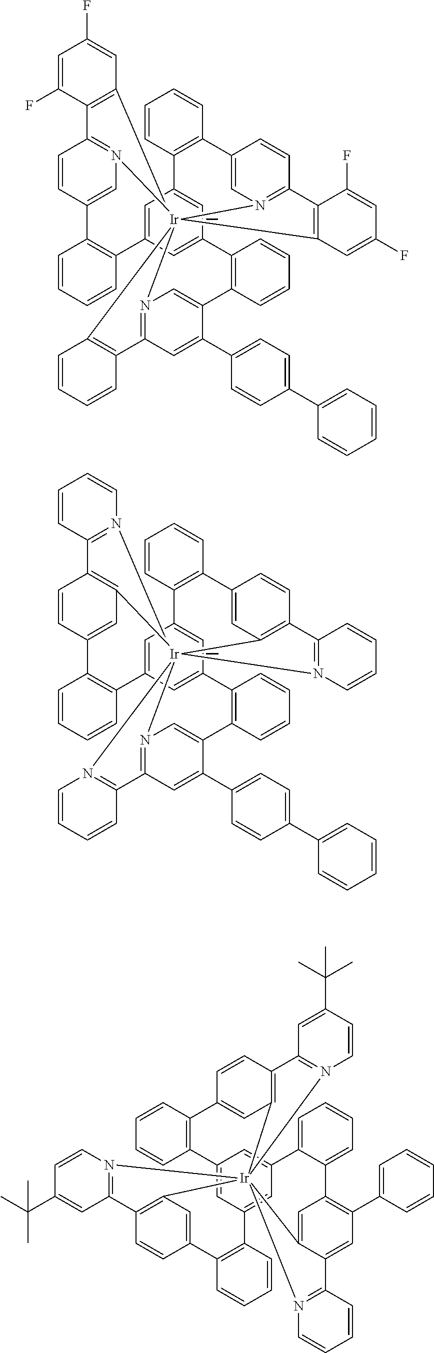

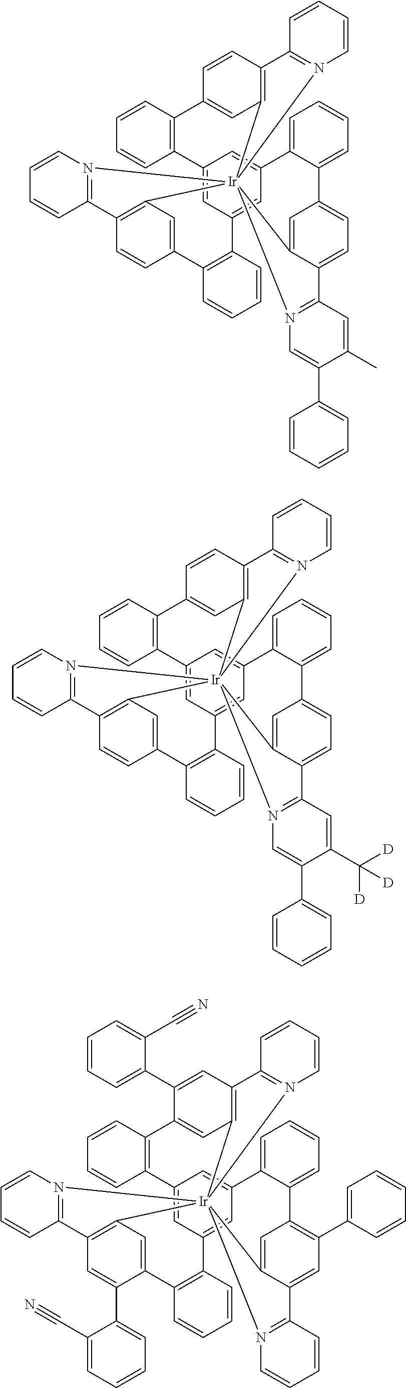

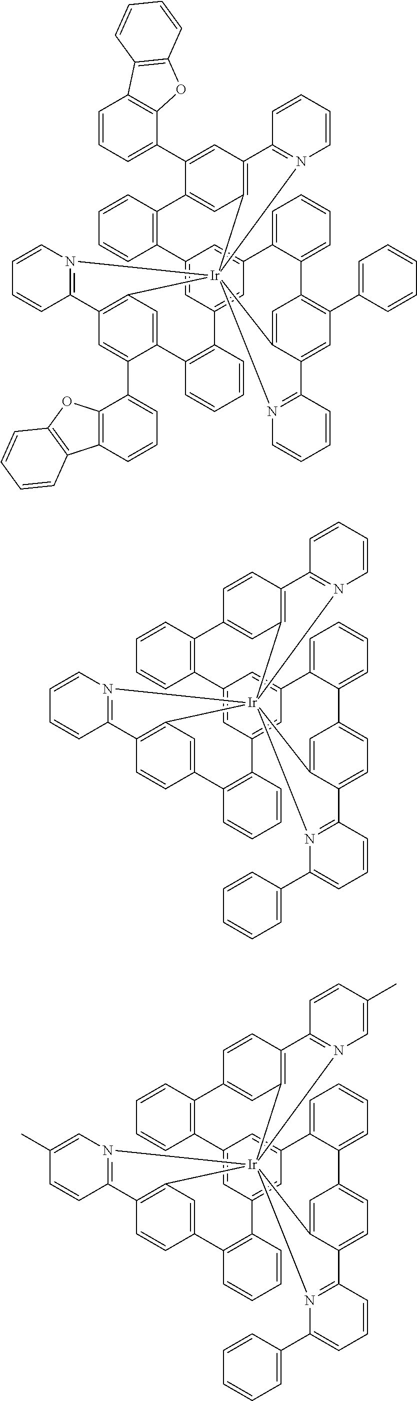

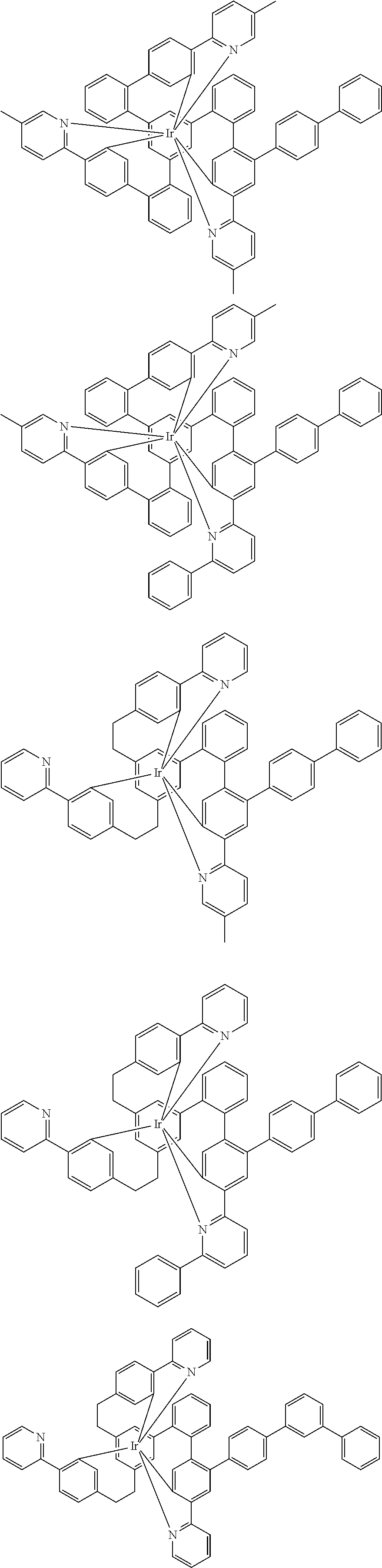

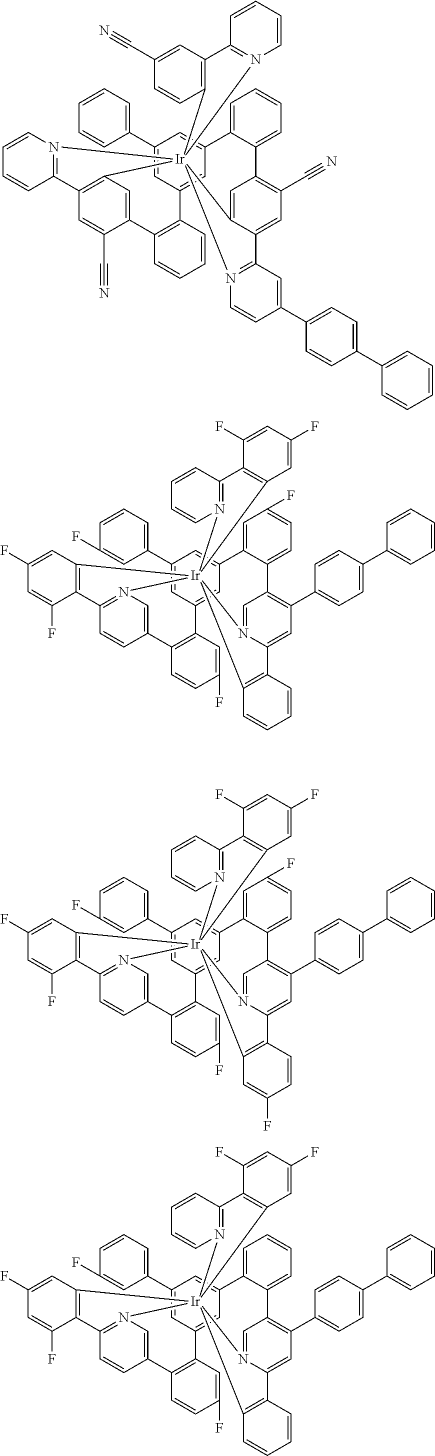

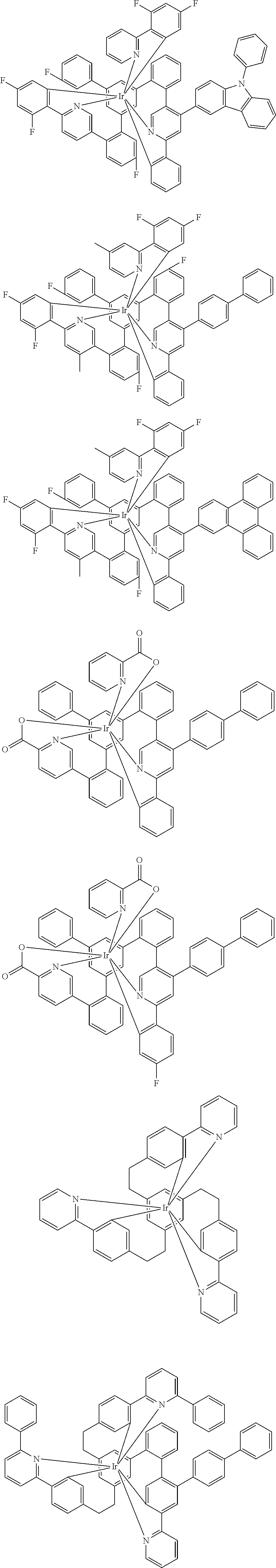

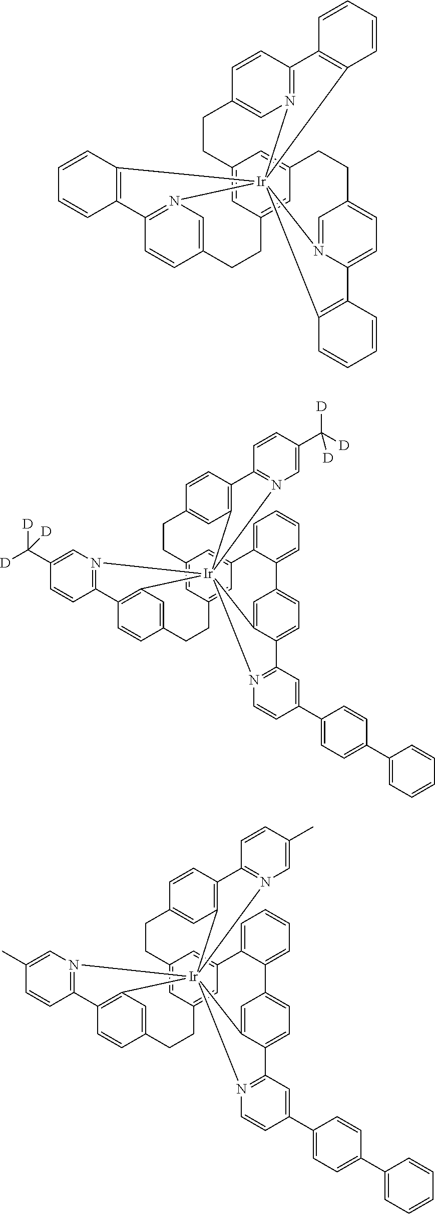

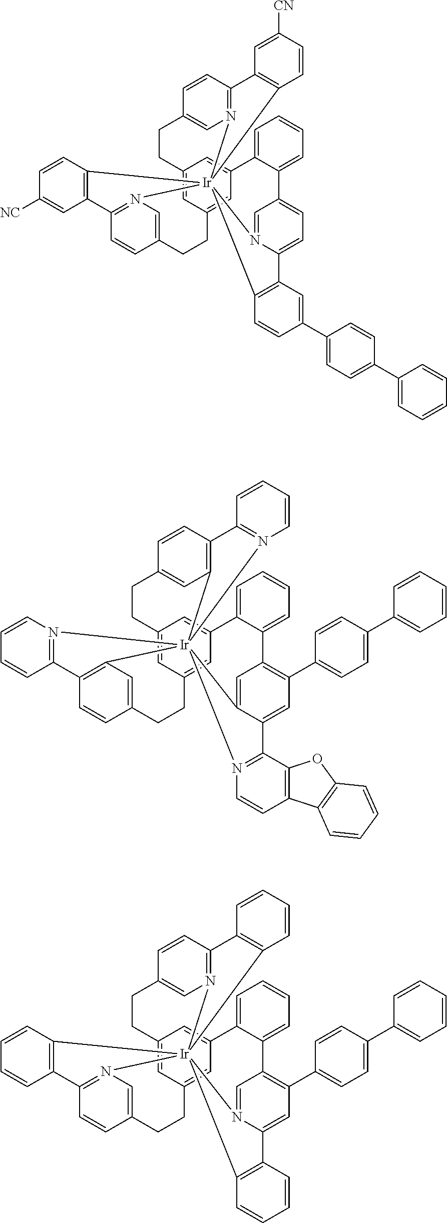

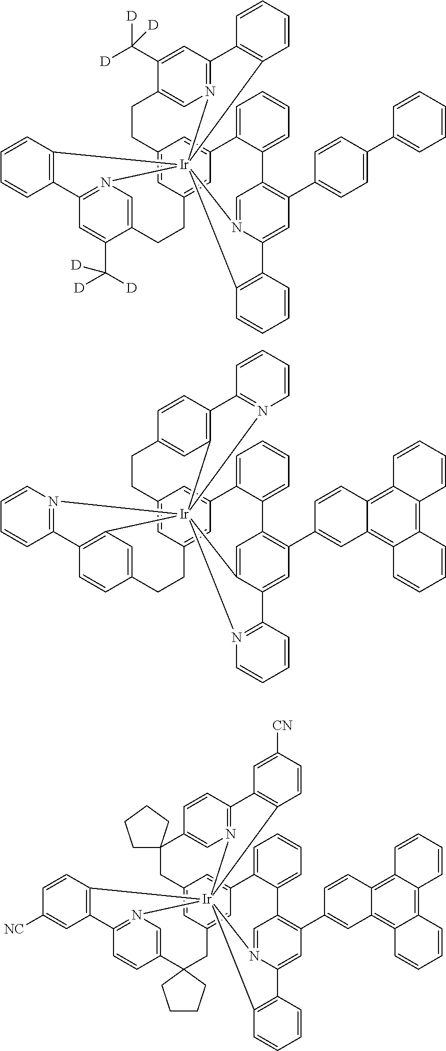

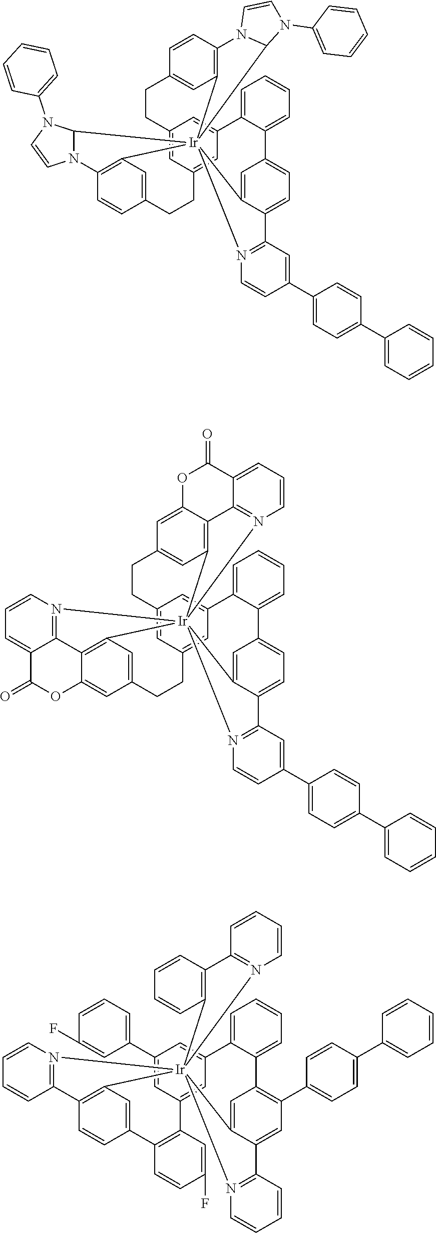

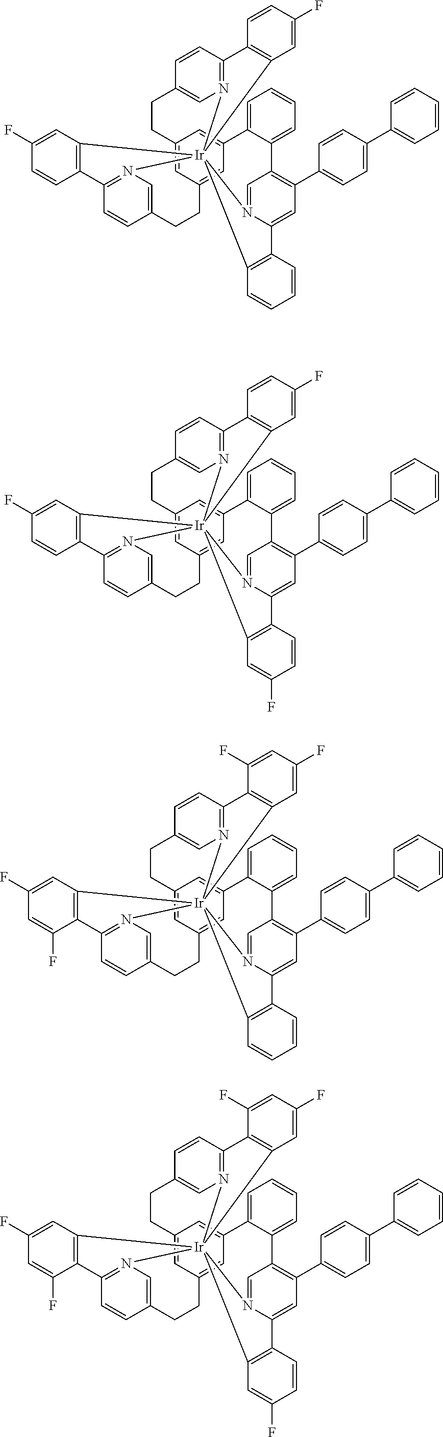

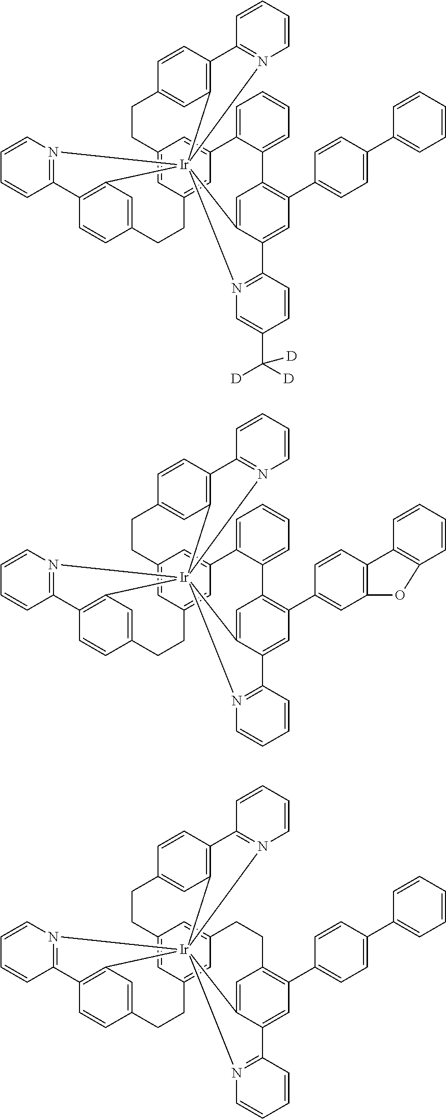

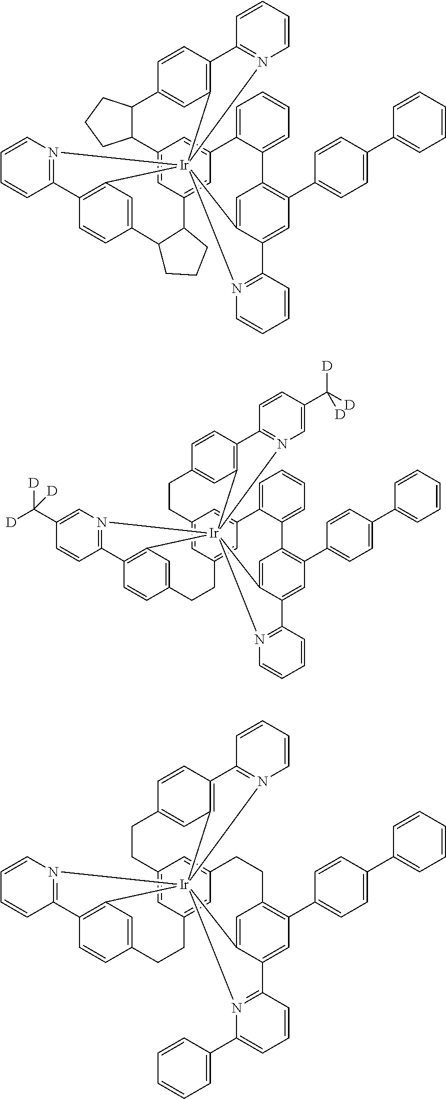

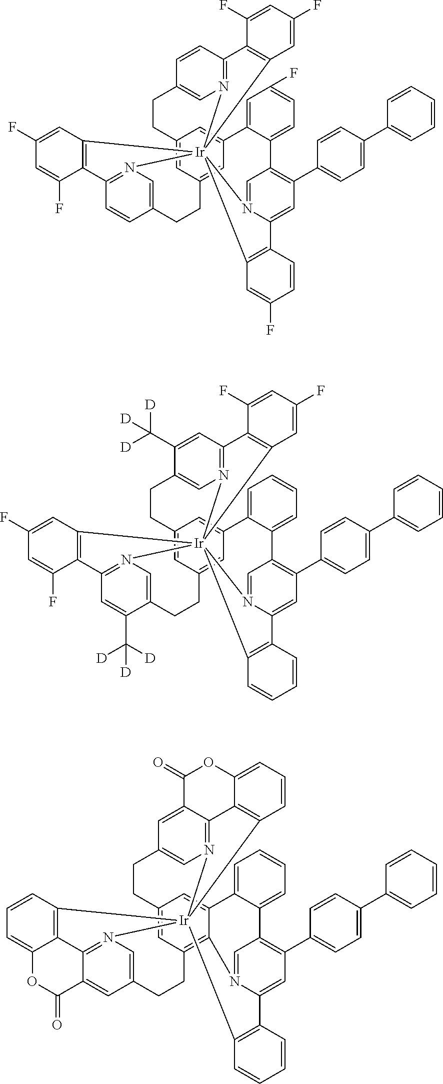

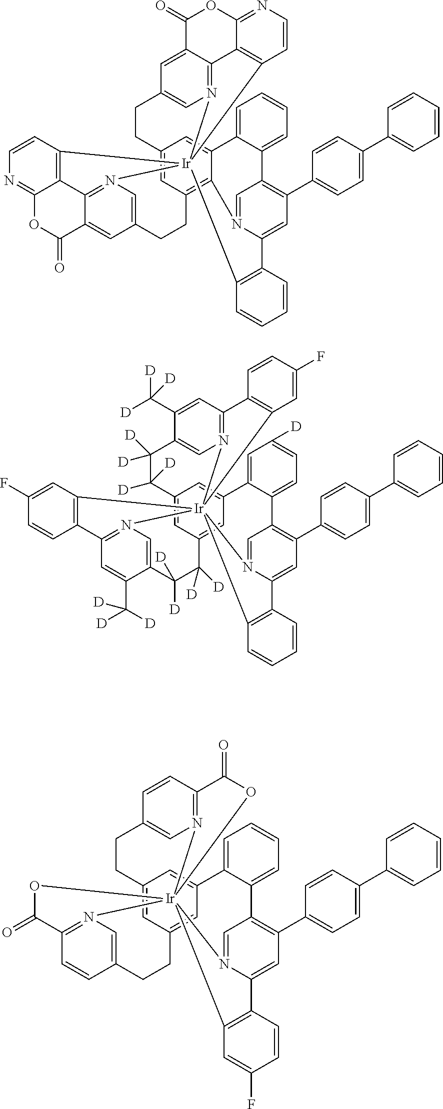

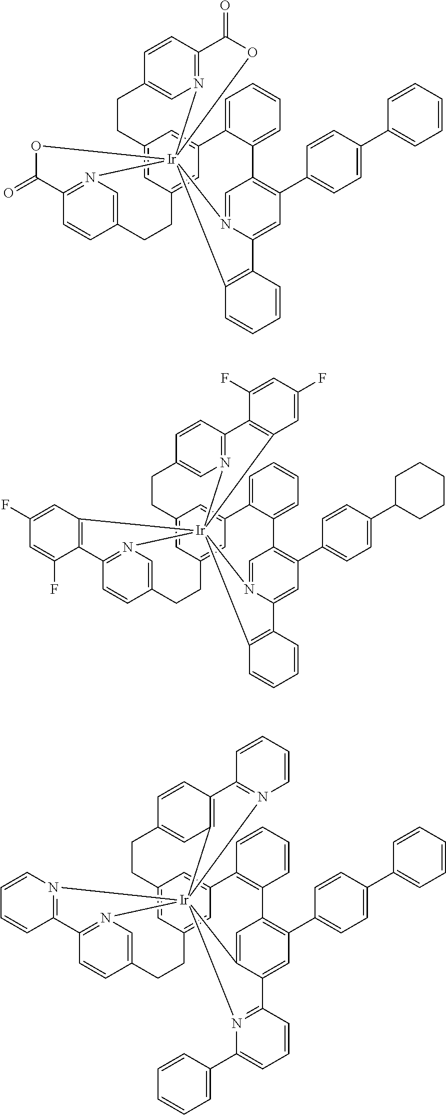

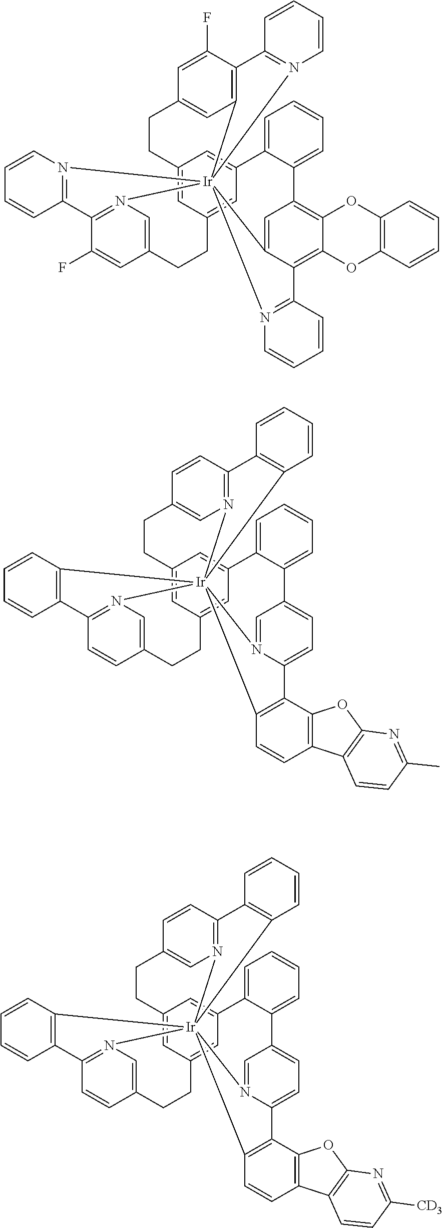

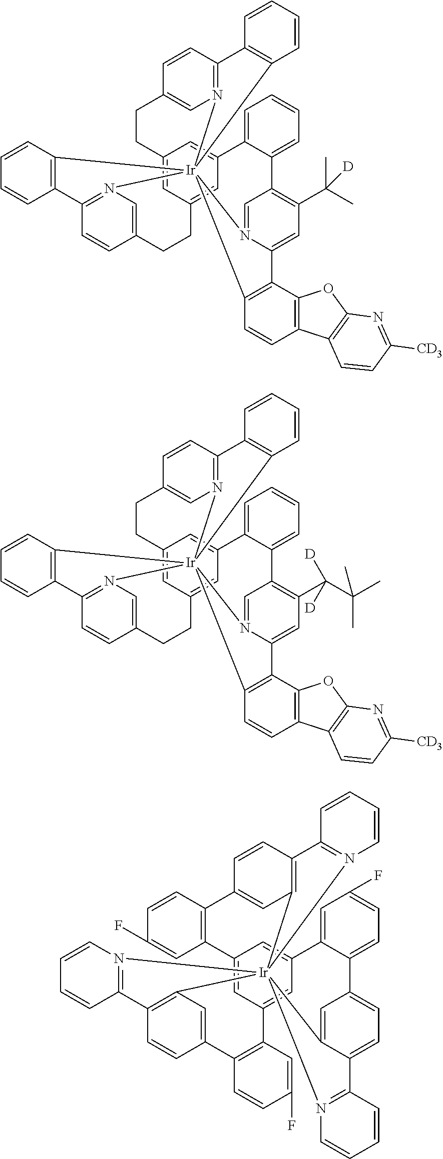

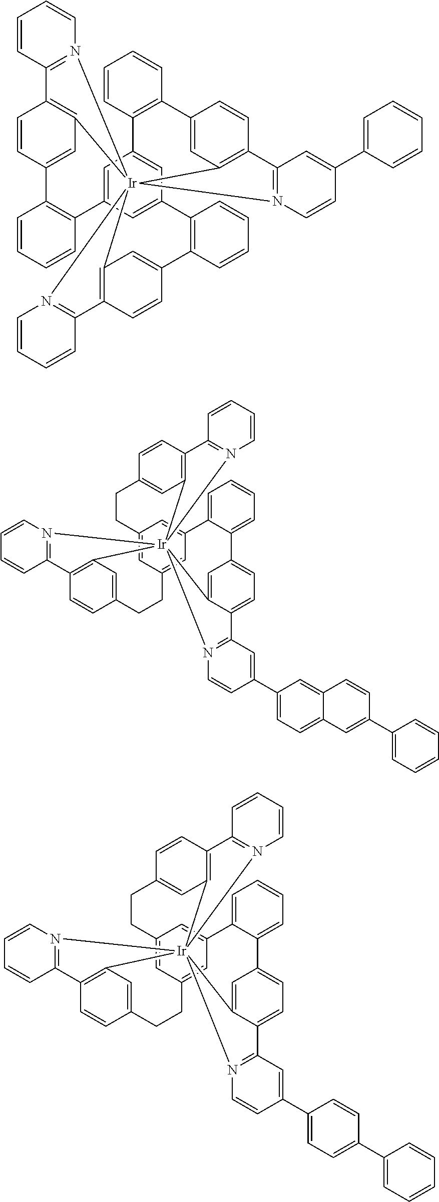

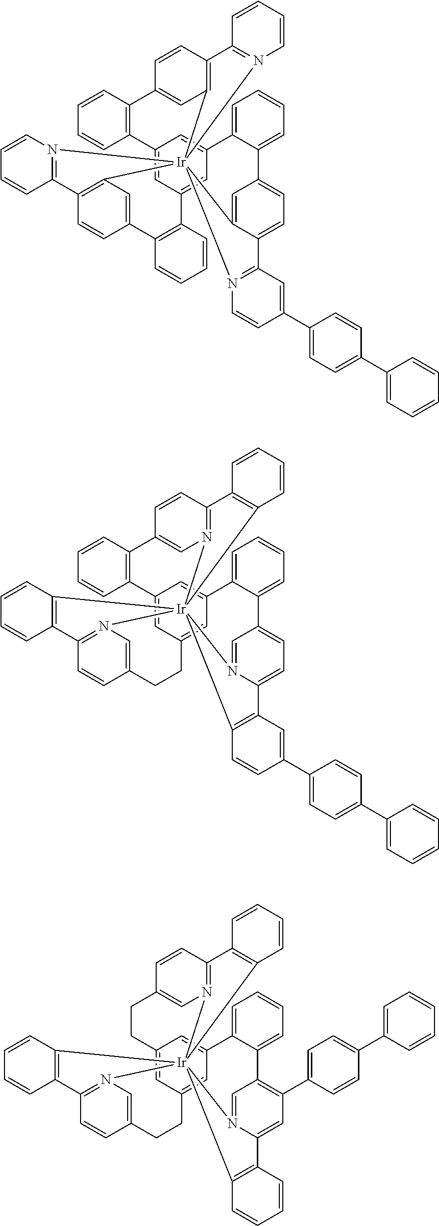

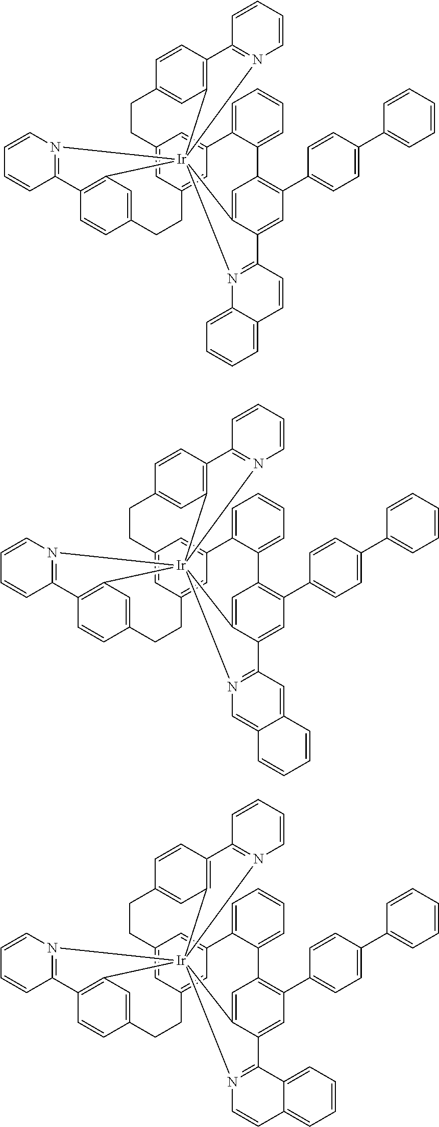

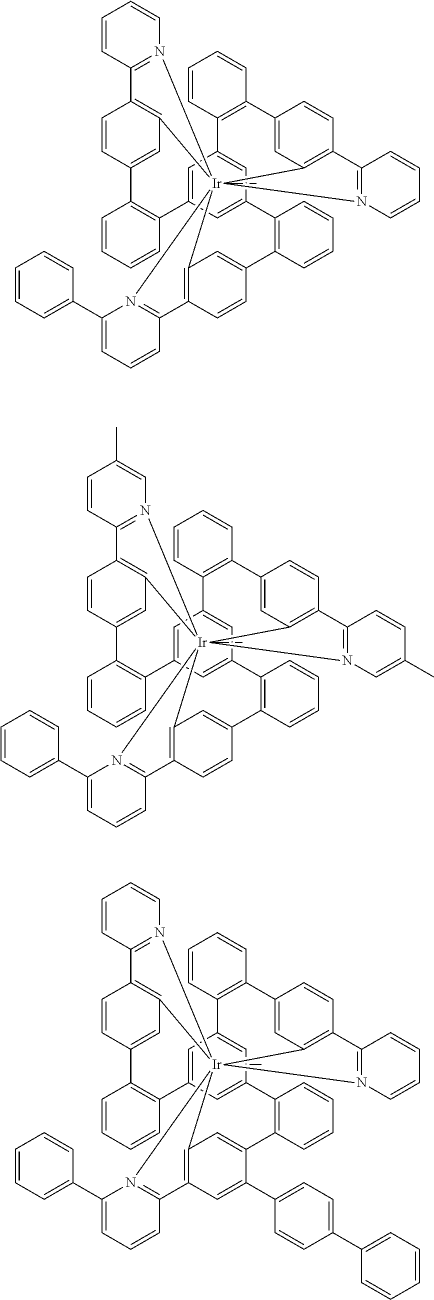

In the further layers of the organic electroluminescent device of the invention, it is possible to use any materials as typically used according to the prior art. The person skilled in the art will therefore be able, without exercising inventive skill, to use any materials known for organic electroluminescent devices in combination with the inventive compounds of formula (I) or the above-recited preferred embodiments.

In hole-transporting layers of the device, such as hole injection layers, hole transport layers and electron blocker layers, preference is given to using indenofluoreneamine derivatives, amine derivatives, hexaazatriphenylene derivatives, amine derivatives with fused aromatic systems, monobenzoindenofluoreneamines, dibenzoindenofluoreneamines, spirobifluoreneamines, fluoreneamines, spirodibenzopyranamines, dihydroacridine derivatives, spirodibenzofurans and spirodibenzothiophenes, phenanthrenediarylamines, spirotribenzotropolones, spirobifluorenes having meta-phenyldiamine groups, spirobisacridines, xanthenediarylamines, and 9,10-dihydroanthracene spiro compounds having diarylamino groups. Explicit examples of compounds for use in hole-transporting layers are shown in the following table:

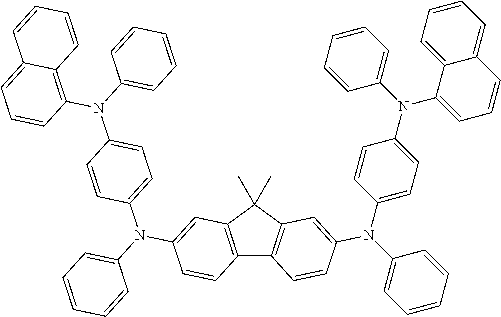 [SMILES: CC1(C)c2cc(N(c3ccccc3)c3ccc(N(c4ccccc4)c4cccc5ccccc45)cc3)ccc2-c2ccc(N(c3ccccc3)c3ccc(N(c4ccccc4)c4cccc5ccccc45)cc3)cc21]

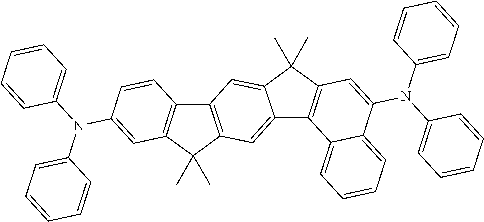 [SMILES: CC1(C)c2cc(N(c3ccccc3)c3ccccc3)ccc2-c2cc3c(cc21)-c1c(cc(N(c2ccccc2)c2ccccc2)c2ccccc12)C3(C)C]

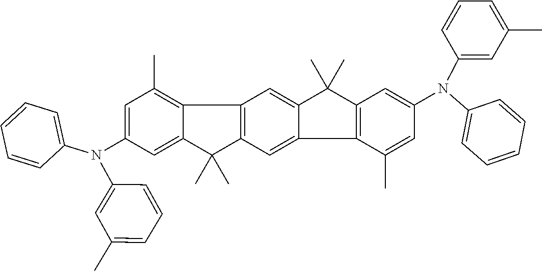 [SMILES: Cc1cccc(N(c2ccccc2)c2cc(C)c3c(c2)C(C)(C)c2cc4c(cc2-3)C(C)(C)c2cc(N(c3ccccc3)c3cccc(C)c3)cc(C)c2-4)c1]

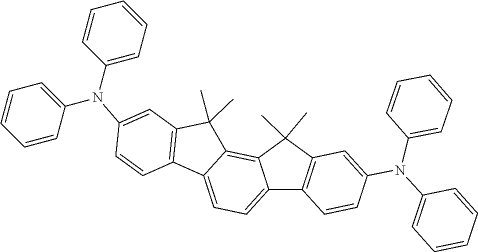 [SMILES: CC1(C)c2cc(N(c3ccccc3)c3ccccc3)ccc2-c2ccc3c(c21)C(C)(C)c1cc(N(c2ccccc2)c2ccccc2)ccc1-3]

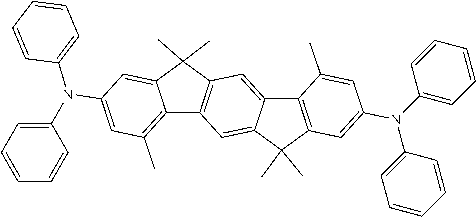 [SMILES: Cc1cc(N(c2ccccc2)c2ccccc2)cc2c1-c1cc3c(cc1C2(C)C)-c1c(C)cc(N(c2ccccc2)c2ccccc2)cc1C3(C)C]

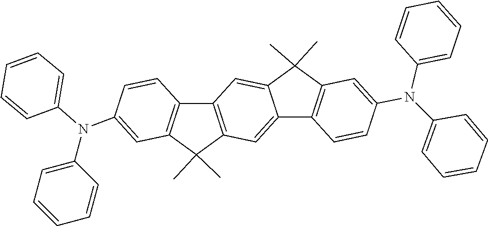 [SMILES: CC1(C)c2cc(N(c3ccccc3)c3ccccc3)ccc2-c2cc3c(cc21)-c1ccc(N(c2ccccc2)c2ccccc2)cc1C3(C)C]

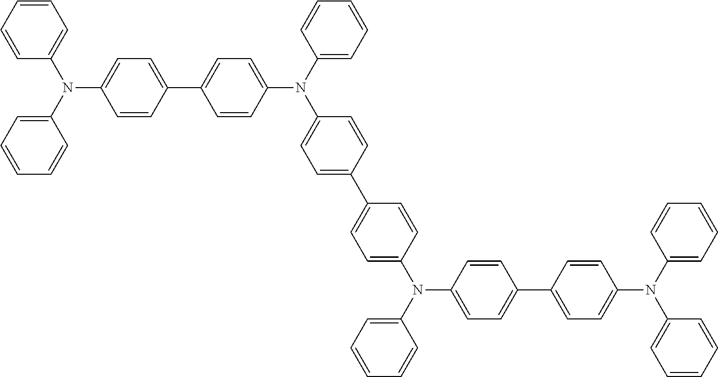 [SMILES: c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1]

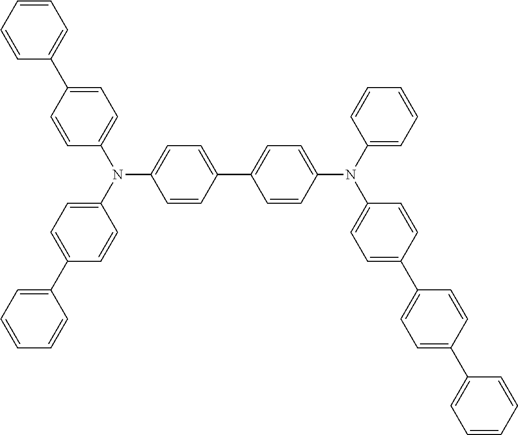 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

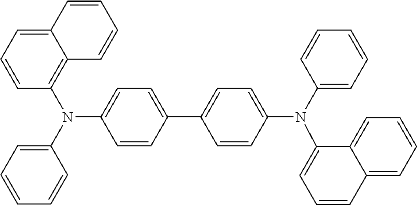 [SMILES: c1ccc(N(c2ccc(-c3ccc(N(c4ccccc4)c4cccc5ccccc45)cc3)cc2)c2cccc3ccccc23)cc1]

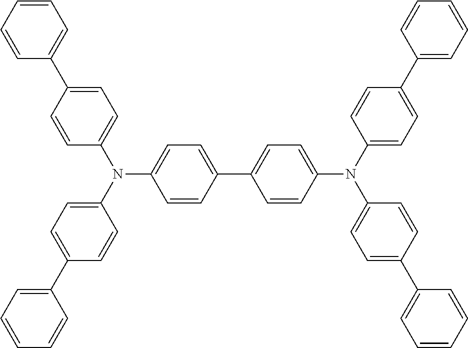 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

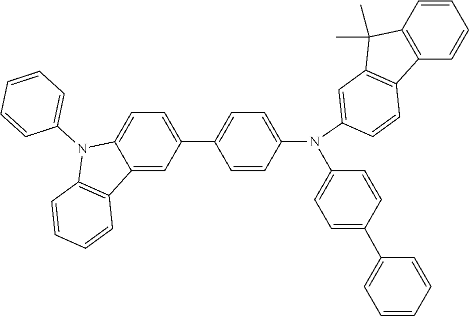 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]

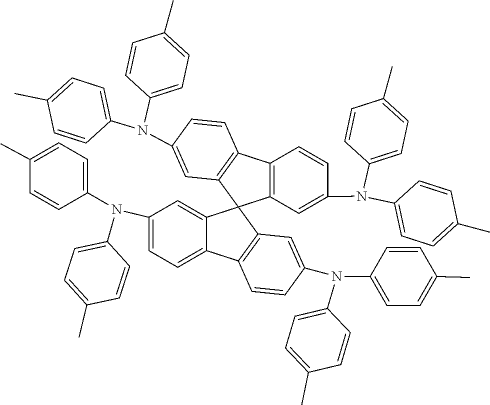

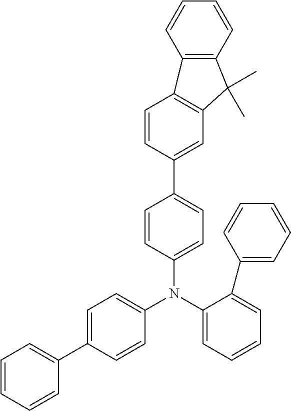 [SMILES: CC1(C)c2ccccc2-c2ccc(-c3ccc(N(c4ccc(-c5ccccc5)cc4)c4ccccc4-c4ccccc4)cc3)cc21]

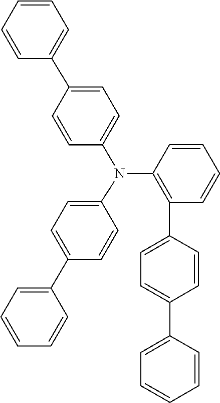 [SMILES: c1ccc(-c2ccc(-c3ccccc3N(c3ccc(-c4ccccc4)cc3)c3ccc(-c4ccccc4)cc3)cc2)cc1]

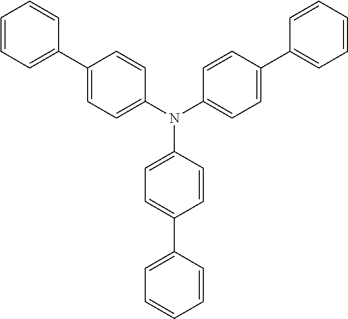 [SMILES: c1ccc(-c2ccc(N(c3ccc(-c4ccccc4)cc3)c3ccc(-c4ccccc4)cc3)cc2)cc1]

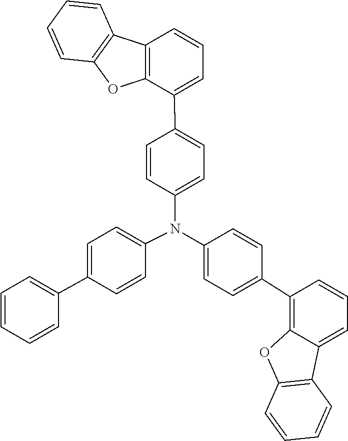 [SMILES: c1ccc(-c2ccc(N(c3ccc(-c4cccc5c4oc4ccccc45)cc3)c3ccc(-c4cccc5c4oc4ccccc45)cc3)cc2)cc1]

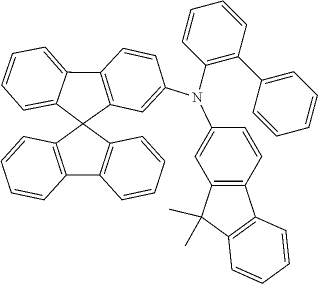 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

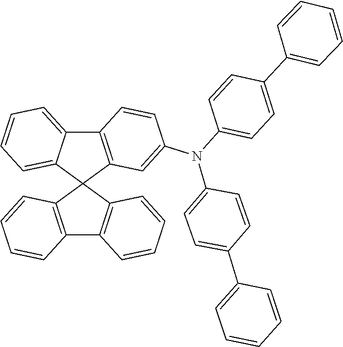 [SMILES: c1ccc(-c2ccc(N(c3ccc(-c4ccccc4)cc3)c3ccc4c(c3)C3(c5ccccc5-c5ccccc53)c3ccccc3-4)cc2)cc1]

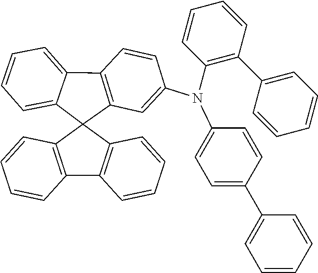 [SMILES: c1ccc(-c2ccc(N(c3ccc4c(c3)C3(c5ccccc5-c5ccccc53)c3ccccc3-4)c3ccccc3-c3ccccc3)cc2)cc1]

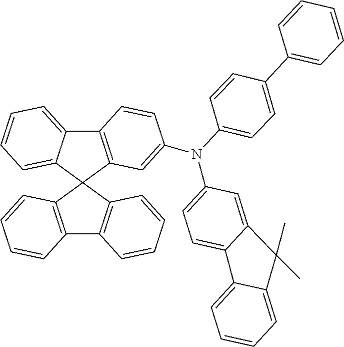 [SMILES: CC1(C)c2ccccc2-c2ccc(N(c3ccc(-c4ccccc4)cc3)c3ccc4c(c3)C3(c5ccccc5-c5ccccc53)c3ccccc3-4)cc21]

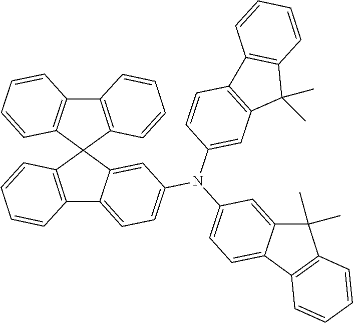 [SMILES: CC1(C)c2ccccc2-c2ccc(N(c3ccc4c(c3)C(C)(C)c3ccccc3-4)c3ccc4c(c3)C3(c5ccccc5-c5ccccc53)c3ccccc3-4)cc21]

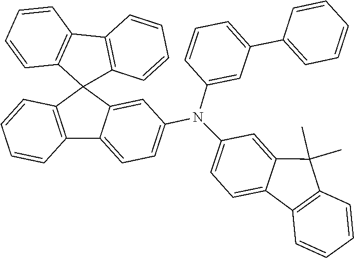 [SMILES: CC1(C)c2ccccc2-c2ccc(N(c3cccc(-c4ccccc4)c3)c3ccc4c(c3)C3(c5ccccc5-c5ccccc53)c3ccccc3-4)cc21]

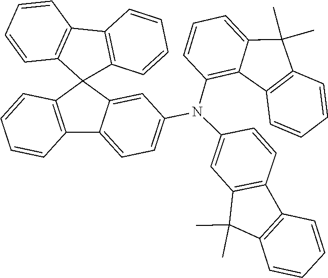 [SMILES: CC1(C)c2ccccc2-c2ccc(N(c3ccc4c(c3)C3(c5ccccc5-c5ccccc53)c3ccccc3-4)c3cccc4c3-c3ccccc3C4(C)C)cc21]

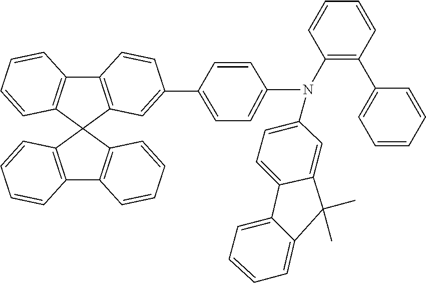 [SMILES: CC1(C)c2ccccc2-c2ccc(N(c3ccc(-c4ccc5c(c4)C4(c6ccccc6-c6ccccc64)c4ccccc4-5)cc3)c3ccccc3-c3ccccc3)cc21]

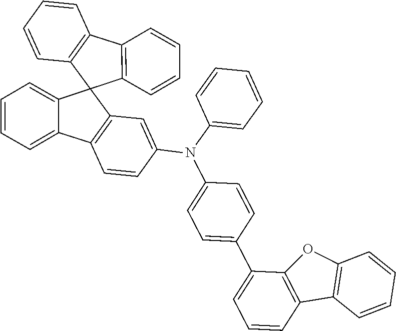 [SMILES: c1ccc(N(c2ccc(-c3cccc4c3oc3ccccc34)cc2)c2ccc3c(c2)C2(c4ccccc4-c4ccccc42)c2ccccc2-3)cc1]

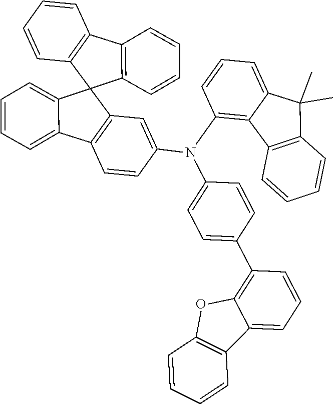 [SMILES: CC1(C)c2ccccc2-c2c(N(c3ccc(-c4cccc5c4oc4ccccc45)cc3)c3ccc4c(c3)C3(c5ccccc5-c5ccccc53)c3ccccc3-4)cccc21]

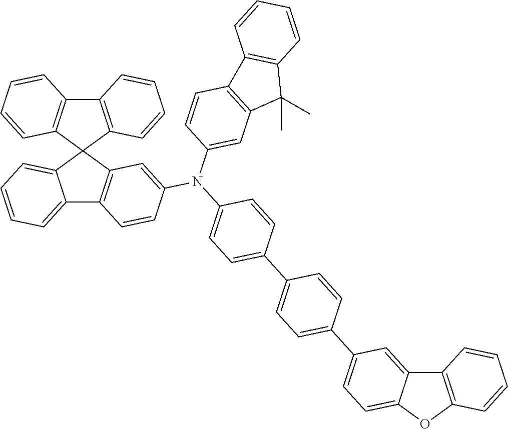 [SMILES: CC1(C)c2ccccc2-c2ccc(N(c3ccc(-c4ccc(-c5ccc6oc7ccccc7c6c5)cc4)cc3)c3ccc4c(c3)C3(c5ccccc5-c5ccccc53)c3ccccc3-4)cc21]

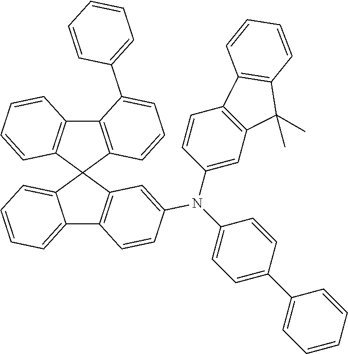 [SMILES: CC1(C)c2ccccc2-c2ccc(N(c3ccc(-c4ccccc4)cc3)c3ccc4c(c3)C3(c5ccccc5-4)c4ccccc4-c4c(-c5ccccc5)cccc43)cc21]

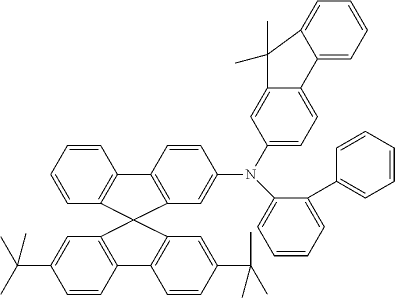 [SMILES: CC(C)(C)c1ccc2c(c1)C1(c3ccccc3-c3ccc(N(c4ccc5c(c4)C(C)(C)c4ccccc4-5)c4ccccc4-c4ccccc4)cc31)c1cc(C(C)(C)C)ccc1-2]

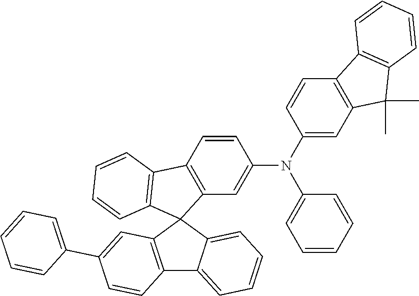 [SMILES: CC1(C)c2ccccc2-c2ccc(N(c3ccccc3)c3ccc4c(c3)C3(c5ccccc5-c5ccc(-c6ccccc6)cc53)c3ccccc3-4)cc21]

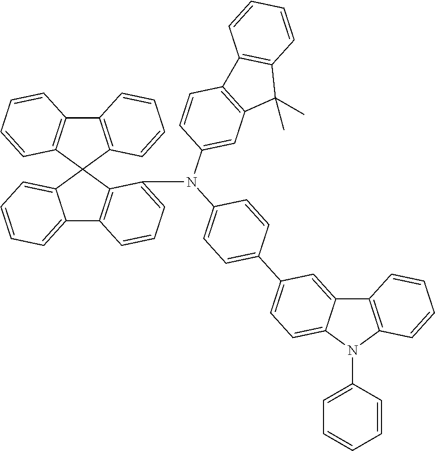 [SMILES: CC1(C)c2ccccc2-c2ccc(N(c3ccc(-c4ccc5c(c4)c4ccccc4n5-c4ccccc4)cc3)c3cccc4c3C3(c5ccccc5-c5ccccc53)c3ccccc3-4)cc21]

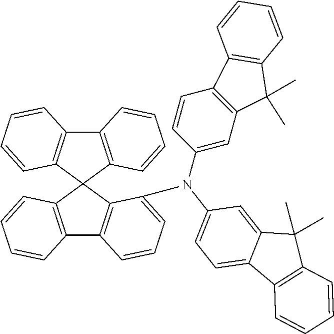 [SMILES: CC1(C)c2ccccc2-c2ccc(N(c3ccc4c(c3)C(C)(C)c3ccccc3-4)c3cccc4c3C3(c5ccccc5-c5ccccc53)c3ccccc3-4)cc21]

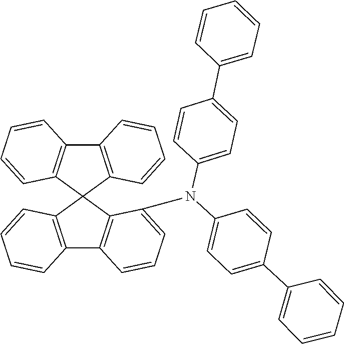 [SMILES: c1ccc(-c2ccc(N(c3ccc(-c4ccccc4)cc3)c3cccc4c3C3(c5ccccc5-c5ccccc53)c3ccccc3-4)cc2)cc1]

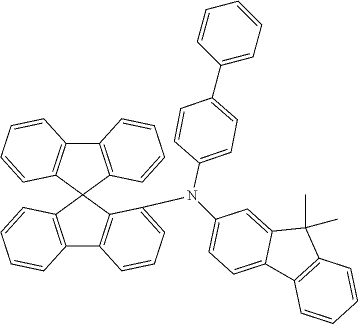 [SMILES: CC1(C)c2ccccc2-c2ccc(N(c3ccc(-c4ccccc4)cc3)c3cccc4c3C3(c5ccccc5-c5ccccc53)c3ccccc3-4)cc21]

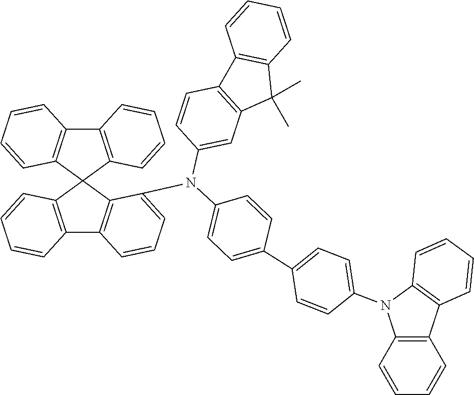 [SMILES: CC1(C)c2ccccc2-c2ccc(N(c3ccc(-c4ccc(-n5c6ccccc6c6ccccc65)cc4)cc3)c3cccc4c3C3(c5ccccc5-c5ccccc53)c3ccccc3-4)cc21]

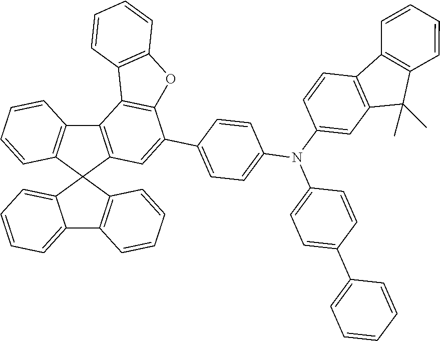 [SMILES: CC1(C)c2ccccc2-c2ccc(N(c3ccc(-c4ccccc4)cc3)c3ccc(-c4cc5c(c6c4oc4ccccc46)-c4ccccc4C54c5ccccc5-c5ccccc54)cc3)cc21]

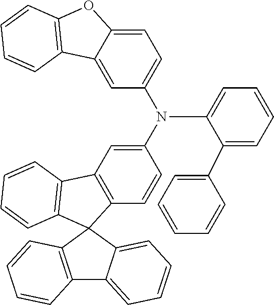 [SMILES: c1ccc(-c2ccccc2N(c2ccc3c(c2)-c2ccccc2C32c3ccccc3-c3ccccc32)c2ccc3oc4ccccc4c3c2)cc1]

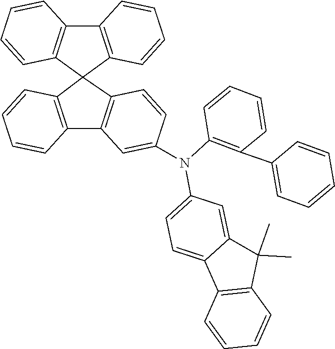 [SMILES: CC1(C)c2ccccc2-c2ccc(N(c3ccc4c(c3)-c3ccccc3C43c4ccccc4-c4ccccc43)c3ccccc3-c3ccccc3)cc21]

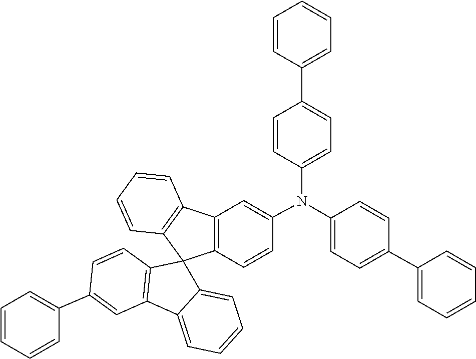 [SMILES: c1ccc(-c2ccc(N(c3ccc(-c4ccccc4)cc3)c3ccc4c(c3)-c3ccccc3C43c4ccccc4-c4cc(-c5ccccc5)ccc43)cc2)cc1]

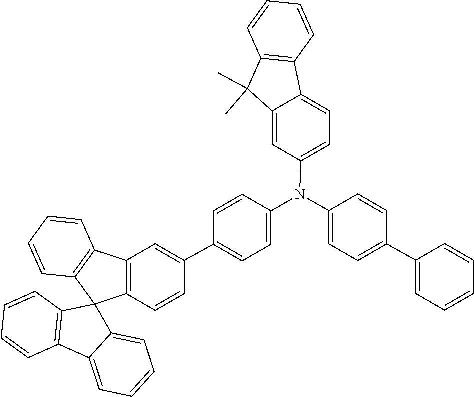 [SMILES: CC1(C)c2ccccc2-c2ccc(N(c3ccc(-c4ccccc4)cc3)c3ccc(-c4ccc5c(c4)-c4ccccc4C54c5ccccc5-c5ccccc54)cc3)cc21]

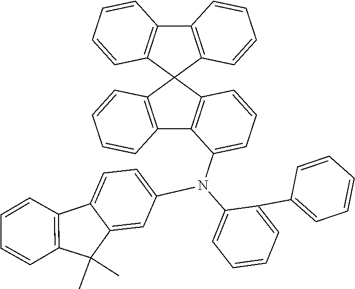 [SMILES: CC1(C)c2ccccc2-c2ccc(N(c3ccccc3-c3ccccc3)c3cccc4c3-c3ccccc3C43c4ccccc4-c4ccccc43)cc21]

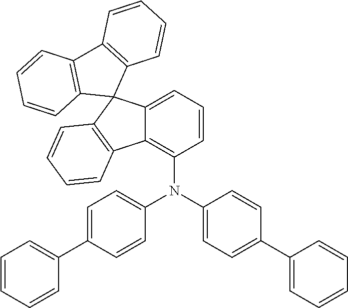 [SMILES: c1ccc(-c2ccc(N(c3ccc(-c4ccccc4)cc3)c3cccc4c3-c3ccccc3C43c4ccccc4-c4ccccc43)cc2)cc1]

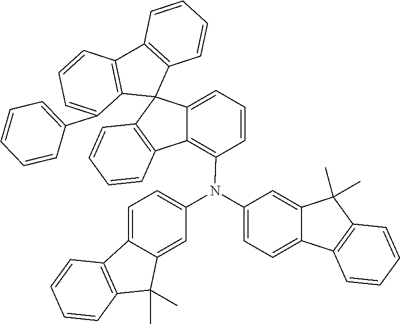 [SMILES: CC1(C)c2ccccc2-c2ccc(N(c3ccc4c(c3)C(C)(C)c3ccccc3-4)c3cccc4c3-c3ccccc3C43c4ccccc4-c4cccc(-c5ccccc5)c43)cc21]

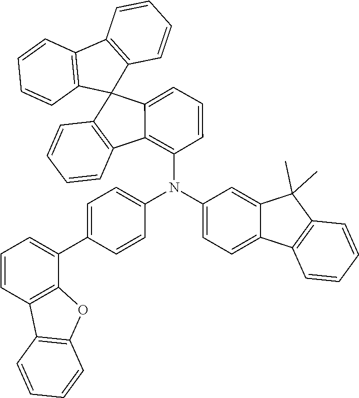 [SMILES: CC1(C)c2ccccc2-c2ccc(N(c3ccc(-c4cccc5c4oc4ccccc45)cc3)c3cccc4c3-c3ccccc3C43c4ccccc4-c4ccccc43)cc21]

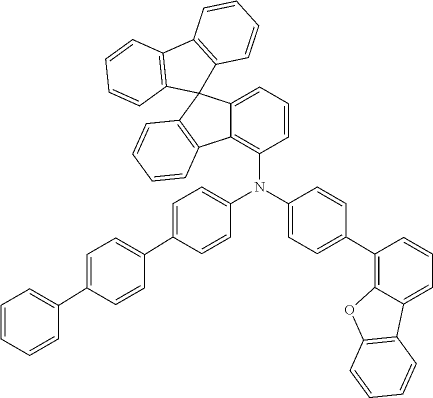 [SMILES: c1ccc(-c2ccc(-c3ccc(N(c4ccc(-c5cccc6c5oc5ccccc56)cc4)c4cccc5c4-c4ccccc4C54c5ccccc5-c5ccccc54)cc3)cc2)cc1]

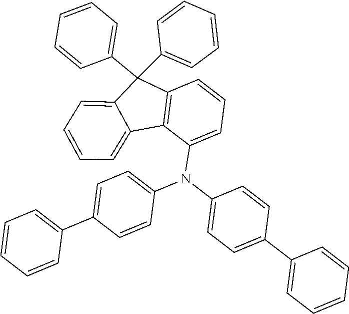 [SMILES: c1ccc(-c2ccc(N(c3ccc(-c4ccccc4)cc3)c3cccc4c3-c3ccccc3C4(c3ccccc3)c3ccccc3)cc2)cc1]

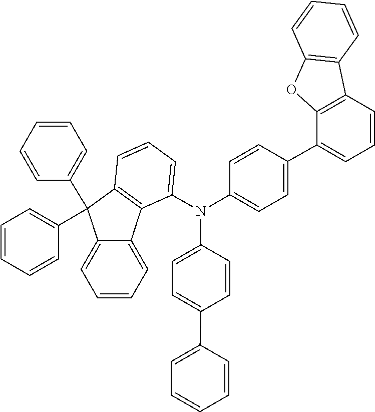 [SMILES: c1ccc(-c2ccc(N(c3ccc(-c4cccc5c4oc4ccccc45)cc3)c3cccc4c3-c3ccccc3C4(c3ccccc3)c3ccccc3)cc2)cc1]

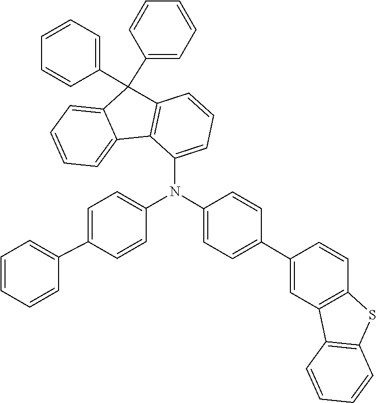 [SMILES: c1ccc(-c2ccc(N(c3ccc(-c4ccc5sc6ccccc6c5c4)cc3)c3cccc4c3-c3ccccc3C4(c3ccccc3)c3ccccc3)cc2)cc1]

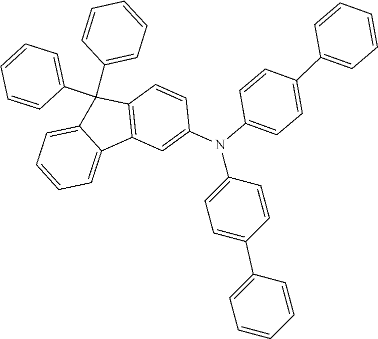 [SMILES: c1ccc(-c2ccc(N(c3ccc(-c4ccccc4)cc3)c3ccc4c(c3)-c3ccccc3C4(c3ccccc3)c3ccccc3)cc2)cc1]

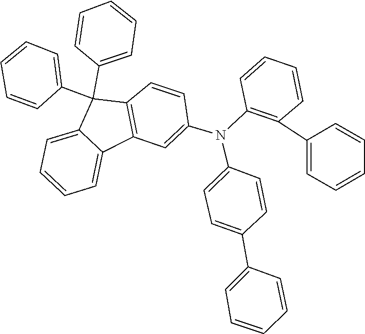 [SMILES: c1ccc(-c2ccc(N(c3ccc4c(c3)-c3ccccc3C4(c3ccccc3)c3ccccc3)c3ccccc3-c3ccccc3)cc2)cc1]

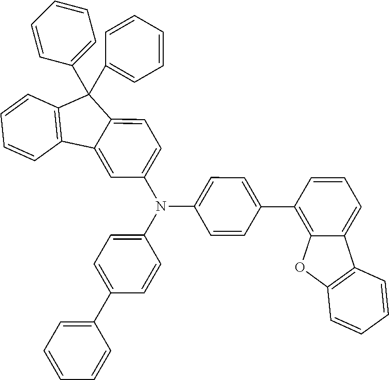 [SMILES: c1ccc(-c2ccc(N(c3ccc(-c4cccc5c4oc4ccccc45)cc3)c3ccc4c(c3)-c3ccccc3C4(c3ccccc3)c3ccccc3)cc2)cc1]

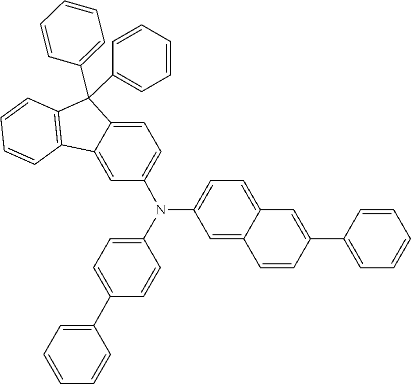 [SMILES: c1ccc(-c2ccc(N(c3ccc4c(c3)-c3ccccc3C4(c3ccccc3)c3ccccc3)c3ccc4cc(-c5ccccc5)ccc4c3)cc2)cc1]

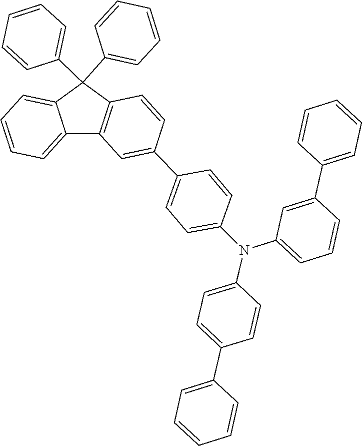 [SMILES: c1ccc(-c2ccc(N(c3ccc(-c4ccc5c(c4)-c4ccccc4C5(c4ccccc4)c4ccccc4)cc3)c3cccc(-c4ccccc4)c3)cc2)cc1]

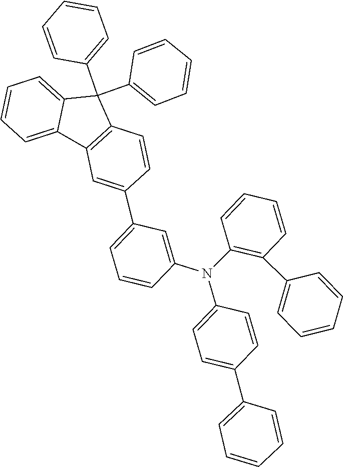 [SMILES: c1ccc(-c2ccc(N(c3cccc(-c4ccc5c(c4)-c4ccccc4C5(c4ccccc4)c4ccccc4)c3)c3ccccc3-c3ccccc3)cc2)cc1]

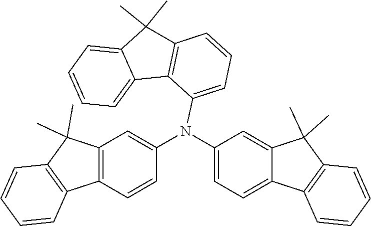 [SMILES: CC1(C)c2ccccc2-c2ccc(N(c3ccc4c(c3)C(C)(C)c3ccccc3-4)c3cccc4c3-c3ccccc3C4(C)C)cc21]

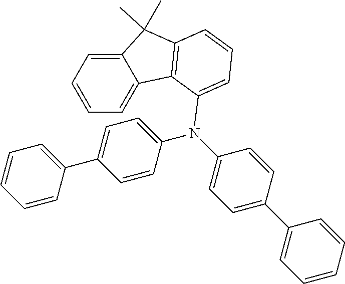 [SMILES: CC1(C)c2ccccc2-c2c(N(c3ccc(-c4ccccc4)cc3)c3ccc(-c4ccccc4)cc3)cccc21]

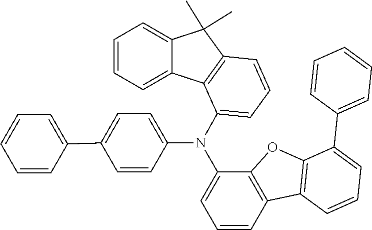 [SMILES: CC1(C)c2ccccc2-c2c(N(c3ccc(-c4ccccc4)cc3)c3cccc4c3oc3c(-c5ccccc5)cccc34)cccc21]

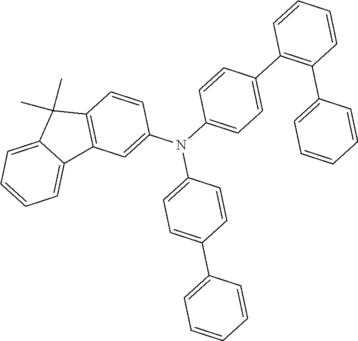 [SMILES: CC1(C)c2ccccc2-c2cc(N(c3ccc(-c4ccccc4)cc3)c3ccc(-c4ccccc4-c4ccccc4)cc3)ccc21]

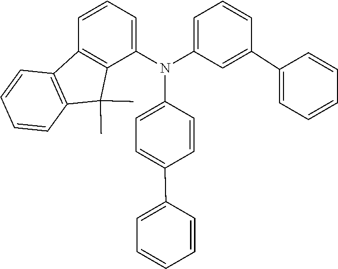 [SMILES: CC1(C)c2ccccc2-c2cccc(N(c3ccc(-c4ccccc4)cc3)c3cccc(-c4ccccc4)c3)c21]

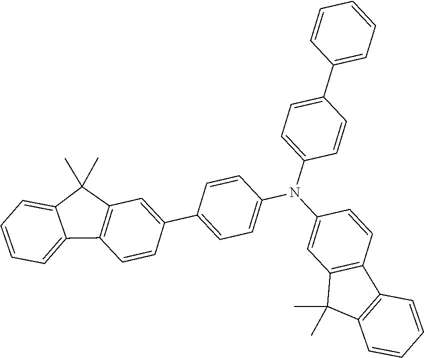 [SMILES: CC1(C)c2ccccc2-c2ccc(-c3ccc(N(c4ccc(-c5ccccc5)cc4)c4ccc5c(c4)C(C)(C)c4ccccc4-5)cc3)cc21]

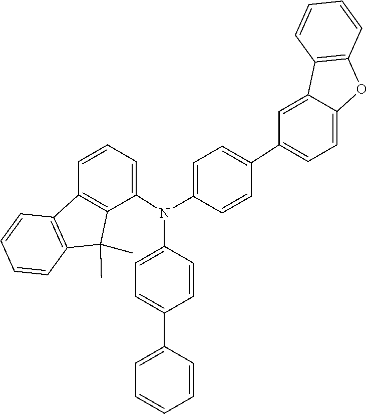 [SMILES: CC1(C)c2ccccc2-c2cccc(N(c3ccc(-c4ccccc4)cc3)c3ccc(-c4ccc5oc6ccccc6c5c4)cc3)c21]

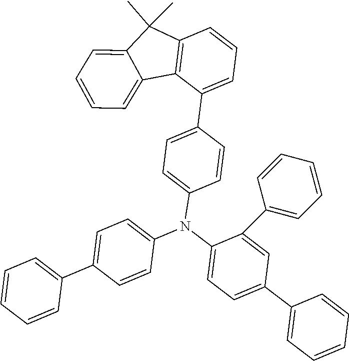 [SMILES: CC1(C)c2ccccc2-c2c(-c3ccc(N(c4ccc(-c5ccccc5)cc4)c4ccc(-c5ccccc5)cc4-c4ccccc4)cc3)cccc21]

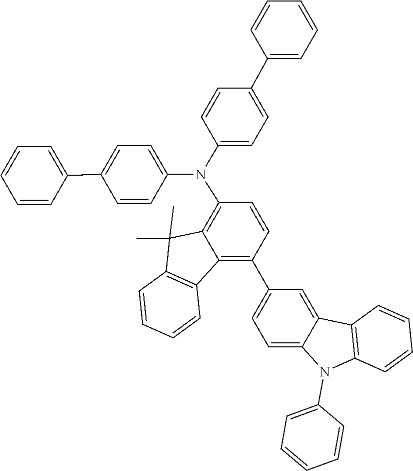 [SMILES: CC1(C)c2ccccc2-c2c(-c3ccc4c(c3)c3ccccc3n4-c3ccccc3)ccc(N(c3ccc(-c4ccccc4)cc3)c3ccc(-c4ccccc4)cc3)c21]

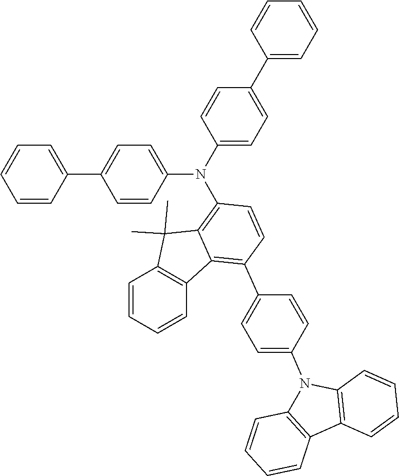 [SMILES: CC1(C)c2ccccc2-c2c(-c3ccc(-n4c5ccccc5c5ccccc54)cc3)ccc(N(c3ccc(-c4ccccc4)cc3)c3ccc(-c4ccccc4)cc3)c21]

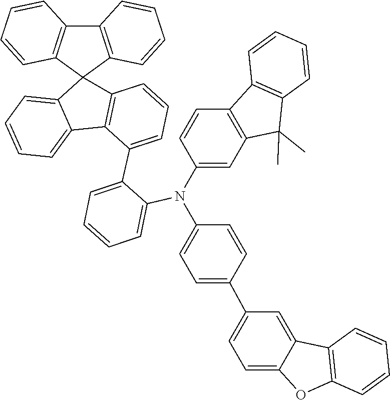 [SMILES: CC1(C)c2ccccc2-c2ccc(N(c3ccc(-c4ccc5oc6ccccc6c5c4)cc3)c3ccccc3-c3cccc4c3-c3ccccc3C43c4ccccc4-c4ccccc43)cc21]

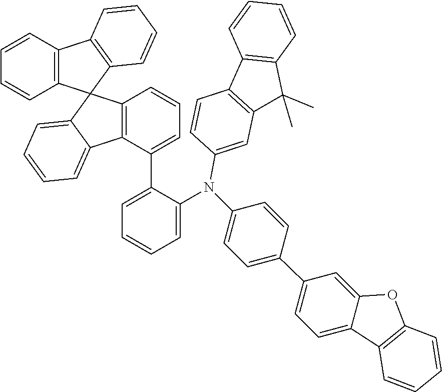 [SMILES: CC1(C)c2ccccc2-c2ccc(N(c3ccc(-c4ccc5c(c4)oc4ccccc45)cc3)c3ccccc3-c3cccc4c3-c3ccccc3C43c4ccccc4-c4ccccc43)cc21]

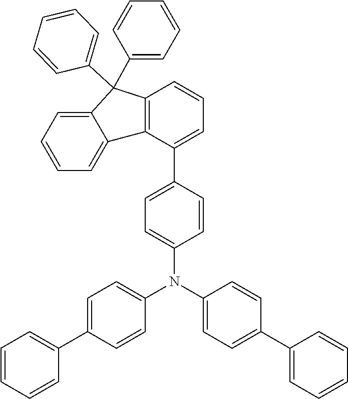 [SMILES: c1ccc(-c2ccc(N(c3ccc(-c4ccccc4)cc3)c3ccc(-c4cccc5c4-c4ccccc4C5(c4ccccc4)c4ccccc4)cc3)cc2)cc1]

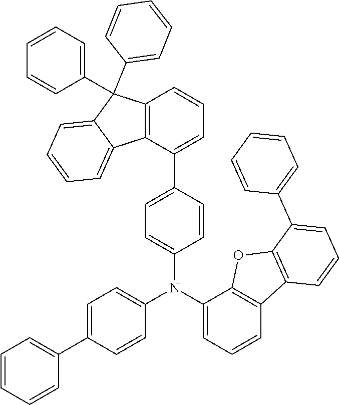 [SMILES: c1ccc(-c2ccc(N(c3ccc(-c4cccc5c4-c4ccccc4C5(c4ccccc4)c4ccccc4)cc3)c3cccc4c3oc3c(-c5ccccc5)cccc34)cc2)cc1]

In addition, the following compounds HT-1 to HT-13 are suitable for use in a layer having a hole-transporting function, especially in a hole injection layer, a hole transport layer and/or an electron blocker layer, or for use in an emitting layer as matrix material, especially as matrix material in an emitting layer comprising one or more phosphorescent emitters:

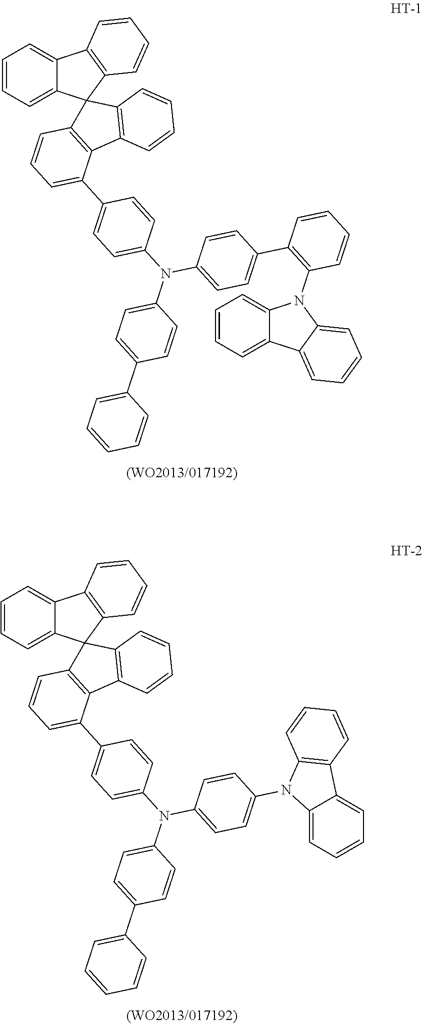 [SMILES: c1ccc(-c2ccc(N(c3ccc(-c4ccccc4-n4c5ccccc5c5ccccc54)cc3)c3ccc(-c4cccc5c4-c4ccccc4C54c5ccccc5-c5ccccc54)cc3)cc2)cc1 ; c1ccc(-c2ccc(N(c3ccc(-c4cccc5c4-c4ccccc4C54c5ccccc5-c5ccccc54)cc3)c3ccc(-n4c5ccccc5c5ccccc54)cc3)cc2)cc1]

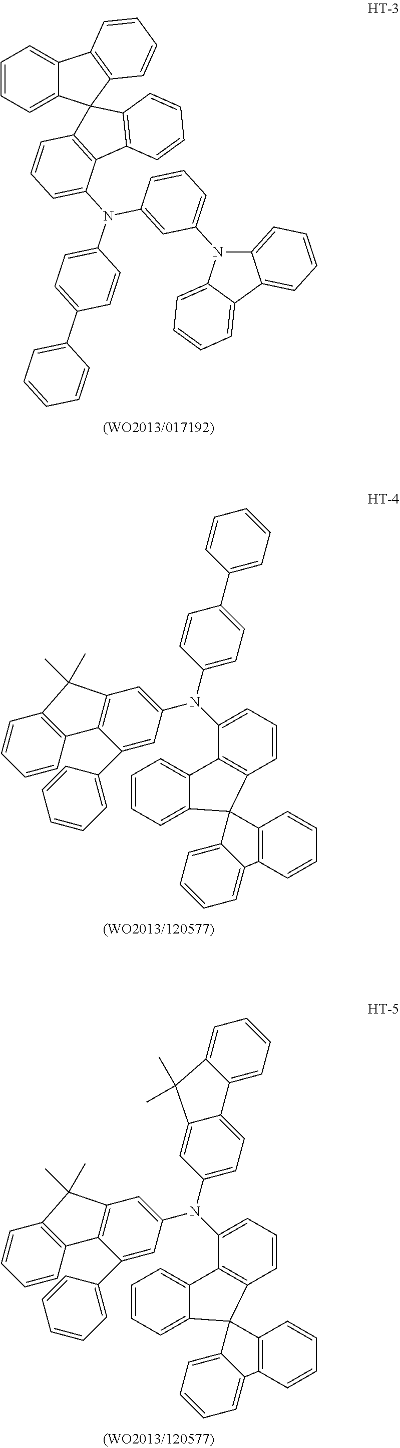 [SMILES: c1ccc(-c2ccc(N(c3cccc(-n4c5ccccc5c5ccccc54)c3)c3cccc4c3-c3ccccc3C43c4ccccc4-c4ccccc43)cc2)cc1 ; CC1(C)c2ccccc2-c2c(-c3ccccc3)cc(N(c3ccc(-c4ccccc4)cc3)c3cccc4c3-c3ccccc3C43c4ccccc4-c4ccccc43)cc21 ; CC1(C)c2ccccc2-c2ccc(N(c3cc(-c4ccccc4)c4c(c3)C(C)(C)c3ccccc3-4)c3cccc4c3-c3ccccc3C43c4ccccc4-c4ccccc43)cc21]

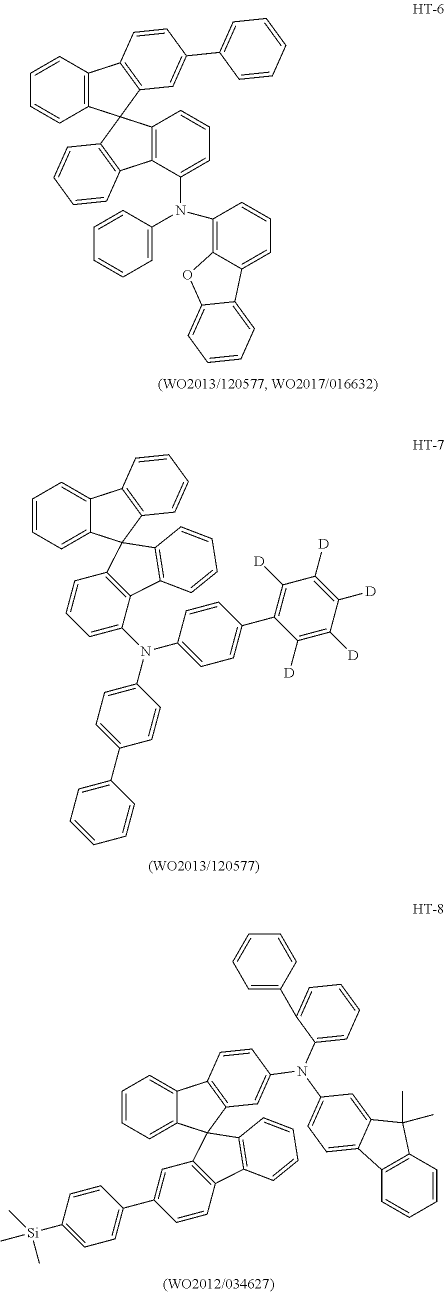 [SMILES: c1ccc(-c2ccc3c(c2)C2(c4ccccc4-3)c3ccccc3-c3c(N(c4ccccc4)c4cccc5c4oc4ccccc45)cccc32)cc1 ; [2H]c1c([2H])c([2H])c(-c2ccc(N(c3ccc(-c4ccccc4)cc3)c3cccc4c3-c3ccccc3C43c4ccccc4-c4ccccc43)cc2)c([2H])c1[2H] ; CC1(C)c2ccccc2-c2ccc(N(c3ccc4c(c3)C3(c5ccccc5-c5ccc(-c6ccc([Si](C)(C)C)cc6)cc53)c3ccccc3-4)c3ccccc3-c3ccccc3)cc21]

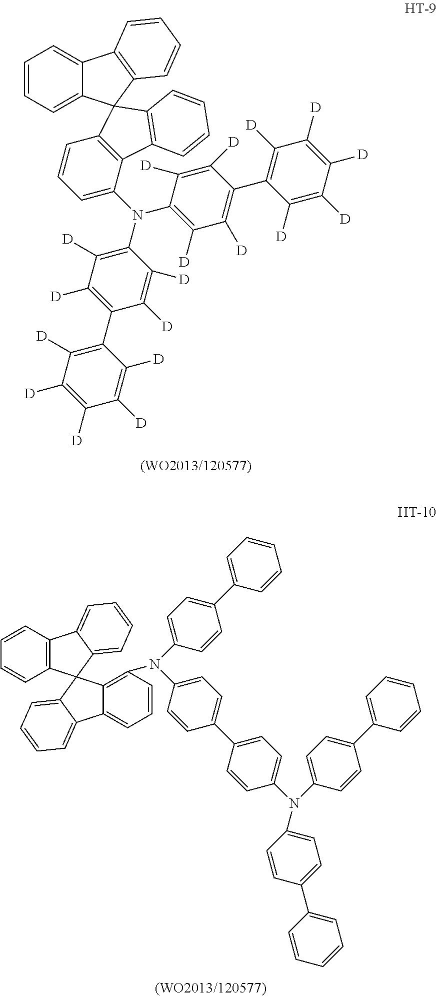 [SMILES: [2H]c1c([2H])c([2H])c(-c2c([2H])c([2H])c(N(c3cccc4c3-c3ccccc3C43c4ccccc4-c4ccccc43)c3c([2H])c([2H])c(-c4c([2H])c([2H])c([2H])c([2H])c4[2H])c([2H])c3[2H])c([2H])c2[2H])c([2H])c1[2H] ; c1ccc(-c2ccc(N(c3ccc(-c4ccccc4)cc3)c3ccc(-c4ccc(N(c5ccc(-c6ccccc6)cc5)c5cccc6c5C5(c7ccccc7-c7ccccc75)c5ccccc5-6)cc4)cc3)cc2)cc1]

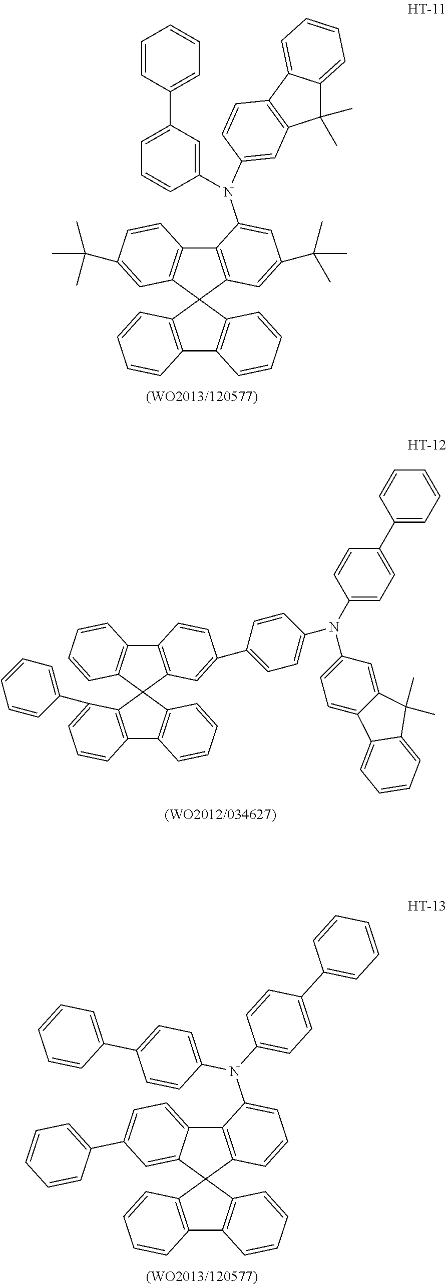 [SMILES: CC(C)(C)c1ccc2c(c1)C1(c3ccccc3-c3ccccc31)c1cc(C(C)(C)C)cc(N(c3cccc(-c4ccccc4)c3)c3ccc4c(c3)C(C)(C)c3ccccc3-4)c1-2 ; CC1(C)c2ccccc2-c2ccc(N(c3ccc(-c4ccccc4)cc3)c3ccc(-c4ccc5c(c4)C4(c6ccccc6-5)c5ccccc5-c5cccc(-c6ccccc6)c54)cc3)cc21 ; c1ccc(-c2ccc(N(c3ccc(-c4ccccc4)cc3)c3cccc4c3-c3ccc(-c5ccccc5)cc3C43c4ccccc4-c4ccccc43)cc2)cc1]

The compounds HT-1 to HT-13 are generally of good suitability for the abovementioned uses in OLEDs of any design and composition, not just in OLEDs according to the present application. Processes for preparing these compounds and the further relevant disclosure relating to the use of these compounds are disclosed in the published specifications that are each cited in brackets in the table beneath the respective compounds. The compounds show good performance data in OLEDs, especially good lifetime and good efficiency.

Additionally preferred is an organic electroluminescent device, characterized in that one or more layers are coated by a sublimation process. In this case, the materials are applied by vapour deposition in vacuum sublimation systems at an initial pressure of less than 10⁻⁵ mbar, preferably less than 10⁻⁶ mbar. However, it is also possible that the initial pressure is even lower, for example less than 10⁻⁷ mbar.

Preference is likewise given to an organic electroluminescent device, characterized in that one or more layers are coated by the OVPD (organic vapour phase deposition) method or with the aid of a carrier gas sublimation.

In this case, the materials are applied at a pressure between 10⁻⁵ mbar and 1 bar. A special case of this method is the OVJP (organic vapour jet printing) method, in which the materials are applied directly by a nozzle and thus structured.

Preference is additionally given to an organic electroluminescent device, characterized in that one or more layers are produced from solution, for example by spin-coating, or by any printing method, for example screen printing, flexographic printing, offset printing, LITI (light-induced thermal imaging, thermal transfer printing), inkjet printing or nozzle printing. For this purpose, soluble compounds are needed, which are obtained, for example, through suitable substitution.

In addition, hybrid methods are possible, in which, for example, one or more layers are applied from solution and one or more further layers are applied by vapour deposition.

Those skilled in the art are generally aware of these methods and are able to apply them without exercising inventive skill to organic electroluminescent devices comprising the compounds of the invention.

### EXAMPLES

### A) Synthesis of Compounds of the Formula (I)

### a) 9-Phenyl-3-(2-phenyl-1H-benzimidazol-5-yl)carbazole

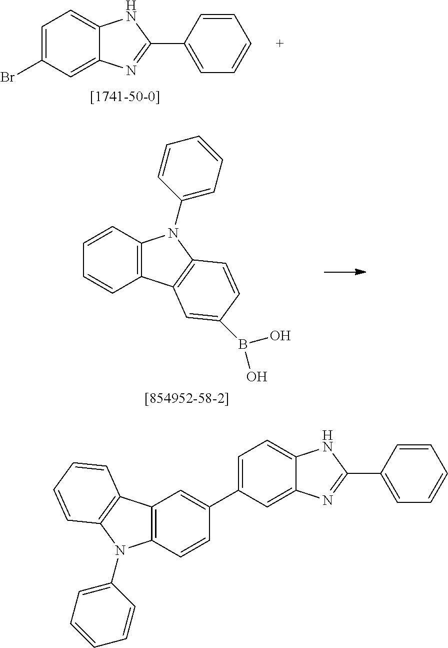 [SMILES: Brc1ccc2[nH]c(-c3ccccc3)nc2c1 ; OB(O)c1ccc2c(c1)c1ccccc1n2-c1ccccc1 ; c1ccc(-c2nc3cc(-c4ccc5c(c4)c4ccccc4n5-c4ccccc4)ccc3[nH]2)cc1]

31.5 g (110.0 mmol) of phenylcarbazole-3-boronic acid, 30 g (110.0 mmol) of 6-bromo-2-phenyl-1H-benzimidazole and 44.6 g (210.0 mmol) of tripotassium phosphate are suspended in 500 ml of toluene, 500 ml of dioxane and 500 ml of water. To this suspension are added 913 mg (3.0 mmol) of tri-o-tolylphosphine and then 112 mg (0.5 mmol) of palladium(II) acetate, and the reaction mixture is heated under reflux for 16 h. After cooling, the organic phase is removed, filtered through silica gel, washed three times with 200 ml each time of water and then concentrated to dryness. The residue is recrystallized from toluene and from dichloromethane/iso-propanol. The yield is 39.6 g (91 mmol), corresponding to 83% of theory.

The following compounds can be obtained analogously:

| Ex. | Reactant 1 | Reactant 2 | Product | Yield |
|---|---|---|---|---|
| 1a | 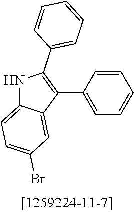 [SMILES: Brc1ccc2[nH]c(-c3ccccc3)c(-c3ccccc3)c2c1] | 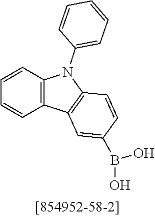 [SMILES: OB(O)c1ccc2c(c1)c1ccccc1n2-c1ccccc1] | 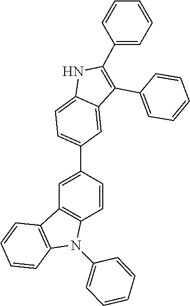 [SMILES: c1ccc(-c2[nH]c3ccc(-c4ccc5c(c4)c4ccccc4n5-c4ccccc4)cc3c2-c2ccccc2)cc1] | 74% |
| 2a | 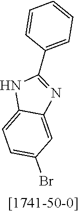 [SMILES: Brc1ccc2[nH]c(-c3ccccc3)nc2c1] | 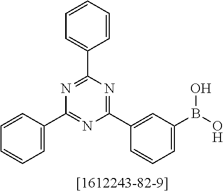 [SMILES: OB(O)c1cccc(-c2nc(-c3ccccc3)nc(-c3ccccc3)n2)c1] | 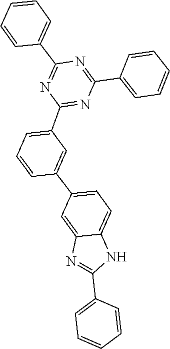 [SMILES: c1ccc(-c2nc(-c3ccccc3)nc(-c3cccc(-c4ccc5[nH]c(-c6ccccc6)nc5c4)c3)n2)cc1] | 72% |
| 3a | 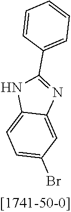 [SMILES: Brc1ccc2[nH]c(-c3ccccc3)nc2c1] | 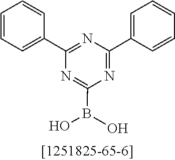 [SMILES: OB(O)c1nc(-c2ccccc2)nc(-c2ccccc2)n1] | 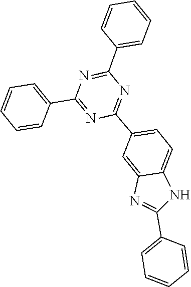 [SMILES: c1ccc(-c2nc(-c3ccccc3)nc(-c3ccc4[nH]c(-c5ccccc5)nc4c3)n2)cc1] | 66% |
| 4a | 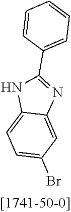 [SMILES: Brc1ccc2[nH]c(-c3ccccc3)nc2c1] | 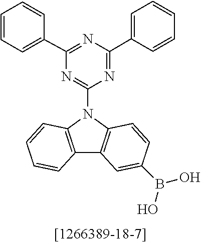 [SMILES: OB(O)c1ccc2c(c1)c1ccccc1n2-c1nc(-c2ccccc2)nc(-c2ccccc2)n1] | 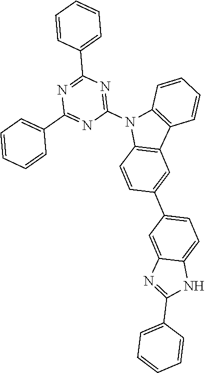 [SMILES: c1ccc(-c2nc(-c3ccccc3)nc(-n3c4ccccc4c4cc(-c5ccc6[nH]c(-c7ccccc7)nc6c5)ccc43)n2)cc1] | 76% |
| 5a | 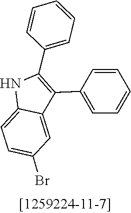 [SMILES: Brc1ccc2[nH]c(-c3ccccc3)c(-c3ccccc3)c2c1] | 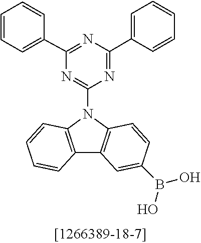 [SMILES: OB(O)c1ccc2c(c1)c1ccccc1n2-c1nc(-c2ccccc2)nc(-c2ccccc2)n1] | 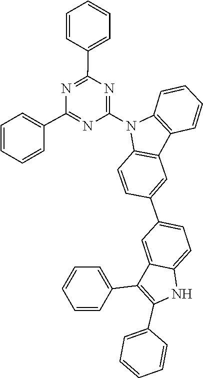 [SMILES: c1ccc(-c2nc(-c3ccccc3)nc(-n3c4ccccc4c4cc(-c5ccc6[nH]c(-c7ccccc7)c(-c7ccccc7)c6c5)ccc43)n2)cc1] | 65% |
| 6a | 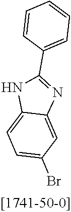 [SMILES: Brc1ccc2[nH]c(-c3ccccc3)nc2c1] | 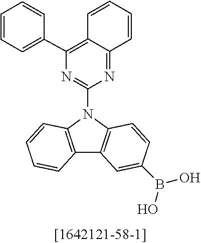 [SMILES: OB(O)c1ccc2c(c1)c1ccccc1n2-c1nc(-c2ccccc2)c2ccccc2n1] | 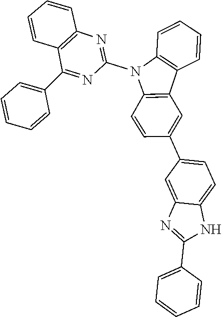 [SMILES: c1ccc(-c2nc3cc(-c4ccc5c(c4)c4ccccc4n5-c4nc(-c5ccccc5)c5ccccc5n4)ccc3[nH]2)cc1] | 60% |
| 7a | 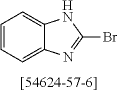 | 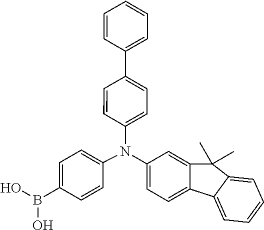 [SMILES: CC1(C)c2ccccc2-c2ccc(N(c3ccc(B(O)O)cc3)c3ccc(-c4ccccc4)cc3)cc21] | 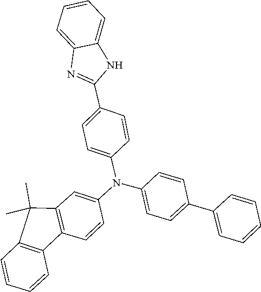 [SMILES: CC1(C)c2ccccc2-c2ccc(N(c3ccc(-c4ccccc4)cc3)c3ccc(-c4nc5ccccc5[nH]4)cc3)cc21] | 59% |
| 8a | 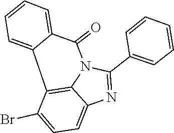 [SMILES: O=c1c2ccccc2c2c(Br)ccc3nc(-c4ccccc4)n1c32] | 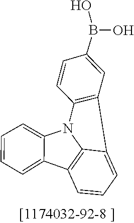 [SMILES: OB(O)c1ccc2c(c1)c1cccc3c4ccccc4n2c31] | 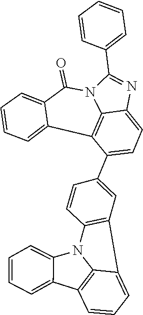 [SMILES: O=c1c2ccccc2c2c(-c3ccc4c(c3)c3cccc5c6ccccc6n4c53)ccc3nc(-c4ccccc4)n1c32] | 62% |
| 9a | 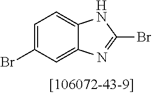 [SMILES: Brc1ccc2[nH]c(Br)nc2c1] | 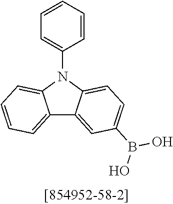 [SMILES: OB(O)c1ccc2c(c1)c1ccccc1n2-c1ccccc1] | 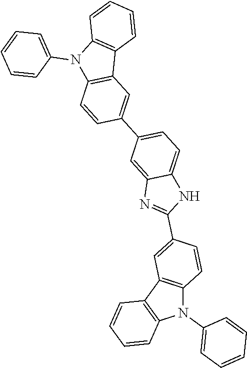 [SMILES: c1ccc(-n2c3ccccc3c3cc(-c4ccc5[nH]c(-c6ccc7c(c6)c6ccccc6n7-c6ccccc6)nc5c4)ccc32)cc1] | 67% |
| 10a | 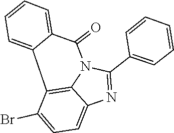 [SMILES: O=c1c2ccccc2c2c(Br)ccc3nc(-c4ccccc4)n1c32] | 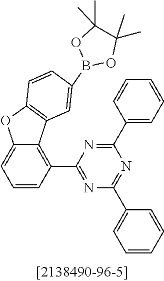 [SMILES: CC1(C)OB(c2ccc3oc4cccc(-c5nc(-c6ccccc6)nc(-c6ccccc6)n5)c4c3c2)OC1(C)C] | 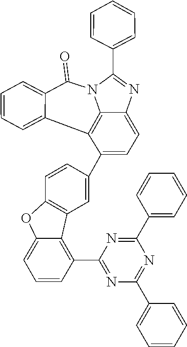 [SMILES: O=c1c2ccccc2c2c(-c3ccc4oc5cccc(-c6nc(-c7ccccc7)nc(-c7ccccc7)n6)c5c4c3)ccc3nc(-c4ccccc4)n1c32] | 80% |
| 11a | 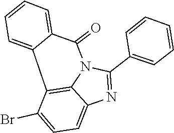 [SMILES: O=c1c2ccccc2c2c(Br)ccc3nc(-c4ccccc4)n1c32] | 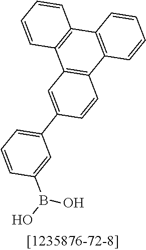 [SMILES: OB(O)c1cccc(-c2ccc3c4ccccc4c4ccccc4c3c2)c1] | 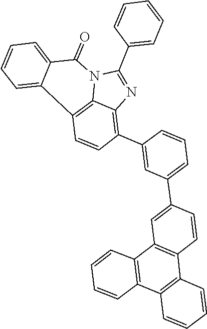 [SMILES: O=c1c2ccccc2c2ccc(-c3cccc(-c4ccc5c6ccccc6c6ccccc6c5c4)c3)c3nc(-c4ccccc4)n1c32] | 82% |
| 12a | 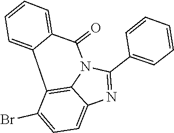 [SMILES: O=c1c2ccccc2c2c(Br)ccc3nc(-c4ccccc4)n1c32] | 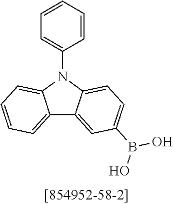 [SMILES: OB(O)c1ccc2c(c1)c1ccccc1n2-c1ccccc1] | 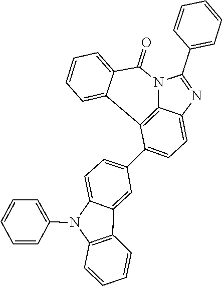 [SMILES: O=c1c2ccccc2c2c(-c3ccc4c(c3)c3ccccc3n4-c3ccccc3)ccc3nc(-c4ccccc4)n1c32] | 80% |
| 13a | 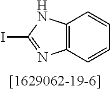 | 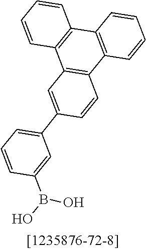 [SMILES: OB(O)c1cccc(-c2ccc3c4ccccc4c4ccccc4c3c2)c1] | 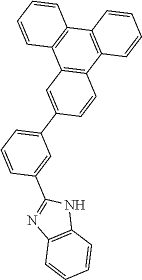 [SMILES: c1cc(-c2ccc3c4ccccc4c4ccccc4c3c2)cc(-c2nc3ccccc3[nH]2)c1] | 85% |
| 14a | 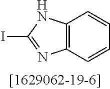 | 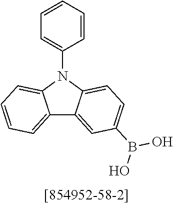 [SMILES: OB(O)c1ccc2c(c1)c1ccccc1n2-c1ccccc1] | 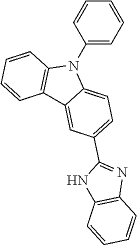 [SMILES: c1ccc(-n2c3ccccc3c3cc(-c4nc5ccccc5[nH]4)ccc32)cc1] | 81% |
| 15a | 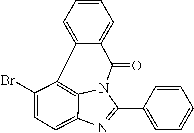 [SMILES: O=c1c2ccccc2c2c(Br)ccc3nc(-c4ccccc4)n1c32] | 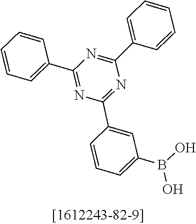 [SMILES: OB(O)c1cccc(-c2nc(-c3ccccc3)nc(-c3ccccc3)n2)c1] | 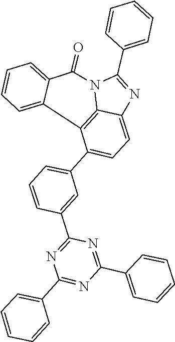 [SMILES: O=c1c2ccccc2c2c(-c3cccc(-c4nc(-c5ccccc5)nc(-c5ccccc5)n4)c3)ccc3nc(-c4ccccc4)n1c32] | 80% |
| 16a | 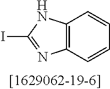 | 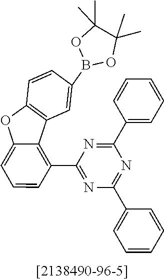 [SMILES: CC1(C)OB(c2ccc3oc4cccc(-c5nc(-c6ccccc6)nc(-c6ccccc6)n5)c4c3c2)OC1(C)C] | 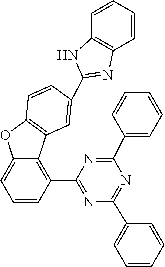 [SMILES: c1ccc(-c2nc(-c3ccccc3)nc(-c3cccc4oc5ccc(-c6nc7ccccc7[nH]6)cc5c34)n2)cc1] | 83% |
| 17a | 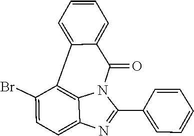 [SMILES: O=c1c2ccccc2c2c(Br)ccc3nc(-c4ccccc4)n1c32] | 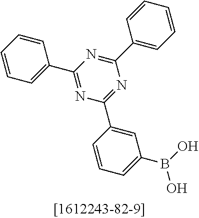 [SMILES: OB(O)c1cccc(-c2nc(-c3ccccc3)nc(-c3ccccc3)n2)c1] | 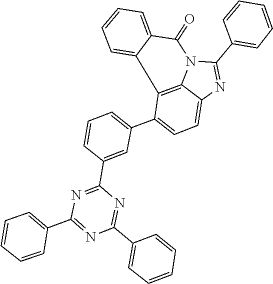 [SMILES: O=c1c2ccccc2c2c(-c3cccc(-c4nc(-c5ccccc5)nc(-c5ccccc5)n4)c3)ccc3nc(-c4ccccc4)n1c32] | 80% |
| 18a | 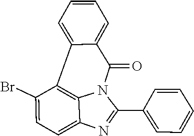 [SMILES: O=c1c2ccccc2c2c(Br)ccc3nc(-c4ccccc4)n1c32] | 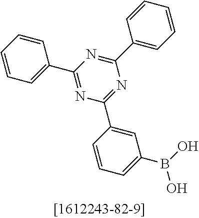 [SMILES: OB(O)c1cccc(-c2nc(-c3ccccc3)nc(-c3ccccc3)n2)c1] | 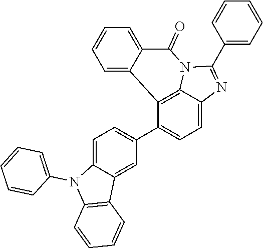 [SMILES: O=c1c2ccccc2c2c(-c3ccc4c(c3)c3ccccc3n4-c3ccccc3)ccc3nc(-c4ccccc4)n1c32] | 82% |
| 19a | 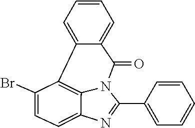 [SMILES: O=c1c2ccccc2c2c(Br)ccc3nc(-c4ccccc4)n1c32] | 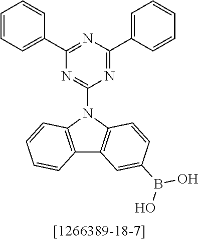 [SMILES: OB(O)c1ccc2c(c1)c1ccccc1n2-c1nc(-c2ccccc2)nc(-c2ccccc2)n1] | 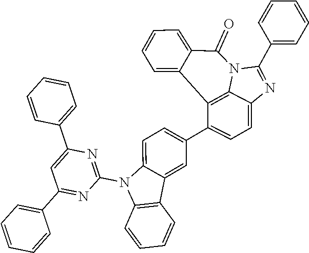 [SMILES: O=c1c2ccccc2c2c(-c3ccc4c(c3)c3ccccc3n4-c3nc(-c4ccccc4)cc(-c4ccccc4)n3)ccc3nc(-c4ccccc4)n1c32] | 79% |
| 20a | 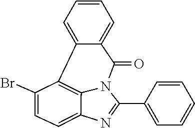 [SMILES: O=c1c2ccccc2c2c(Br)ccc3nc(-c4ccccc4)n1c32] | 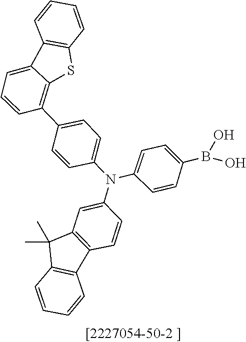 [SMILES: CC1(C)c2ccccc2-c2ccc(N(c3ccc(B(O)O)cc3)c3ccc(-c4cccc5c4sc4ccccc45)cc3)cc21] | 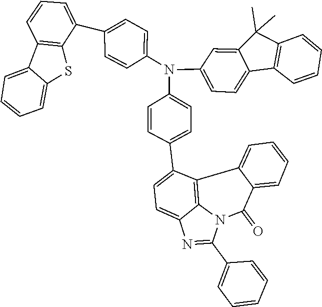 [SMILES: CC1(C)c2ccccc2-c2ccc(N(c3ccc(-c4cccc5c4sc4ccccc45)cc3)c3ccc(-c4ccc5nc(-c6ccccc6)n6c(=O)c7ccccc7c4c56)cc3)cc21] | 78% |
| 21a | 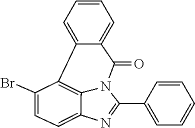 [SMILES: O=c1c2ccccc2c2c(Br)ccc3nc(-c4ccccc4)n1c32] | 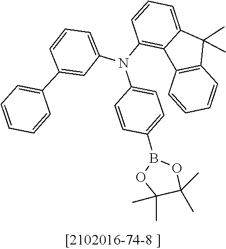 [SMILES: CC1(C)c2ccccc2-c2c(N(c3ccc(B4OC(C)(C)C(C)(C)O4)cc3)c3cccc(-c4ccccc4)c3)cccc21] | 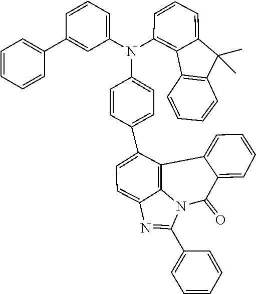 [SMILES: CC1(C)c2ccccc2-c2c(N(c3ccc(-c4ccc5nc(-c6ccccc6)n6c(=O)c7ccccc7c4c56)cc3)c3cccc(-c4ccccc4)c3)cccc21] | 79% |
| 22a | 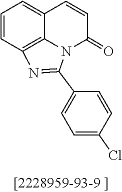 [SMILES: O=c1ccc2cccc3nc(-c4ccc(Cl)cc4)n1c23] | 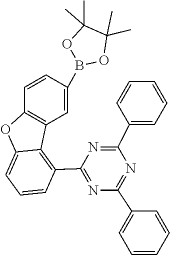 [SMILES: CC1(C)OB(c2ccc3oc4cccc(-c5nc(-c6ccccc6)nc(-c6ccccc6)n5)c4c3c2)OC1(C)C] | 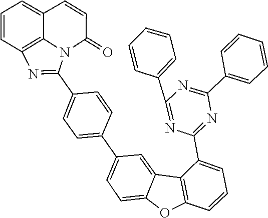 [SMILES: O=c1ccc2cccc3nc(-c4ccc(-c5ccc6oc7cccc(-c8nc(-c9ccccc9)nc(-c9ccccc9)n8)c7c6c5)cc4)n1c23] | 70% |

### b)1-[(2-bromophenyl)methyl]-2-phenylbenzimidazole

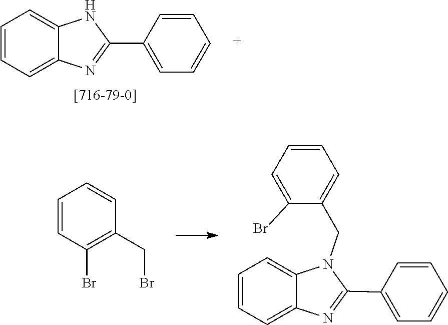 [SMILES: c1ccc(-c2nc3ccccc3[nH]2)cc1 ; BrCc1ccccc1Br ; Brc1ccccc1Cn1c(-c2ccccc2)nc2ccccc21]

13.3 g (334 mmol) of 60% NaH in mineral oil is dissolved in 1000 ml of dimethylformamide under protective atmosphere. 50 g (257 mmol) of 2-phenylbenzimidazole is dissolved in 500 ml of DMF and added dropwise to the reaction mixture. After 1 h at room temperature, a solution of 70 g (283 mmol) of 2-bromobenzyl bromide in 500 ml of DMF is added dropwise. The reaction mixture is then stirred at room temperature for 1 h. After this time, the reaction mixture is poured onto ice and extracted three times with dichloromethane. The combined organic phases are dried over Na₂SO₄ and concentrated. The residue is subjected to hot extraction with toluene and recrystallized from toluene/n-heptane.

Yield: 75 g (207 mmol), 80%.

The following compounds can be obtained analogously:

| Ex. | Reactant 1 | Reactant 2 | Product | Yield |
|---|---|---|---|---|
| 1b | 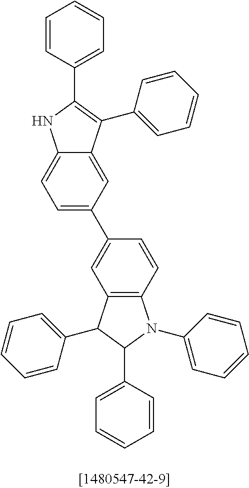 [SMILES: c1ccc(-c2[nH]c3ccc(-c4ccc5c(c4)C(c4ccccc4)C(c4ccccc4)N5c4ccccc4)cc3c2-c2ccccc2)cc1] | 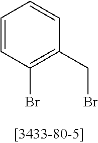 | 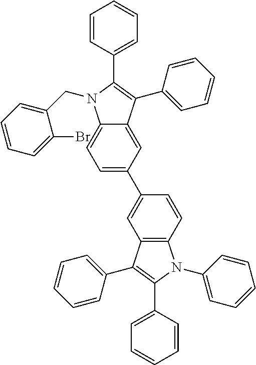 [SMILES: Brc1ccccc1Cn1c(-c2ccccc2)c(-c2ccccc2)c2cc(-c3ccc4c(c3)c(-c3ccccc3)c(-c3ccccc3)n4-c3ccccc3)ccc21] | 78% |
| 2b | 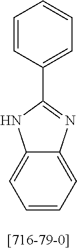 [SMILES: c1ccc(-c2nc3ccccc3[nH]2)cc1] | 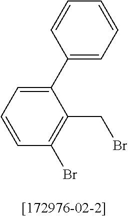 [SMILES: BrCc1c(Br)cccc1-c1ccccc1] | 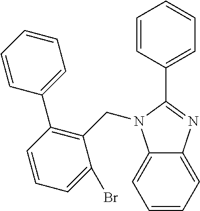 [SMILES: Brc1cccc(-c2ccccc2)c1Cn1c(-c2ccccc2)nc2ccccc21] | 80% |
| 3b | 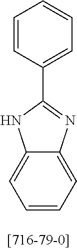 [SMILES: c1ccc(-c2nc3ccccc3[nH]2)cc1] | 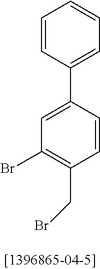 [SMILES: BrCc1ccc(-c2ccccc2)cc1Br] | 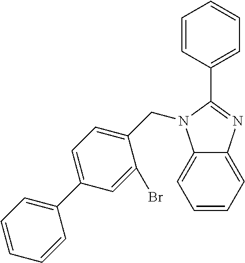 [SMILES: Brc1cc(-c2ccccc2)ccc1Cn1c(-c2ccccc2)nc2ccccc21] | 83% |
| 4b | 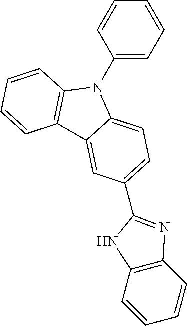 [SMILES: c1ccc(-n2c3ccccc3c3cc(-c4nc5ccccc5[nH]4)ccc32)cc1] | 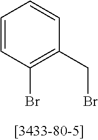 | 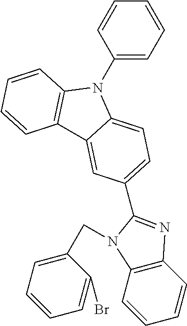 [SMILES: Brc1ccccc1Cn1c(-c2ccc3c(c2)c2ccccc2n3-c2ccccc2)nc2ccccc21] | 83% |
| 5b | 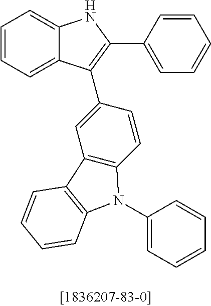 [SMILES: c1ccc(-c2[nH]c3ccccc3c2-c2ccc3c(c2)c2ccccc2n3-c2ccccc2)cc1] | 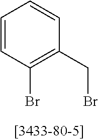 | 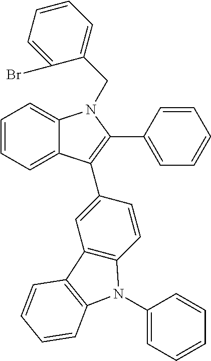 [SMILES: Brc1ccccc1Cn1c(-c2ccccc2)c(-c2ccc3c(c2)c2ccccc2n3-c2ccccc2)c2ccccc21] | 84% |
| 6b | 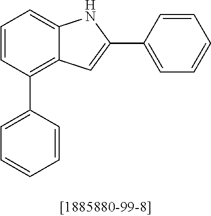 [SMILES: c1ccc(-c2cc3c(-c4ccccc4)cccc3[nH]2)cc1] | 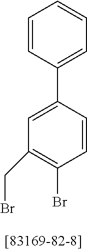 [SMILES: BrCc1cc(-c2ccccc2)ccc1Br] | 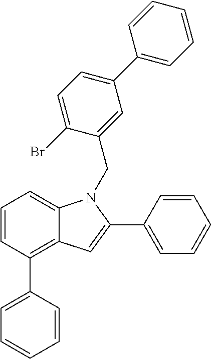 [SMILES: Brc1ccc(-c2ccccc2)cc1Cn1c(-c2ccccc2)cc2c(-c3ccccc3)cccc21] | 79% |
| 7b | 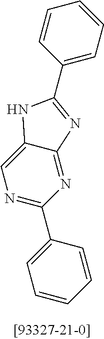 [SMILES: c1ccc(-c2ncc3[nH]c(-c4ccccc4)nc3n2)cc1] | 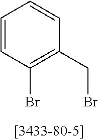 | 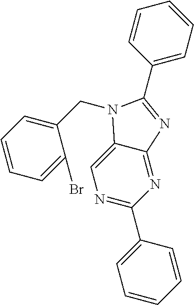 [SMILES: Brc1ccccc1Cn1c(-c2ccccc2)nc2nc(-c3ccccc3)ncc21] | 80% |
| 8b | 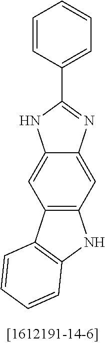 [SMILES: c1ccc(-c2nc3cc4[nH]c5ccccc5c4cc3[nH]2)cc1] | 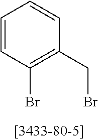 | 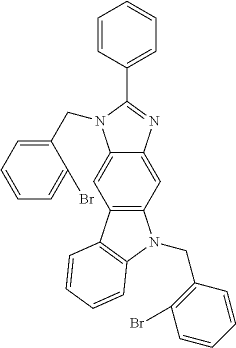 [SMILES: Brc1ccccc1Cn1c(-c2ccccc2)nc2cc3c(cc21)c1ccccc1n3Cc1ccccc1Br] | 77% |
| 9b | 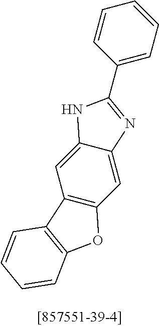 [SMILES: c1ccc(-c2nc3cc4oc5ccccc5c4cc3[nH]2)cc1] | 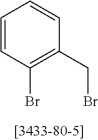 | 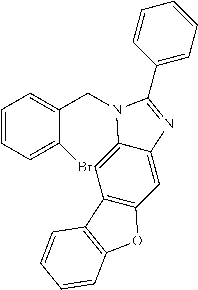 [SMILES: Brc1ccccc1Cn1c(-c2ccccc2)nc2cc3oc4ccccc4c3cc21] | 79% |
| 10b | 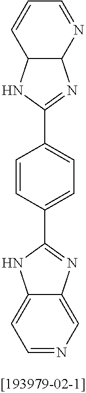 [SMILES: C1=CC2NC(c3ccc(-c4nc5cnccc5[nH]4)cc3)=NC2N=C1] | 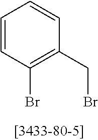 | 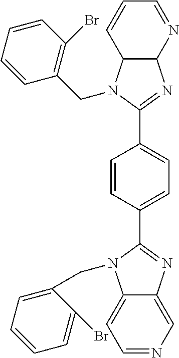 [SMILES: Brc1ccccc1CN1C(c2ccc(-c3nc4cnccc4n3Cc3ccccc3Br)cc2)=NC2N=CC=CC21] | 81% |
| 11b | 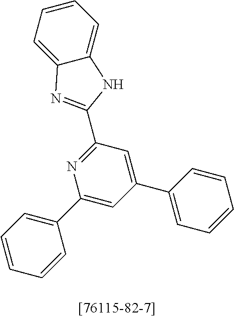 [SMILES: c1ccc(-c2cc(-c3ccccc3)nc(-c3nc4ccccc4[nH]3)c2)cc1] | 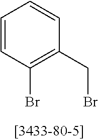 | 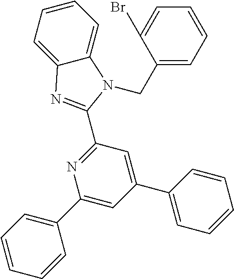 [SMILES: Brc1ccccc1Cn1c(-c2cc(-c3ccccc3)cc(-c3ccccc3)n2)nc2ccccc21] | 85% |
| 12b | 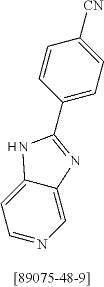 [SMILES: N#Cc1ccc(-c2nc3cnccc3[nH]2)cc1] | 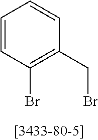 | 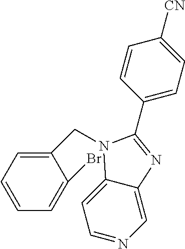 [SMILES: N#Cc1ccc(-c2nc3cnccc3n2Cc2ccccc2Br)cc1] | 78% |
| 13b | 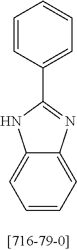 [SMILES: c1ccc(-c2nc3ccccc3[nH]2)cc1] | 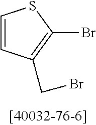 | 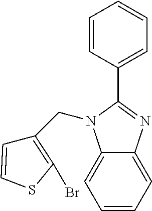 [SMILES: Brc1sccc1Cn1c(-c2ccccc2)nc2ccccc21] | 82% |
| 14b | 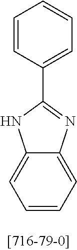 [SMILES: c1ccc(-c2nc3ccccc3[nH]2)cc1] | 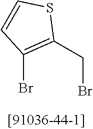 | 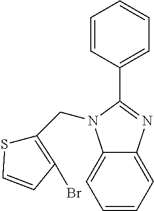 [SMILES: Brc1ccsc1Cn1c(-c2ccccc2)nc2ccccc21] | 80% |
| 15b | 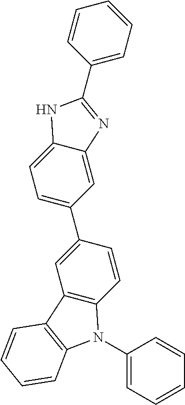 [SMILES: c1ccc(-c2nc3cc(-c4ccc5c(c4)c4ccccc4n5-c4ccccc4)ccc3[nH]2)cc1] | 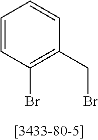 | 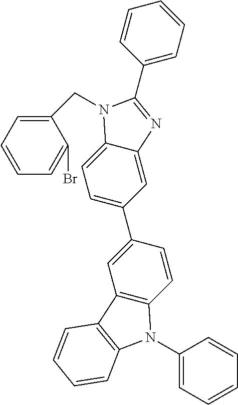 [SMILES: Brc1ccccc1Cn1c(-c2ccccc2)nc2cc(-c3ccc4c(c3)c3ccccc3n4-c3ccccc3)ccc21] | 87% |
| 16b | 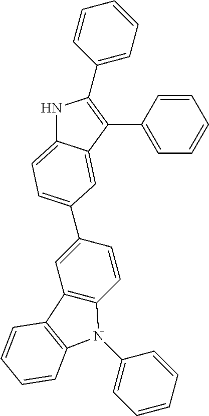 [SMILES: c1ccc(-c2[nH]c3ccc(-c4ccc5c(c4)c4ccccc4n5-c4ccccc4)cc3c2-c2ccccc2)cc1] | 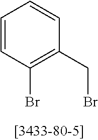 | 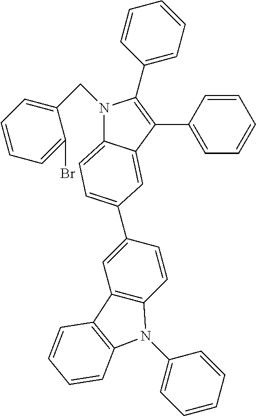 [SMILES: Brc1ccccc1Cn1c(-c2ccccc2)c(-c2ccccc2)c2cc(-c3ccc4c(c3)c3ccccc3n4-c3ccccc3)ccc21] | 88% |
| 17b | 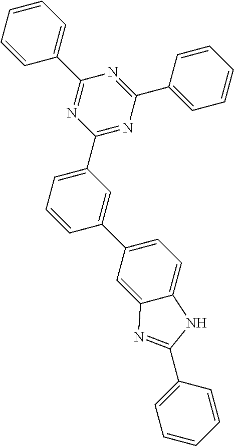 [SMILES: c1ccc(-c2nc(-c3ccccc3)nc(-c3cccc(-c4ccc5[nH]c(-c6ccccc6)nc5c4)c3)n2)cc1] | 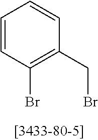 | 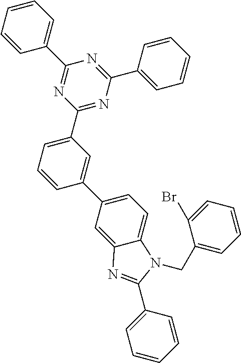 [SMILES: Brc1ccccc1Cn1c(-c2ccccc2)nc2cc(-c3cccc(-c4nc(-c5ccccc5)nc(-c5ccccc5)n4)c3)ccc21] | 86% |
| 18b | 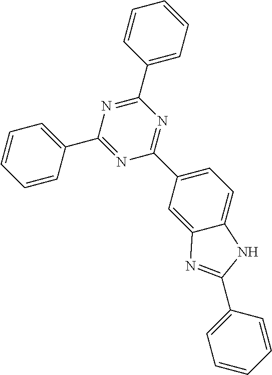 [SMILES: c1ccc(-c2nc(-c3ccccc3)nc(-c3ccc4[nH]c(-c5ccccc5)nc4c3)n2)cc1] | 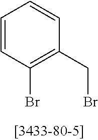 | 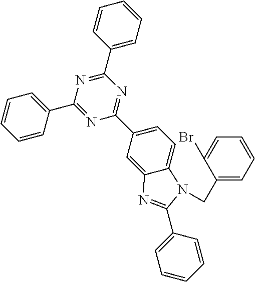 [SMILES: Brc1ccccc1Cn1c(-c2ccccc2)nc2cc(-c3nc(-c4ccccc4)nc(-c4ccccc4)n3)ccc21] | 83% |
| 19b | 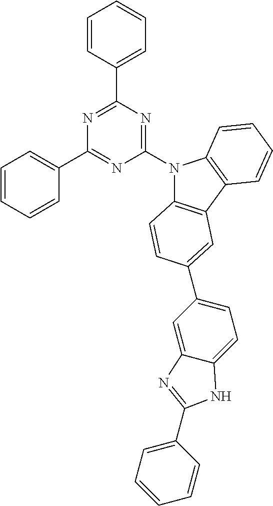 [SMILES: c1ccc(-c2nc(-c3ccccc3)nc(-n3c4ccccc4c4cc(-c5ccc6[nH]c(-c7ccccc7)nc6c5)ccc43)n2)cc1] | 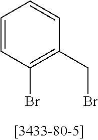 | 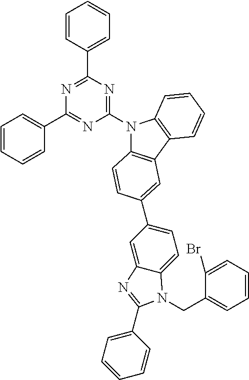 [SMILES: Brc1ccccc1Cn1c(-c2ccccc2)nc2cc(-c3ccc4c(c3)c3ccccc3n4-c3nc(-c4ccccc4)nc(-c4ccccc4)n3)ccc21] | 84% |
| 20b | 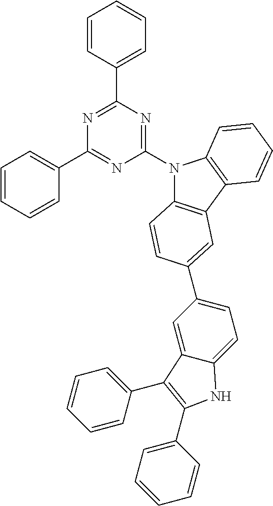 [SMILES: c1ccc(-c2nc(-c3ccccc3)nc(-n3c4ccccc4c4cc(-c5ccc6[nH]c(-c7ccccc7)c(-c7ccccc7)c6c5)ccc43)n2)cc1] | 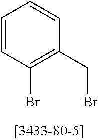 | 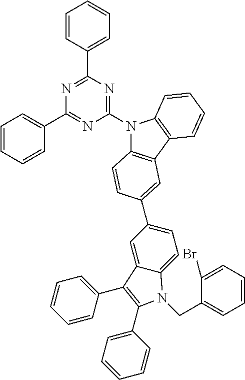 [SMILES: Brc1ccccc1Cn1c(-c2ccccc2)c(-c2ccccc2)c2cc(-c3ccc4c(c3)c3ccccc3n4-c3nc(-c4ccccc4)nc(-c4ccccc4)n3)ccc21] | 80% |
| 21b | 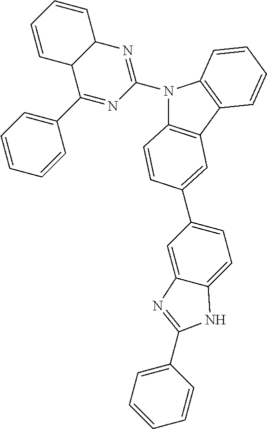 [SMILES: C1=CC2N=C(n3c4ccccc4c4cc(-c5ccc6[nH]c(-c7ccccc7)nc6c5)ccc43)N=C(c3ccccc3)C2C=C1] | 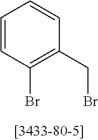 | 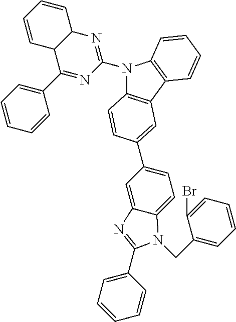 [SMILES: Brc1ccccc1Cn1c(-c2ccccc2)nc2cc(-c3ccc4c(c3)c3ccccc3n4C3=NC4C=CC=CC4C(c4ccccc4)=N3)ccc21] | 76% |
| 22b | 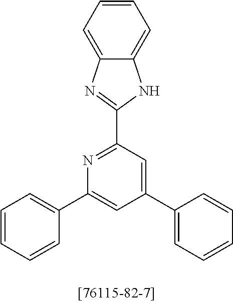 [SMILES: c1ccc(-c2cc(-c3ccccc3)nc(-c3nc4ccccc4[nH]3)c2)cc1] | 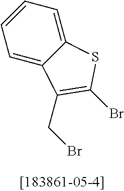 [SMILES: BrCc1c(Br)sc2ccccc12] | 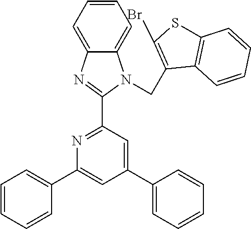 [SMILES: Brc1sc2ccccc2c1Cn1c(-c2cc(-c3ccccc3)cc(-c3ccccc3)n2)nc2ccccc21] | 78% |
| 23b | 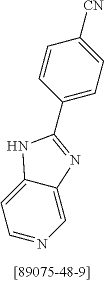 [SMILES: N#Cc1ccc(-c2nc3cnccc3[nH]2)cc1] | 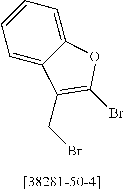 [SMILES: BrCc1c(Br)oc2ccccc12] | 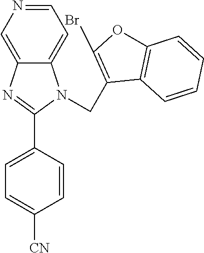 [SMILES: N#Cc1ccc(-c2nc3cnccc3n2Cc2c(Br)oc3ccccc23)cc1] | 70% |
| 24b | 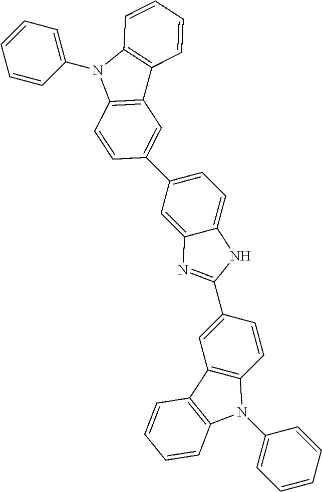 [SMILES: c1ccc(-n2c3ccccc3c3cc(-c4ccc5[nH]c(-c6ccc7c(c6)c6ccccc6n7-c6ccccc6)nc5c4)ccc32)cc1] | 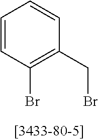 | 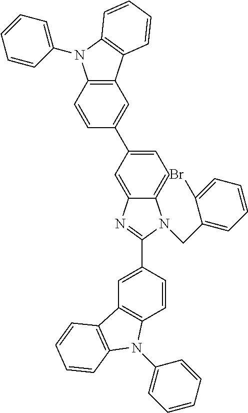 [SMILES: Brc1ccccc1Cn1c(-c2ccc3c(c2)c2ccccc2n3-c2ccccc2)nc2cc(-c3ccc4c(c3)c3ccccc3n4-c3ccccc3)ccc21] | 77% |
| 25b | 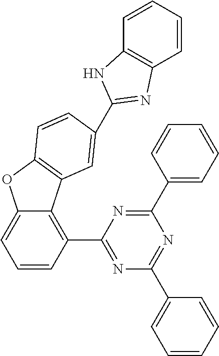 [SMILES: c1ccc(-c2nc(-c3ccccc3)nc(-c3cccc4oc5ccc(-c6nc7ccccc7[nH]6)cc5c34)n2)cc1] | 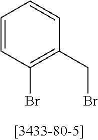 | 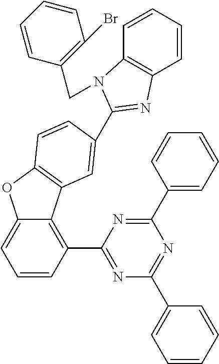 [SMILES: Brc1ccccc1Cn1c(-c2ccc3oc4cccc(-c5nc(-c6ccccc6)nc(-c6ccccc6)n5)c4c3c2)nc2ccccc21] | 80% |
| 26b | 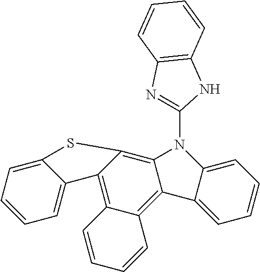 [SMILES: c1ccc2[nH]c(-n3c4ccccc4c4c5ccccc5c5c6ccccc6sc5c43)nc2c1] | 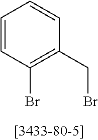 | 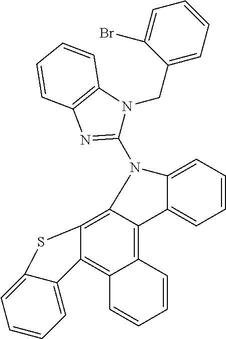 [SMILES: Brc1ccccc1Cn1c(-n2c3ccccc3c3c4ccccc4c4c5ccccc5sc4c32)nc2ccccc21] | 78% |
| 27b | 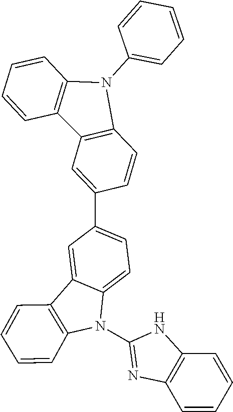 [SMILES: c1ccc(-n2c3ccccc3c3cc(-c4ccc5c(c4)c4ccccc4n5-c4nc5ccccc5[nH]4)ccc32)cc1] | 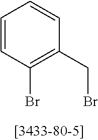 | 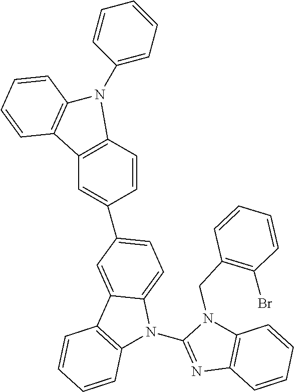 [SMILES: Brc1ccccc1Cn1c(-n2c3ccccc3c3cc(-c4ccc5c(c4)c4ccccc4n5-c4ccccc4)ccc32)nc2ccccc21] | 81% |
| 28b | 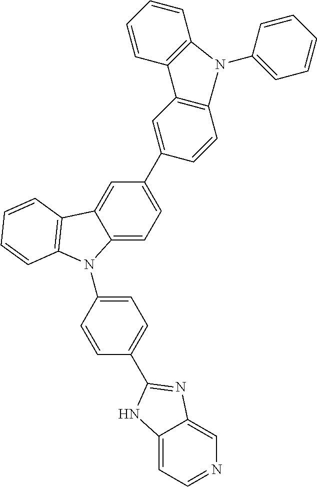 [SMILES: c1ccc(-n2c3ccccc3c3cc(-c4ccc5c(c4)c4ccccc4n5-c4ccc(-c5nc6cnccc6[nH]5)cc4)ccc32)cc1] | 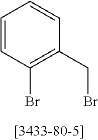 | 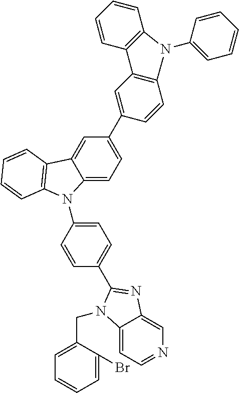 [SMILES: Brc1ccccc1Cn1c(-c2ccc(-n3c4ccccc4c4cc(-c5ccc6c(c5)c5ccccc5n6-c5ccccc5)ccc43)cc2)nc2cnccc21] | 80% |
| 29b | 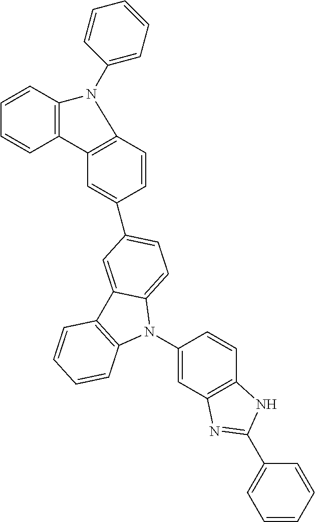 [SMILES: c1ccc(-c2nc3cc(-n4c5ccccc5c5cc(-c6ccc7c(c6)c6ccccc6n7-c6ccccc6)ccc54)ccc3[nH]2)cc1] | 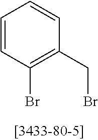 | 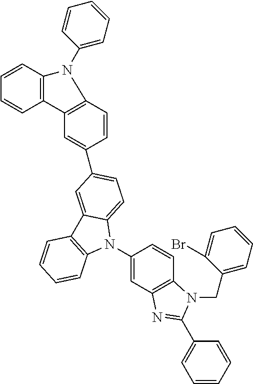 [SMILES: Brc1ccccc1Cn1c(-c2ccccc2)nc2cc(-n3c4ccccc4c4cc(-c5ccc6c(c5)c5ccccc5n6-c5ccccc5)ccc43)ccc21] | 77% |
| 30b | 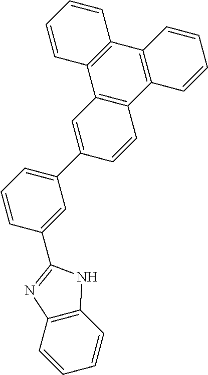 [SMILES: c1cc(-c2ccc3c4ccccc4c4ccccc4c3c2)cc(-c2nc3ccccc3[nH]2)c1] | 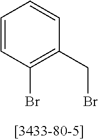 | 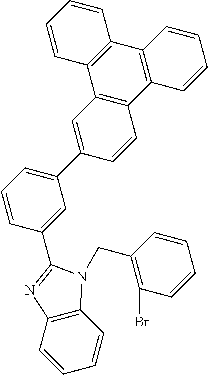 [SMILES: Brc1ccccc1Cn1c(-c2cccc(-c3ccc4c5ccccc5c5ccccc5c4c3)c2)nc2ccccc21] | 87% |
| 31b | 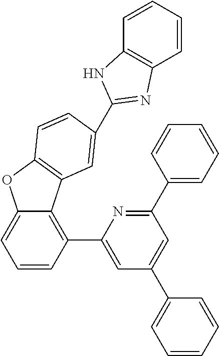 [SMILES: c1ccc(-c2cc(-c3ccccc3)nc(-c3cccc4oc5ccc(-c6nc7ccccc7[nH]6)cc5c34)c2)cc1] | 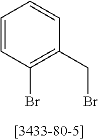 | 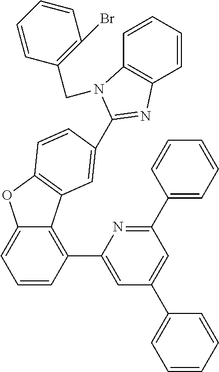 [SMILES: Brc1ccccc1Cn1c(-c2ccc3oc4cccc(-c5cc(-c6ccccc6)cc(-c6ccccc6)n5)c4c3c2)nc2ccccc21] | 80% |

### c) 5-Phenyl-1H-imidazo[4,5,1-de]phenanthridine

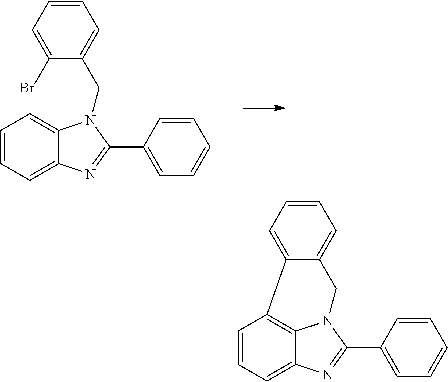 [SMILES: Brc1ccccc1Cn1c(-c2ccccc2)nc2ccccc21 ; c1ccc(-c2nc3cccc4c3n2Cc2ccccc2-4)cc1]

68 g (187 mol) of 1-[(2-bromophenyl)methyl]-2-phenylbenzimidazole is dissolved in 500 ml of dimethylformamide under protective atmosphere. 38 g (394 mmol) of potassium acetate is added to this solution, which is stirred for 30 min, and then 21 g (18.7 mmol) of Pd(PPh₃)₄ is added, and stirring of the mixture is continued at 110° C. for 5 days. After this time, the reaction mixture is cooled to room temperature and extracted with dichloromethane. The combined organic phases are dried over Na₂SO₄ and concentrated. The residue is recrystallized from acetone. Yield: 42 g (151 mmol), 81%.

The following compounds can be obtained analogously:

| Ex. | Reactant 1 | Product | Yield |
|---|---|---|---|
| 1c | 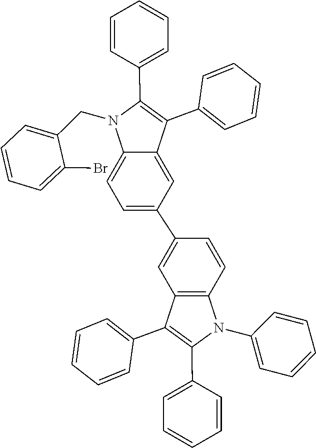 [SMILES: Brc1ccccc1Cn1c(-c2ccccc2)c(-c2ccccc2)c2cc(-c3ccc4c(c3)c(-c3ccccc3)c(-c3ccccc3)n4-c3ccccc3)ccc21] | 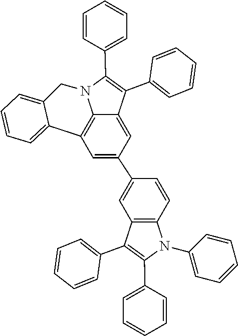 [SMILES: c1ccc(-c2c(-c3ccccc3)n3c4c(cc(-c5ccc6c(c5)c(-c5ccccc5)c(-c5ccccc5)n6-c5ccccc5)cc24)-c2ccccc2C3)cc1] | 80% |
| 2c | 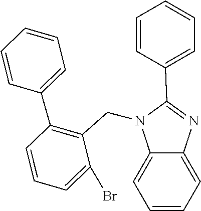 [SMILES: Brc1cccc(-c2ccccc2)c1Cn1c(-c2ccccc2)nc2ccccc21] | 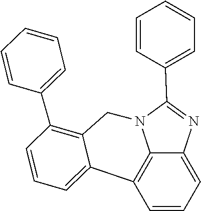 [SMILES: c1ccc(-c2cccc3c2Cn2c(-c4ccccc4)nc4cccc-3c42)cc1] | 84% |
| 3c | 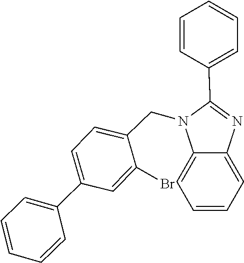 [SMILES: Brc1cc(-c2ccccc2)ccc1Cn1c(-c2ccccc2)nc2ccccc21] | 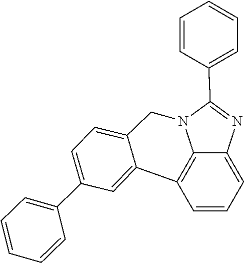 [SMILES: c1ccc(-c2ccc3c(c2)-c2cccc4nc(-c5ccccc5)n(c24)C3)cc1] | 85% |
| 4c | 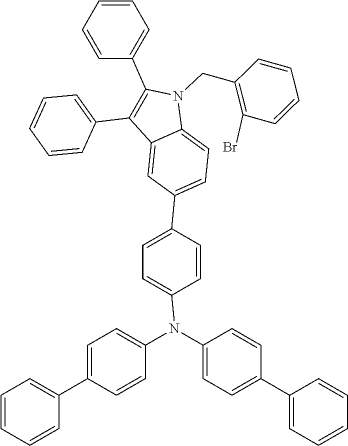 [SMILES: Brc1ccccc1Cn1c(-c2ccccc2)c(-c2ccccc2)c2cc(-c3ccc(N(c4ccc(-c5ccccc5)cc4)c4ccc(-c5ccccc5)cc4)cc3)ccc21] | 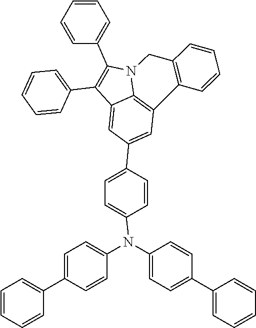 [SMILES: c1ccc(-c2ccc(N(c3ccc(-c4ccccc4)cc3)c3ccc(-c4cc5c6c(c4)c(-c4ccccc4)c(-c4ccccc4)n6Cc4ccccc4-5)cc3)cc2)cc1] | 81% |
| 5c | 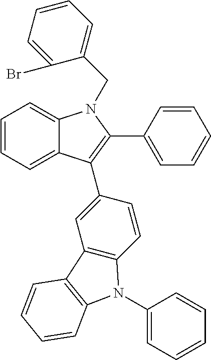 [SMILES: Brc1ccccc1Cn1c(-c2ccccc2)c(-c2ccc3c(c2)c2ccccc2n3-c2ccccc2)c2ccccc21] | 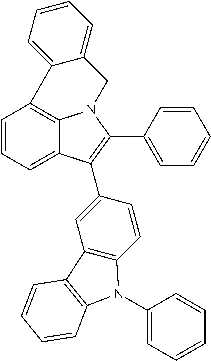 [SMILES: c1ccc(-c2c(-c3ccc4c(c3)c3ccccc3n4-c3ccccc3)c3cccc4c3n2Cc2ccccc2-4)cc1] | 83% |
| 6c | 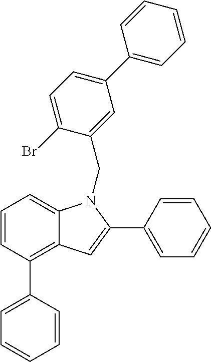 [SMILES: Brc1ccc(-c2ccccc2)cc1Cn1c(-c2ccccc2)cc2c(-c3ccccc3)cccc21] | 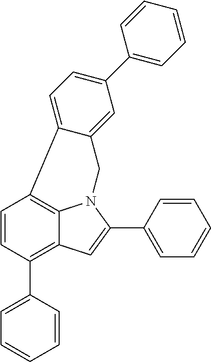 [SMILES: c1ccc(-c2ccc3c(c2)Cn2c(-c4ccccc4)cc4c(-c5ccccc5)ccc-3c42)cc1] | 79% |
| 7c | 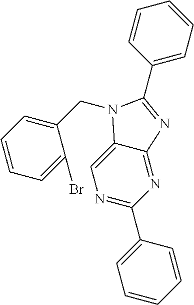 [SMILES: Brc1ccccc1Cn1c(-c2ccccc2)nc2nc(-c3ccccc3)ncc21] | 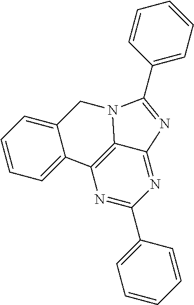 [SMILES: c1ccc(-c2nc3c4c(n2)nc(-c2ccccc2)n4Cc2ccccc2-3)cc1] | 77% |
| 8c | 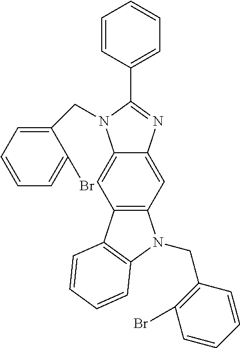 [SMILES: Brc1ccccc1Cn1c(-c2ccccc2)nc2cc3c(cc21)c1ccccc1n3Cc1ccccc1Br] | 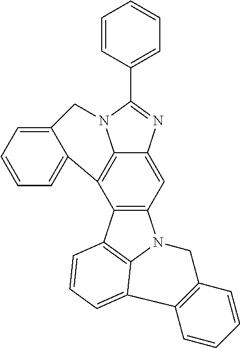 [SMILES: c1ccc(-c2nc3cc4c(c5c3n2Cc2ccccc2-5)c2cccc3c2n4Cc2ccccc2-3)cc1] | 76% |
| 9c | 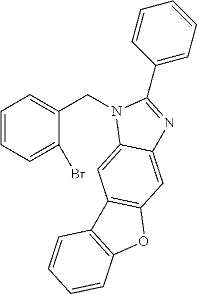 [SMILES: Brc1ccccc1Cn1c(-c2ccccc2)nc2cc3oc4ccccc4c3cc21] | 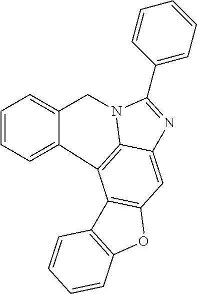 [SMILES: c1ccc(-c2nc3cc4oc5ccccc5c4c4c3n2Cc2ccccc2-4)cc1] | 81% |
| 10c | 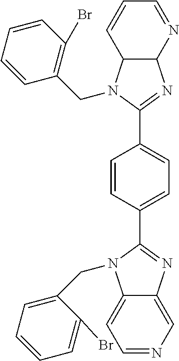 [SMILES: Brc1ccccc1CN1C(c2ccc(-c3nc4cnccc4n3Cc3ccccc3Br)cc2)=NC2N=CC=CC21] | 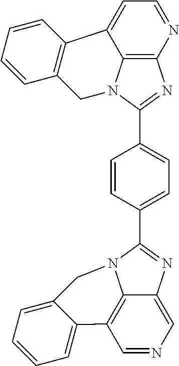 [SMILES: c1ccc2c(c1)Cn1c(-c3ccc(-c4nc5nccc6c5n4Cc4ccccc4-6)cc3)nc3cncc-2c31] | 68% |
| 11c | 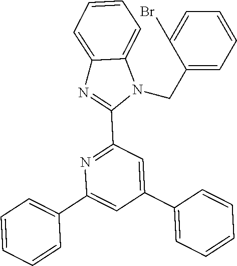 [SMILES: Brc1ccccc1Cn1c(-c2cc(-c3ccccc3)cc(-c3ccccc3)n2)nc2ccccc21] | 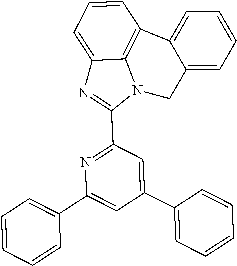 [SMILES: c1ccc(-c2cc(-c3ccccc3)nc(-c3nc4cccc5c4n3Cc3ccccc3-5)c2)cc1] | 60% |
| 12c | 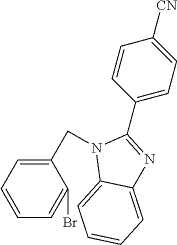 [SMILES: N#Cc1ccc(-c2nc3ccccc3n2Cc2ccccc2Br)cc1] | 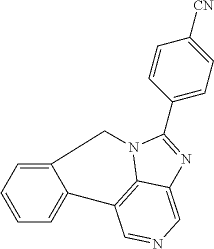 [SMILES: N#Cc1ccc(-c2nc3cncc4c3n2Cc2ccccc2-4)cc1] | 69% |
| 13c | 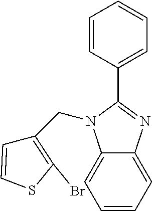 [SMILES: Brc1sccc1Cn1c(-c2ccccc2)nc2ccccc21] | 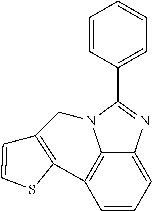 [SMILES: c1ccc(-c2nc3cccc4c3n2Cc2ccsc2-4)cc1] | 71% |
| 14c | 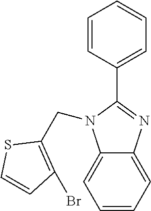 [SMILES: Brc1ccsc1Cn1c(-c2ccccc2)nc2ccccc21] | 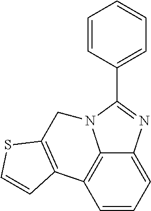 [SMILES: c1ccc(-c2nc3cccc4c3n2Cc2sccc2-4)cc1] | 71% |
| 15c | 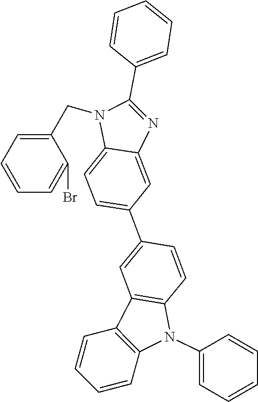 [SMILES: Brc1ccccc1Cn1c(-c2ccccc2)nc2cc(-c3ccc4c(c3)c3ccccc3n4-c3ccccc3)ccc21] | 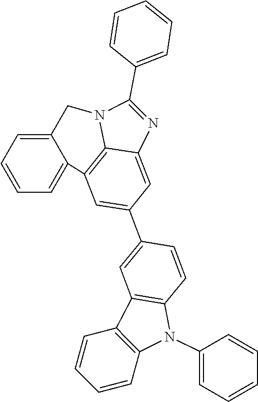 [SMILES: c1ccc(-c2nc3cc(-c4ccc5c(c4)c4ccccc4n5-c4ccccc4)cc4c3n2Cc2ccccc2-4)cc1] | 76% |
| 16c | 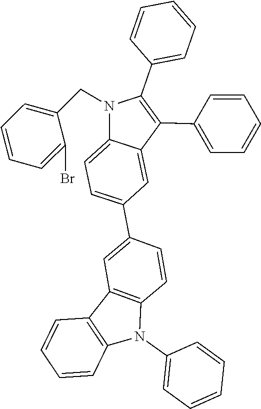 [SMILES: Brc1ccccc1Cn1c(-c2ccccc2)c(-c2ccccc2)c2cc(-c3ccc4c(c3)c3ccccc3n4-c3ccccc3)ccc21] | 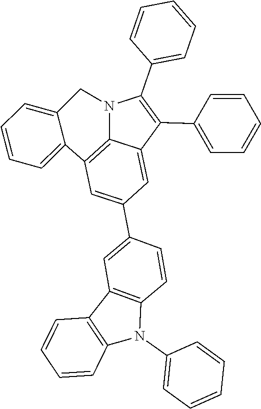 [SMILES: c1ccc(-c2c(-c3ccccc3)n3c4c(cc(-c5ccc6c(c5)c5ccccc5n6-c5ccccc5)cc24)-c2ccccc2C3)cc1] | 83% |
| 17c | 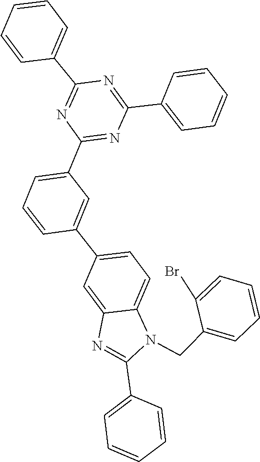 [SMILES: Brc1ccccc1Cn1c(-c2ccccc2)nc2cc(-c3cccc(-c4nc(-c5ccccc5)nc(-c5ccccc5)n4)c3)ccc21] | 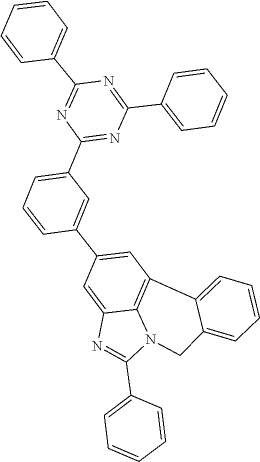 [SMILES: c1ccc(-c2nc(-c3ccccc3)nc(-c3cccc(-c4cc5c6c(c4)nc(-c4ccccc4)n6Cc4ccccc4-5)c3)n2)cc1] | 85% |
| 18c | 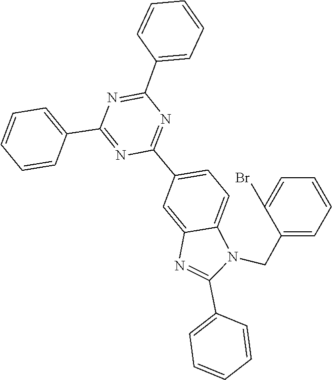 [SMILES: Brc1ccccc1Cn1c(-c2ccccc2)nc2cc(-c3nc(-c4ccccc4)nc(-c4ccccc4)n3)ccc21] | 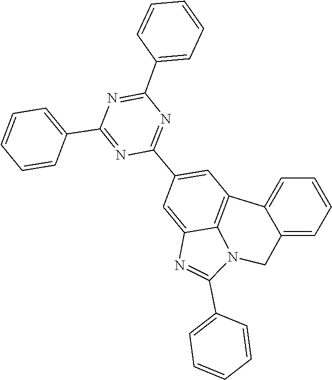 [SMILES: c1ccc(-c2nc(-c3ccccc3)nc(-c3cc4c5c(c3)nc(-c3ccccc3)n5Cc3ccccc3-4)n2)cc1] | 69% |
| 19c | 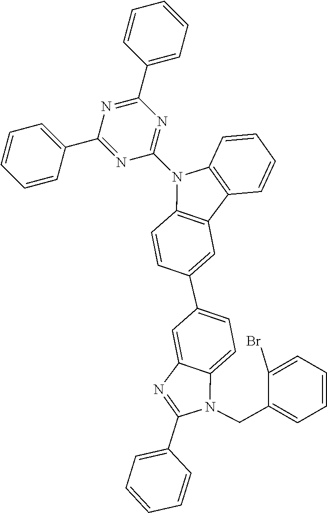 [SMILES: Brc1ccccc1Cn1c(-c2ccccc2)nc2cc(-c3ccc4c(c3)c3ccccc3n4-c3nc(-c4ccccc4)nc(-c4ccccc4)n3)ccc21] | 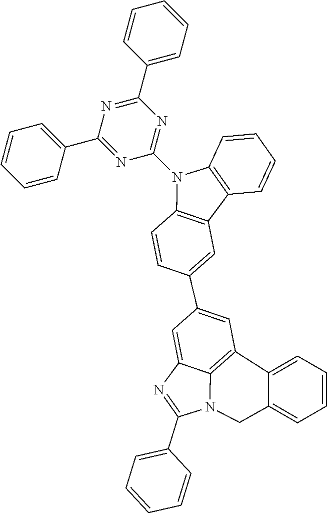 [SMILES: c1ccc(-c2nc(-c3ccccc3)nc(-n3c4ccccc4c4cc(-c5cc6c7c(c5)nc(-c5ccccc5)n7Cc5ccccc5-6)ccc43)n2)cc1] | 61% |
| 20c | 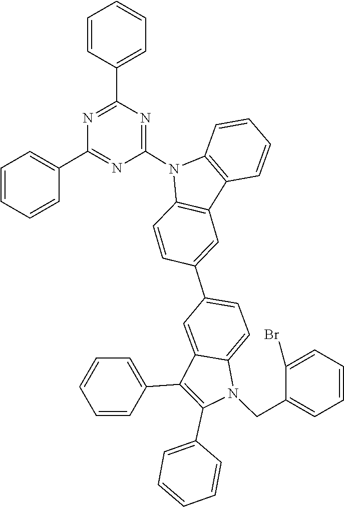 [SMILES: Brc1ccccc1Cn1c(-c2ccccc2)c(-c2ccccc2)c2cc(-c3ccc4c(c3)c3ccccc3n4-c3nc(-c4ccccc4)nc(-c4ccccc4)n3)ccc21] | 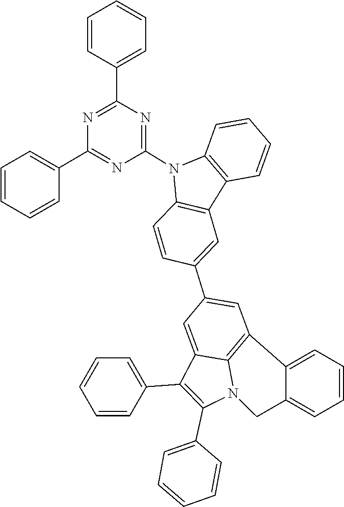 [SMILES: c1ccc(-c2nc(-c3ccccc3)nc(-n3c4ccccc4c4cc(-c5cc6c7c(c5)c(-c5ccccc5)c(-c5ccccc5)n7Cc5ccccc5-6)ccc43)n2)cc1] | 72% |
| 21c | 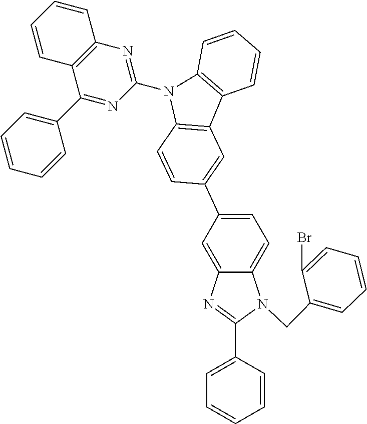 [SMILES: Brc1ccccc1Cn1c(-c2ccccc2)nc2cc(-c3ccc4c(c3)c3ccccc3n4-c3nc(-c4ccccc4)c4ccccc4n3)ccc21] | 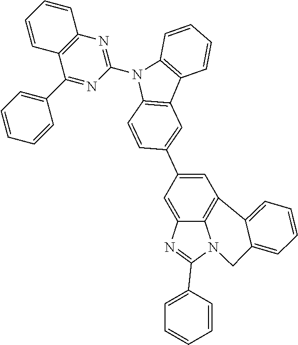 [SMILES: c1ccc(-c2nc(-n3c4ccccc4c4cc(-c5cc6c7c(c5)nc(-c5ccccc5)n7Cc5ccccc5-6)ccc43)nc3ccccc23)cc1] | 76% |
| 22c | 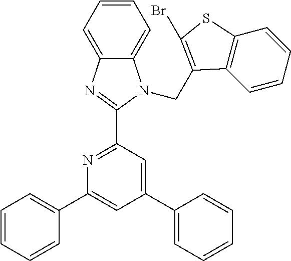 [SMILES: Brc1sc2ccccc2c1Cn1c(-c2cc(-c3ccccc3)cc(-c3ccccc3)n2)nc2ccccc21] | 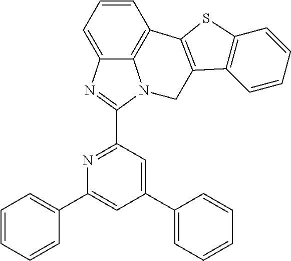 [SMILES: c1ccc(-c2cc(-c3ccccc3)nc(-c3nc4cccc5c4n3Cc3c-5sc4ccccc34)c2)cc1] | 76% |
| 33c | 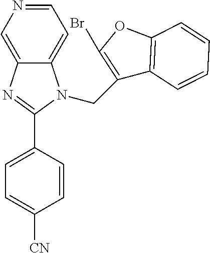 [SMILES: N#Cc1ccc(-c2nc3cnccc3n2Cc2c(Br)oc3ccccc23)cc1] | 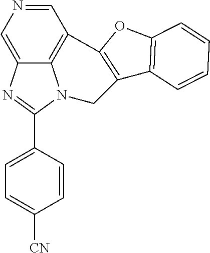 [SMILES: N#Cc1ccc(-c2nc3cncc4c3n2Cc2c-4oc3ccccc23)cc1] | 74% |
| 24c | 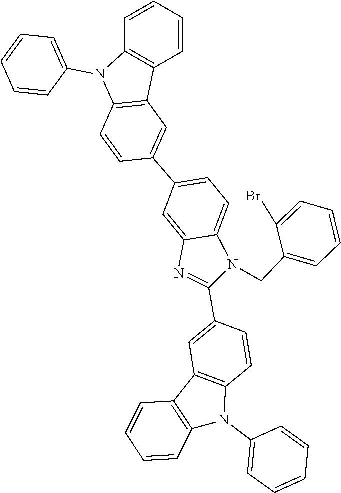 [SMILES: Brc1ccccc1Cn1c(-c2ccc3c(c2)c2ccccc2n3-c2ccccc2)nc2cc(-c3ccc4c(c3)c3ccccc3n4-c3ccccc3)ccc21] | 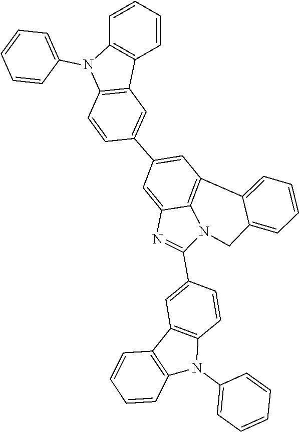 [SMILES: c1ccc(-n2c3ccccc3c3cc(-c4cc5c6c(c4)nc(-c4ccc7c(c4)c4ccccc4n7-c4ccccc4)n6Cc4ccccc4-5)ccc32)cc1] | 81% |
| 25c | 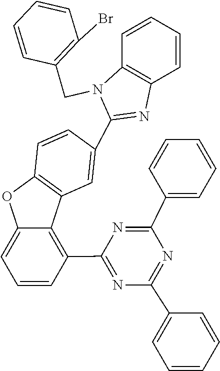 [SMILES: Brc1ccccc1Cn1c(-c2ccc3oc4cccc(-c5nc(-c6ccccc6)nc(-c6ccccc6)n5)c4c3c2)nc2ccccc21] | 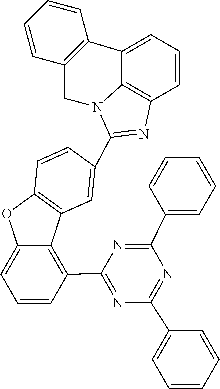 [SMILES: c1ccc(-c2nc(-c3ccccc3)nc(-c3cccc4oc5ccc(-c6nc7cccc8c7n6Cc6ccccc6-8)cc5c34)n2)cc1] | 66% |
| 26c | 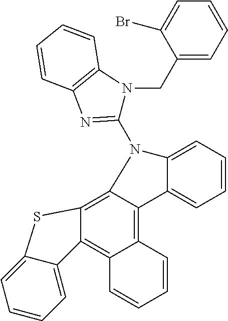 [SMILES: Brc1ccccc1Cn1c(-n2c3ccccc3c3c4ccccc4c4c5ccccc5sc4c32)nc2ccccc21] | 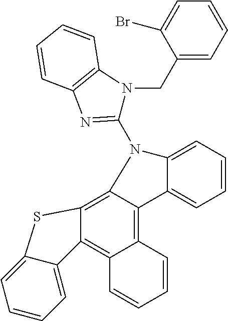 [SMILES: Brc1ccccc1Cn1c(-n2c3ccccc3c3c4ccccc4c4c5ccccc5sc4c32)nc2ccccc21] | 76% |
| 27c | 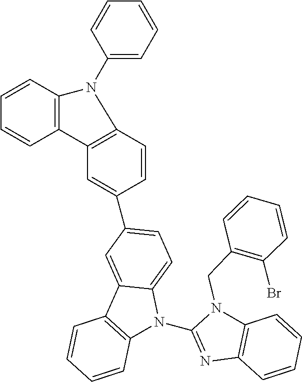 [SMILES: Brc1ccccc1Cn1c(-n2c3ccccc3c3cc(-c4ccc5c(c4)c4ccccc4n5-c4ccccc4)ccc32)nc2ccccc21] | 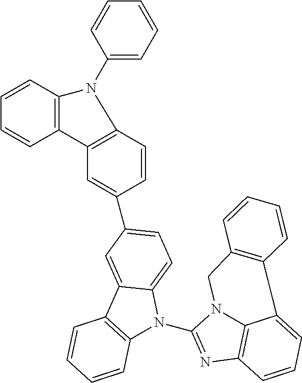 [SMILES: c1ccc(-n2c3ccccc3c3cc(-c4ccc5c(c4)c4ccccc4n5-c4nc5cccc6c5n4Cc4ccccc4-6)ccc32)cc1] | 77% |
| 28c | 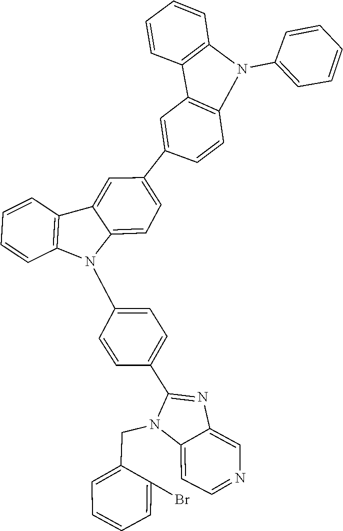 [SMILES: Brc1ccccc1Cn1c(-c2ccc(-n3c4ccccc4c4cc(-c5ccc6c(c5)c5ccccc5n6-c5ccccc5)ccc43)cc2)nc2cnccc21] | 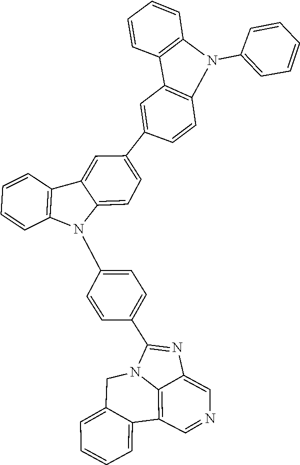 [SMILES: c1ccc(-n2c3ccccc3c3cc(-c4ccc5c(c4)c4ccccc4n5-c4ccc(-c5nc6cncc7c6n5Cc5ccccc5-7)cc4)ccc32)cc1] | 69% |
| 29c | 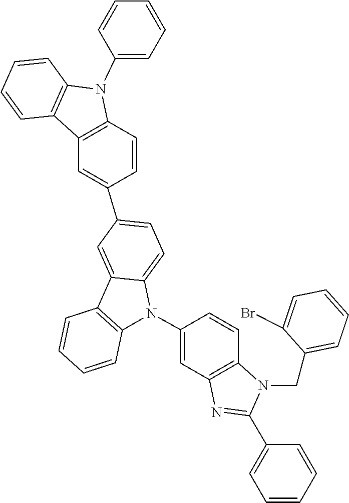 [SMILES: Brc1ccccc1Cn1c(-c2ccccc2)nc2cc(-n3c4ccccc4c4cc(-c5ccc6c(c5)c5ccccc5n6-c5ccccc5)ccc43)ccc21] | 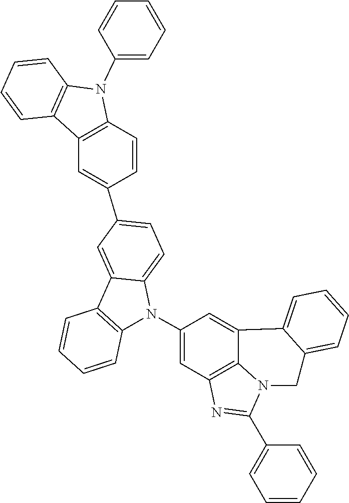 [SMILES: c1ccc(-c2nc3cc(-n4c5ccccc5c5cc(-c6ccc7c(c6)c6ccccc6n7-c6ccccc6)ccc54)cc4c3n2Cc2ccccc2-4)cc1] | 71% |
| 30c | 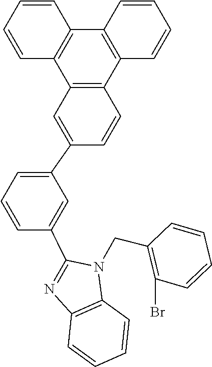 [SMILES: Brc1ccccc1Cn1c(-c2cccc(-c3ccc4c5ccccc5c5ccccc5c4c3)c2)nc2ccccc21] | 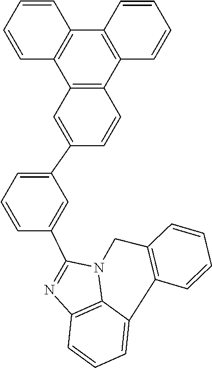 [SMILES: c1cc(-c2ccc3c4ccccc4c4ccccc4c3c2)cc(-c2nc3cccc4c3n2Cc2ccccc2-4)c1] | 76% |
| 31c | 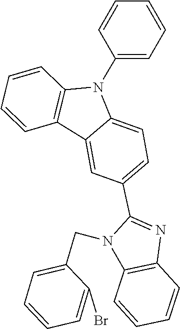 [SMILES: Brc1ccccc1Cn1c(-c2ccc3c(c2)c2ccccc2n3-c2ccccc2)nc2ccccc21] | 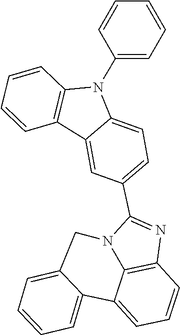 [SMILES: c1ccc(-n2c3ccccc3c3cc(-c4nc5cccc6c5n4Cc4ccccc4-6)ccc32)cc1] | 77% |

### d) 5-Phenyl-1H-imidazo[4,5,1-de]phenanthridin-7-one

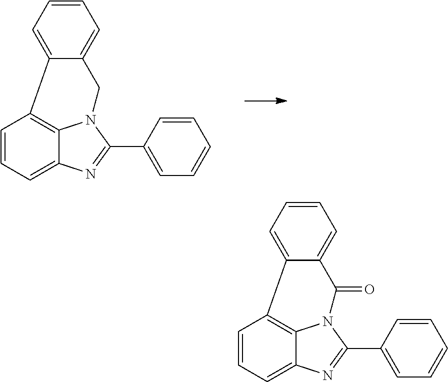 [SMILES: c1ccc(-c2nc3cccc4c3n2Cc2ccccc2-4)cc1 ; O=c1c2ccccc2c2cccc3nc(-c4ccccc4)n1c32]

34 g (120 mmol) of 5-phenyl-1H-imidazo[4,5,1-de]phenanthridine is dissolved in 600 ml of dichloromethane and 600 ml of water. 13 g (120 mmol) of 18-crown-16 and 28 g (181 mmol) of potassium permanganate are added to this solution in portions, and the mixture is stirred at room temperature for two days. After this time, the rest of the potassium permanganate is filtered off, and the solution is concentrated and purified by chromatography (eluent: heptane/dichloromethane, 5:1). The residue is recrystallized from toluene and from dichloromethane and finally sublimed under high vacuum; purity is 99.9%.

Yield: 121 g (71 mmol), 59%.

The following compounds can be obtained analogously:

| Ex. | Reactant 1 | Product | Yield |
|---|---|---|---|
| 1d | 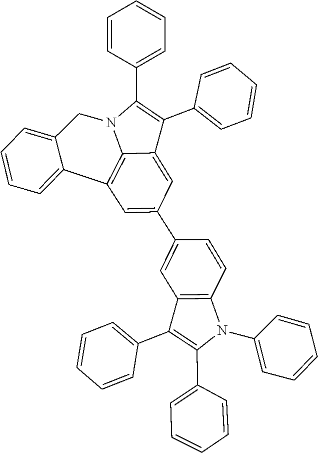 [SMILES: c1ccc(-c2c(-c3ccccc3)n3c4c(cc(-c5ccc6c(c5)c(-c5ccccc5)c(-c5ccccc5)n6-c5ccccc5)cc24)-c2ccccc2C3)cc1] | 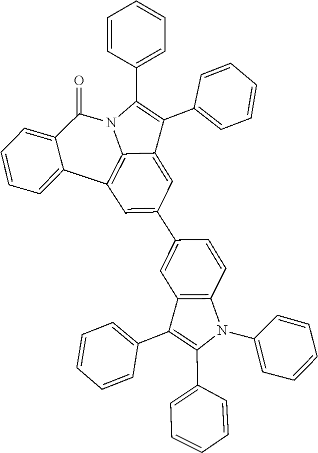 [SMILES: O=c1c2ccccc2c2cc(-c3ccc4c(c3)c(-c3ccccc3)c(-c3ccccc3)n4-c3ccccc3)cc3c(-c4ccccc4)c(-c4ccccc4)n1c32] | 83% |
| 2b | 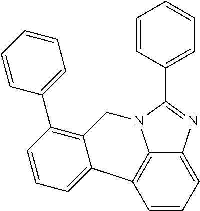 [SMILES: c1ccc(-c2cccc3c2Cn2c(-c4ccccc4)nc4cccc-3c42)cc1] | 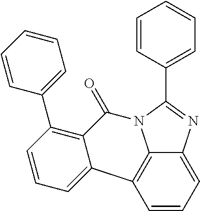 [SMILES: O=c1c2c(-c3ccccc3)cccc2c2cccc3nc(-c4ccccc4)n1c32] | 76% |
| 3d | 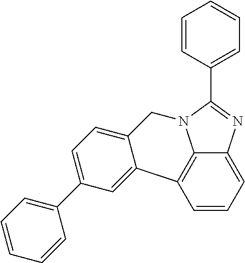 [SMILES: c1ccc(-c2ccc3c(c2)-c2cccc4nc(-c5ccccc5)n(c24)C3)cc1] | 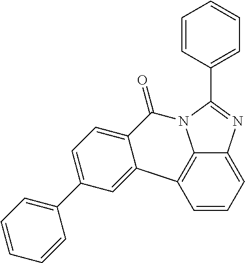 [SMILES: O=c1c2ccc(-c3ccccc3)cc2c2cccc3nc(-c4ccccc4)n1c32] | 86% |
| 4d | 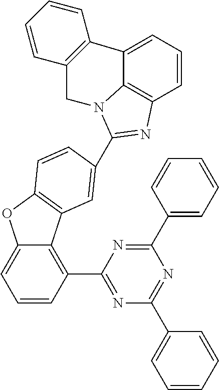 [SMILES: c1ccc(-c2nc(-c3ccccc3)nc(-c3cccc4oc5ccc(-c6nc7cccc8c7n6Cc6ccccc6-8)cc5c34)n2)cc1] | 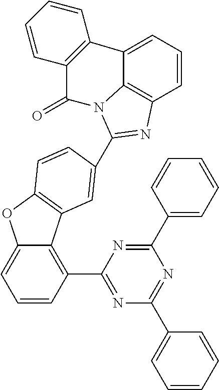 [SMILES: O=c1c2ccccc2c2cccc3nc(-c4ccc5oc6cccc(-c7nc(-c8ccccc8)nc(-c8ccccc8)n7)c6c5c4)n1c32] | 66% |
| 5d | 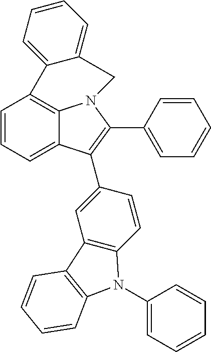 [SMILES: c1ccc(-c2c(-c3ccc4c(c3)c3ccccc3n4-c3ccccc3)c3cccc4c3n2Cc2ccccc2-4)cc1] | 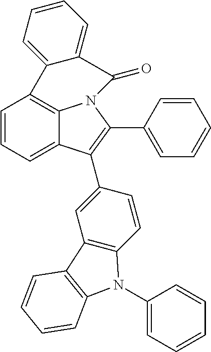 [SMILES: O=c1c2ccccc2c2cccc3c(-c4ccc5c(c4)c4ccccc4n5-c4ccccc4)c(-c4ccccc4)n1c32] | 82% |
| 6d | 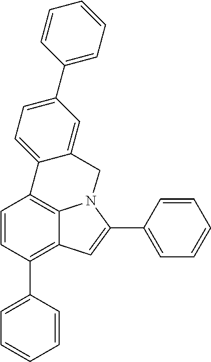 [SMILES: c1ccc(-c2ccc3c(c2)Cn2c(-c4ccccc4)cc4c(-c5ccccc5)ccc-3c42)cc1] | 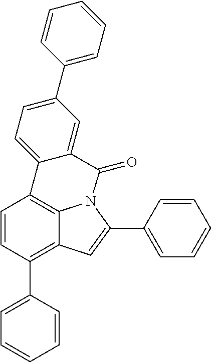 [SMILES: O=c1c2cc(-c3ccccc3)ccc2c2ccc(-c3ccccc3)c3cc(-c4ccccc4)n1c32] | 92% |
| 7d | 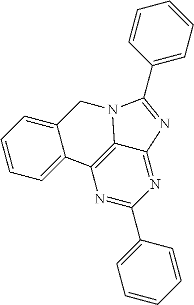 [SMILES: c1ccc(-c2nc3c4c(n2)nc(-c2ccccc2)n4Cc2ccccc2-3)cc1] | 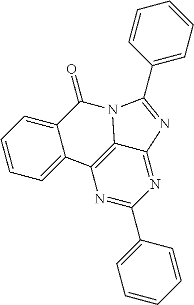 [SMILES: O=c1c2ccccc2c2nc(-c3ccccc3)nc3nc(-c4ccccc4)n1c32] | 66% |
| 8d | 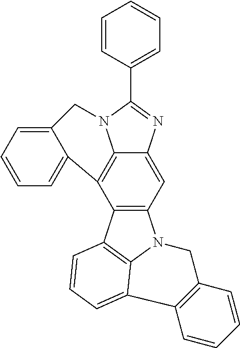 [SMILES: c1ccc(-c2nc3cc4c(c5c3n2Cc2ccccc2-5)c2cccc3c2n4Cc2ccccc2-3)cc1] | 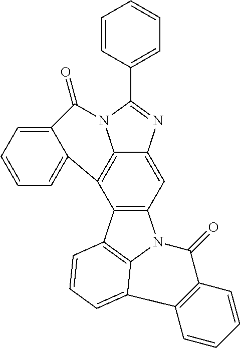 [SMILES: O=c1c2ccccc2c2c3c4cccc5c6ccccc6c(=O)n(c3cc3nc(-c6ccccc6)n1c32)c54] | 69% |
| 9d | 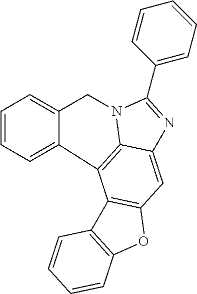 [SMILES: c1ccc(-c2nc3cc4oc5ccccc5c4c4c3n2Cc2ccccc2-4)cc1] | 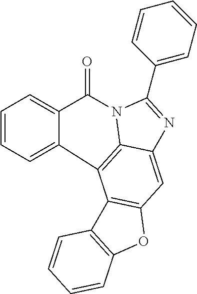 [SMILES: O=c1c2ccccc2c2c3c(cc4nc(-c5ccccc5)n1c42)oc1ccccc13] | 71% |
| 10d | 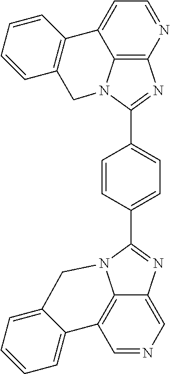 [SMILES: c1ccc2c(c1)Cn1c(-c3ccc(-c4nc5nccc6c5n4Cc4ccccc4-6)cc3)nc3cncc-2c31] | 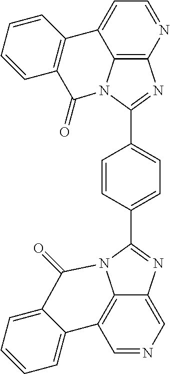 [SMILES: O=c1c2ccccc2c2cncc3nc(-c4ccc(-c5nc6nccc7c8ccccc8c(=O)n5c67)cc4)n1c32] | 78% |
| 11d | 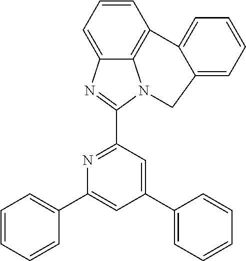 [SMILES: c1ccc(-c2cc(-c3ccccc3)nc(-c3nc4cccc5c4n3Cc3ccccc3-5)c2)cc1] | 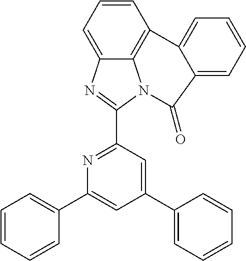 [SMILES: O=c1c2ccccc2c2cccc3nc(-c4cc(-c5ccccc5)cc(-c5ccccc5)n4)n1c32] | 72% |
| 12d | 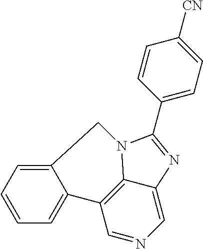 [SMILES: N#Cc1ccc(-c2nc3cncc4c3n2Cc2ccccc2-4)cc1] | 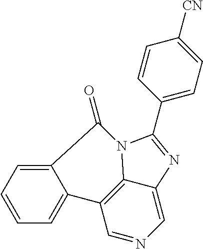 [SMILES: N#Cc1ccc(-c2nc3cncc4c5ccccc5c(=O)n2c34)cc1] | 79% |
| 13d | 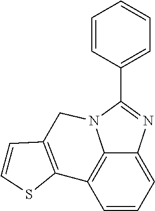 [SMILES: c1ccc(-c2nc3cccc4c3n2Cc2ccsc2-4)cc1] | 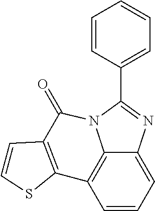 [SMILES: O=c1c2ccsc2c2cccc3nc(-c4ccccc4)n1c32] | 76% |
| 14d | 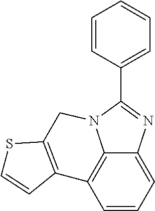 [SMILES: c1ccc(-c2nc3cccc4c3n2Cc2sccc2-4)cc1] | 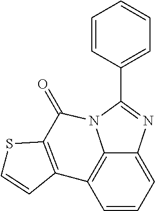 [SMILES: O=c1c2sccc2c2cccc3nc(-c4ccccc4)n1c32] | 72% |
| 15d | 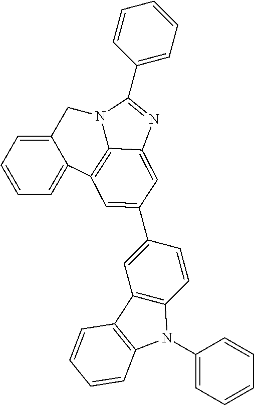 [SMILES: c1ccc(-c2nc3cc(-c4ccc5c(c4)c4ccccc4n5-c4ccccc4)cc4c3n2Cc2ccccc2-4)cc1] | 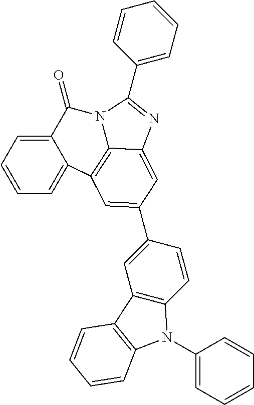 [SMILES: O=c1c2ccccc2c2cc(-c3ccc4c(c3)c3ccccc3n4-c3ccccc3)cc3nc(-c4ccccc4)n1c32] | 83% |
| 16d | 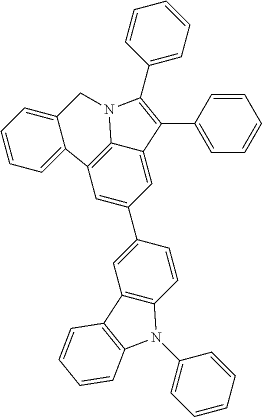 [SMILES: c1ccc(-c2c(-c3ccccc3)n3c4c(cc(-c5ccc6c(c5)c5ccccc5n6-c5ccccc5)cc24)-c2ccccc2C3)cc1] | 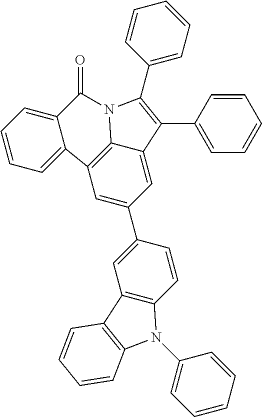 [SMILES: O=c1c2ccccc2c2cc(-c3ccc4c(c3)c3ccccc3n4-c3ccccc3)cc3c(-c4ccccc4)c(-c4ccccc4)n1c32] | 75% |
| 17d | 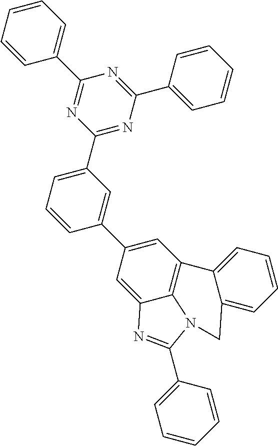 [SMILES: c1ccc(-c2nc(-c3ccccc3)nc(-c3cccc(-c4cc5c6c(c4)nc(-c4ccccc4)n6Cc4ccccc4-5)c3)n2)cc1] | 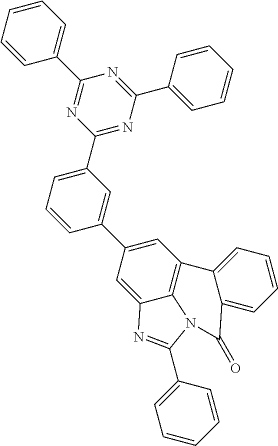 [SMILES: O=c1c2ccccc2c2cc(-c3cccc(-c4nc(-c5ccccc5)nc(-c5ccccc5)n4)c3)cc3nc(-c4ccccc4)n1c32] | 75% |
| 18d | 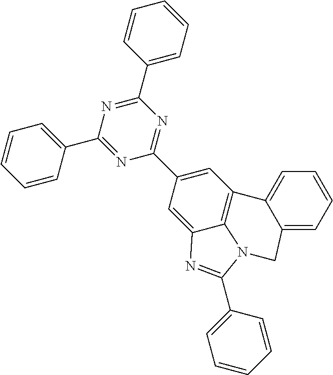 [SMILES: c1ccc(-c2nc(-c3ccccc3)nc(-c3cc4c5c(c3)nc(-c3ccccc3)n5Cc3ccccc3-4)n2)cc1] | 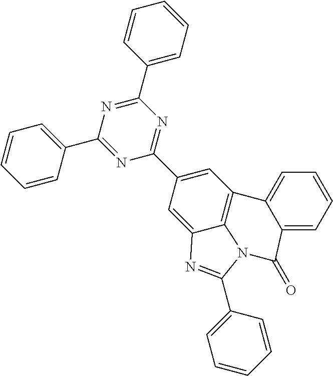 [SMILES: O=c1c2ccccc2c2cc(-c3nc(-c4ccccc4)nc(-c4ccccc4)n3)cc3nc(-c4ccccc4)n1c32] | 76% |
| 19d | 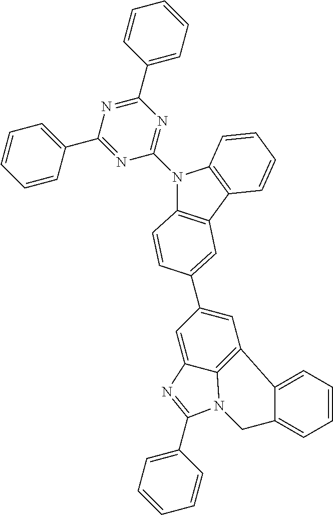 [SMILES: c1ccc(-c2nc(-c3ccccc3)nc(-n3c4ccccc4c4cc(-c5cc6c7c(c5)nc(-c5ccccc5)n7Cc5ccccc5-6)ccc43)n2)cc1] | 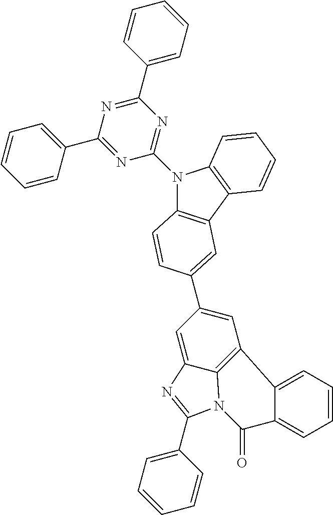 [SMILES: O=c1c2ccccc2c2cc(-c3ccc4c(c3)c3ccccc3n4-c3nc(-c4ccccc4)nc(-c4ccccc4)n3)cc3nc(-c4ccccc4)n1c32] | 74% |
| 20d | 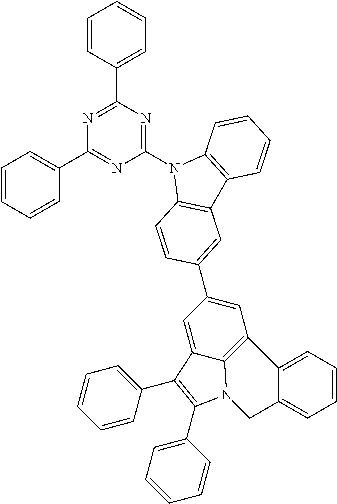 [SMILES: c1ccc(-c2nc(-c3ccccc3)nc(-n3c4ccccc4c4cc(-c5cc6c7c(c5)c(-c5ccccc5)c(-c5ccccc5)n7Cc5ccccc5-6)ccc43)n2)cc1] | 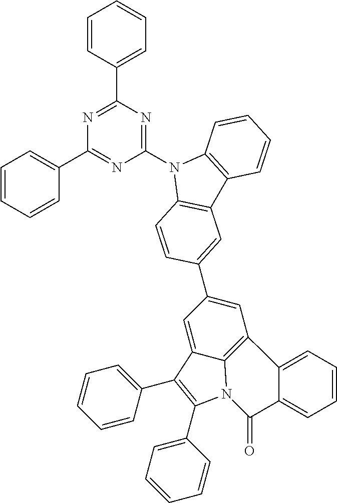 [SMILES: O=c1c2ccccc2c2cc(-c3ccc4c(c3)c3ccccc3n4-c3nc(-c4ccccc4)nc(-c4ccccc4)n3)cc3c(-c4ccccc4)c(-c4ccccc4)n1c32] | 81% |
| 21d | 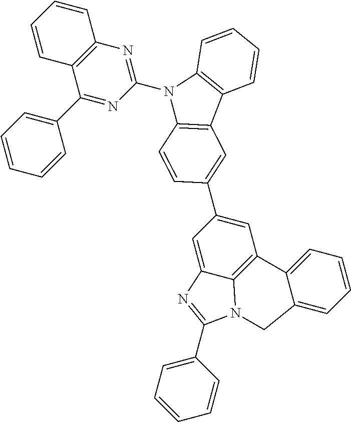 [SMILES: c1ccc(-c2nc(-n3c4ccccc4c4cc(-c5cc6c7c(c5)nc(-c5ccccc5)n7Cc5ccccc5-6)ccc43)nc3ccccc23)cc1] | 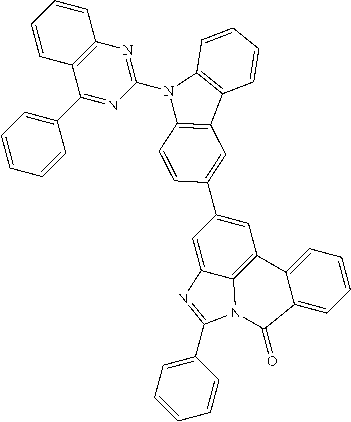 [SMILES: O=c1c2ccccc2c2cc(-c3ccc4c(c3)c3ccccc3n4-c3nc(-c4ccccc4)c4ccccc4n3)cc3nc(-c4ccccc4)n1c32] | 79% |
| 22d | 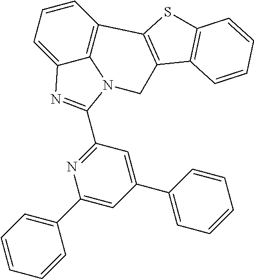 [SMILES: c1ccc(-c2cc(-c3ccccc3)nc(-c3nc4cccc5c4n3Cc3c-5sc4ccccc34)c2)cc1] | 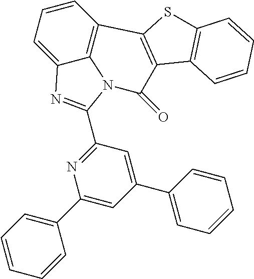 [SMILES: O=c1c2c3ccccc3sc2c2cccc3nc(-c4cc(-c5ccccc5)cc(-c5ccccc5)n4)n1c32] | 69% |
| 23d | 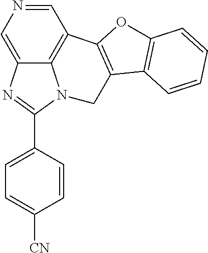 [SMILES: N#Cc1ccc(-c2nc3cncc4c3n2Cc2c-4oc3ccccc23)cc1] | 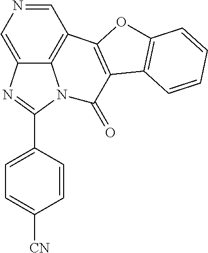 [SMILES: N#Cc1ccc(-c2nc3cncc4c5oc6ccccc6c5c(=O)n2c34)cc1] | 73% |
| 24d | 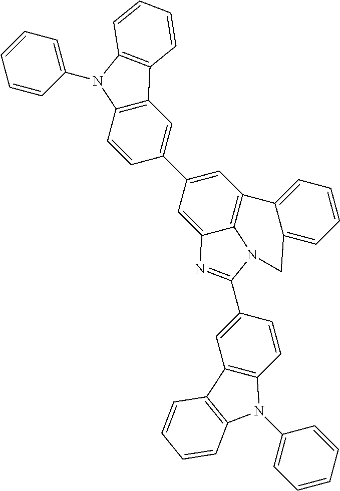 [SMILES: c1ccc(-n2c3ccccc3c3cc(-c4cc5c6c(c4)nc(-c4ccc7c(c4)c4ccccc4n7-c4ccccc4)n6Cc4ccccc4-5)ccc32)cc1] | 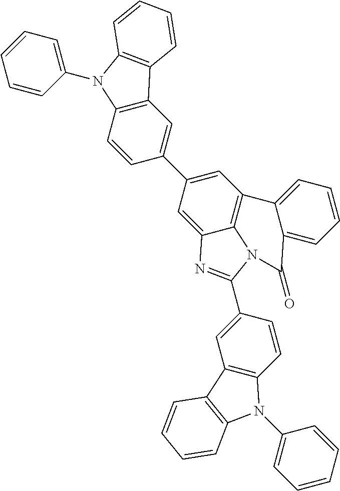 [SMILES: O=c1c2ccccc2c2cc(-c3ccc4c(c3)c3ccccc3n4-c3ccccc3)cc3nc(-c4ccc5c(c4)c4ccccc4n5-c4ccccc4)n1c32] | 86% |
| 25d | 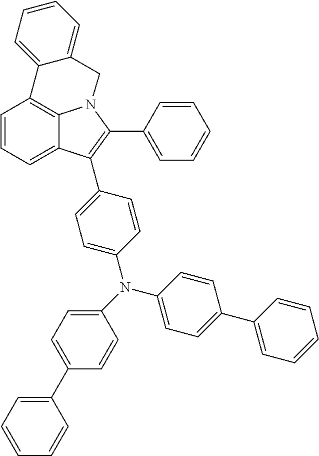 [SMILES: c1ccc(-c2ccc(N(c3ccc(-c4ccccc4)cc3)c3ccc(-c4c(-c5ccccc5)n5c6c(cccc46)-c4ccccc4C5)cc3)cc2)cc1] | 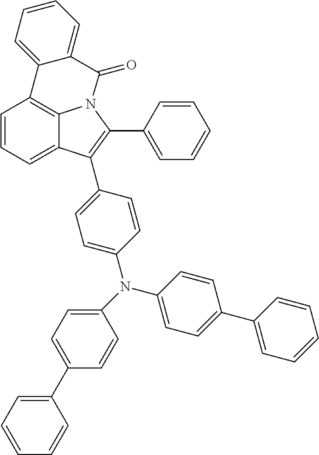 [SMILES: O=c1c2ccccc2c2cccc3c(-c4ccc(N(c5ccc(-c6ccccc6)cc5)c5ccc(-c6ccccc6)cc5)cc4)c(-c4ccccc4)n1c32] | 77% |
| 26d | 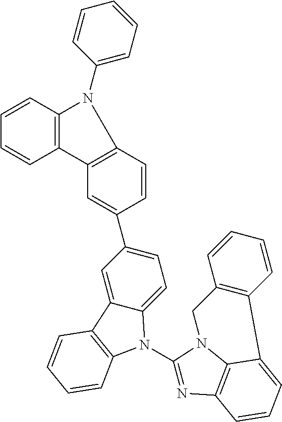 [SMILES: c1ccc(-n2c3ccccc3c3cc(-c4ccc5c(c4)c4ccccc4n5-c4nc5cccc6c5n4Cc4ccccc4-6)ccc32)cc1] | 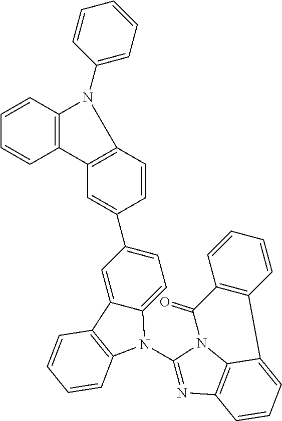 [SMILES: O=c1c2ccccc2c2cccc3nc(-n4c5ccccc5c5cc(-c6ccc7c(c6)c6ccccc6n7-c6ccccc6)ccc54)n1c32] | 76% |
| 27d | 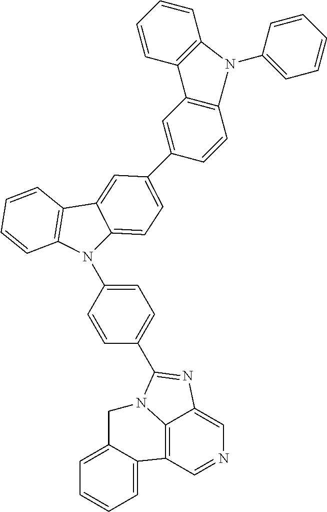 [SMILES: c1ccc(-n2c3ccccc3c3cc(-c4ccc5c(c4)c4ccccc4n5-c4ccc(-c5nc6cncc7c6n5Cc5ccccc5-7)cc4)ccc32)cc1] | 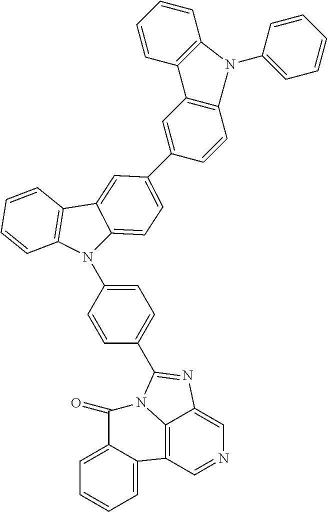 [SMILES: O=c1c2ccccc2c2cncc3nc(-c4ccc(-n5c6ccccc6c6cc(-c7ccc8c(c7)c7ccccc7n8-c7ccccc7)ccc65)cc4)n1c32] | 78% |
| 28d | 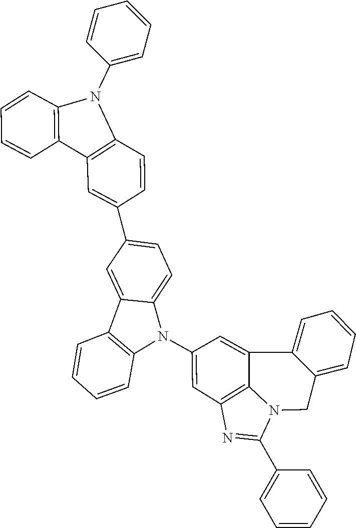 [SMILES: c1ccc(-c2nc3cc(-n4c5ccccc5c5cc(-c6ccc7c(c6)c6ccccc6n7-c6ccccc6)ccc54)cc4c3n2Cc2ccccc2-4)cc1] | 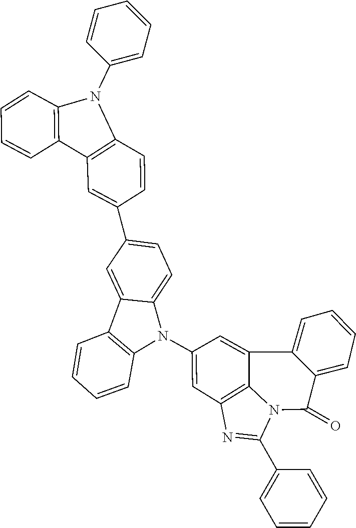 [SMILES: O=c1c2ccccc2c2cc(-n3c4ccccc4c4cc(-c5ccc6c(c5)c5ccccc5n6-c5ccccc5)ccc43)cc3nc(-c4ccccc4)n1c32] | 80% |
| 29d | 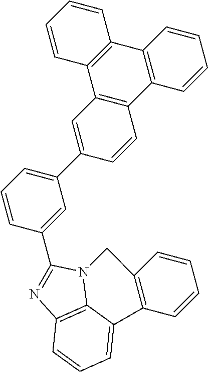 [SMILES: c1cc(-c2ccc3c4ccccc4c4ccccc4c3c2)cc(-c2nc3cccc4c3n2Cc2ccccc2-4)c1] | 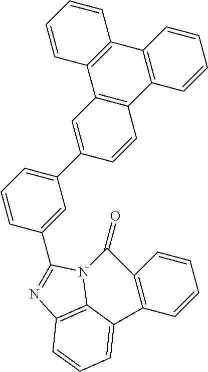 [SMILES: O=c1c2ccccc2c2cccc3nc(-c4cccc(-c5ccc6c7ccccc7c7ccccc7c6c5)c4)n1c32] | 83% |
| 30d | 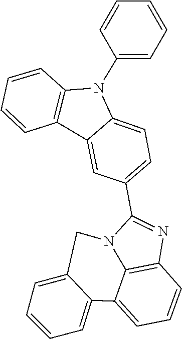 [SMILES: c1ccc(-n2c3ccccc3c3cc(-c4nc5cccc6c5n4Cc4ccccc4-6)ccc32)cc1] | 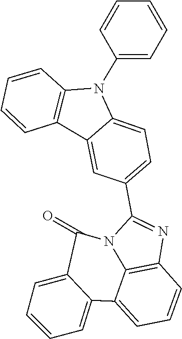 [SMILES: O=c1c2ccccc2c2cccc3nc(-c4ccc5c(c4)c4ccccc4n5-c4ccccc4)n1c32] | 78% |

### e) 1-[2-(5-Bromo-2-phenyl-3H-benzimidazol-4-yl)phenyl]ethanone

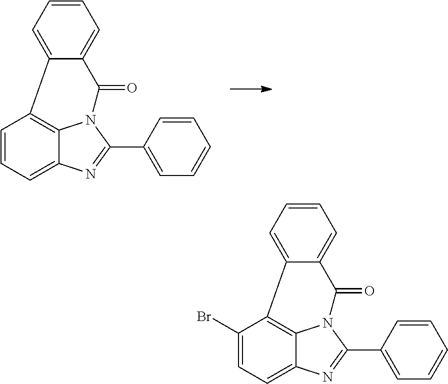 [SMILES: O=c1c2ccccc2c2cccc3nc(-c4ccccc4)n1c32 ; O=c1c2ccccc2c2c(Br)ccc3nc(-c4ccccc4)n1c32]

6.5 g (22 mmol) of 5-phenyl-1H-imidazo[4,5,1-de]phenanthridin-7-one is initially charged in 160 ml of DMF. Subsequently, a solution of 4 g (22.5 mmol) of NBS in 100 ml of DMF is added dropwise in the dark at room temperature, the mixture is allowed to come to room temperature and stirring is continued at this temperature for 4 h. Subsequently, 150 ml of water are added to the mixture and extraction is effected with CH₂Cl₂. The organic phase is dried over MgSO₄ and the solvents are removed under reduced pressure. The product is subjected to extractive stirring with hot hexane and filtered off with suction. Yield: 5 g (13 mmol), 61% of theory, purity by ¹H NMR about 98%.

The following compounds are obtained in an analogous manner:

| Ex. | Reactant 1 | Product | Yield |
|---|---|---|---|
| 1e | 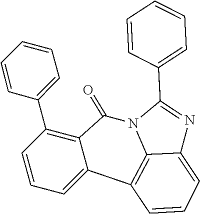 [SMILES: O=c1c2c(-c3ccccc3)cccc2c2cccc3nc(-c4ccccc4)n1c32] | 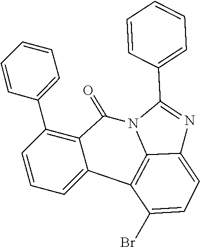 [SMILES: O=c1c2c(-c3ccccc3)cccc2c2c(Br)ccc3nc(-c4ccccc4)n1c32] | 65% |
| 2e | 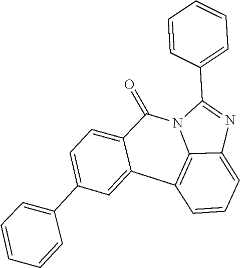 [SMILES: O=c1c2ccc(-c3ccccc3)cc2c2cccc3nc(-c4ccccc4)n1c32] | 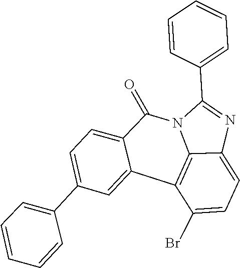 [SMILES: O=c1c2ccc(-c3ccccc3)cc2c2c(Br)ccc3nc(-c4ccccc4)n1c32] | 62% |
| 3e | 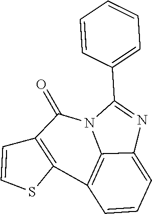 [SMILES: O=c1c2ccsc2c2cccc3nc(-c4ccccc4)n1c32] | 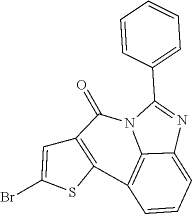 [SMILES: O=c1c2cc(Br)sc2c2cccc3nc(-c4ccccc4)n1c32] | 71% |
| 4e | 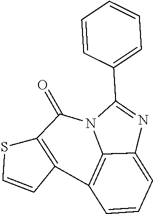 [SMILES: O=c1c2sccc2c2cccc3nc(-c4ccccc4)n1c32] | 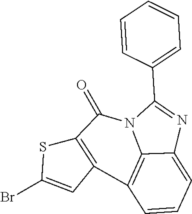 [SMILES: O=c1c2sc(Br)cc2c2cccc3nc(-c4ccccc4)n1c32] | 79% |

### f) 3-[9-(1H-benzimidazol-2-yl)carbazol-3-yl]-9-phenylcarbazole

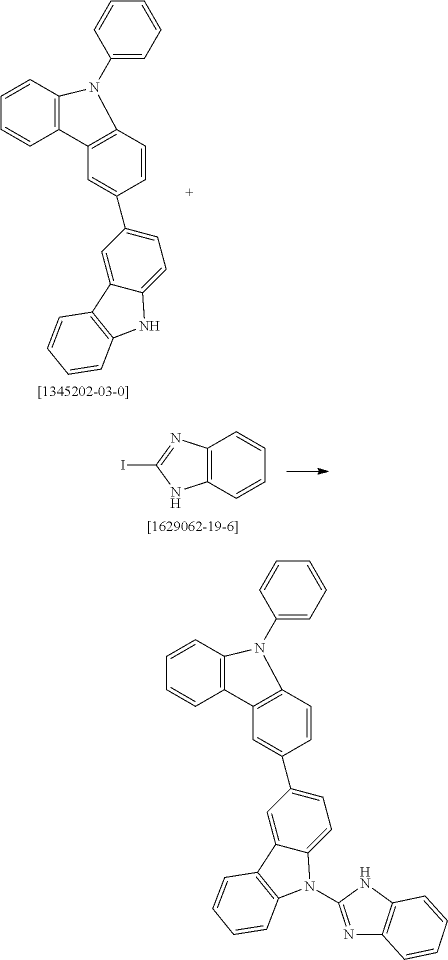 [SMILES: c1ccc(-n2c3ccccc3c3cc(-c4ccc5[nH]c6ccccc6c5c4)ccc32)cc1 ; Ic1nc2ccccc2[nH]1 ; c1ccc(-n2c3ccccc3c3cc(-c4ccc5c(c4)c4ccccc4n5-c4nc5ccccc5[nH]4)ccc32)cc1]

16.3 g (40 mmol) of 3-(9H-carbazol-3-yl)-9-phenylcarbazole and 11 g (45 mmol) of 2-iodo-1H-benzimidazole and 44.7 g (320 mmol) of potassium carbonate, 3 g (16 mmol) of copper(I) iodide and 3.6 g (16 mmol) of 1,3-di(pyridin-2-yl)propane-1,3-dione are stirred in 100 ml of DMF at 150° C. for h. The solution is diluted with water and extracted twice with ethyl acetate, and the combined organic phases are dried over Na₂SO₄ and concentrated by rotary evaporation. The residue is purified by chromatography (EtOAc/hexane: ⅔). The purity is 99.9%.

The yield is 13 g (25 mmol), 63% of theory.

The following compounds can be prepared analogously:

| | Reactant 1 | Reactant 2 | Product | Yield |
|---|---|---|---|---|
| 1f | 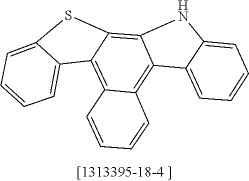 [SMILES: c1ccc2c(c1)[nH]c1c3sc4ccccc4c3c3ccccc3c21] | 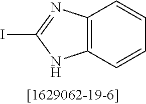 | 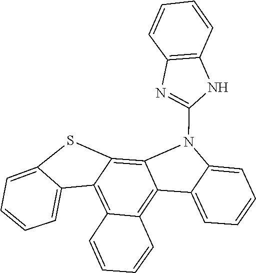 [SMILES: c1ccc2[nH]c(-n3c4ccccc4c4c5ccccc5c5c6ccccc6sc5c43)nc2c1] | 66% |

### g) 9-Phenyl-3-[9-(2-phenyl-1H-benzimidazol-5-yl)carbazol-3-yl]carbazole

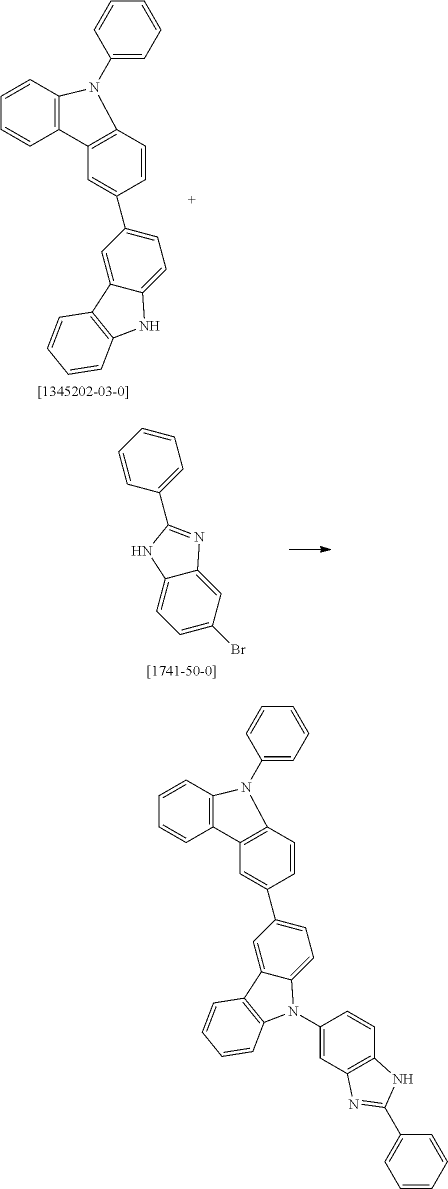 [SMILES: c1ccc(-n2c3ccccc3c3cc(-c4ccc5[nH]c6ccccc6c5c4)ccc32)cc1 ; Brc1ccc2[nH]c(-c3ccccc3)nc2c1 ; c1ccc(-c2nc3cc(-n4c5ccccc5c5cc(-c6ccc7c(c6)c6ccccc6n7-c6ccccc6)ccc54)ccc3[nH]2)cc1]

27 g (66 mmol) of 3-(9H-carbazol-3-yl)-9-phenylcarbazole, 19.11 g (70 mmol) of 5-bromo-2-phenyl-1H-benzimidazole and 19 g of NaOtBu are suspended in 1 I of p-xylene. To this suspension are added 0.3 g (1.33 mmol) of Pd(OAc)₂ and 1.0 ml of a 1M tri-tert-butylphosphine solution. The reaction mixture is heated under reflux for 16 h. After cooling, methylene chloride is added, and the organic phase is removed and washed three times with 200 ml of water and then concentrated to dryness. The residue is subjected to hot extraction with toluene and recrystallized from toluene; purity is 99.9% by HPLC. The yield is 29 g (49 mmol; 75%).

The following compounds can be prepared analogously:

| | Reactant 1 | Reactant 2 | Product | Yield |
|---|---|---|---|---|
| 1g | 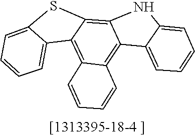 [SMILES: c1ccc2c(c1)[nH]c1c3sc4ccccc4c3c3ccccc3c21] | 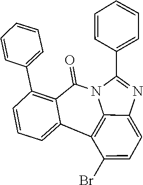 [SMILES: O=c1c2c(-c3ccccc3)cccc2c2c(Br)ccc3nc(-c4ccccc4)n1c32] | 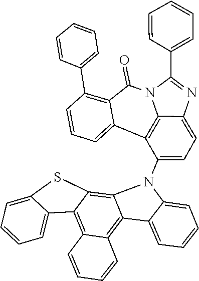 [SMILES: O=c1c2c(-c3ccccc3)cccc2c2c(-n3c4ccccc4c4c5ccccc5c5c6ccccc6sc5c43)ccc3nc(-c4ccccc4)n1c32] | 79% |
| 2g | 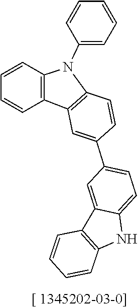 [SMILES: c1ccc(-n2c3ccccc3c3cc(-c4ccc5[nH]c6ccccc6c5c4)ccc32)cc1] | 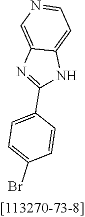 [SMILES: Brc1ccc(-c2nc3cnccc3[nH]2)cc1] | 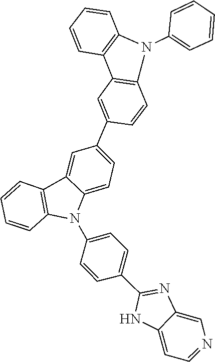 [SMILES: c1ccc(-n2c3ccccc3c3cc(-c4ccc5c(c4)c4ccccc4n5-c4ccc(-c5nc6cnccc6[nH]5)cc4)ccc32)cc1] | 80% |
| 3g | 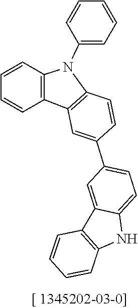 [SMILES: c1ccc(-n2c3ccccc3c3cc(-c4ccc5[nH]c6ccccc6c5c4)ccc32)cc1] | 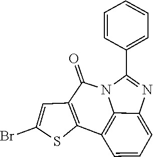 [SMILES: O=c1c2cc(Br)sc2c2cccc3nc(-c4ccccc4)n1c32] | 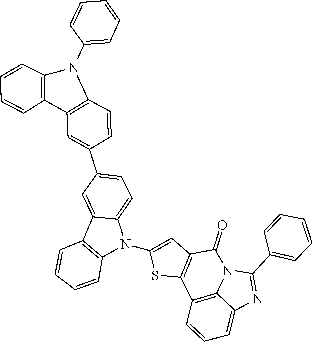 [SMILES: O=c1c2cc(-n3c4ccccc4c4cc(-c5ccc6c(c5)c5ccccc5n6-c5ccccc5)ccc43)sc2c2cccc3nc(-c4ccccc4)n1c32] | 85% |
| 4g | 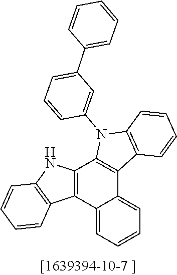 [SMILES: c1ccc(-c2cccc(-n3c4ccccc4c4c5ccccc5c5c6ccccc6[nH]c5c43)c2)cc1] | 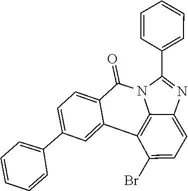 [SMILES: O=c1c2ccc(-c3ccccc3)cc2c2c(Br)ccc3nc(-c4ccccc4)n1c32] | 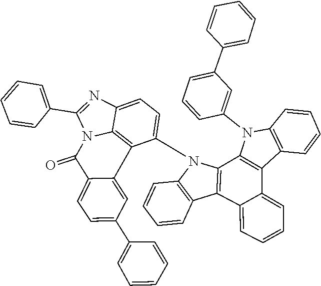 [SMILES: O=c1c2ccc(-c3ccccc3)cc2c2c(-n3c4ccccc4c4c5ccccc5c5c6ccccc6n(-c6cccc(-c7ccccc7)c6)c5c43)ccc3nc(-c4ccccc4)n1c32] | 80% |
| 5g | 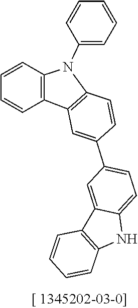 [SMILES: c1ccc(-n2c3ccccc3c3cc(-c4ccc5[nH]c6ccccc6c5c4)ccc32)cc1] | 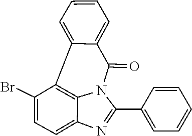 [SMILES: O=c1c2ccccc2c2c(Br)ccc3nc(-c4ccccc4)n1c32] | 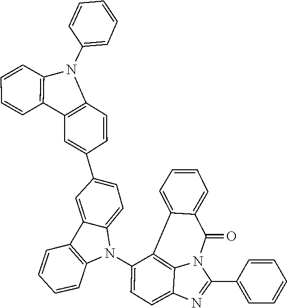 [SMILES: O=c1c2ccccc2c2c(-n3c4ccccc4c4cc(-c5ccc6c(c5)c5ccccc5n6-c5ccccc5)ccc43)ccc3nc(-c4ccccc4)n1c32] | 77% |

### B) Device Examples

Examples E1 to E14 which follow (see table 1) present the use of the materials of the invention in OLEDs.

Glass plates coated with structured ITO (indium tin oxide) of thickness 50 nm are treated prior to coating with an oxygen plasma, followed by an argon plasma. These plasma-treated glass plates form the substrates to which the OLEDs are applied.

The OLEDs basically have the following layer structure: substrate/optional interlayer (IL)/hole injection layer (HIL)/hole transport layer (HTL)/electron blocker layer (EBL)/emission layer (EML)/optional hole blocker layer (HBL)/electron transport layer (ETL)/optional electron injection layer (EIL) and finally a cathode. The cathode is formed by an aluminium layer of thickness 100 nm. The exact structure of the OLEDs can be found in table 1. The materials required for production of the OLEDs are shown in table 2. The data of the OLEDs are listed in tables 3 and 4.

All materials are applied by thermal vapour deposition in a vacuum chamber. In this case, the emission layer always consists of at least one matrix material (host material) and an emitting dopant (emitter) which is added to the matrix material(s) in a particular proportion by volume by co-evaporation. Details given in such a form as IC1:EG1:TEG1 (45%:45%:10%) mean here that the material IC1 is present in the layer in a proportion by volume of 45%, EG1 in a proportion of 45% and TEG1 in a proportion of 10%. Analogously, the electron transport layer may also consist of a mixture of two materials.

The OLEDs are characterized in a standard manner. For this purpose, electroluminescence spectra, current efficiency (CE, measured in cd/A) and external quantum efficiency (EQE, measured in %) are determined as a function of luminance, calculated from current-voltage-luminance characteristics assuming Lambertian emission characteristics. Electroluminescence spectra are determined at a luminance of 1000 cd/m², and these are used to calculate the CIE 1931 x and y colour coordinates. The results thus obtained can be found in tables 3 and 4.

Use of Compounds of the Formula (I) as Matrix Materials in the Emitting Layer

The inventive compounds EG1 to EG7 are used in examples E1 to E9 as matrix material in the emission layer of phosphorescent green OLEDs (table 3). Low voltage and good efficiency occur here.

The inventive compounds EG8, EG9 and EG10 are used in examples E10 to E13 as matrix material in the emission layer of phosphorescent red OLEDs (table 3). Low voltage and good efficiency occur here.

Use of Compounds of the Formula (I) as Electron Transport Materials in the Emitting Layer

When the inventive compound EG5 is used as electron transport material in example E14, low voltage and good efficiency are obtained (table 4).

TABLE 1
Structure of the OLEDs

| | | HIL | HTL | EBL | EML | HBL | ETL | EIL |
|---|---|---|---|---|---|---|---|---|
| Ex. | IL | thickness | thickness | thickness | thickness | thickness | thickness | thickness |
| E1 | | HATCN | SpMAl | SpMA2 | EG1:TEG1 | ST2 | ST2:LiQ | LiQ |
| | | 5 nm | 230 nm | 20 nm | (88%:12%) 40 nm | 5 nm | (50%:50%) 30 nm | 1 nm |
| E2 | | HATCN | SpMAl | SpMA2 | EG2:TEG1 | ST2 | ST2:LiQ | LiQ |
| | | 5 nm | 230 nm | 20 nm | (88%:12%) 40 nm | 5 nm | (50%:50%) 30 nm | 1 nm |
| E3 | | HATCN | SpMAl | SpMA2 | EG3:TEG1 | ST2 | ST2:LiQ | LiQ |
| | | 5 nm | 230 nm | 20 nm | (88%:12%) 40 nm | 5 nm | (50%:50%) 30 nm | 1 nm |
| E4 | | HATCN | SpMAl | SpMA2 | EG1:IC1:TEG1 | ST2 | ST2:LiQ | LiQ |
| | | 5 nm | 230 nm | 20 nm | (49%:44%:7%) 40 nm | 5 nm | (50%:50%) 30 nm | 1 nm |
| E5 | | HATCN | SpMAl | SpMA2 | EG3:IC2:TEG1 | ST2 | ST2:LiQ | LiQ |
| | | 5 nm | 230 nm | 20 nm | (44%:44%:12%) 40 nm | 5 nm | (50%:50%) 30 nm | 1 nm |
| E6 | | HATCN | SpMAl | SpMA2 | EG4:IC3:TEG1 | ST2 | ST2:LiQ | LiQ |
| | | 5 nm | 230 nm | 20 nm | (49%:44%:7%) 40 nm | 5 nm | (50%:50%) 30 nm | 1 nm |
| E7 | | HATCN | SpMAl | SpMA2 | EG5:IC4:TEG1 | ST2 | ST2:LiQ | LiQ |
| | | 5 nm | 230 nm | 20 nm | (49%:44%:7%) 40 nm | 5 nm | (50%:50%) 30 nm | 1 nm |
| E8 | | HATCN | SpMAl | SpMA2 | EG6:IC1:TEG1 | ST2 | ST2:LiQ | LiQ |
| | | 5 nm | 230 nm | 20 nm | (49%:44%:7%) 40 nm | 5 nm | (50%:50%) 30 nm | 1 nm |
| E9 | | HATCN | SpMAl | SpMA2 | EG7:IC1:TEG1 | ST2 | ST2:LiQ | LiQ |
| | | 5 nm | 230 nm | 20 nm | (49%:44%:7%) 40 nm | 5 nm | (50%:50%) 30 nm | 1 nm |
| E10 | | HATCN | SpMAl | SpMA2 | EG8:TER5 | ST2 | ST2:LiQ | LiQ |
| | | 5 nm | 125 nm | 10 nm | (97%:3%) 35 nm | 10 nm | (50%:50%) 30 nm | 1 nm |
| E11 | | HATCN | SpMAl | SpMA2 | EG9:TER5 | ST2 | ST2:LiQ | LiQ |
| | | 5 nm | 125 nm | 10 nm | (97%:3%) 35 nm | 10 nm | (50%:50%) 30 nm | 1 nm |
| E12 | | HATCN | SpMAl | SpMA2 | EG10:TER5 | ST2 | ST2:LiQ | LiQ |
| | | 5 nm | 125 nm | 10 nm | (97%:3%) 35 nm | 10 nm | (50%:50%) 30 nm | 1 nm |
| E13 | | HATCN | SpMAl | SpMA2 | EG8:IC5:TER5 | ST2 | ST2:LiQ | LiQ |
| | | 5 nm | 125 nm | 10 nm | (72%:25%:3%) 35 nm | 10 nm | (50%:50%) 30 nm | 1 nm |
| E14 | SpAl | | HATCN | SpMAl | IC1:TEG1 | — | EG5:LiQ | |
| | 70 nm | | 5 nm | 90 nm | (90%:10%) 30 nm | | (50%:50%) 40 nm | |

TABLE 2
Structural formulae of the materials for the OLEDs

| | |
|---|---|
| 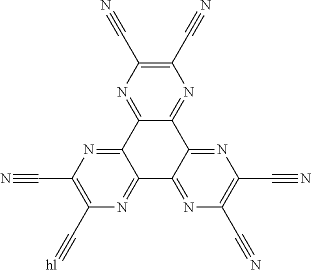 | 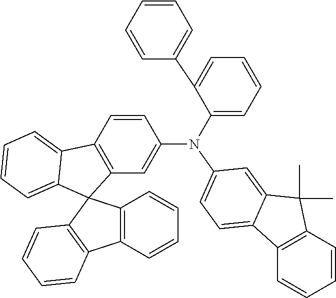 [SMILES: CC1(C)c2ccccc2-c2ccc(N(c3ccc4c(c3)C3(c5ccccc5-c5ccccc53)c3ccccc3-4)c3ccccc3-c3ccccc3)cc21] |
| HATCN | SpMA1 |
| 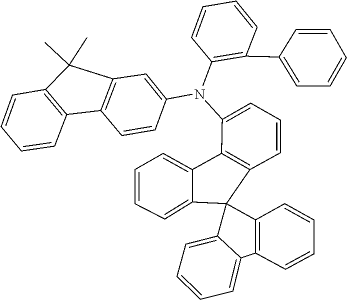 [SMILES: CC1(C)c2ccccc2-c2ccc(N(c3ccccc3-c3ccccc3)c3cccc4c3-c3ccccc3C43c4ccccc4-c4ccccc43)cc21] | 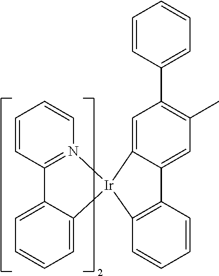 |
| SpMA2 | TEG1 |
| 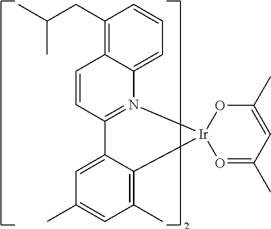 | 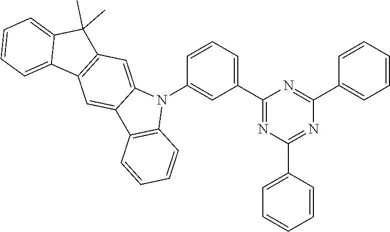 [SMILES: CC1(C)c2ccccc2-c2cc3c4ccccc4n(-c4cccc(-c5nc(-c6ccccc6)nc(-c6ccccc6)n5)c4)c3cc21] |
| TER5 | IC1 |
| 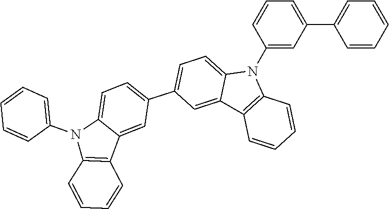 [SMILES: c1ccc(-c2cccc(-n3c4ccccc4c4cc(-c5ccc6c(c5)c5ccccc5n6-c5ccccc5)ccc43)c2)cc1] | 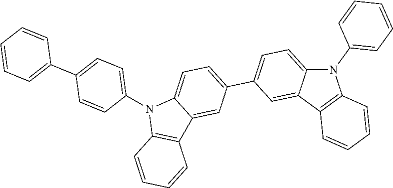 [SMILES: c1ccc(-c2ccc(-n3c4ccccc4c4cc(-c5ccc6c(c5)c5ccccc5n6-c5ccccc5)ccc43)cc2)cc1] |
| IC2 | IC3 |
| 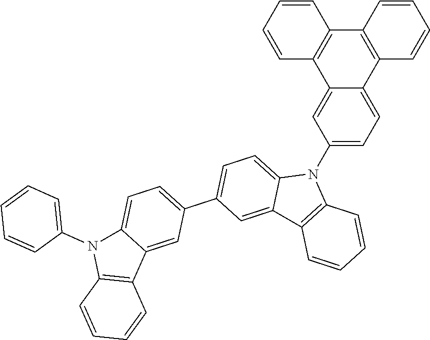 [SMILES: c1ccc(-n2c3ccccc3c3cc(-c4ccc5c(c4)c4ccccc4n5-c4ccc5c6ccccc6c6ccccc6c5c4)ccc32)cc1] | 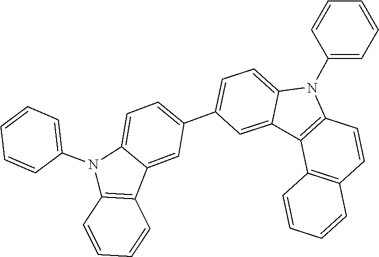 [SMILES: c1ccc(-n2c3ccccc3c3cc(-c4ccc5c(c4)c4c6ccccc6ccc4n5-c4ccccc4)ccc32)cc1] |
| IC4 | IC5 |
| 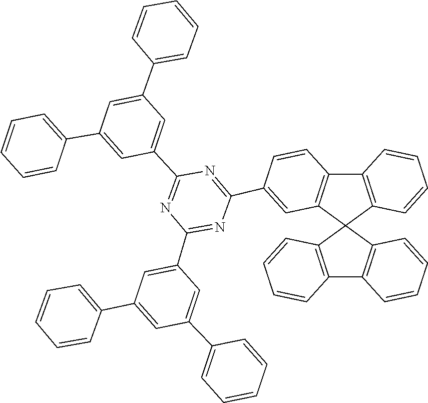 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1] | 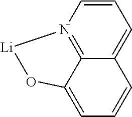 |
| ST2 | LiQ |
| 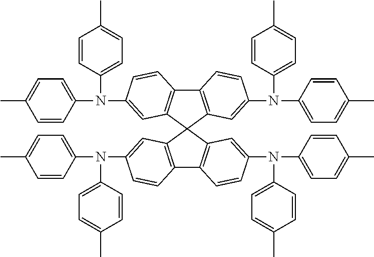 | 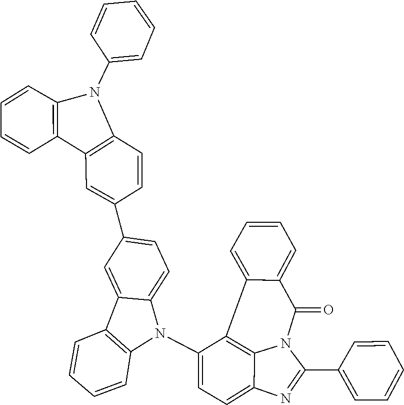 [SMILES: O=c1c2ccccc2c2c(-n3c4ccccc4c4cc(-c5ccc6c(c5)c5ccccc5n6-c5ccccc5)ccc43)ccc3nc(-c4ccccc4)n1c32] |
| SpA1 | EG1 (5g) |
| 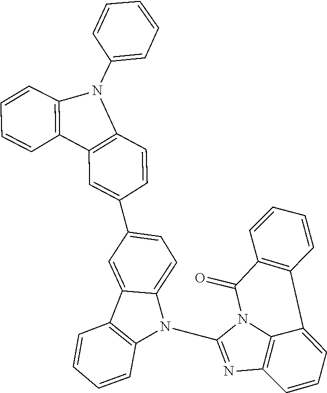 [SMILES: O=c1c2ccccc2c2cccc3nc(-n4c5ccccc5c5cc(-c6ccc7c(c6)c6ccccc6n7-c6ccccc6)ccc54)n1c32] | 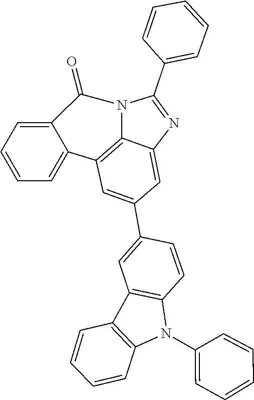 [SMILES: O=c1c2ccccc2c2cc(-c3ccc4c(c3)c3ccccc3n4-c3ccccc3)cc3nc(-c4ccccc4)n1c32] |
| EG2 (15d) | EG3 (26d) |
| 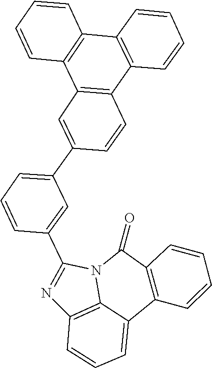 [SMILES: O=c1c2ccccc2c2cccc3nc(-c4cccc(-c5ccc6c7ccccc7c7ccccc7c6c5)c4)n1c32] | 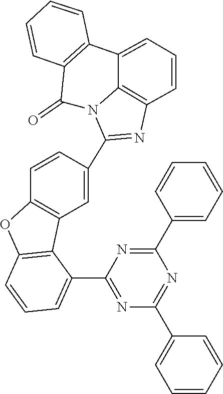 [SMILES: O=c1c2ccccc2c2cccc3nc(-c4ccc5oc6cccc(-c7nc(-c8ccccc8)nc(-c8ccccc8)n7)c6c5c4)n1c32] |
| EG4 (29d) | EG5 (31d) |
| 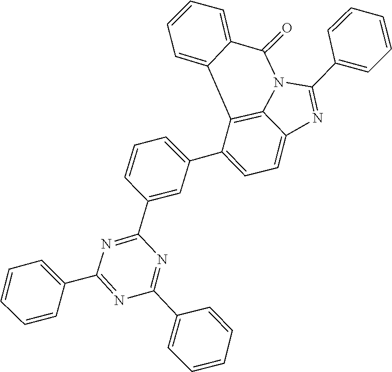 [SMILES: O=c1c2ccccc2c2c(-c3cccc(-c4nc(-c5ccccc5)nc(-c5ccccc5)n4)c3)ccc3nc(-c4ccccc4)n1c32] | 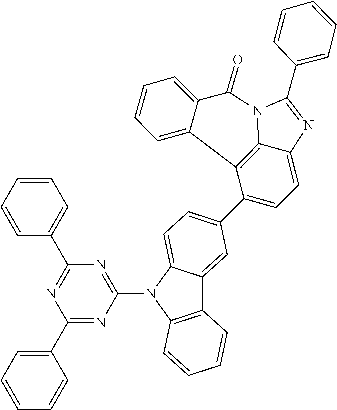 [SMILES: O=c1c2ccccc2c2c(-c3ccc4c(c3)c3ccccc3n4-c3nc(-c4ccccc4)nc(-c4ccccc4)n3)ccc3nc(-c4ccccc4)n1c32] |
| EG6 (17a) | EG7 (19a) |
| 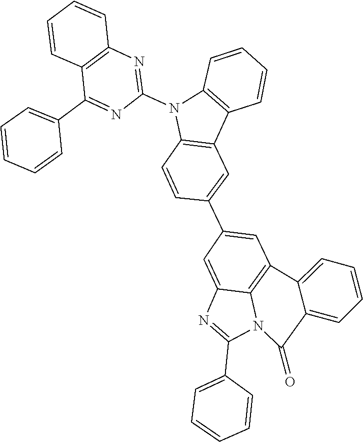 [SMILES: O=c1c2ccccc2c2cc(-c3ccc4c(c3)c3ccccc3n4-c3nc(-c4ccccc4)c4ccccc4n3)cc3nc(-c4ccccc4)n1c32] | 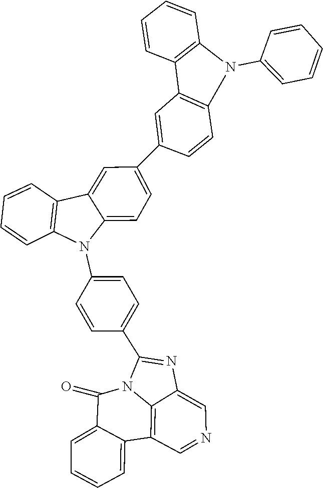 [SMILES: O=c1c2ccccc2c2cncc3nc(-c4ccc(-n5c6ccccc6c6cc(-c7ccc8c(c7)c7ccccc7n8-c7ccccc7)ccc65)cc4)n1c32] |
| EG8 (21d) | EG9 (27d) |
| 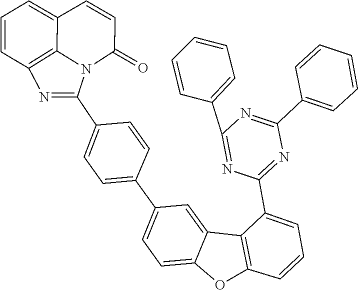 [SMILES: O=c1ccc2cccc3nc(-c4ccc(-c5ccc6oc7cccc(-c8nc(-c9ccccc9)nc(-c9ccccc9)n8)c7c6c5)cc4)n1c23] | |
| EG 10 (22a) | |

TABLE 3
Data of the OLEDs

| | U1000 | SE1000 | EQE 1000 | CIE x/y at |
|---|---|---|---|---|
| Ex. | (V) | (cd/A) | (%) | 1000 cd/m² |
| E1 | 4.2 | 66 | 15.5 | 0.33/0.62 |
| E2 | 4.4 | 67 | 15 | 0.34/0.62 |
| E3 | 4.6 | 58 | 16 | 0.34/0.61 |
| E4 | 3.3 | 62 | 17 | 0.35/0.60 |
| E5 | 3.5 | 64 | 17 | 0.34/0.63 |
| E6 | 3.1 | 67 | 18 | 0.35/0.61 |
| E7 | 3.2 | 72 | 19 | 0.33/0.62 |
| E8 | 3.1 | 67 | 18 | 0.34/0.61 |
| E9 | 3.1 | 68 | 18 | 0.33/0.62 |
| E10 | 3.9 | 23 | 19.5 | 0.67/0.33 |
| E11 | 3.8 | 24 | 20 | 0.66/0.34 |
| E12 | 3.2 | 23 | 21 | 0.67/0.33 |
| E13 | 3.5 | 23 | 18.2 | 0.67/0.33 |

TABLE 4
Data of the OLEDs

| | U1000 | SE1000 | EQE 1000 | CIE x/y at |
|---|---|---|---|---|
| Ex. | (V) | (cd/A) | (%) | 1000 cd/m² |
| E14 | 3.8 | 65 | 16 | 0.31/0.64 |

1.-24. (canceled)

25. A compound of a formula (I) 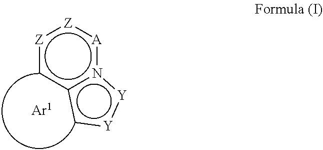
where
A is C═O, C═S, C═NR⁰, P(═O)R⁰, SO or SO₂;
Y is the same or different at each instance and is selected from N and CR¹;
Ar¹ is an aromatic ring system which has 6 to 40 aromatic ring atoms and is substituted by R² radicals, and which is fused onto the rest of the formula (I) via the three carbon atoms shown in formula (I), or a heteroaromatic ring system which has 5 to 40 aromatic ring atoms and is substituted by R² radicals, and which is fused onto the rest of the formula (I) via the three carbon atoms shown in formula (I);
Z is the same or different at each instance and is selected from CR⁴ and N, or the Z—Z unit represents a unit of formula (Ar²) 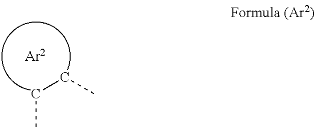
where Ar² is selected from aromatic ring systems which have 6 to 40 aromatic ring atoms and are substituted by R³ radicals, and which include the C—C unit, and heteroaromatic ring systems which have 5 to 40 aromatic ring atoms and are substituted by R³ radicals, and which include the C—C unit, and where the dotted lines are the bonds of the Z—Z unit to the rest of the formula;
R⁰ is the same or different at each instance and is selected from straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R¹ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R² is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more radicals selected from R², R³ and R⁴ may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R³ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more radicals selected from R², R³ and R⁴ may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R⁴ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R², R³ and R⁴ radicals may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R⁵ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁵ radicals may be joined to one another and may form a ring; where the alkyl, alkoxy, alkenyl and alkynyl groups mentioned and the aromatic ring systems and heteroaromatic ring systems mentioned are each substituted by R⁶ radicals; and where one or more CH₂ groups in the alkyl, alkoxy, alkenyl and alkynyl groups mentioned may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;
R⁶ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁶ radicals may be joined to one another and may form a ring; and where the alkyl, alkoxy, alkenyl and alkynyl groups, aromatic ring systems and heteroaromatic ring systems mentioned may be substituted by one or more radicals selected from F and CN;
where, when the two Y groups in formula (I) are CR¹, either
a) at least one group selected from the R¹, R², R³ and R⁴ groups is selected from aromatic ring systems which have 7 to 40 aromatic ring atoms and are each substituted by R⁵ radicals; and heteroaromatic ring systems which have 5 to 40 aromatic ring atoms and are each substituted by R⁵ radicals; or
b) at least two groups selected from the R¹, R², R³ and R⁴ groups are selected from aromatic ring systems which have 6 to 40 aromatic ring atoms and are each substituted by R⁵ radicals; and heteroaromatic ring systems which have 5 to 40 aromatic ring atoms and are each substituted by R⁵ radicals.

26. The compound according to claim 25, wherein A is C═O.

27. The compound according to claim 25, wherein Ar¹ is selected from benzene, pyridine, pyrimidine, pyridazine, naphthalene, quinoline, quinazoline, phenanthrene, anthracene, triphenylene, fluorene, carbazole, dibenzofuran and dibenzothiophene.

28. The compound according to claim 25, wherein the Z—Z unit is a unit of the formula (Ar²) 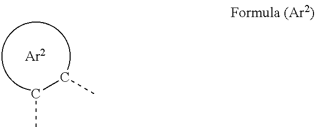
where Ar² is selected from aromatic ring systems which have 6 to 40 aromatic ring atoms and are substituted by R³ radicals, and which include the C—C unit, and heteroaromatic ring systems which have 5 to 40 aromatic ring atoms and are substituted by R³ radicals, and which include the C—C unit, and where the dotted lines are the bonds of the Z—Z unit to the rest of the formula.

29. The compound according to claim 25, wherein Ar² is selected from benzene, pyridine, pyrimidine, pyridazine, pyrazine, naphthalene, thiophene, furan, pyrrole, imidazole, thiazole, oxazole, benzothiophene, benzofuran, indole and indane, each of which is substituted by R³ radicals, and which include the C—C unit in formula (Ar²).

30. The compound according to claim 25, wherein formula (I) includes at least one R¹ group selected from N(R⁵)₂, aromatic ring systems which have 6 to 40 aromatic ring atoms and are substituted by R⁵ radicals, and heteroaromatic ring systems which have 5 to 40 aromatic ring atoms and are substituted by R⁵ radicals.

31. The compound according to claim 25, wherein formula (I) includes at least one R¹ group selected from the R-1 to R-82 groups 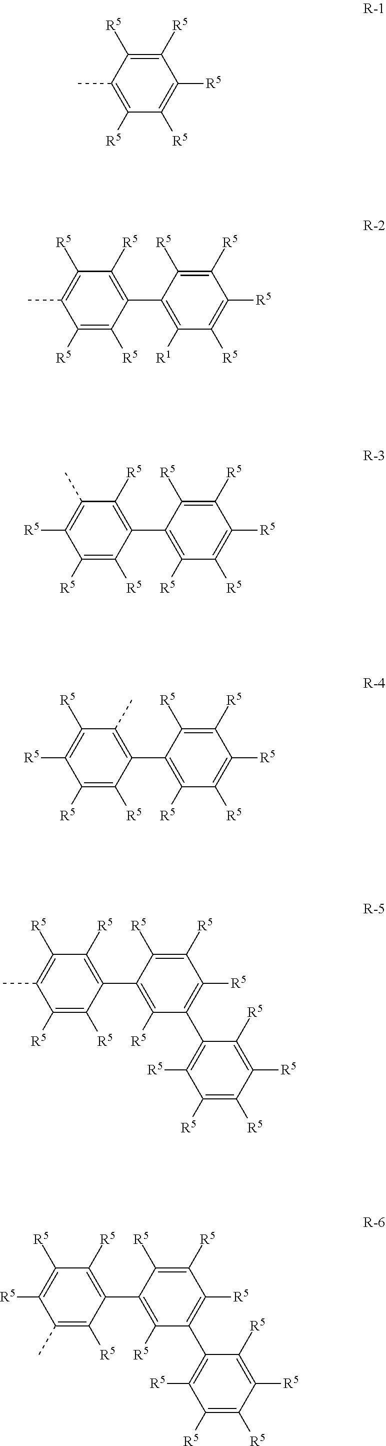 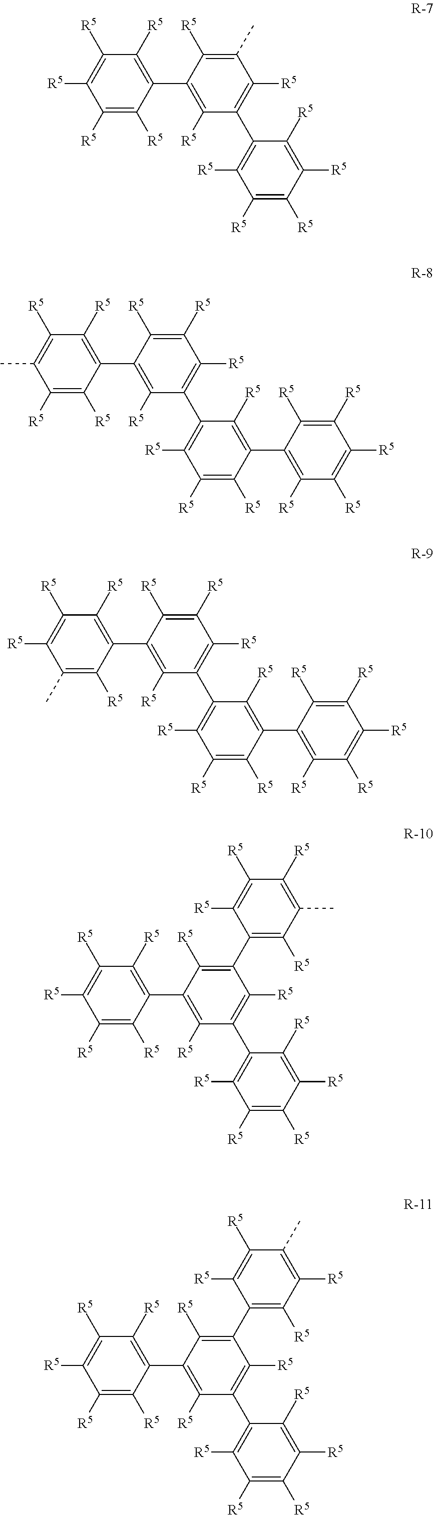 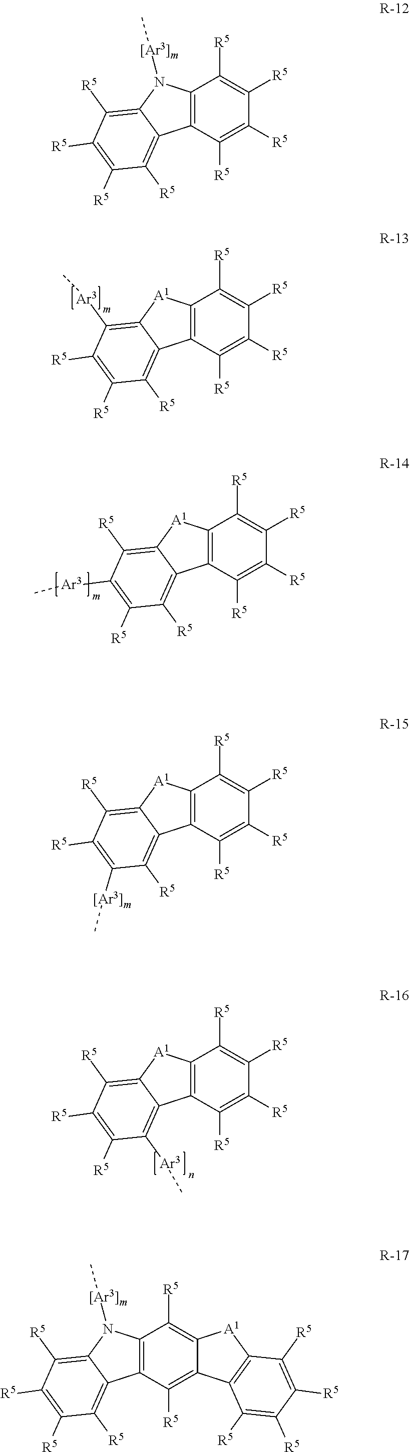 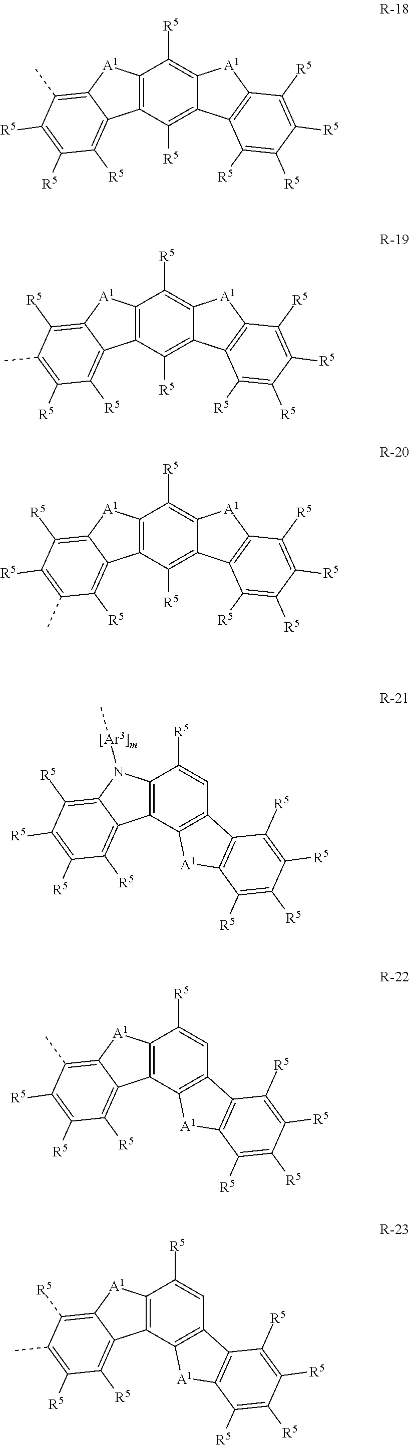 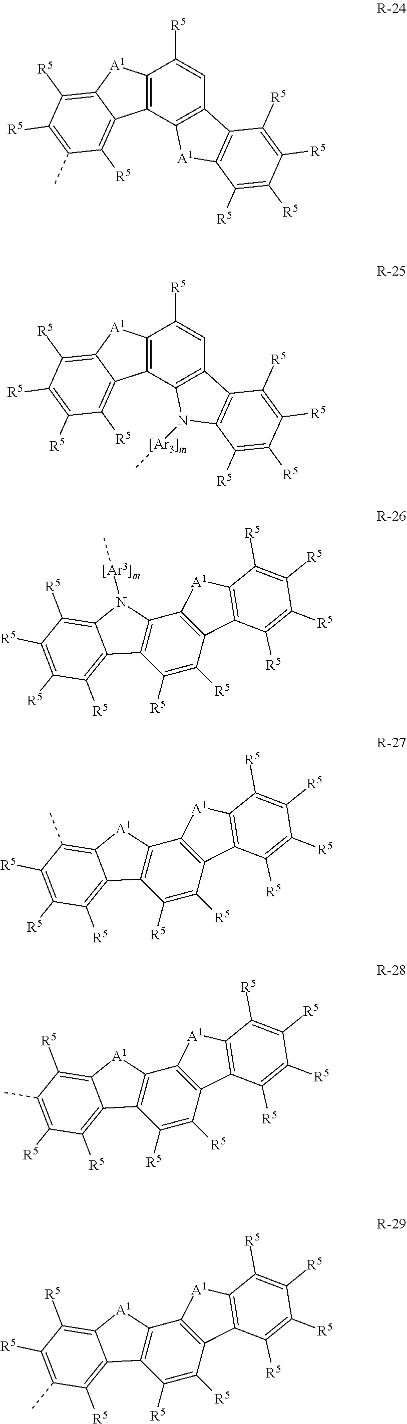 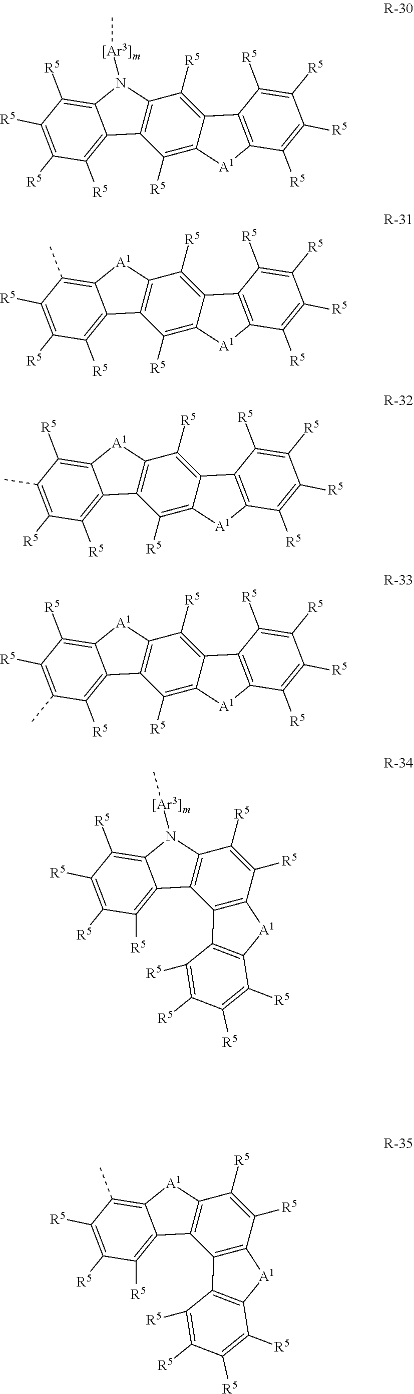 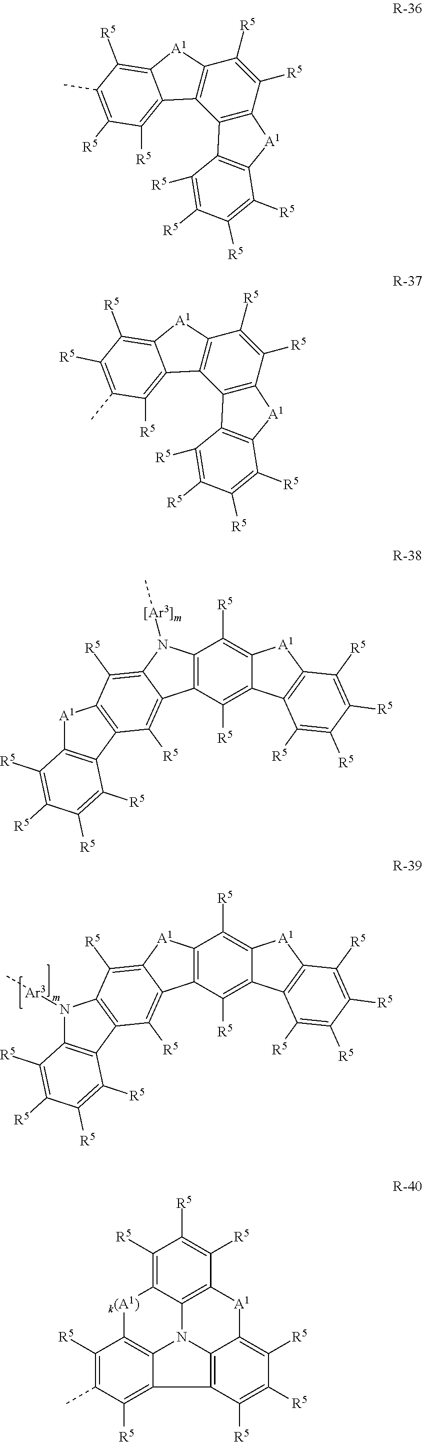 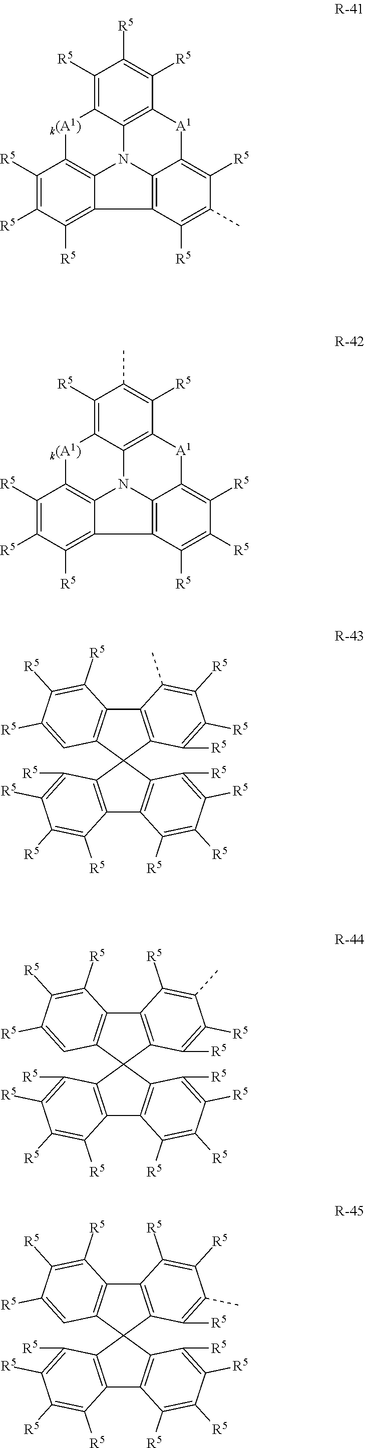 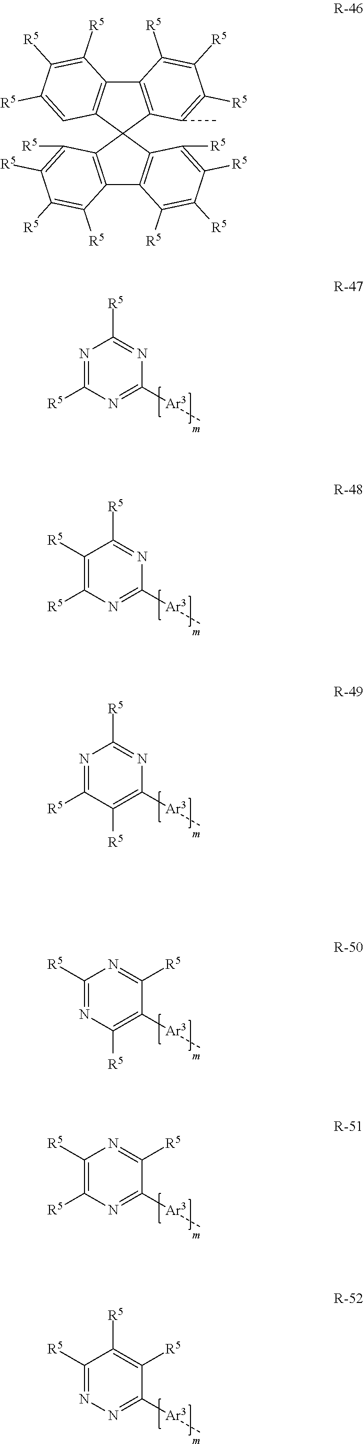 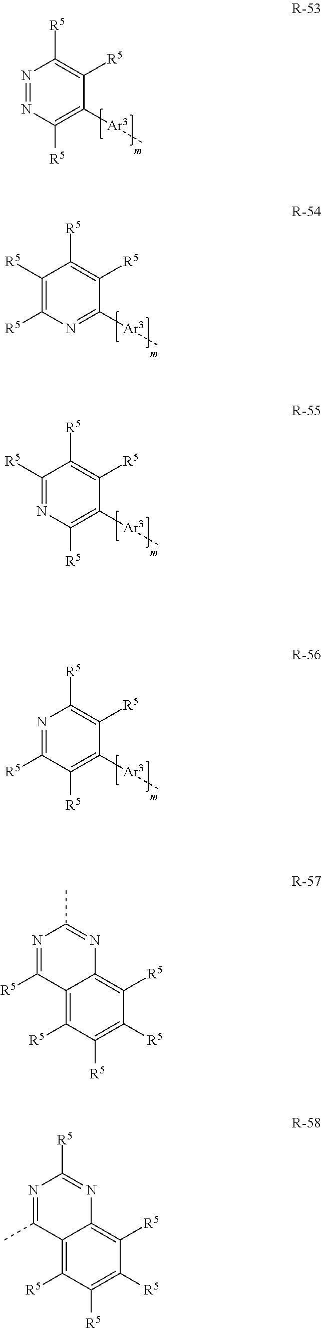 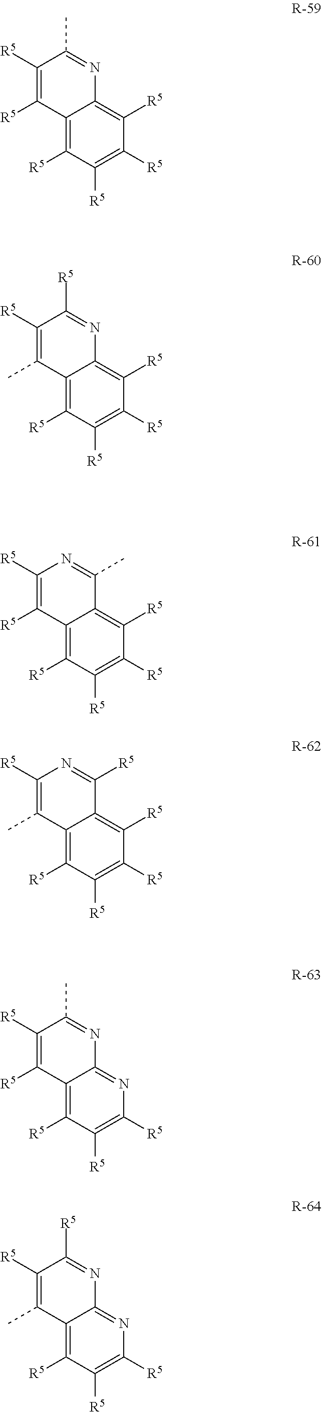 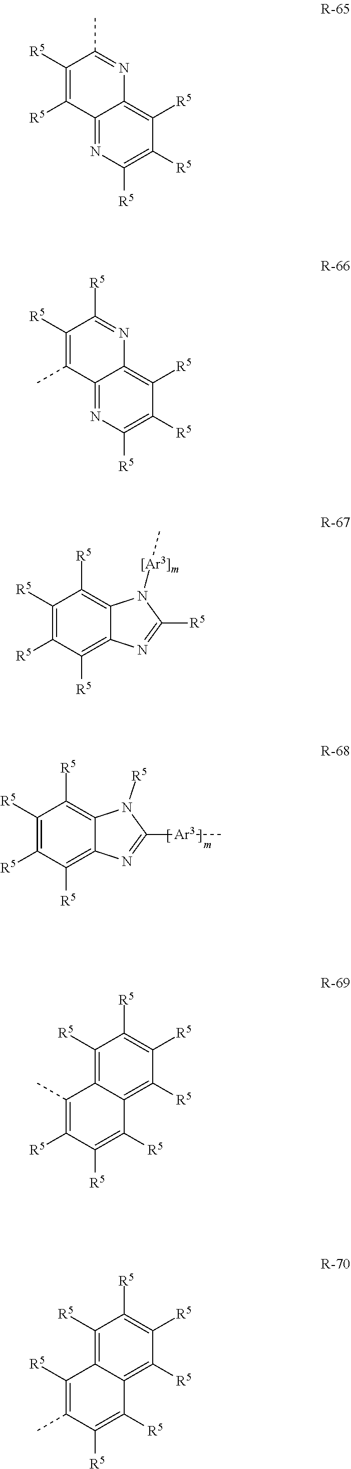 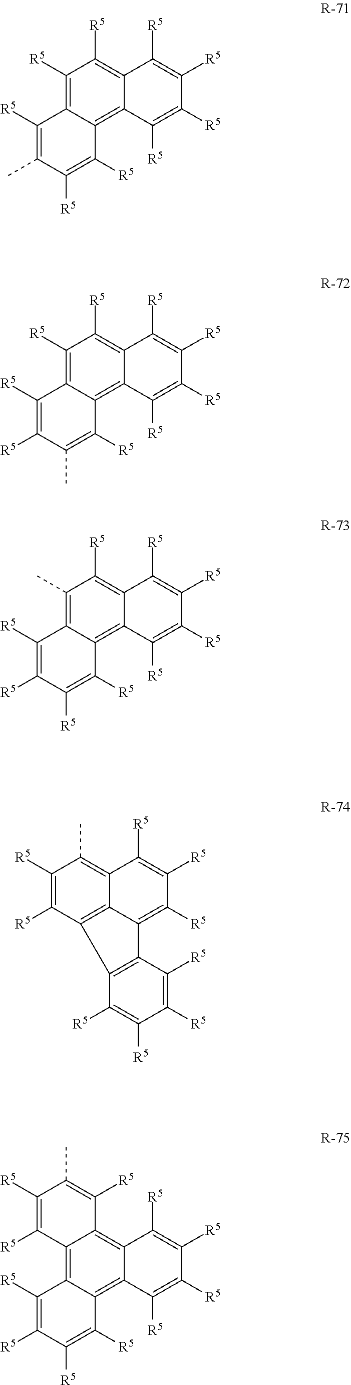 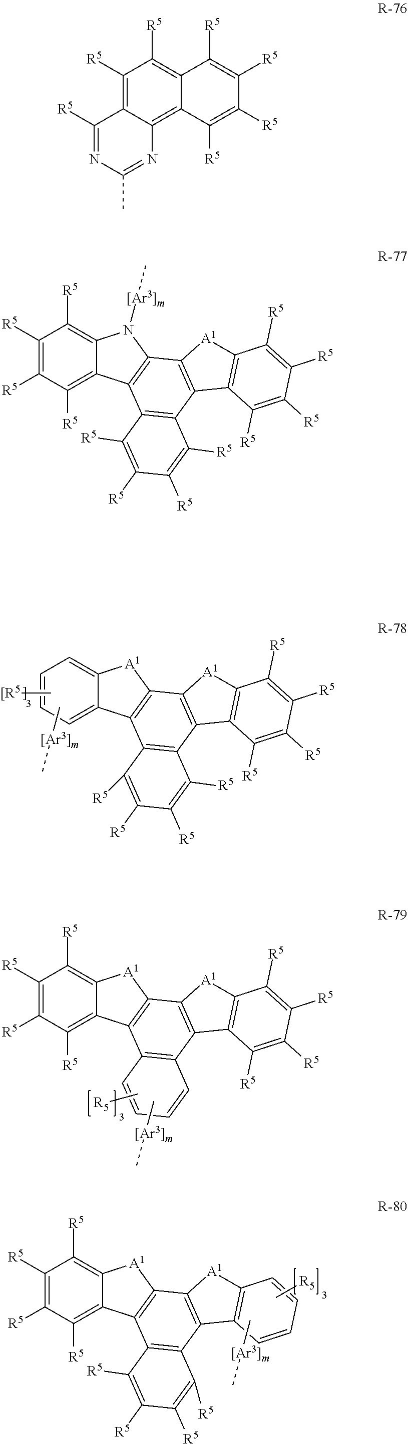 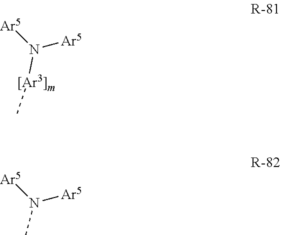
where the dotted bond represents the bond to the base skeleton of the formula (I), and in addition:
Ar³ is the same or different at each instance and is a bivalent aromatic or heteroaromatic ring system which has 6 to 12 aromatic ring atoms and is substituted in each case by R⁵ radicals;
Ar⁵ is the same or different at each instance and is an aromatic ring system which has 6 to 40 aromatic ring atoms and is substituted by R⁶ radicals, or a heteroaromatic ring system which has 6 to 40 aromatic ring atoms and is substituted by R⁶ radicals;
A¹ is the same or different at each instance and is C(R⁵)₂, NR⁵, O or S;
k is 0 or 1, where k=0 means that no A³ group is bonded at this position and R⁵ radicals are bonded to the corresponding carbon atoms instead;
m is 0 or 1, where m=0 means that the Ar³ group is absent and that the corresponding aromatic or heteroaromatic group is bonded directly to the base skeleton of the formula (I).

32. The compound according to claim 25, wherein formula (I) includes at least one R² group selected from N(R⁵)₂, aromatic ring systems which have 6 to 40 aromatic ring atoms and are substituted by R⁵ radicals, and heteroaromatic ring systems which have 5 to 40 aromatic ring atoms and are substituted by R⁵ radicals.

33. The compound according to claim 25, wherein formula (I) includes at least one R² group selected from the R-1 to R-82 groups

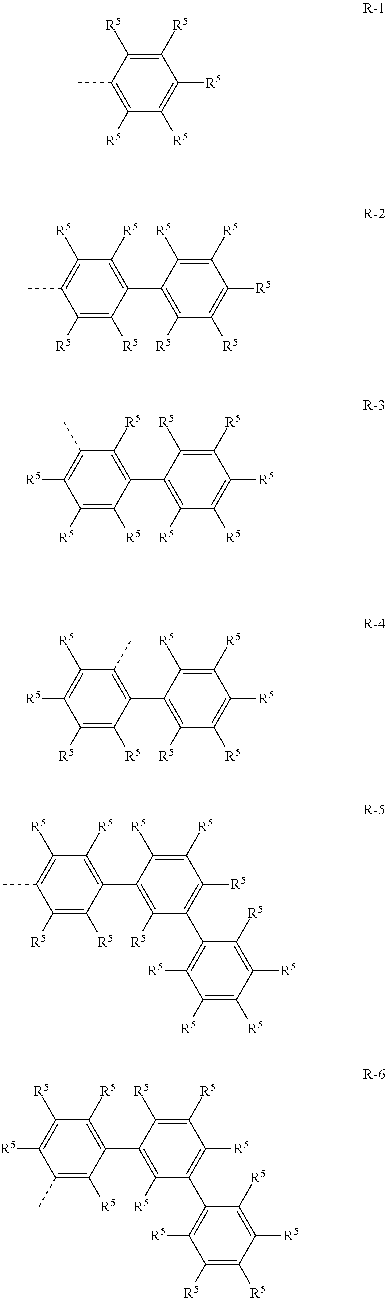

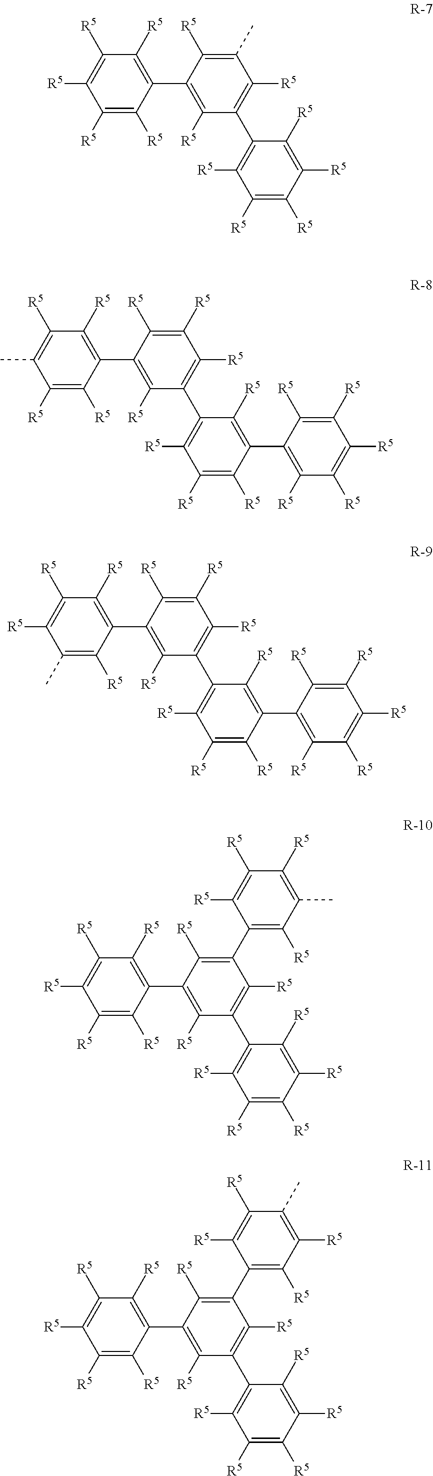

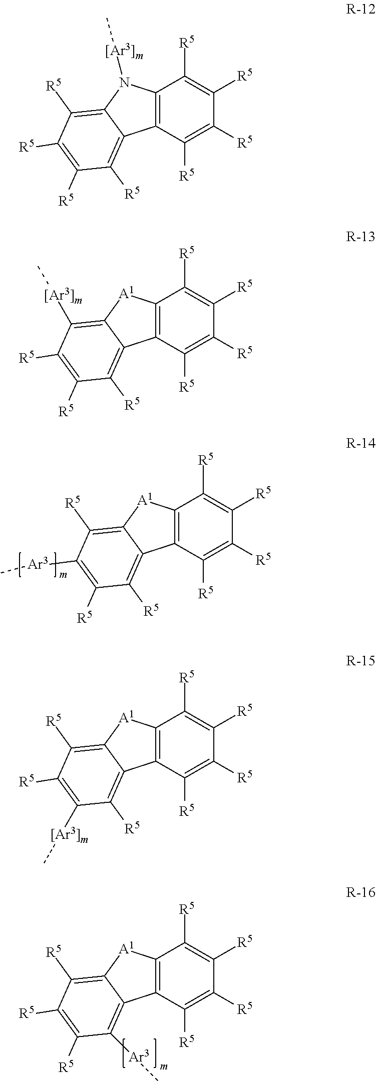

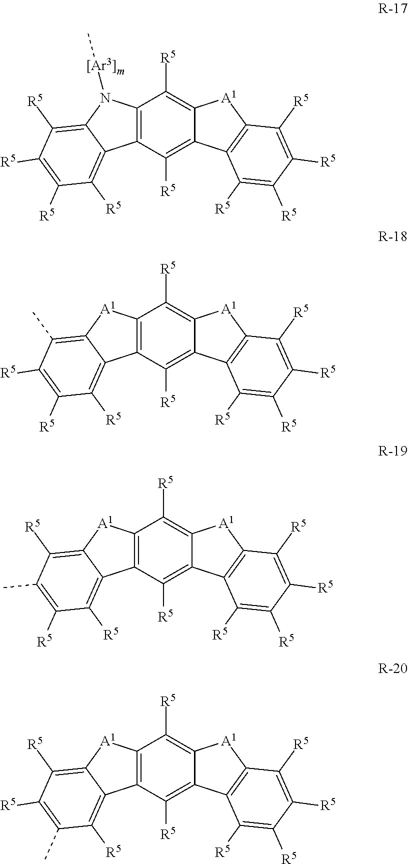

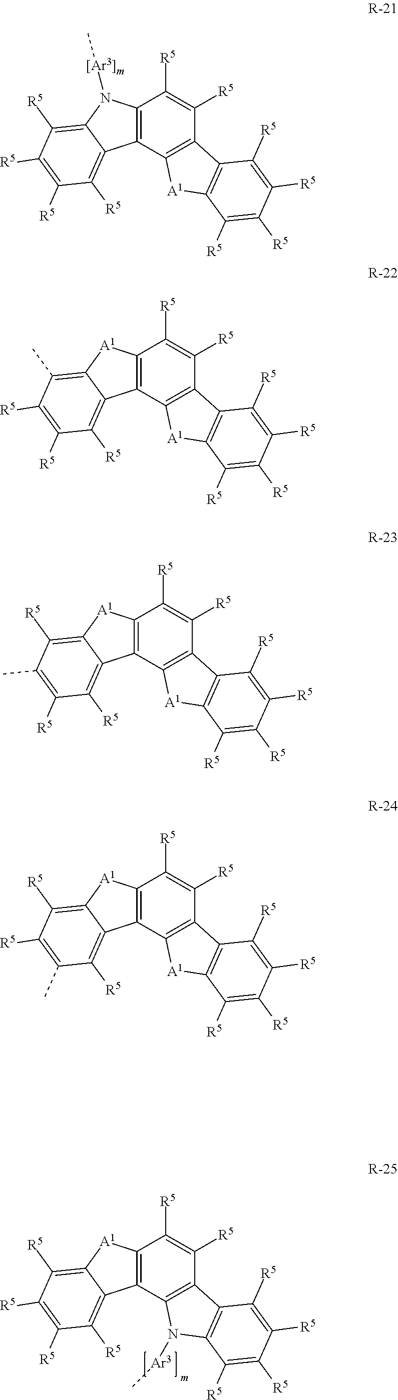

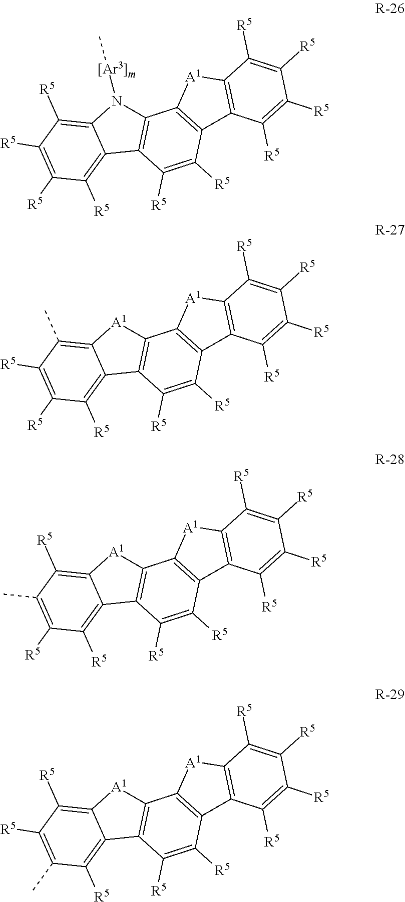

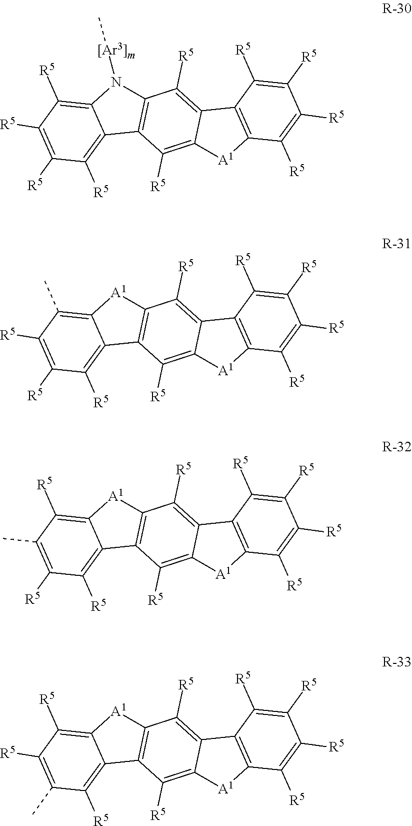

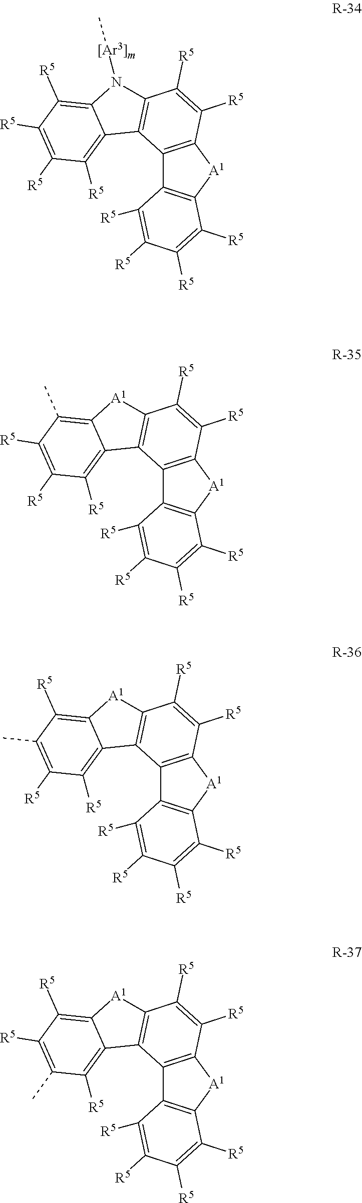

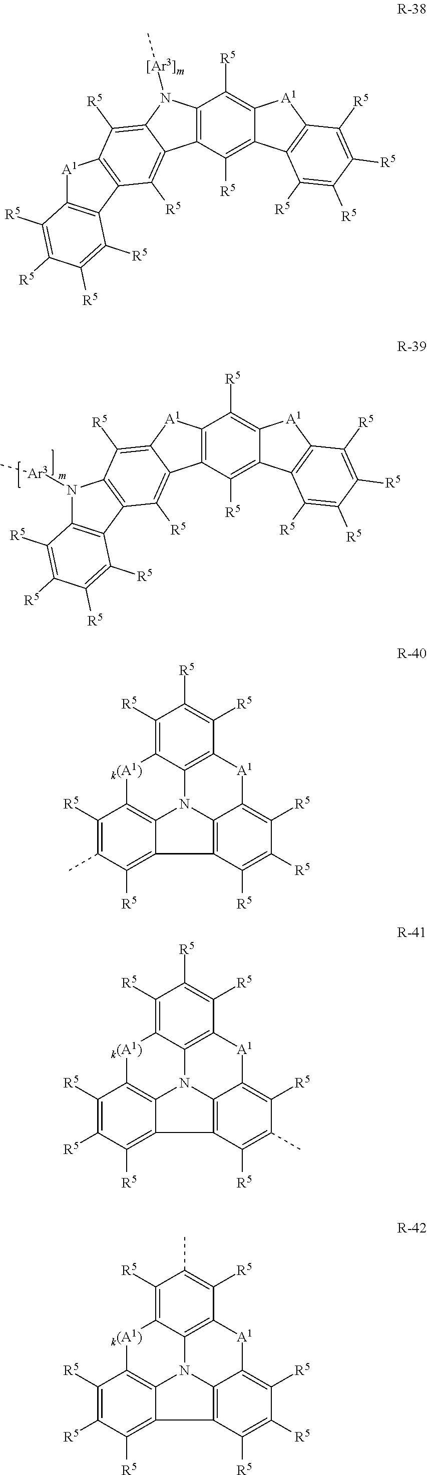

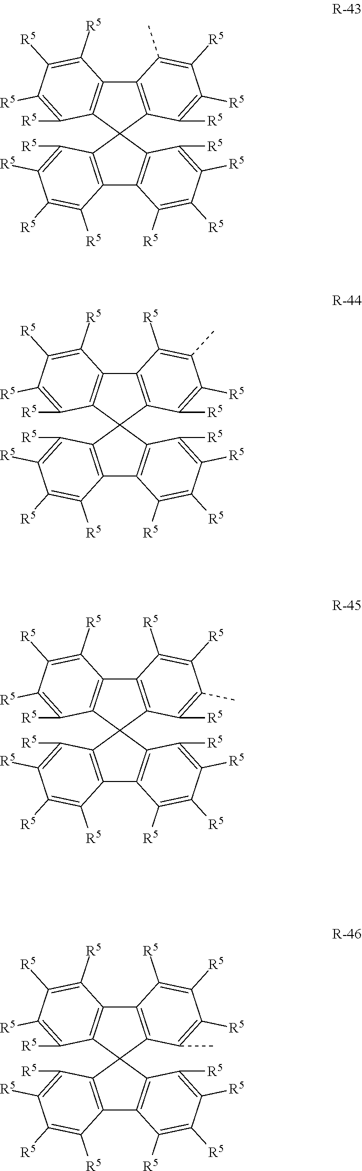

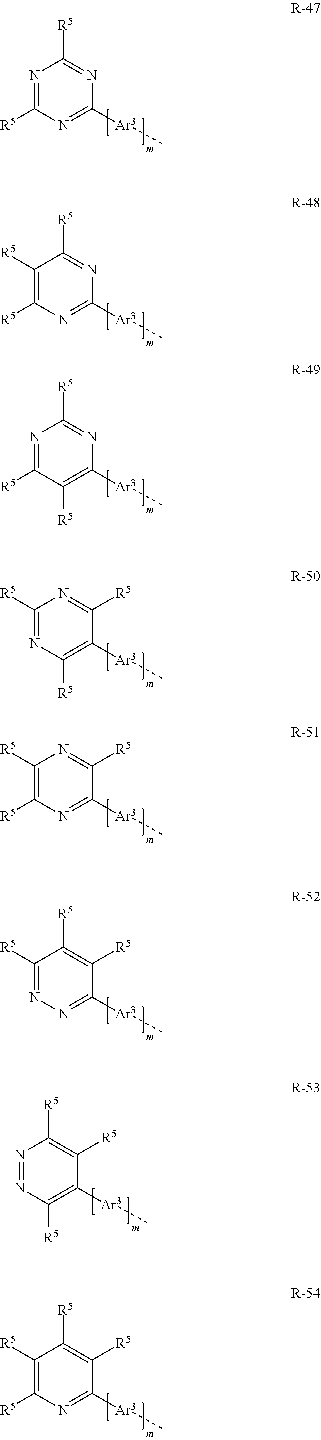

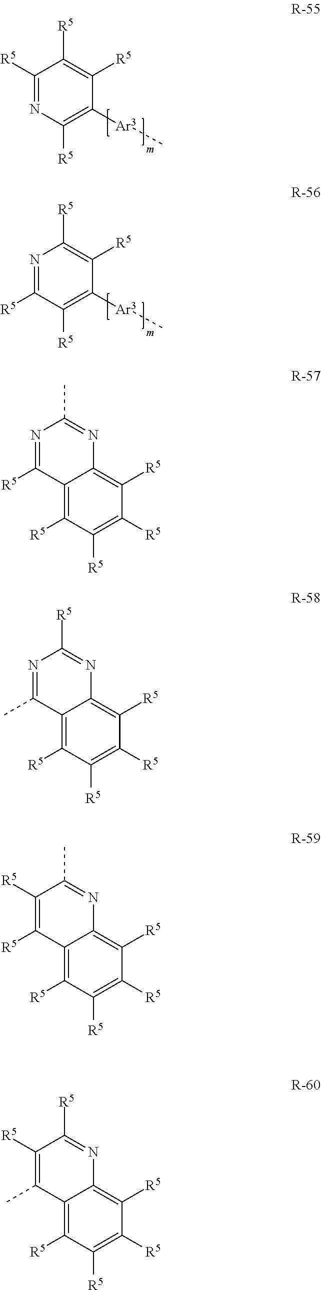

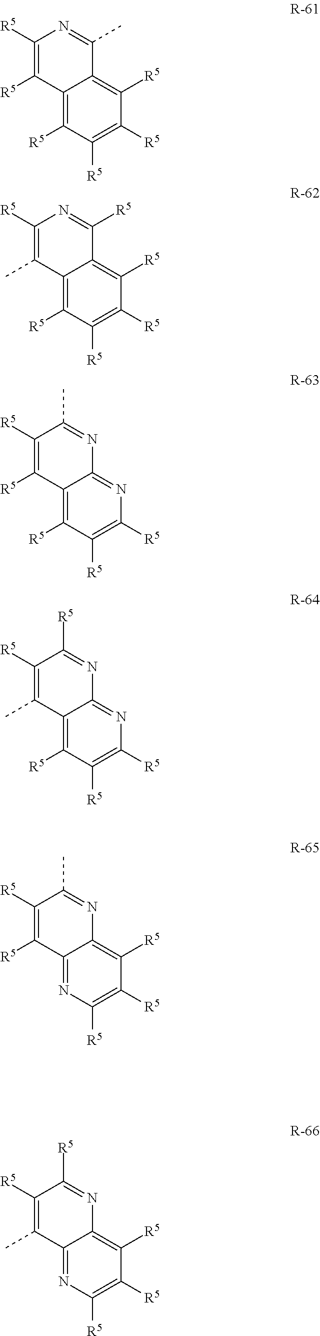

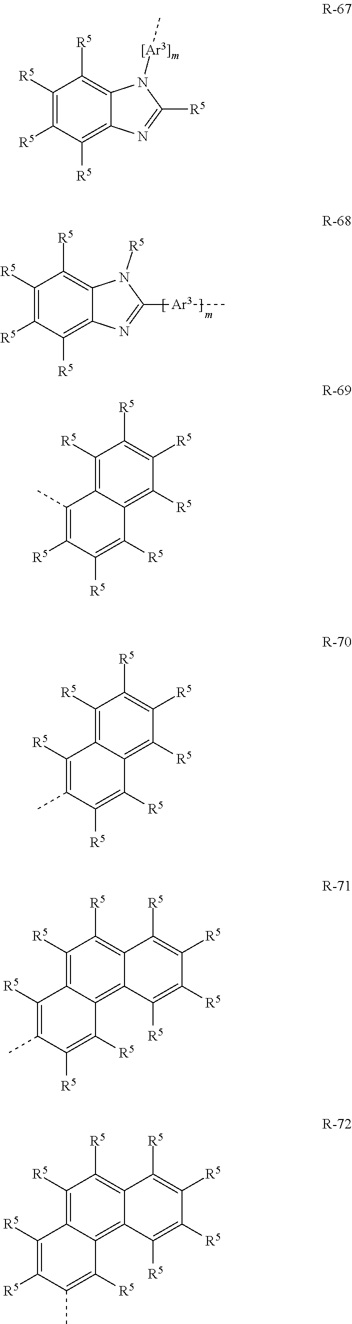

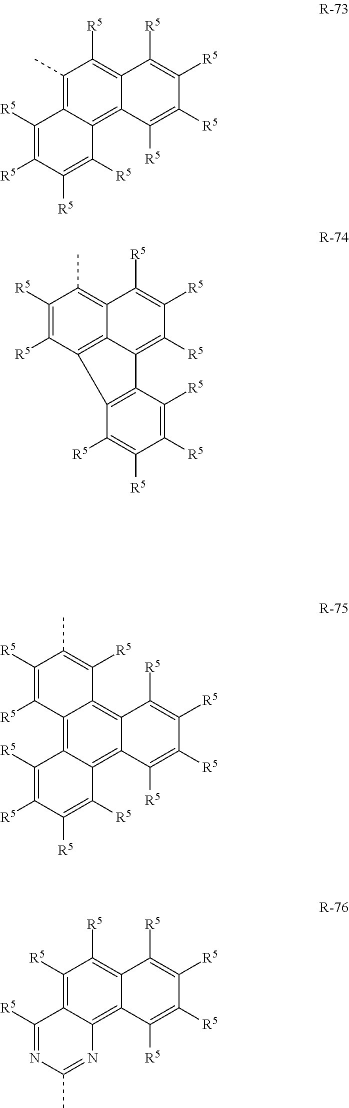

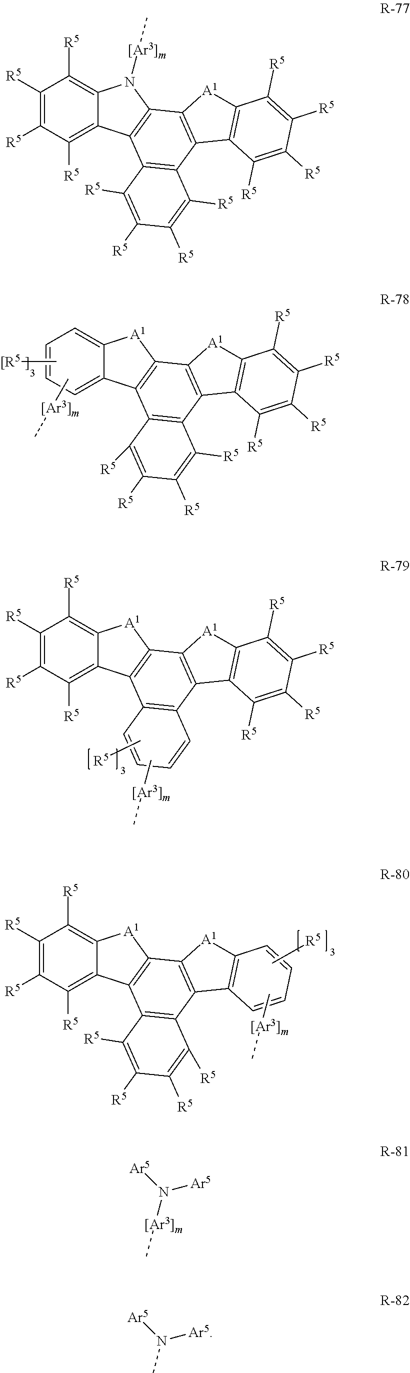

34. The compound according to claim 25, wherein formula (I) includes at least one R³ group selected from N(R⁵)₂, aromatic ring systems which have 6 to 40 aromatic ring atoms and are substituted by R⁵ radicals, and heteroaromatic ring systems which have 5 to 40 aromatic ring atoms and are substituted by R⁵ radicals.

35. The compound according to claim 25, wherein formula (I) includes at least one R³ group selected from the R-1 to R-82 groups

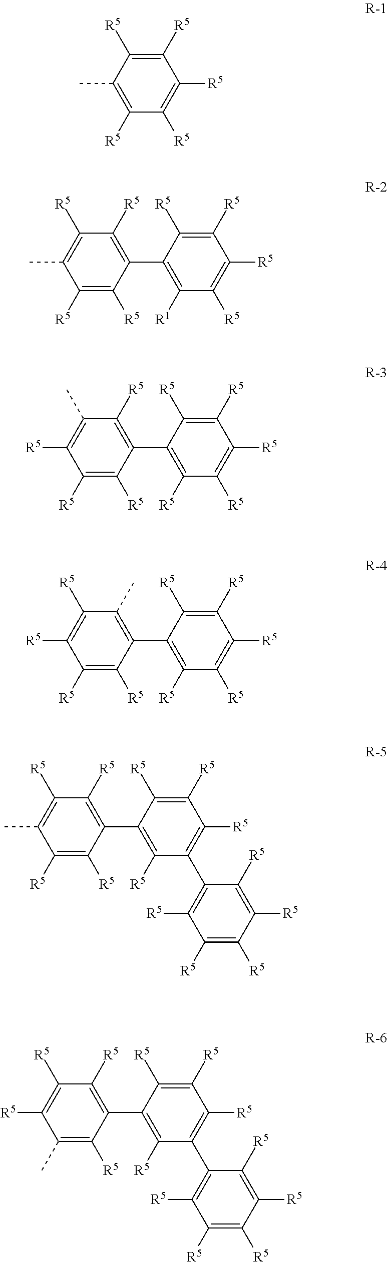

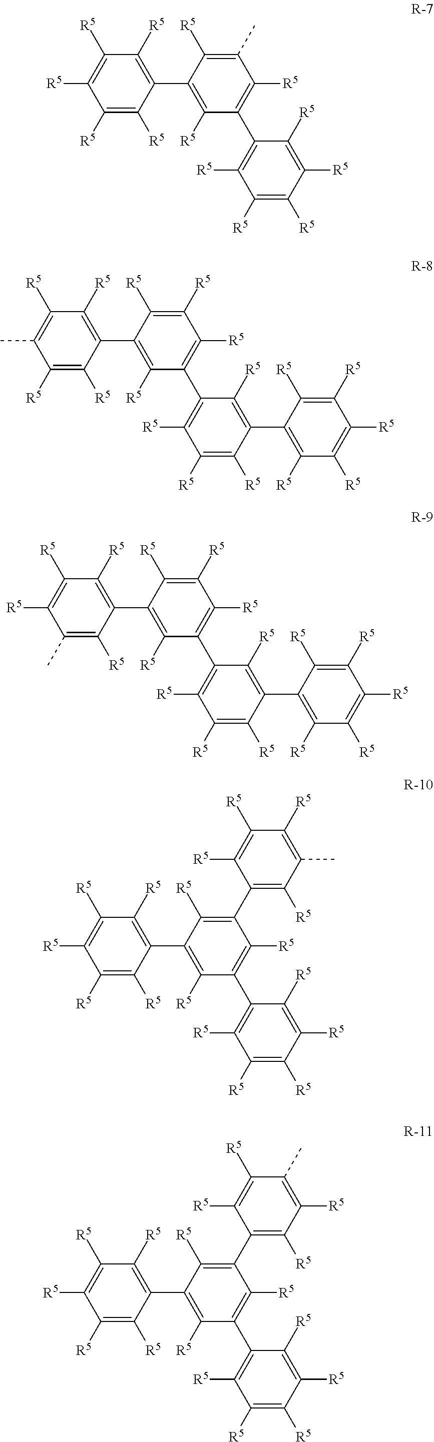

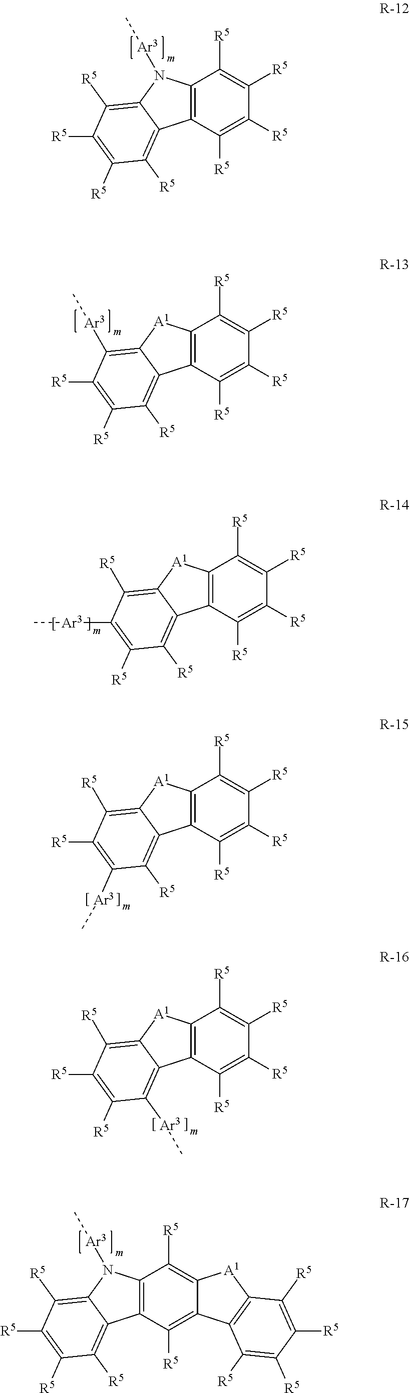

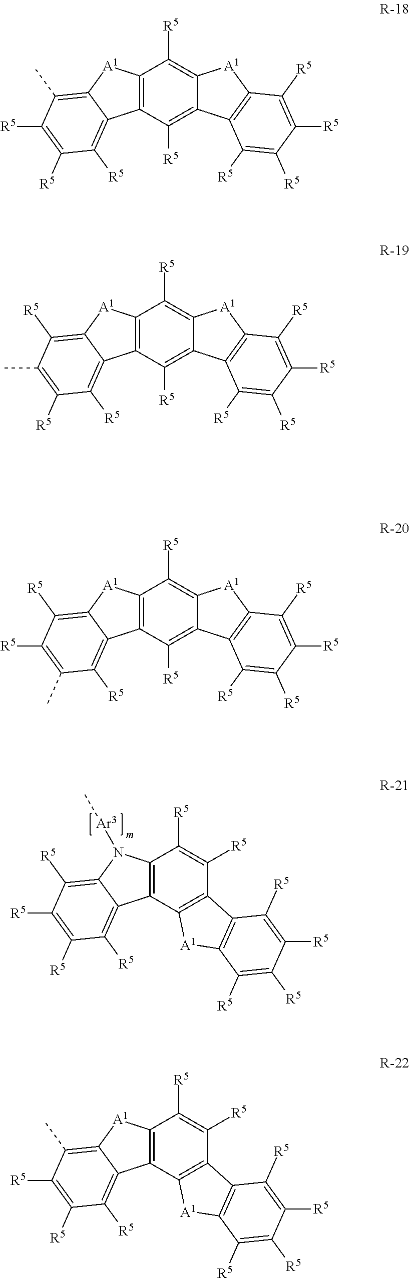

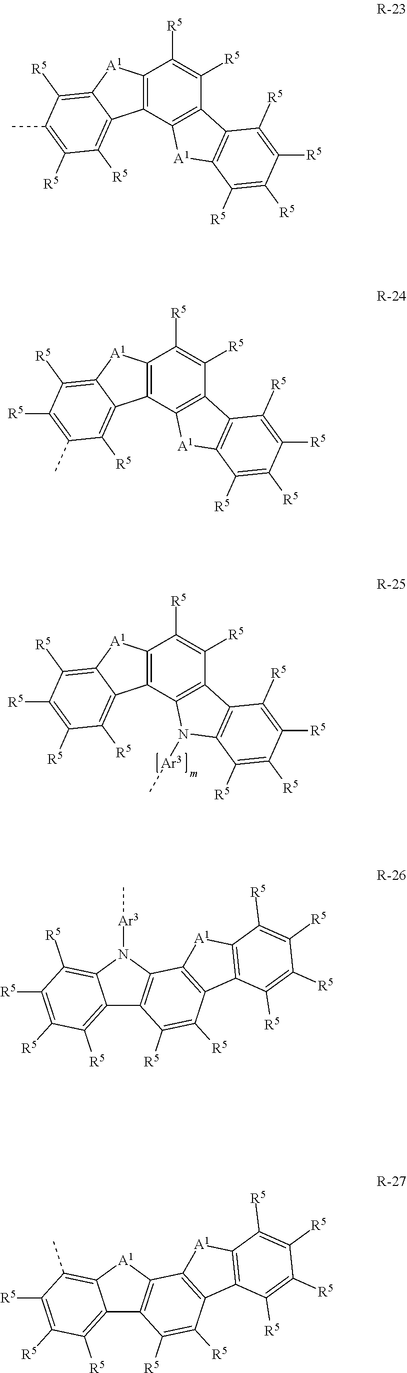

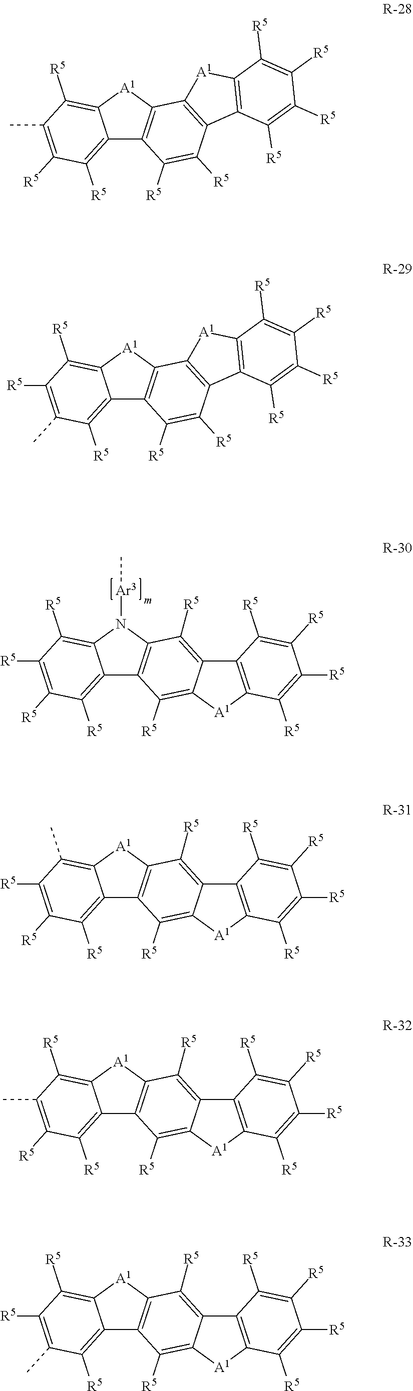

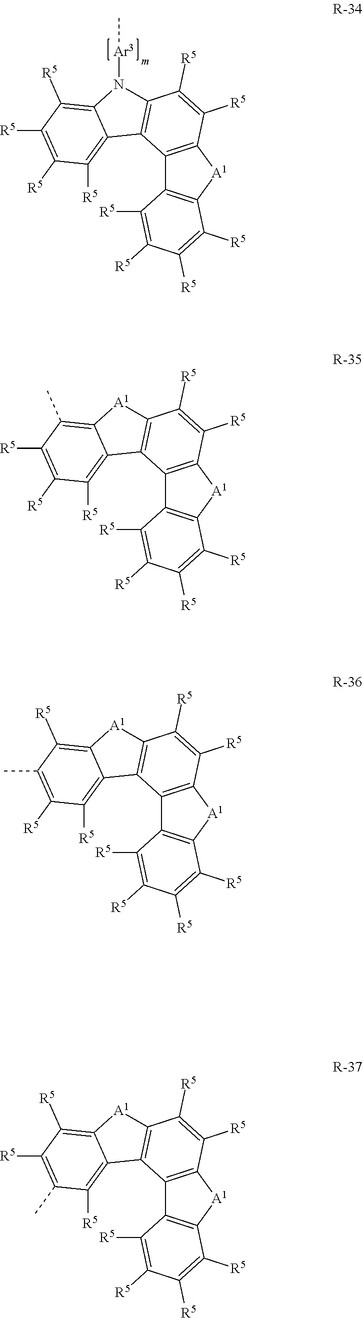

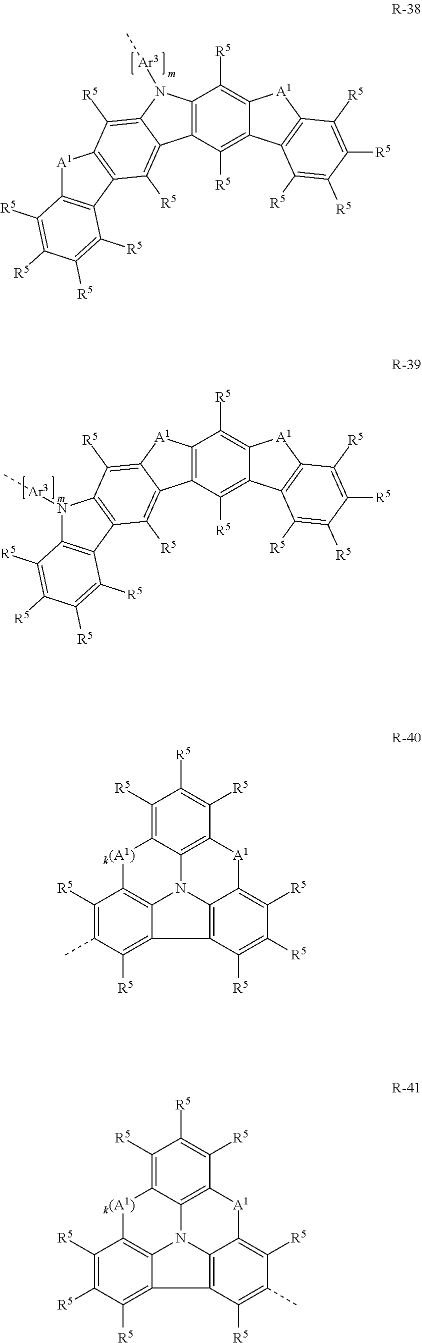

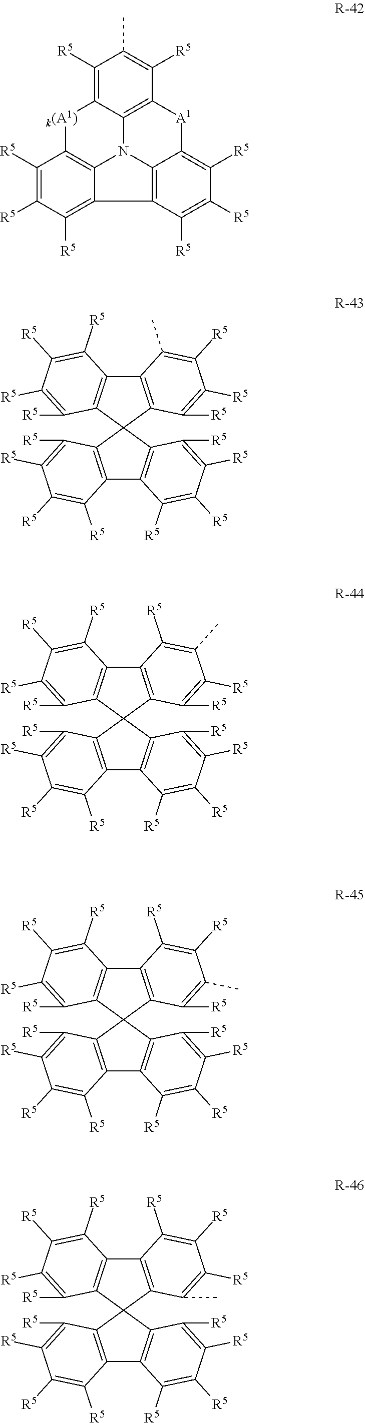

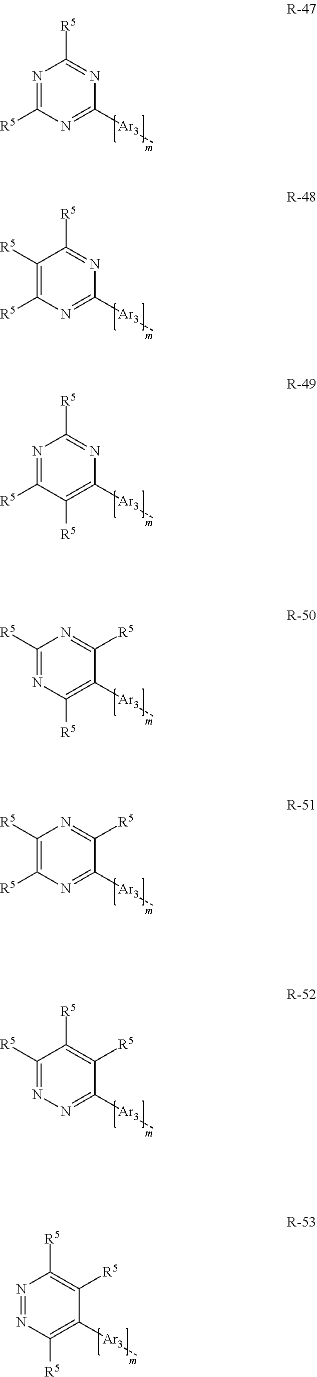

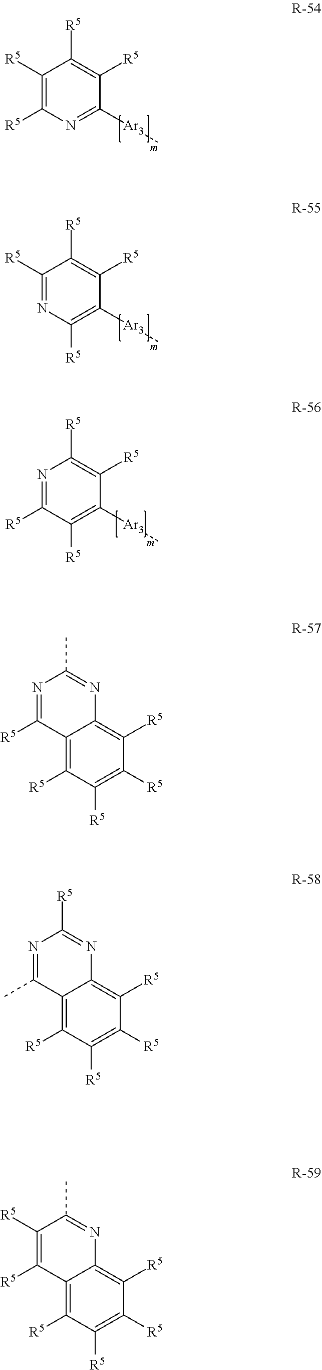

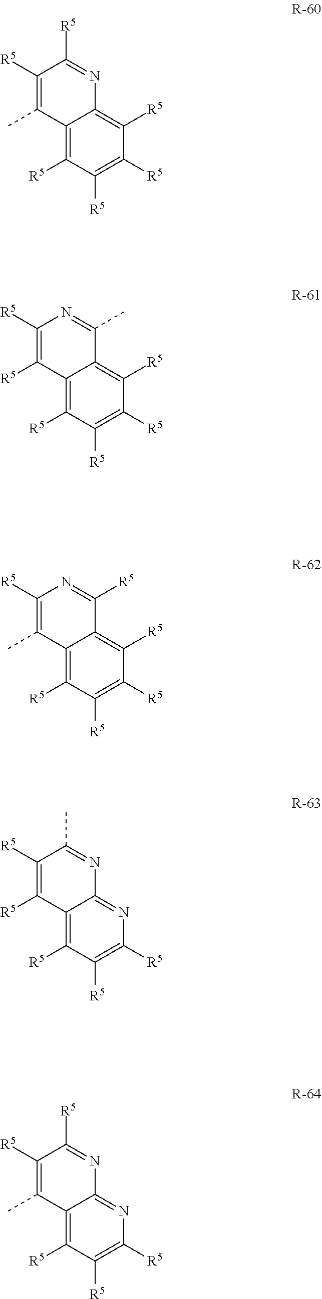

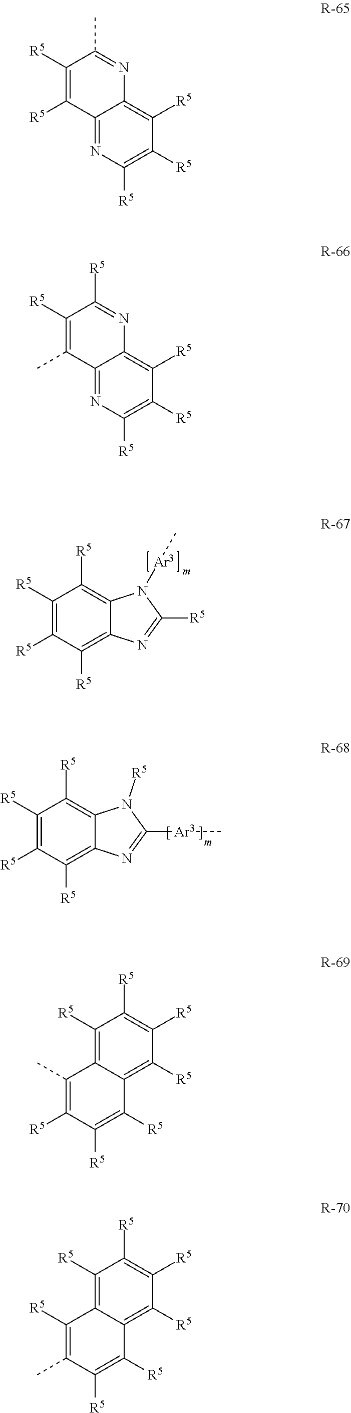

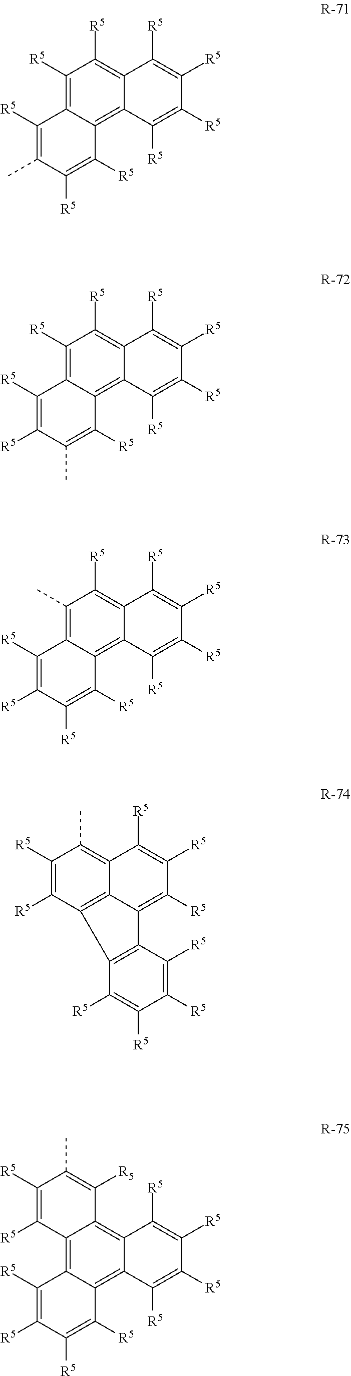

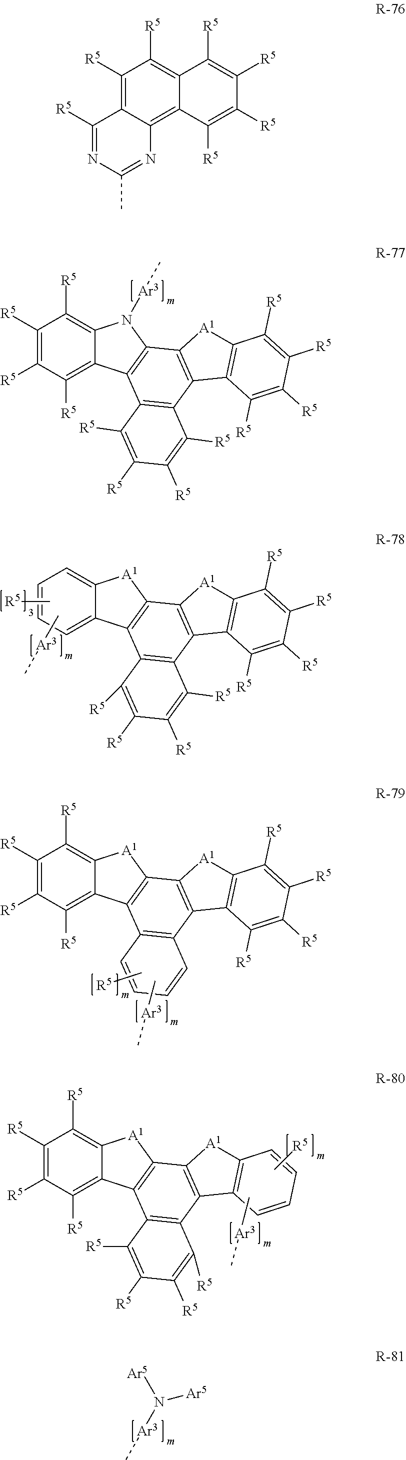

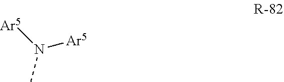

36. The compound according to claim 25, wherein formula (I) includes at least one group selected from the R¹, R², and R³ groups which is selected from aromatic ring systems which have 7 to 40 aromatic ring atoms and are each substituted by R⁵ radicals, and heteroaromatic ring systems which have 5 to 40 aromatic ring atoms and are each substituted by R⁵ radicals.

37. Compound according to claim 25, wherein formula (I) includes at least one group selected from the R², and R³ groups which is selected from the R-1 to R-81 groups

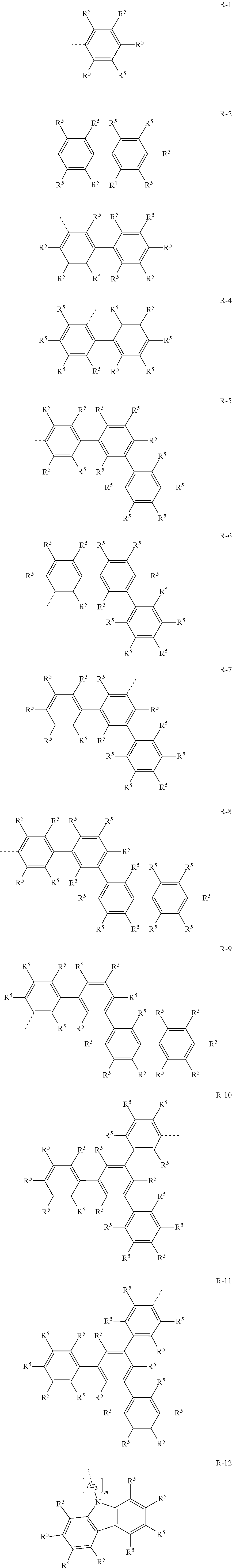

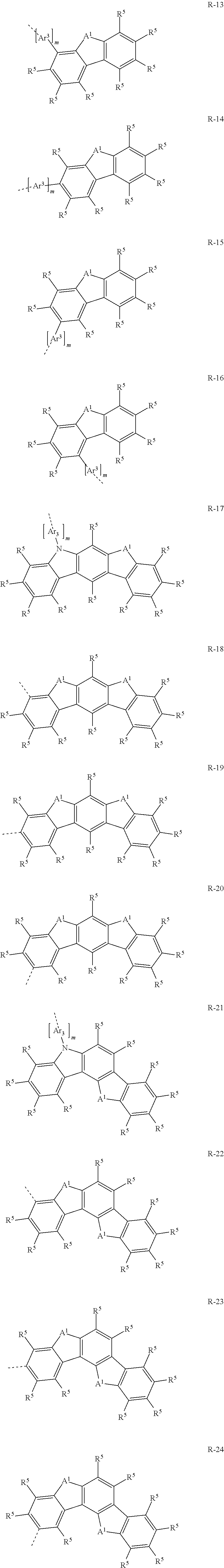

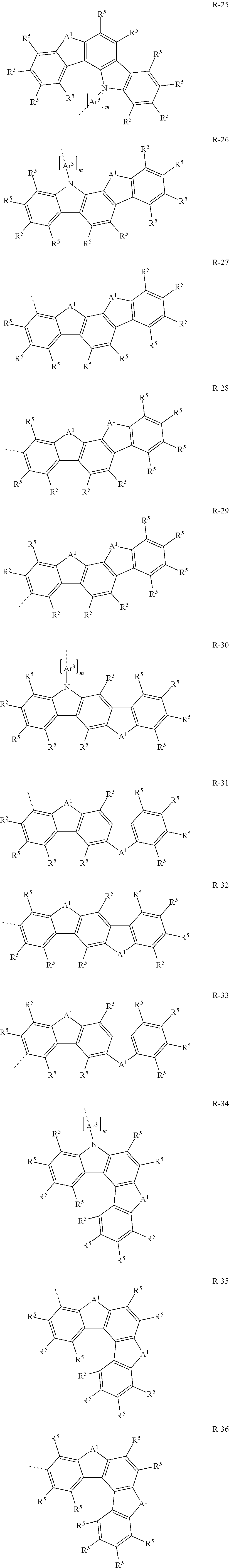

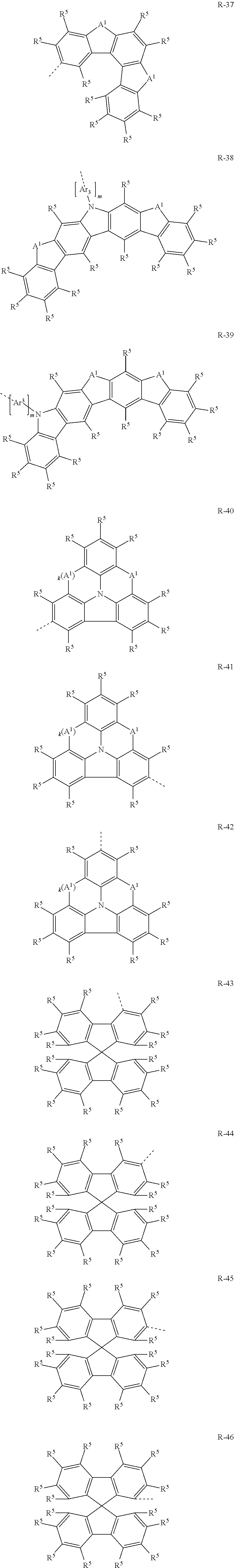

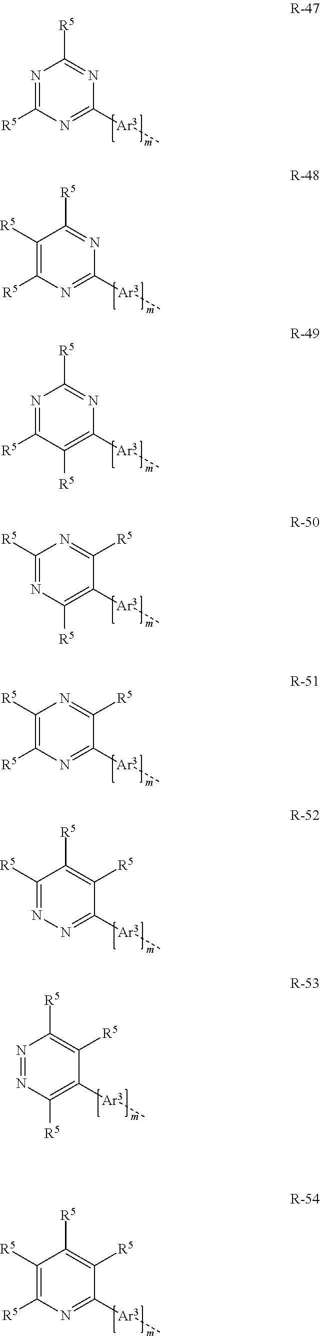

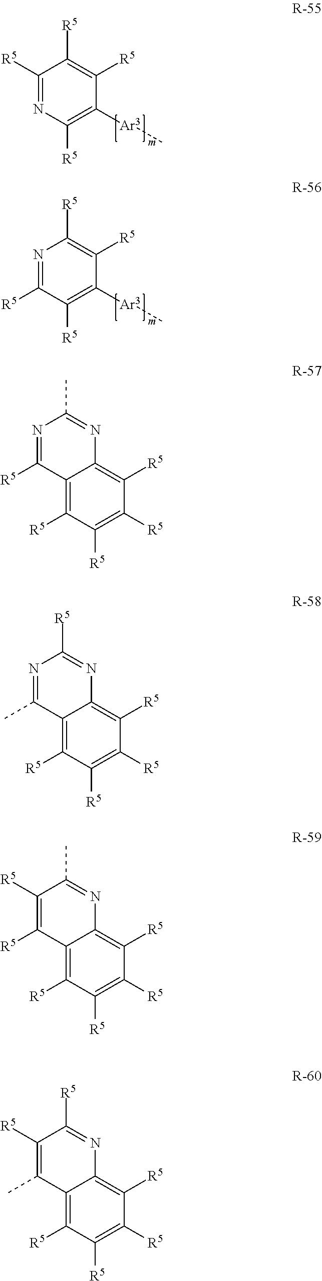

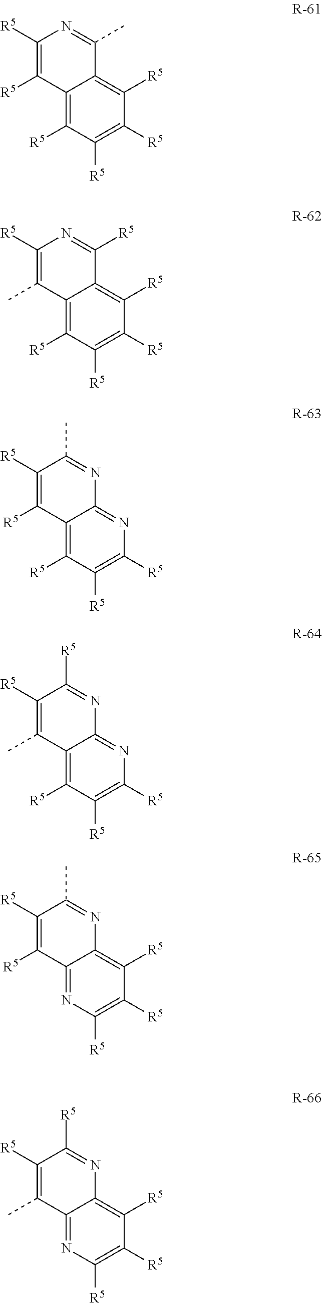

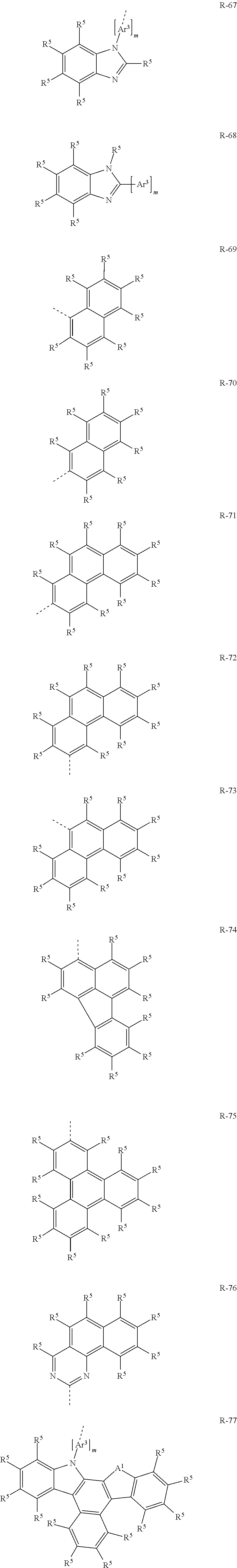

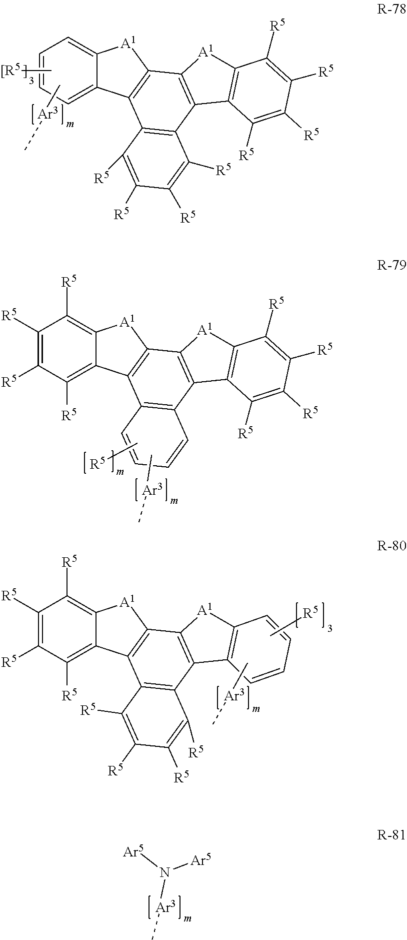

38. The compound according to claim 25, wherein the compound has one of the formulae (I-A-1) to (I-A-3) 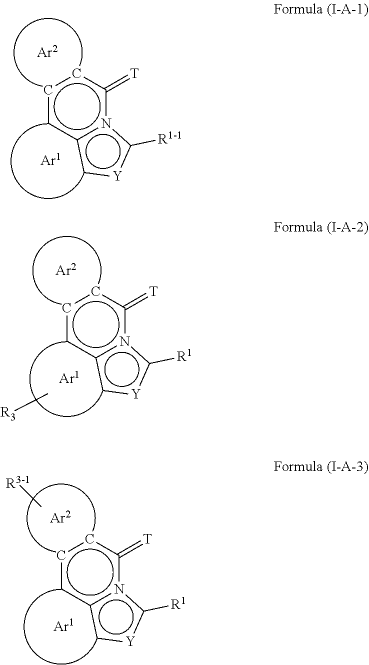
where R¹⁻¹, R²⁻¹ and R³⁻¹ are selected from aromatic ring systems which have 7 to 40 aromatic ring atoms and are each substituted by R⁵ radicals; and heteroaromatic ring systems which have 5 to 40 aromatic ring atoms and are each substituted by R⁵ radicals.

39. The compound according to claim 25, wherein the compound conforms to one of the following formulae: 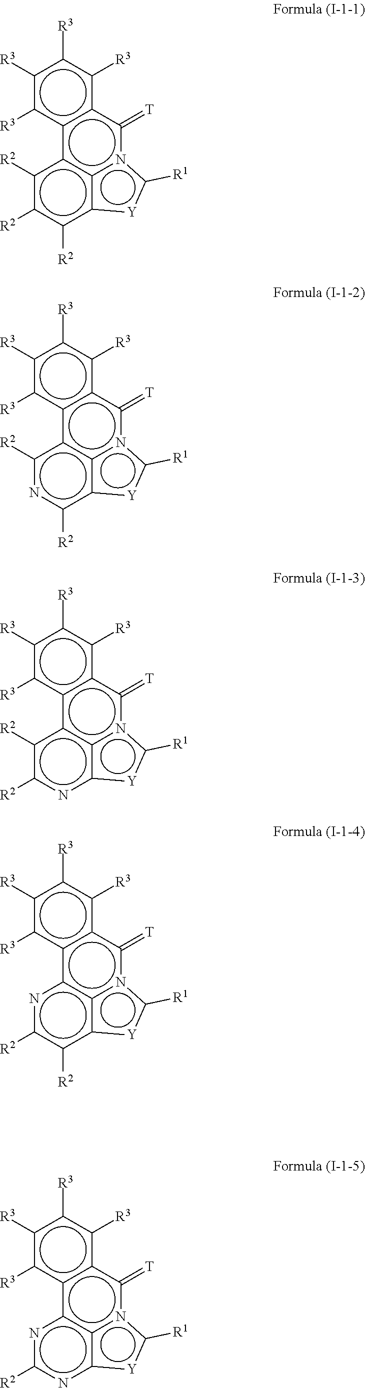 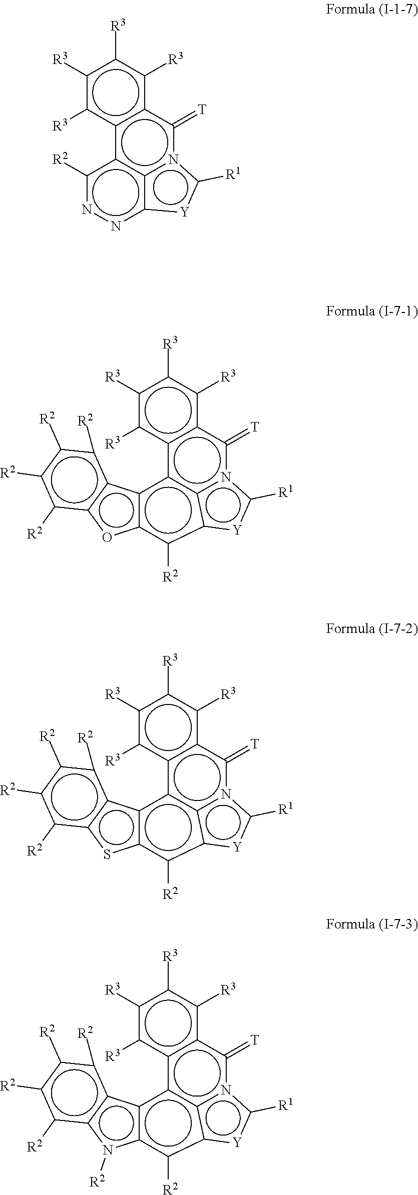 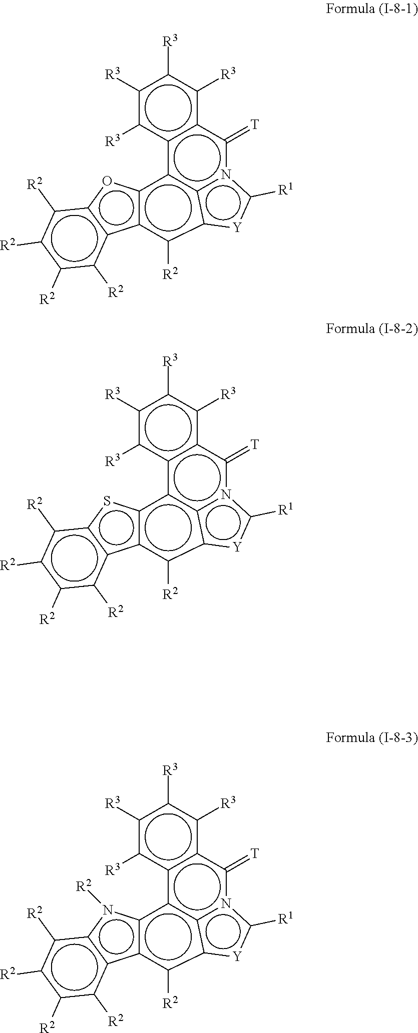
where the variables are as defined in claim 25, and where, when Y is CR¹, either
a) at least one group selected from the R¹, R², R³ and R⁴ groups is selected from aromatic ring systems which have 7 to 40 aromatic ring atoms and are each substituted by R⁵ radicals; and heteroaromatic ring systems which have 5 to 40 aromatic ring atoms and are each substituted by R⁵ radicals; or
b) at least two groups selected from the R¹, R², R³ and R⁴ groups are selected from aromatic ring systems which have 6 to 40 aromatic ring atoms and are each substituted by R⁵ radicals; and heteroaromatic ring systems which have 5 to 40 aromatic ring atoms and are each substituted by R⁵ radicals.

40. The compound according to claim 39, wherein the formulae each include at least one group selected from the R¹, R² and R³ groups which is selected from aromatic ring systems which have 7 to 40 aromatic ring atoms and are each substituted by R⁵ radicals, and heteroaromatic ring systems which have 5 to 40 aromatic ring atoms and are each substituted by R⁵ radicals.

41. A compound of one of the following structural formulae HT-1 to HT-13:

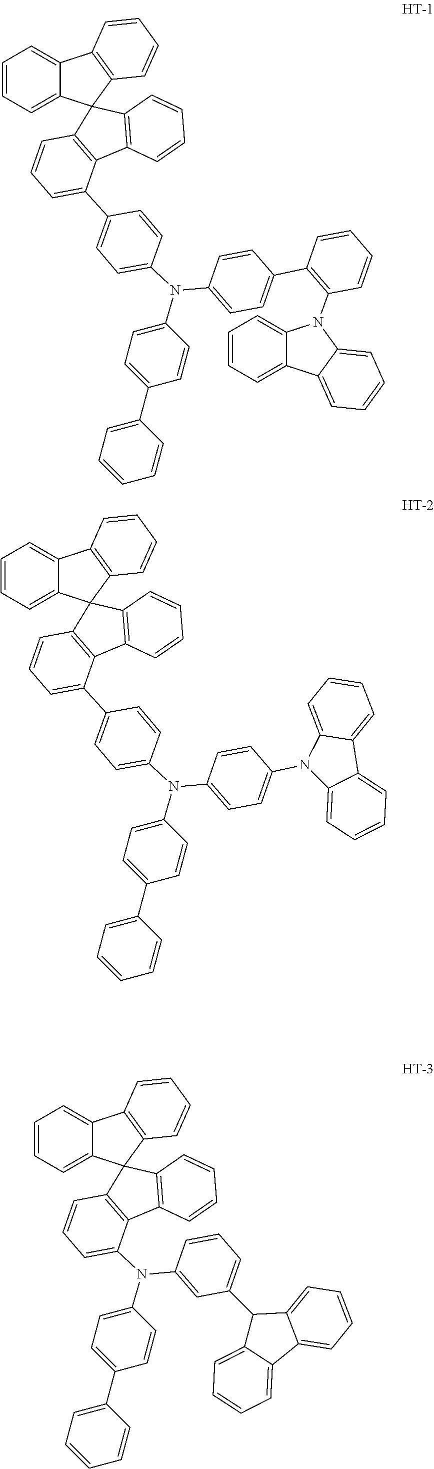

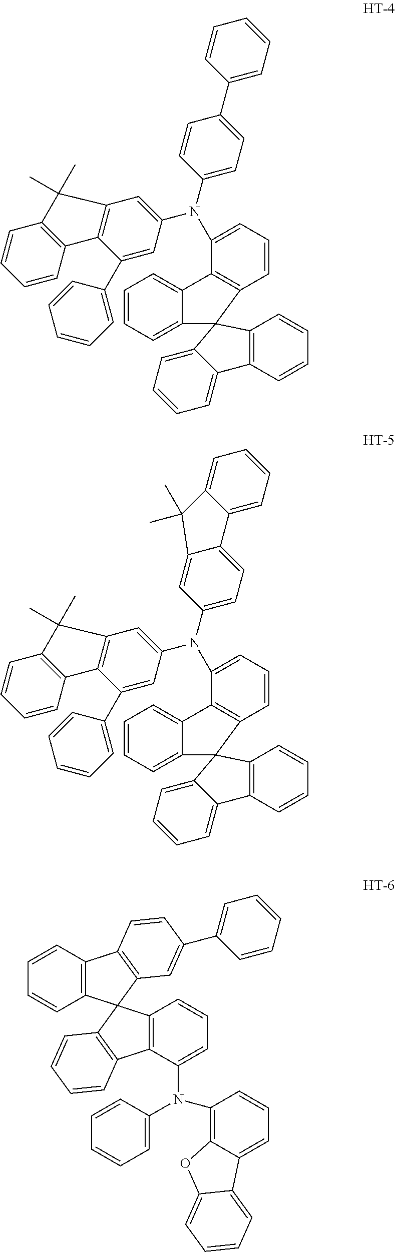 [SMILES: CC1(C)c2ccccc2-c2c(-c3ccccc3)cc(N(c3ccc(-c4ccccc4)cc3)c3cccc4c3-c3ccccc3C43c4ccccc4-c4ccccc43)cc21 ; CC1(C)c2ccccc2-c2ccc(N(c3cc(-c4ccccc4)c4c(c3)C(C)(C)c3ccccc3-4)c3cccc4c3-c3ccccc3C43c4ccccc4-c4ccccc43)cc21 ; c1ccc(-c2ccc3c(c2)C2(c4ccccc4-3)c3ccccc3-c3c(N(c4ccccc4)c4cccc5c4oc4ccccc45)cccc32)cc1]

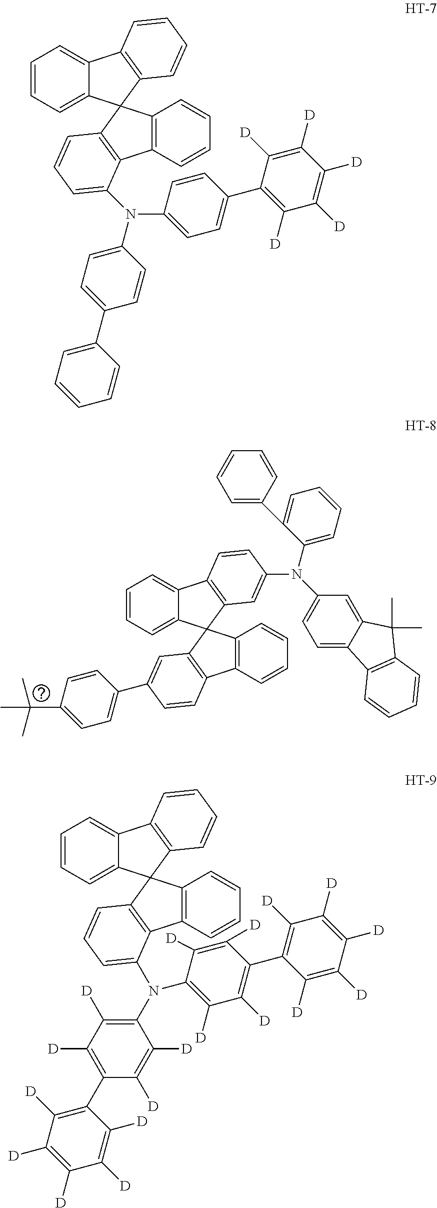 [SMILES: [2H]c1c([2H])c([2H])c(-c2ccc(N(c3ccc(-c4ccccc4)cc3)c3cccc4c3-c3ccccc3C43c4ccccc4-c4ccccc43)cc2)c([2H])c1[2H] ; CC(C)(C)c1ccc(-c2ccc3c(c2)C2(c4ccccc4-3)c3ccccc3-c3ccc(N(c4ccc5c(c4)C(C)(C)c4ccccc4-5)c4ccccc4-c4ccccc4)cc32)cc1 ; [2H]c1c([2H])c([2H])c(-c2c([2H])c([2H])c(N(c3cccc4c3-c3ccccc3C43c4ccccc4-c4ccccc43)c3c([2H])c([2H])c(-c4c([2H])c([2H])c([2H])c([2H])c4[2H])c([2H])c3[2H])c([2H])c2[2H])c([2H])c1[2H]]

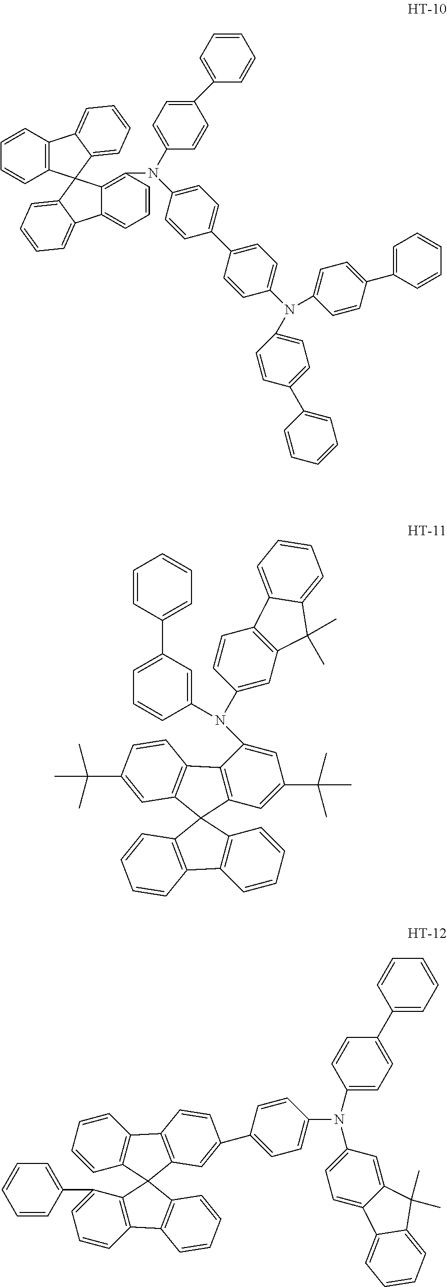 [SMILES: c1ccc(-c2ccc(N(c3ccc(-c4ccccc4)cc3)c3ccc(-c4ccc(N(c5ccc(-c6ccccc6)cc5)c5cccc6c5C5(c7ccccc7-c7ccccc75)c5ccccc5-6)cc4)cc3)cc2)cc1 ; CC(C)(C)c1ccc2c(c1)C1(c3ccccc3-c3ccccc31)c1cc(C(C)(C)C)cc(N(c3cccc(-c4ccccc4)c3)c3ccc4c(c3)C(C)(C)c3ccccc3-4)c1-2 ; CC1(C)c2ccccc2-c2ccc(N(c3ccc(-c4ccccc4)cc3)c3ccc(-c4ccc5c(c4)C4(c6ccccc6-5)c5ccccc5-c5cccc(-c6ccccc6)c54)cc3)cc21]

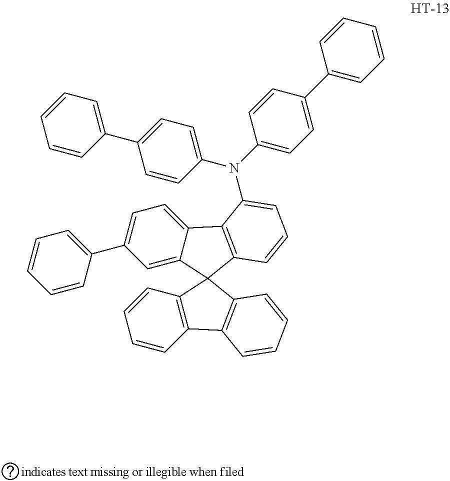 [SMILES: c1ccc(-c2ccc(N(c3ccc(-c4ccccc4)cc3)c3cccc4c3-c3ccc(-c5ccccc5)cc3C43c4ccccc4-c4ccccc43)cc2)cc1]

42. A process for preparing the compound according to claim 25, comprising i) reacting an imidazole or benzimidazole derivative with an aryl or heteroaryl compound having a halogen in a benzyl position, and ii) conducting a ring closure reaction under Pd catalysis, and iii) oxidating a methylene group in the ring formed to a carbonyl group; or iv) reacting an imidazole or benzimidazole derivative with an aryl or heteroaryl compound having a carbonyl halide group, and v) conducting a ring closure reaction.

43. A formulation comprising at least one compound according to claim 25, and at least one further compound and/or at least one solvent.

44. A method comprising providing a compound of the formula (I) 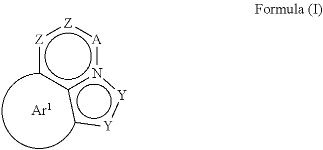
where the variables that occur are as follows:
A is C═O, C═S, C═NR⁰, P(═O)R⁰, SO or SO₂;
Y is the same or different at each instance and is selected from N and CR¹;
Ar¹ is an aromatic ring system which has 6 to 40 aromatic ring atoms and is substituted by R² radicals, and which is fused onto the rest of the formula (I) via the three carbon atoms shown in formula (I), or a heteroaromatic ring system which has 5 to 40 aromatic ring atoms and is substituted by R² radicals, and which is fused onto the rest of the formula (I) via the three carbon atoms shown in formula (I);
Z is the same or different at each instance and is selected from CR⁴ and N, or the Z—Z unit represents a unit of formula (Ar²) 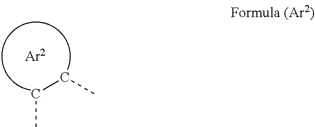
where Ar² is selected from aromatic ring systems which have 6 to 40 aromatic ring atoms and are substituted by R³ radicals, and which include the C—C unit, and heteroaromatic ring systems which have 5 to 40 aromatic ring atoms and are substituted by R³ radicals, and which include the C—C unit, and where the dotted lines are the bonds of the Z—Z unit to the rest of the formula;
R⁰ is the same or different at each instance and is selected from straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R¹ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R² is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more radicals selected from R², R³ and R⁴ may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R³ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more radicals selected from R², R³ and R⁴ may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R⁴ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R², R³ and R⁴ radicals may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R⁵ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁵ radicals may be joined to one another and may form a ring; where the alkyl, alkoxy, alkenyl and alkynyl groups mentioned and the aromatic ring systems and heteroaromatic ring systems mentioned are each substituted by R⁶ radicals; and where one or more CH₂ groups in the alkyl, alkoxy, alkenyl and alkynyl groups mentioned may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;
R⁶ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁶ radicals may be joined to one another and may form a ring; and where the alkyl, alkoxy, alkenyl and alkynyl groups, aromatic ring systems and heteroaromatic ring systems mentioned may be substituted by one or more radicals selected from F and CN;
and incorporating the compound in an electronic device.

45. An electronic device comprising at least one compound as defined in claim 41.

46. The electronic device according to claim 45, wherein it is an organic electroluminescent device, and wherein the compound is used in an emitting layer as matrix material for phosphorescent emitters or for emitters that exhibit TADF (thermally activated delayed fluorescence), or in an electron transport layer and/or in a hole blocker layer and/or in a hole transport layer and/or in an electron blocker layer.

47. A material comprising at least one compound as defined in claim 43 and at least one further compound selected from the group of the biscarbazoles, the bridged carbazoles, the triarylamines, the dibenzofuranyl-carbazole derivatives, the dibenzofuranyl-amine derivatives, and the carbazoleamines.

48. An organic electroluminescent device comprising the material according to claim 47 in a layer.
